SEQUENCE LISTING (167 sequences)

SEQ ID NO: 1 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 3 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 4 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 6 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 7 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 8 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 9 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 10 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 11 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 12 - DNA, 8791 bp, Artificial Sequence
  Description of Artificial Sequence: Synthetic polynucleotide
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccgccaccatgcttctcctggtgacaagccttctgctctgtgagttaccacacccagcattcctcctgatcccagacattcagcaggtccagctccagcagtctggccctgaactcgaaaaacctggcgctagcgtgaaaatttcctgtaaagcctccggctactcttttactggctacacaatgaattgggtgaaacagtctcacggcaaatccctcgaatggatcggactcatcacaccctacaatggcgcctcttcctacaaccagaaattccggggcaaggcaacactcactgtggacaaatcatcctctaccgcctacatggatctgctctccctcacatctgaggactccgctgtctacttttgtgcccgaggaggatacgacggacgaggattcgattactggggacagggaacaactgtgaccgtgtctagtggcggcggagggagtggaggcggaggatcttctggcgggggatccgatattgaactcacacagtctcccgctatcatgtctgcttctcccggcgagaaagtgactatgacttgctctgcttcctcttctgtgtcctacatgcactggtaccagcagaaatctggcacatcccctaaacggtggatctacgatactagcaaactggcatccggcgtgcctgggcgattctctggctctggctctggcaactcttactctctcacaatctcatctgtcgaggctgaggacgatgccacatactactgtcagcagtggtctaaacacccactcacattcggcgctggcactaaactggaaataaaagcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 13 - protein, 470 aa, Artificial Sequence
  Description of Artificial Sequence: Synthetic polypeptide
MLLLVTSLLLCELPHPAFLLIPDIQQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 14 - protein, 112 aa, Artificial Sequence
  Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSGSGTDFTLKITRVEAEDLGVFFCSQSTHVPFTFGSGTKLEIK

SEQ ID NO: 15 - DNA, 336 bp, Artificial Sequence
  Description of Artificial Sequence: Synthetic polynucleotide
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggactgatttcacactcaagatcaccagagtggaggctgaggatctgggagtttttttctgctctcaaagtacacatgttccattcacgttcggctcggggacaaagttggaaataaaa

SEQ ID NO: 16 - protein, 120 aa, Artificial Sequence
  Description of Artificial Sequence: Synthetic polypeptide
QVQLQQSGAELVRPGASVTLSCKASGYTFFDYEMHWVKQTPVHGLEWIGAIDPEIDGTAYNQKFKGKAILTADKSSSTAYMELRSLTSEDSAVYYCTDYYGSSYWYFDVWGTGTTVTVSS

SEQ ID NO: 17 - DNA, 359 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacattttttgactatgaaatgcactgggtgaagcagacacctgtgcatggcctggaatggattggagctattgatcctgaaattgatggtactgcctacaatcagaagttcaagggcaaggccatactgactgcagacaaatcctccagcacagcctacatggagctccgcagcctgacatctgaggactctgccgtctattactgtacagattactacggtagtagctactggtacttcgatgtctggggcacagggaccacggtcaccgtctcctc

SEQ ID NO: 18 - protein, 112 aa, Artificial Sequence
  Description of Artificial Sequence: Synthetic polypeptide
DVMMTQTPLSLPVSLGDQASISCRSSQSLVHSNGNTYLHWFLQKPGQSPKLLIYKVSNRFSGVPDRFSGSGSGTDFTLKISRVEAEDLGVYFCSQTTHVPLTFGAGTKLELK

SEQ ID NO: 19 - DNA, 336 bp, Artificial Sequence
  Description of Artificial Sequence: Synthetic polynucleotide
gatgttatgatgacccaaactccactctccctgcctgtcagtcttggagatcaagcctccatctcttgcagatctagtcagagccttgtacacagtaatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgctcacgttcggtgctgggaccaagctggagctgaaa

SEQ ID NO: 20 - protein, 120 aa, Artificial Sequence
  Description of Artificial Sequence: Synthetic polypeptide
QVQLQQSGAELVRPGASVTLSCKASGYTFTDYEMHWVKQTPVHGLEWIGAIDPEIAGTAYNQKFKGKAILTADKSSSTAYMELRSLTSEDSAVYYCSRYGGNYLYYFDYWGQGTTLTVSS

SEQ ID NO: 21 - DNA, 360 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacttttactgactatgaaatgcactgggtgaagcagacacctgtccatggcctggaatggattggagctattgatcctgaaattgctggtactgcctacaatcagaagttcaagggcaaggccatactgactgcagacaaatcctccagcacagcctacatggagctccgcagcctgacatctgaggactctgccgtctattactgttcaagatacggtggtaactacctttactactttgactactggggccaaggcaccactctcacagtctcctca

SEQ ID NO: 22 - protein, 112 aa, Artificial Sequence
  Description of Artificial Sequence: Synthetic polypeptide
DVLMTQIPLSLPVSLGDQASISCRSSQNIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 23 - DNA, 336 bp, Artificial Sequence
  Description of Artificial Sequence: Synthetic polynucleotide
gatgttttgatgacccaaattccactctccctgcctgtcagtcttggagatcaagcctccatctcttgcagatctagtcagaacattgtgtatagtaatggaaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 24 - protein, 118 aa, Artificial Sequence
  Description of Artificial Sequence: Synthetic polypeptide
QVQLQQSGAELVRPGASVTLSCKASGYTFTDYEMHWVKQTPVHGLEWIGAIDPEIGGSAYNQKFKGRAILTADKSSSTAYMELRSLTSEDSAVYYCTGYDGYFWFAYWGQGTLVTVSS

SEQ ID NO: 25 - DNA, 354 bp, Artificial Sequence
  Description of Artificial Sequence: Synthetic polynucleotide
caggttcaactgcagcagtccggggctgagctggtgaggcctggggcttcagtgacgctgtcctgcaaggcttcgggctacacatttactgactatgaaatgcactgggtgaagcagacacctgtgcatggcctggaatggattggagctattgatcctgaaattggtggttctgcctacaatcagaagttcaagggcagggccatattgactgcagacaaatcctccagcacagcctacatggagctccgcagcctgacatctgaggactctgccgtctattattgtacgggctatgatggttacttttggtttgcttactggggccaagggactctggtcactgtctcttca

SEQ ID NO: 26 - protein, 106 aa, Artificial Sequence
  Description of Artificial Sequence: Synthetic polypeptide
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIK

SEQ ID NO: 27 - DNA, 318 bp, Artificial Sequence
  Description of Artificial Sequence: Synthetic polynucleotide
gaaaatgttctcacccagtctccagcaatcatgtccgcatctccaggggaaaaggtcaccatgacctgcagtgctagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaa

SEQ ID NO: 28 - protein, 116 aa, Artificial Sequence
  Description of Artificial Sequence: Synthetic polypeptide
QVQLQQSGAELVRPGASVTLSCKASGYTFTDYEMHWVKQTPVHGLEWIGGIDPETGGTAYNQKFKGKAILTADKSSSTAYMELRSLTSEDSAVYYCTSYYGSRVFWGTGTTVTVSS

SEQ ID NO: 29 - DNA, 348 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggaggtattgatcctgaaactggtggtactgcctacaatcagaagttcaagggtaaggccatactgactgcagacaaatcctccagcacagcctacatggagctccgcagcctgacatctgaggactctgccgtctattactgtacaagttactatggtagtagagtcttctggggcacagggaccacggtcaccgtctcctca

SEQ ID NO: 30 - protein, 108 aa, Artificial Sequence
  Description of Artificial Sequence: Synthetic polypeptide
QIVLSQSPAILSAFPGEKVTMTCRASSSVSYMHWYQQKPGSSPKPWIYATSNLASGVPARFSGSGSGTSYSLTISSVEAEDAATYYCQQWSSNPPTLTFGAGTKLELK

SEQ ID NO: 31 - DNA, 324 bp, Artificial Sequence
  Description of Artificial Sequence: Synthetic polynucleotide
caaattgttctctcccagtctccagcaatcctgtctgcatttccaggggagaaggtcactatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagtgtggaggctgaagatgctgccacttattactgccagcagtggagtagtaacccacccacgctcacgttcggtgctgggaccaagctggagctgaaa

SEQ ID NO: 32 - protein, 124 aa, Artificial Sequence
  Description of Artificial Sequence: Synthetic polypeptide
QVQLQQSGAELARPGASVKLSCKASGYTFTSYGISWVKQRTGQGLEWIGEIYPRSGNTYYNESFKGKVTLTADKSSGTAYMELRSLTSEDSAVYFCARWGSYGSPPFYYGMDYWGQGTSVTVSS

SEQ ID NO: 33 - DNA, 372 bp, Artificial Sequence
  Description of Artificial Sequence: Synthetic polynucleotide
caggttcagctgcagcagtctggagctgagctggcgaggcctggggcttcagtgaagctgtcctgcaaggcttctggctacaccttcacaagctatggtataagctgggtgaagcagaggactggacagggccttgagtggattggagagatttatcctagaagtggtaatacttactacaatgagagcttcaagggcaaggtcacactgaccgcagacaaatcttccggcacagcgtacatggagctccgcagcctgacatctgaggactctgcggtctatttctgtgcaagatggggctcctacggtagtccccccttttactatggtatggactactggggtcaaggaacctcagtcaccgtctcctca

SEQ ID NO: 34 - protein, 112 aa, Artificial Sequence
  Description of Artificial Sequence: Synthetic polypeptide
DVLMTQTPLSLPVSLGNQASISCRSSQSIVHSSGSTYLEWYLQKPGQSPKLLIYKVSNRFSGVPDRFSGSGSGTDFTLKISRVEAEDLGVYYCFQGSHVPYTFGGGTKLEIK

SEQ ID NO: 35 - DNA, 336 bp, Artificial Sequence
  Description of Artificial Sequence: Synthetic polynucleotide
gatgttttgatgacccaaactccactctccctgcctgtcagtcttggaaatcaagcctccatctcttgcagatctagtcagagcattgtacatagtagtggaagcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggctcacatgttccatacacgttcggaggggggaccaagctggaaataaaa

SEQ ID NO: 36 - protein, 123 aa, Artificial Sequence
  Description of Artificial Sequence: Synthetic polypeptide
QVQLQQSGAELARPGTSVKVSCKASGYTFTSYGISWVKQRIGQGLEWIGEIHPRSGNSYYNEKIRGKATLTADKSSSTAYMELRSLISEDSAVYFCARLITTVVANYYAMDYWGQGTSVTVSS

SEQ ID NO: 37 - DNA, 369 bp, Artificial Sequence
  Description of Artificial Sequence: Synthetic polynucleotide
caggttcagctgcagcagtctggagctgagctggcgaggcctgggacttcagtgaaggtgtcctgcaaggcttctggctataccttcacaagttatggtataagctgggtgaagcagagaattggacagggccttgagtggattggagagattcatcctagaagtggtaatagttactataatgagaagatcaggggcaaggccacactgactgcagacaaatcctccagcacagcgtacatggagctccgcagcctgatatctgaggactctgcggtctatttctgtgcaaggctgattactacggtagttgctaattactatgctatggactactggggtcaaggaacctcagtcaccgtctcctca

SEQ ID NO: 38 - protein, 112 aa, Artificial Sequence
  Description of Artificial Sequence: Synthetic polypeptide
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLVTFGAGTKLELK

SEQ ID NO: 39 - DNA, 336 bp, Artificial Sequence
  Description of Artificial Sequence: Synthetic polynucleotide
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatctggtcacgttcggtgctgggaccaagctggagctgaaa

SEQ ID NO: 40 - protein, 120 aa, Artificial Sequence; identical to SEQ ID NO: 16
  Description of Artificial Sequence: Synthetic polypeptide

SEQ ID NO: 41 - DNA, 359 bp, Artificial Sequence; identical to SEQ ID NO: 17
  Description of Artificial Sequence: Synthetic polynucleotide

SEQ ID NO: 42 - protein, 106 aa, Artificial Sequence
  Description of Artificial Sequence: Synthetic polypeptide
QIVLTQSPAIMSASPGEKVTISCSASSSVSYMYWYQQKPGSSPKPWIYRTSNLASGVPARFSGSGSGTSYSLTISSMEAEDAATYYCQQYHSYPLTFGAGTKLELK

SEQ ID NO: 43 - DNA, 318 bp, Artificial Sequence
  Description of Artificial Sequence: Synthetic polynucleotide
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccactcacgttcggtgctgggaccaagctggagctgaaa

SEQ ID NO: 44 - protein, 109 aa, Artificial Sequence
  Description of Artificial Sequence: Synthetic polypeptide
QIVLTQSPAIMSASPGERVTMTCSASSSVSSSYLYWYQQKSGSSPKLWIYSISNLASGVPARFSGSGSGTSYSLTINSMEAEDAATYYCQQWSSNPQLTFGAGTKLELK

SEQ ID NO: 45 - DNA, 327 bp, Artificial Sequence
  Description of Artificial Sequence: Synthetic polynucleotide
caaattgttctcacccagtctccagcaatcatgtctgcatctcctggggaacgggtcaccatgacctgcagtgccagctcaagtgtaagttccagctacttgtactggtaccagcagaagtcaggatcctccccaaaactctggatttatagcatatccaacctggcttctggagtcccagctcgcttcagtggcagtgggtctgggacctcttactctctcacaatcaacagcatggaggctgaagatgctgccacttattactgccagcagtggagtagtaacccacagctcacgttcggtgctgggaccaagctggagctgaaa

SEQ ID NO: 46 - protein, 121 aa, Artificial Sequence
  Description of Artificial Sequence: Synthetic polypeptide
QVQLKQSGAELVKPGASVKISCKASGYTFTDYYINWVKQRPGQGLEWIGKIGPGSGSTYYNEKFKGKATLTADKSSSTAYMQLSSLTSEDSAVYFCARTGYYVGYYAMDYWGQGTSVTVSS

SEQ ID NO: 47 - DNA, 363 bp, Artificial Sequence
  Description of Artificial Sequence: Synthetic polynucleotide
caggtccagctgaagcagtctggagctgagctggtgaagcctggggcttcagtgaagatatcctgcaaggcttctggctacaccttcactgactactatataaactgggtgaagcagaggcctggacagggccttgagtggattggaaagattggtcctggaagtggtagtacttactacaatgagaagttcaagggcaaggccacactgactgcagacaaatcctccagcacagcctacatgcagctcagcagcctgacatctgaggactctgcagtctatttctgtgcaagaactggttactacgttggttactatgctatggactactggggtcaaggaacctcagtcaccgtctcctca

SEQ ID NO: 48 - protein, 118 aa, Artificial Sequence
  Description of Artificial Sequence: Synthetic polypeptide
QVQLQQSGAELARPGASVKLSCKASGYTFTIYGISWVKQRTGQGLEWIGEIYPRSDNTYYNEKFKGKATLTADKSSSTAYMELRSLTSEDSAVYFCARWYSFYAMDYWGQGTSVTVSS

SEQ ID NO: 49 - DNA, 354 bp, Artificial Sequence
  Description of Artificial Sequence: Synthetic polynucleotide
caggttcagctgcagcagtctggagctgagctggcgaggcctggggcttcagtgaagctgtcctgcaaggcttctggctacaccttcacaatctatggtataagctgggtgaaacagagaactggacagggccttgagtggattggagagatttatcctagaagtgataatacttactacaatgagaagttcaagggcaaggccacactgactgcagacaaatcctccagcacagcgtacatggagctccgcagcctgacatctgaggactctgcggtctatttctgtgcaagatggtactcgttctatgctatggactactggggtcaaggaacctcagtcaccgtctcctca

SEQ ID NO: 50 - protein, 119 aa, Artificial Sequence
  Description of Artificial Sequence: Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 51 - protein, 116 aa, Artificial Sequence
  Description of Artificial Sequence: Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 52 - DNA, 8147 bp, Artificial Sequence
  Description of Artificial Sequence: Synthetic polynucleotide
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgccgccaccatgctccagatggctggccagtgcagccagaacgagtacttcgacagcctgctgcacgcctgcatcccttgccagctgcggtgcagcagcaacaccccacccctgacctgccagcggtactgcaacgccagcgtgaccaacagcgtgaagggcaccaacgccatcctgtggacctgcctgggcctgagcctgatcatcagcctggccgtgttcgtgctgatgttcctgctgcggaagatcaacagcgagcccctgaaggacgagttcaagaacaccggcagcggcctgctgggcatggccaacatcgacctggaaaagagccggaccggcgacgagatcatcctgcccagaggcctggagtacaccgtggaagagtgtacctgcgaggactgcatcaagagcaagcccaaggtggacagcgaccactgcttccctctgcccgccatggaagagggcgccaccatcctggtgacaacaaagaccaacgactactgcaagagcctgcctgccgccctgagcgccaccgagatcgagaagtccatcagcgccagatgaggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtcgagatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccgcgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 53 - DNA, 48 bp, Artificial Sequence
  Description of Artificial Sequence: Synthetic oligonucleotide
aaaagcagccagagcctggtgcatagcaacggcaacacctatctgcat

SEQ ID NO: 54 - protein, 16 aa, Artificial Sequence
  Description of Artificial Sequence: Synthetic peptide
KSSQSLVHSNGNTYLH

SEQ ID NO: 55 - DNA, 21 bp, Artificial Sequence
  Description of Artificial Sequence: Synthetic oligonucleotide
aaagtgagcaaccgctttagc

SEQ ID NO: 56 - protein, 7 aa, Artificial Sequence
  Description of Artificial Sequence: Synthetic peptide
KVSNRFS

SEQ ID NO: 57 - DNA, 27 bp, Artificial Sequence
  Description of Artificial Sequence: Synthetic oligonucleotide
gcggaaaccagccatgtgccgtggacc

SEQ ID NO: 58 - protein, 9 aa, Artificial Sequence
  Description of Artificial Sequence: Synthetic peptide
AETSHVPWT

SEQ ID NO: 59 - DNA, 39 bp, Artificial Sequence
  Description of Artificial Sequence: Synthetic oligonucleotide
aaagcgagcggctatagctttccggattattatattaac

SEQ ID NO: 60 - protein, 13 aa, Artificial Sequence
  Description of Artificial Sequence: Synthetic peptide
KASGYSFPDYYIN

SEQ ID NO: 61 - DNA, 51 bp, Artificial Sequence
  Description of Artificial Sequence: Synthetic oligonucleotide
tggatttattttgcgagcggcaacagcgaatataaccagaaatttaccggc

SEQ ID NO: 62 - protein, 17 aa, Artificial Sequence
  Description of Artificial Sequence: Synthetic peptide
WIYFASGNSEYNQKFTG

SEQ ID NO: 63 - DNA, 30 bp, Artificial Sequence
  Description of Artificial Sequence: Synthetic oligonucleotide
ctgtatgattatgattggtattttgatgtg

SEQ ID NO: 64 - protein, 10 aa, Artificial Sequence
  Description of Artificial Sequence: Synthetic peptide
LYDYDWYFDV

SEQ ID NO: 65 - DNA, 339 bp, Artificial Sequence
  Description of Artificial Sequence: Synthetic polynucleotide
gatattgtgatgacccagaccccgctgagcctgagcgtgaccccgggcgaaccggcgagcattagctgcaaaagcagccagagcctggtgcatagcaacggcaacacctatctgcattggtatctgcagaaaccgggccagagcccgcagctgctgatttataaagtgagcaaccgctttagcggcgtgccggatcgctttagcggcagcggcagcggcgcggattttaccctgaaaattagccgcgtggaagcggaagatgtgggcgtgtattattgcgcggaaaccagccatgtgccgtggacctttggccagggcaccaaactggaaattaaaagc

SEQ ID NO: 66 - protein, 113 aa, Artificial Sequence
  Description of Artificial Sequence: Synthetic polypeptide
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKS

SEQ ID NO: 67 - DNA, 357 bp, Artificial Sequence
  Description of Artificial Sequence: Synthetic polynucleotide
caggtgcagctggtgcagagcggcgcggaagtgaaaaaaccgggcgcgagcgtgaaagtgagctgcaaagcgagcggctatagctttccggattattatattaactgggtgcgccaggcgccgggccagggcctggaatggatgggctggatttattttgcgagcggcaacagcgaatataaccagaaatttaccggccgcgtgaccatgacccgcgataccagcagcagcaccgcgtatatggaactgagcagcctgcgcagcgaagataccgcggtgtatttttgcgcgagcctgtatgattatgattggtattttgatgtgtggggccagggcaccatggtgaccgtgagcagc

SEQ ID NO: 68 - protein, 119 aa, Artificial Sequence
  Description of Artificial Sequence: Synthetic polypeptide
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 69 - DNA, 567 bp, Artificial Sequence
  Description of Artificial Sequence: Synthetic polynucleotide
atggcggtggtcctggctgctctactacaaggtgtccaggctcaggtgcacccggtggagtctgggggaggcttggtgcagactggggggtctctgagactctcctgtgcagcctctgctggtatcttcagtatcaatgtcatgggctggtaccgccaggctccagggaagcagcgcgaattggtcgcgagtataactagtcgtggtgatacaacgtatgcgaactccgtgaagggccgattcaccatctccagagacaacgccaagaacacggtatatctgcaaatgaacgccctgaaacctgaggacacagccgtctattactgtaatttaaaggggacagactatagtggtacatccacccagaccttcgacagacagggccaggggacccaggtcaccgtctcttcggaacccaagacaccaaaaccacaaccacaaccacaaccacaaccacaacccaatcctacaacagaatccaagtgtcccaaatgtccagcccctgagctcctgggagggccctcagtcttcatcttccccccgaaacccaaggacgtcctctccatc

SEQ ID NO: 70 - protein, 189 aa, Artificial Sequence
  Description of Artificial Sequence: Synthetic polypeptide
MAVVLAALLQGVQAQVHPVESGGGLVQTGGSLRLSCAASAGIFSINVMGWYRQAPGKQRELVASITSRGDTTYANSVKGRFTISRDNAKNTVYLQMNALKPEDTAVYYCNLKGTDYSGTSTQTFDRQGQGTQVTVSSEPKTPKPQPQPQPQPQPNPTTESKCPKCPAPELLGGPSVFIFPPKPKDVLSI

SEQ ID NO: 71 - protein, 5 aa, Artificial Sequence
  Description of Artificial Sequence: Synthetic peptide
INVMG

SEQ ID NO: 72 - protein, 16 aa, Artificial Sequence
  Description of Artificial Sequence: Synthetic peptide
SITSRGDTTYANSVKG

SEQ ID NO: 73 - protein, 16 aa, Artificial Sequence
  Description of Artificial Sequence: Synthetic peptide
LKGTDYSGTSTQTFDR

SEQ ID NO: 74 - protein, 173 aa, Artificial Sequence
  Description of Artificial Sequence: Synthetic polypeptide
QVQLVESGGGLVQPGESLRLSCAASTNIFSISPMGWYRQAPGKQRELVAAIHGFSTLYADSVKGRFTISRDNAKNTIYLQMNSLKPEDTAVYYCNKVPWGDYHPRNVYWGQGTQVTVSSEPKTPKPQPQPQPQPQPQPNPTTESKCPKCPAPELLGGPSVFIFPPKPKDVLSI

SEQ ID NO: 75 - protein, 5 aa, Artificial Sequence
  Description of Artificial Sequence: Synthetic peptide
ISPMG

SEQ ID NO: 76 - protein, 15 aa, Artificial Sequence
  Description of Artificial Sequence: Synthetic peptide
AIHGFSTLYADSVKG

SEQ ID NO: 77 - protein, 12 aa, Artificial Sequence
  Description of Artificial Sequence: Synthetic peptide
VPWGDYHPRNVY

SEQ ID NO: 78 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 79 - protein, 418 aa, Artificial Sequence
  Description of Artificial Sequence: Synthetic polypeptide
NSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSLENSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMS

SEQ ID NO: 80 - protein, 496 aa, Artificial Sequence
  Description of Artificial Sequence: Synthetic polypeptide
NSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSLENSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 81 - DNA, 7546 bp, Artificial Sequence
  Description of Artificial Sequence: Synthetic polynucleotide
acgcgtgtagtcttatgcaatactctgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatgcttctcctggtgacaagccttctgctctgtgagttaccacacccagcattcctcctgatcccaaactccctcttcaaccaggaggtgcagatccccctcacagagagctactgcgggccctgtccaaagaattggatatgttacaagaacaattgctaccagttcttcgatgagtcaaaaaattggtatgagagccaagcttcctgcatgtctcagaatgccagccttctgaaggtgtactcaaaagaagaccaggacttgctgaaactggtcaagtcttaccactggatggggctcgtgcacattccaacgaacggtagctggcagtgggaagatggctccatattgtctcctaaccttctcaccataatagagatgcagaagggtgattgcgctctgtacgctagtagcttcaagggctatattgagaattgtagtacacccaacacatacatttgtatgcagagaaccgtgggaggtggtggcagcggtggcggtggaagtggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgaatttaaatcggatccgcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 82 - DNA, 8017 bp, Artificial Sequence
  Description of Artificial Sequence: Synthetic polynucleotide
acgcgtgtagtcttatgcaatactctgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagagccgccaccatgcttctcctggtgacaagccttctgctctgtgagttaccacacccagcattcctcctgatcccaaactccctcttcaaccaggaggtgcagatccccctcacagagagctactgcgggccctgtccaaagaattggatatgttacaagaacaattgctaccagttcttcgatgagtcaaaaaattggtatgagagccaagcttcctgcatgtctcagaatgccagccttctgaaggtgtactcaaaagaagaccaggacttgctgaaactggtcaagtcttaccactggatggggctcgtgcacattccaacgaacggtagctggcagtgggaagatggctccatattgtctcctaaccttctcaccataatagagatgcagaagggtgattgcgctctgtacgctagtagcttcaagggctatattgagaattgtagtacacccaacacatacatttgtatgcagagaaccgtgggaggtggtggcagcggtggcggtggaagtggtggcggtggcagtctcgagaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctctagctttaaaggctatatagaaaactgttcaactccaaatacatacatctgcatgcaaaggactgtggcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgaatttaaatcggatccgcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 83 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 84 - protein, 393 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACGK

SEQ ID NO: 85 - protein, 874 aa, Homo sapiens
MNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 86 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 87 - protein, 254 aa, Artificial Sequence
  Description of Artificial Sequence: Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 88 - DNA, 24 bp, Artificial Sequence
  Description of Artificial Sequence: Synthetic oligonucleotide
agggcaagtcaggacattagtaaa

SEQ ID NO: 89 - protein, 8 aa, Artificial Sequence
  Description of Artificial Sequence: Synthetic peptide
RASQDISK

SEQ ID NO: 90 - DNA, 21 bp, Artificial Sequence
  Description of Artificial Sequence: Synthetic oligonucleotide
atctaccatacatcaagatta

SEQ ID NO: 91 - protein, 7 aa, Artificial Sequence
  Description of Artificial Sequence: Synthetic peptide
IYHTSRL

SEQ ID NO: 92 - DNA, 27 bp, Artificial Sequence
  Description of Artificial Sequence: Synthetic oligonucleotide
caacagggtaatacgcttccgtacacg

SEQ ID NO: 93 - protein, 9 aa, Artificial Sequence
  Description of Artificial Sequence: Synthetic peptide
QQGNTLPYT

SEQ ID NO: 94 - DNA, 30 bp, Artificial Sequence
  Description of Artificial Sequence: Synthetic oligonucleotide
ggggtctcattacccgactatggtgtaagc

SEQ ID NO: 95 - protein, 10 aa, Artificial Sequence
  Description of Artificial Sequence: Synthetic peptide
GVSLPDYGVS

SEQ ID NO: 96 - DNA, 42 bp, Artificial Sequence
  Description of Artificial Sequence: Synthetic oligonucleotide
gtaatatggggtagtgaaaccacatactataattcagctctc

SEQ ID NO: 97 - protein, 14 aa, Artificial Sequence
  Description of Artificial Sequence: Synthetic peptide
VIWGSETTYYNSAL

SEQ ID NO: 98 - DNA, 36 bp, Artificial Sequence
  Description of Artificial Sequence: Synthetic oligonucleotide
cattattactacggtggtagctatgctatggactac

SEQ ID NO: 99 - protein, 12 aa, Artificial Sequence
  Description of Artificial Sequence: Synthetic peptide
HYYYGGSYAMDY

SEQ ID NO: 100 - DNA, 321 bp, Artificial Sequence
  Description of Artificial Sequence: Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 101 - protein, 107 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 102 - DNA, 360 bp, Artificial Sequence
  Description of Artificial Sequence: Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 103 - protein, 120 aa, Artificial Sequence
  Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 104 - DNA, 48 bp, Artificial Sequence; identical to SEQ ID NO: 53
  Description of Artificial Sequence: Synthetic oligonucleotide

SEQ ID NO: 105 - protein, 16 aa, Artificial Sequence
  Description of Artificial Sequence: Synthetic peptide
KSSQSLVHSNGNTYLH

SEQ ID NO: 106 - DNA, 21 bp, Artificial Sequence
  Description of Artificial Sequence: Synthetic oligonucleotide
aaagtgagcaaccgctttagc

SEQ ID NO: 107 - protein, 7 aa, Artificial Sequence
  Description of Artificial Sequence: Synthetic peptide
KVSNRFS

SEQ ID NO: 108 - DNA, 27 bp, Artificial Sequence
  Description of Artificial Sequence: Synthetic oligonucleotide
gcggaaaccagccatgtgccgtggacc

SEQ ID NO: 109 - protein, 9 aa, Artificial Sequence
  Description of Artificial Sequence: Synthetic peptide
AETSHVPWT

SEQ ID NO: 110 - DNA, 39 bp, Artificial Sequence; identical to SEQ ID NO: 59
  Description of Artificial Sequence: Synthetic oligonucleotide

SEQ ID NO: 111 - protein, 13 aa, Artificial Sequence
  Description of Artificial Sequence: Synthetic peptide
KASGYSFPDYYIN

SEQ ID NO: 112 - DNA, 51 bp, Artificial Sequence; identical to SEQ ID NO: 61
  Description of Artificial Sequence: Synthetic oligonucleotide

SEQ ID NO: 113 - protein, 17 aa, Artificial Sequence
  Description of Artificial Sequence: Synthetic peptide
WIYFASGNSEYNQKFTG

SEQ ID NO: 114 - DNA, 30 bp, Artificial Sequence; identical to SEQ ID NO: 63
  Description of Artificial Sequence: Synthetic oligonucleotide

SEQ ID NO: 115 - protein, 10 aa, Artificial Sequence
  Description of Artificial Sequence: Synthetic peptide
LYDYDWYFDV

SEQ ID NO: 116 - DNA, 357 bp, Artificial Sequence; identical to SEQ ID NO: 67
  Description of Artificial Sequence: Synthetic polynucleotide

SEQ ID NO: 117 - protein, 119 aa, Artificial Sequence; identical to SEQ ID NO: 68
  Description of Artificial Sequence: Synthetic polypeptide

SEQ ID NO: 118 - DNA, 339 bp, Artificial Sequence; identical to SEQ ID NO: 65
  Description of Artificial Sequence: Synthetic polynucleotide

SEQ ID NO: 119 - protein, 113 aa, Artificial Sequence; identical to SEQ ID NO: 66
  Description of Artificial Sequence: Synthetic polypeptide

SEQ ID NO: 120 - protein, 6 aa, Artificial Sequence
  Description of Artificial Sequence: Synthetic peptide
QDIHGY

SEQ ID NO: 121 - protein, 3 aa, Artificial Sequence
  Description of Artificial Sequence: Synthetic peptide
YTS

SEQ ID NO: 122 - protein, 9 aa, Artificial Sequence
  Description of Artificial Sequence: Synthetic peptide
QQGNTLPWT

SEQ ID NO: 123 - protein, 8 aa, Artificial Sequence
  Description of Artificial Sequence: Synthetic peptide
GFAFSIYD

SEQ ID NO: 124 - protein, 8 aa, Artificial Sequence
  Description of Artificial Sequence: Synthetic peptide
ISSGGGTT

SEQ ID NO: 125 - protein, 16 aa, Artificial Sequence
  Description of Artificial Sequence: Synthetic peptide
ARHSGYGTHWGVLFAY

SEQ ID NO: 126 - protein, 229 aa, Artificial Sequence
  Description of Artificial Sequence: Synthetic polypeptide
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWQGTLVTVSA

SEQ ID NO: 127 - protein, 469 aa, Artificial Sequence
  Description of Artificial Sequence: Synthetic polypeptide
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDAAGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 128 - DNA, 729 bp, Artificial Sequence
  Description of Artificial Sequence: Synthetic polynucleotide
gaagtgcagctgctggaaagcggcggtggtctggttcagccgggtggcagcctgcgtctgagctgtgcggcgagcggctttacctttagcagctatgccatgagctgggtgcgtcaggcaccgggtaaaggcctggaatgggtgagcgcgattagcggcagcggcggcagcacctattatgcggatagcgtgaaaggccgttttaccattagccgtgataacagcaaaaacaccctgtatctgcagatgaacagcctgcgtgcggaagataccgcggtgtattattgcgcgaaagataagggttggtttaactggcaattcgattattggggccagggcaccctggttaccgttagcagcggtggaggcggttctggtggaggcggttcggatggcggaggttcagaaattgtgctgacccagagcccgggcacgctgtctctgagcccgggtgaacgtgcgaccctgagctgtcgtgcgagccaaagcgtgagcagcagctatctggcctggtatcagcagaaaccgggccaggcaccgcgtctgctgatttatggcgcgagcagccgtgcgaccggcattccggatcgttttagcggcagcggtagcggcaccgattttaccctgaccattagccgtctggaaccggaagattttgcggtgtattattgccagcagtatggcagcagcccgtggacctttggccagggcaccaaagtggaaattaaa

SEQ ID NO: 129 - DNA, 360 bp, Artificial Sequence
  Description of Artificial Sequence: Synthetic polynucleotide
gaagtgcaacttctcgagagcggtgggggactcgtccagccgggaggttccctgcgactcagctgtgcagcctcaggctttaccttttccagttacgcaatgagttgggtccggcaggcgcctggtaaaggactcgagtgggtgagtgcaatcagcggaagtggcgggtctacatactatgcggactctgttaaaggcaggttcactatttcaagggacaattccaagaacacgctctacctgcagatgaatagccttagagctgaagacacggccgtgtactattgtgccaaagacaagggatggttcaactggcagttcgactactgggggcagggaactctcgtcaccgtgagctcc

SEQ ID NO: 130 - DNA, 324 bp, Artificial Sequence
  Description of Artificial Sequence: Synthetic polynucleotide
gaaattgttctcacacagtcacccggaaccctttcattgtcccccggcgagcgcgccaccctcagctgtcgggccagtcagagcgtgtctagctcttacctggcctggtaccagcagaaacctgggcaagctcccagactcctgatatatggggccagcagccgggccactggcattccggacaggtttagtggatcaggctctggcactgattttacactgacgatttcaaggttggaacccgaagacttcgcagtgtactattgtcagcagtatgggtctagcccgtggactttcgggcaaggcaccaaggtggaaatcaag

SEQ ID NO: 131 - DNA, 717 bp, Artificial Sequence
  Description of Artificial Sequence: Synthetic polynucleotide
gaagtgcagctgctggaaagcggcggtggtctggttcagccgggtggcagcctgcgtctgagctgtgcggcgagcggctttacctttagcagctatgccatgagctgggtgcgtcaggcaccgggtaaaggcctggaatgggtgagcgcgattagcggcagcggcggcagcacctattatgcggatagcgtgaaaggccgttttaccattagccgtgataacagcaaaaacaccctgtatctgcagatgaacagcctgcgtgcggaagataccgcggtgtattattgcgcgaaaaaacaatatcacttcgattattggggccagggcaccctggttaccgttagcagcggtggaggcggttctggtggaggcggttcgggtggcggaggttcagaaattgtgctgacccagagcccgggcacgctgtctctgagcccgggtgaacgtgcgaccctgagctgtcgtgcgagccagagcgtgagcagcagctatctggcctggtatcagcagaaaccgggccaggcaccgcgtctgctgatttatggcgcgagcagccgtgcgaccggcattccggatcgttttagcggcagcggtagcggcaccgattttaccctgaccattagccgtctggaaccggaagattttgcggtgtattattgccagcagtatggcagcagcccgtggacctttggccagggcaccaaagtggaaattaaa

SEQ ID NO: 132 - DNA, 348 bp, Artificial Sequence
  Description of Artificial Sequence: Synthetic polynucleotide
gaagtgcaacttctcgagagcggtgggggactcgtccagccgggaggttccctgcgactcagctgtgcagcctcaggctttaccttttccagttacgcaatgagttgggtccggcaggcgcctggtaaaggactcgagtgggtgagtgcaatcagcggaagtggcgggtctacatactatgcggactctgttaaaggcaggttcactatttcaagggacaattccaagaacacgctctacctgcagatgaatagccttagagctgaagacacggccgtgtactattgtgccaaaaagcagtaccatttcgactactgggggcagggaactctcgtcaccgtgagctcc

SEQ ID NO: 133 - DNA, 324 bp, Artificial Sequence; identical to SEQ ID NO: 130
  Description of Artificial Sequence: Synthetic polynucleotide

SEQ ID NO: 134 - DNA, 738 bp, Artificial Sequence
  Description of Artificial Sequence: Synthetic polynucleotide
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaagtcatgatttatgatgtcagtaatcggccctcaggggtttctgatcgcttctctggctccaagtctggcaacacggcctccctgaccatctctgggctccaggttgaggacgaggctgattattactgcagctcatattcaaccagcatcaccccagttttcggcggggggaccaagctcaccgtcctaggagagggtaaatcttccggatctggttccgaaagcaaggctagccaggtccagctggtgcagtctggagcagaggtgaaaaagcccggggagtctctgaagatctcctgtgaggcttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagactggaactcggttactactactacggtatggacgtctggggccaaggaaccacggtcaccgtctcctca

SEQ ID NO: 135 - DNA, 45 bp, Artificial Sequence
  Description of Artificial Sequence: Synthetic oligonucleotide
ggagagggtaaatcttccggatctggttccgaaagcaaggctagc

SEQ ID NO: 136 - DNA, 330 bp, Artificial Sequence
  Description of Artificial Sequence: Synthetic polynucleotide
cagtcagctctgacccaacctgcctccgtctctgggagtccaggccagagtatcacaatttcttgtacaggcacctcatctgatgtcggcggttacaattacgttagttggtatcagcaacatccaggtaaggctccaaaggtgatgatctatgacgtctcaaatagaccctctggcgtcagcgacaggtttagtggtagcaaatccgggaacacagcttcacttacaattagcggcctccaagtagaagacgaagctgactattactgctctagttattcaacgtcaattacccctgtgtttggtggcggtacaaaactcacagtgctt

SEQ ID NO: 137 - DNA, 363 bp, Artificial Sequence
  Description of Artificial Sequence: Synthetic polynucleotide
caagtgcaacttgtgcaatcaggagctgaagtcaaaaagccgggagaatccctgaaaataagctgcgaagcaagtggttactcttttacttcttactggattggatgggttcggcagatgcccggaaagggactcgagtggatgggaattatttaccctggagacagcgacacaagatacagcccttcattccaggggcaggtgaccatttctgctgacaaatcaatcagtacagcctatctgcaatggagttccctcaaagccagtgacactgctatgtattactgcgcgcgactggaactgggatactactactacggaatggacgtatggggacagggaaccaccgttactgttagtagc

SEQ ID NO: 138 - skipped

SEQ ID NO: 139 - DNA, 10124 bp, Artificial Sequence
  Description of Artificial Sequence: Synthetic polynucleotide
ccaattaaccaattctgattagaaaaactcatcgagcatcaaatgaaactgcaatttattcacatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcaagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacaacgtggctttcccccccccccccatgacattaacctataaaaataggcgtatcacgaggccagcttgggaaaccataagaccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgatcgagatcgtgatccggatcaagatccagatcgaattggaggctacagtcagtggagaggactttcactgactgactgactgcgtctcaacctcctaggggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcgatcttcagacctggaggaggcaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggaccacgcgtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaagctgagcatccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagagccgccaccatgctcctcctcgtgactagccttctcctgtgcgagctcccacaccctgcattcctcctgatcccagaagtgcaacttctcgagagcggtgggggactcgtccagccgggaggttccctgcgactcagctgtgcagcctcaggctttaccttttccagttacgcaatgagttgggtccggcaggcgcctggtaaaggactcgagtgggtgagtgcaatcagcggaagtggcgggtctacatactatgcggactctgttaaaggcaggttcactatttcaagggacaattccaagaacacgctctacctgcagatgaatagccttagagctgaagacacggccgtgtactattgtgccaaagacaagggatggttcaactggcagttcgactactgggggcagggaactctcgtcaccgtgagctccggcggaggtggaagcgggggagggggctccggtggtgggggatcagaaattgttctcacacagtcacccggaaccctttcattgtcccccggcgagcgcgccaccctcagctgtcgggccagtcagagcgtgtctagctcttacctggcctggtaccagcagaaacctgggcaagctcccagactcctgatatatggggccagcagccgggccactggcattccggacaggtttagtggatcaggctctggcactgattttacactgacgatttcaaggttggaacccgaagacttcgcagtgtactattgtcagcagtatgggtctagcccgtggactttcgggcaaggcaccaaggtggaaatcaaggcagctgctggaggtgggggaagtggcggtggtggctcaggcggcggggggagcctcgaggacggtaatgaagagatggggggcattacacaaaccccgtacaaggtctctatcagtgggacgactgtgattctgacatgcccacagtatccaggttcagaaatcctgtggcagcataatgacaagaacatcggtggggatgaggatgataagaatatcggaagcgacgaagaccacctgtctctcaaagagtttagcgagctggagcagagtgggtattatgtctgctatcctagaggtagcaagccagaggacgcaaacttttacctttacctcagagccagggtctgcgagaactgcatggaaatggacgtgatgagtgttgcaactatagtgatagttgacatttgcatcaccgggggtctgctcctgctggtttactattggagcaagaaccgcaaggctaaagccaagccagtaacacggggcgcaggcgcgggaggcaggcagcgagggcagaataaggagcgccccccacccgtcccgaatccggattacgaacccattcggaaaggccagagggacttgtactcagggctcaaccaaagacggatcgaggggcgaggatccttgctgacatgtggtgacgtggaggagaatcctggtccttctcgcgccgccaccatggtgtctaaaggcgaagagctgttcaccggtgtggtgccgattcttgtagagctggatggagatgttaatggtcacaagttttcagtgtctggggagggcgaaggcgacgcgacctatggtaaactcacgcttaagtttatctgcaccacagggaagctccctgttccatggccaacccttgtgacaacacttacttacggcgtgcagtgtttcagcaggtatcctgaccatatgaagcagcacgatttcttcaagtctgcaatgcccgaggggtacgtacaagagcggacaattttcttcaaggacgacggaaattacaaaactagggcagaggttaagttcgaaggggatacacttgttaataggatcgaactgaaaggcattgatttcaaggaggatggaaacatactcgggcacaaactggaatataactacaattcacataatgtgtatatcatggctgataagcagaaaaacggtatcaaagtgaactttaagatccggcataacattgaagacggtagcgtgcagctcgctgaccactaccagcagaacactccaatcggggacgggccggtcctcctgcccgacaaccactacctcagcacccagagcgcacttagcaaagacccaaacgagaagagagaccatatggtgctgctggagttcgttaccgcagccggaatcaccttgggcatggacgagctctataaatgagaattcgaacggatatcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcagtactggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgatcataatcaagccatatcacatctgtagaggtttacttgctttaaaaaacctccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgtcgacacgaagagacgactgactgactgactggaaagaggaagggctggaagaggaaggagcttgatccagatcccgatctcgatccagatccggatcgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcagcttgggaaaccataagagctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaa

SEQ ID NO: 140 - DNA, 741 bp, Coxsackievirus B3
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 141 - DNA, 696 bp, Encephalomyocarditis virus
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 142 - DNA, 35 bp, Artificial Sequence
  Description of Artificial Sequence: Synthetic oligonucleotide
ggtagtggtgctactaacttcagcctgctgaagca

SEQ ID NO: 143 - DNA, 2621 bp, Artificial Sequence
  Description of Artificial Sequence: Synthetic polynucleotide
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctccgcggccgcgggcggtggaggcagtggtggtggcggctctggcggtggtggtagcctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 144 - DNA, 2465 bp, Artificial Sequence
  Description of Artificial Sequence: Synthetic polynucleotide
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctccgcggccgcgggcggtggaggcagtggtggtggcggctctggcggtggtggtagcctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 145 - DNA, 1604 bp, Anabaena sp.
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 146 - DNA, 1922 bp, Artificial Sequence
  Description of Artificial Sequence: Synthetic polynucleotide
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 147 - DNA, 1721 bp, Artificial Sequence
  Description of Artificial Sequence: Synthetic polynucleotide
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtaggtcgtgtgggttcaagtccctccacccccacgccggaaacgcaatagccgaaaaacaaaaaacaaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaaaaacaaaaaacaaaacggctattatgcgttaccggcgagacg

SEQ ID NO: 148 - DNA, 2621 bp, Artificial Sequence; identical to SEQ ID NO: 143
  Description of Artificial Sequence: Synthetic polynucleotide

SEQ ID NO: 149 - DNA, 2245 bp, Artificial Sequence
  Description of Artificial Sequence: Synthetic polynucleotide
gggagacccaagctggctagcgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgcttctgcttgtgacgtccctcctgctttgtgaactcccgcatcctgccttcctgctcatcccggaagttcagctggtagagtccggtggcgggctggtacagccagggggatctctccgtctctcatgtgctgccagtgggggagattggtccgctaactttatgtactggtatcgtcaagcacctgggaaacaacgcgagctggttgcacggatctctggacgaggggtagtggattacgtggaaagtgtgaaaggacgattcacaatttccagggacaattctaaaaacaccctttatttgcaaatgaatagtctgagagcagaagacacagccgtatattattgtgcagtcgcatcctattgggggcaggggactcttgtcacagtctcgagtgctgcagcaggcggcgggggctcaggagggggtgggtctggaggaggtgggagtctggaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactggcggattgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagggatc

SEQ ID NO: 150 - protein, 10 aa, Artificial Sequence
  Description of Artificial Sequence: Synthetic peptide
GGGGSGGGGS

SEQ ID NO: 151 - DNA, 30 bp, Artificial Sequence
  Description of Artificial Sequence: Synthetic oligonucleotide
ggtggcggaggttctggaggtggaggttcc

SEQ ID NO: 152 - protein, 20 aa, Artificial Sequence
  Description of Artificial Sequence: Synthetic peptide
  MISC_FEATURE (1)..(20): This sequence may encompass 1-4 “Gly Gly Gly Gly Ser” repeating units
GGGGSGGGGSGGGGSGGGGS

SEQ ID NO: 153 - protein, 5 aa, Artificial Sequence
  Description of Artificial Sequence: Synthetic peptide
GGGGS

SEQ ID NO: 154 - protein, 20 aa, Artificial Sequence
  Description of Artificial Sequence: Synthetic peptide
GGGGSGGGGSGGGGSGGGGS

SEQ ID NO: 155 - protein, 15 aa, Artificial Sequence
  Description of Artificial Sequence: Synthetic peptide
GGGGSGGGGSGGGGS

SEQ ID NO: 156 - protein, 4 aa, Artificial Sequence
  Description of Artificial Sequence: Synthetic peptide
  See specification as filed for detailed description of substitutions and preferred embodiments
GGGS

SEQ ID NO: 157 - protein, 20 aa, Artificial Sequence
  Description of Artificial Sequence: Synthetic peptide
  MISC_FEATURE (1)..(20): This sequence may encompass 2-4 “Gly Gly Gly Gly Ser” repeating units
GGGGSGGGGSGGGGSGGGGS

SEQ ID NO: 158 - protein, 15 aa, Artificial Sequence
  Description of Artificial Sequence: Synthetic peptide
  MISC_FEATURE (1)..(15): This sequence may encompass 1-3 “Gly Gly Gly Gly Ser” repeating units
GGGGSGGGGSGGGGS

SEQ ID NO: 159 - RNA, 5000 nt, Artificial Sequence
  Description of Artificial Sequence: Synthetic polynucleotide
  misc_feature (1)..(5000): This sequence may encompass 50-5000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 160 - protein, 30 aa, Artificial Sequence
  Description of Artificial Sequence: Synthetic polypeptide
  MISC_FEATURE (1)..(30): This sequence may encompass 1-6 “Gly Gly Gly Gly Ser” repeating units
  See specification as filed for detailed description of substitutions and preferred embodiments
GGGGSGGGGSGGGGSGGGGSGGGGSGGGGS

SEQ ID NO: 161 - protein, 50 aa, Artificial Sequence
  Description of Artificial Sequence: Synthetic polypeptide
  MISC_FEATURE (1)..(50): This sequence may encompass 2-10 “Gly Gly Gly Gly Ser” repeating units
GGGGSGGGGSGGGGSGGGGSGGGGSGGGGSGGGGSGGGGSGGGGSGGGGS

SEQ ID NO: 162 - DNA, 100 bp, Artificial Sequence
  Description of Artificial Sequence: Synthetic polynucleotide
tttttttttttttttttttttttttttttttttttttttttttttttttttttttttttttttttttttttttttttttttttttttttttttttttttt

SEQ ID NO: 163 - DNA, 5000 bp, Artificial Sequence
  Description of Artificial Sequence: Synthetic polynucleotide
  misc_feature (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 164 - RNA, 5000 nt, Artificial Sequence; identical to SEQ ID NO: 159
  Description of Artificial Sequence: Synthetic polynucleotide
  misc_feature (1)..(5000): This sequence may encompass 100-5000 nucleotides

SEQ ID NO: 165 - RNA, 400 nt, Artificial Sequence
  Description of Artificial Sequence: Synthetic polynucleotide
  misc_feature (1)..(400): This sequence may encompass 100-400 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 166 - protein, 111 aa, Artificial Sequence
  Description of Artificial Sequence: Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 167 - DNA, 19 bp, Artificial Sequence
  Description of Artificial Sequence: Synthetic oligonucleotide
tacggacttaaaatccgtt

(19) United States
(12) Patent Application Publication
(10) Pub. No.: US 2022/0362295 A1
(43) Pub. Date: Nov. 17, 2022

(54) COMPOSITIONS AND METHODS FOR TCR REPROGRAMMING USING FUSION PROTEINS

(71) Applicant: TCR2 THERAPEUTICS INC., Cambridge, MA (US)

(72) Inventors: Philippe KIEFFER-KWON, Somerville, MA (US); Ella LIBERZON, Natick, MA (US); Robert HOFMEISTER, Scituate, MA (US); Patrick Alexander BAEUERLE, Gauting (DE); Daniel GETTS, Stow, MA (US)

(21) Appl. No.: 17/604,857

(22) PCT Filed: Apr. 22, 2020
(86) PCT No.: PCT/US2020/029344

§ 371 Date: Oct. 19, 2021

Related U.S. Application Data
(60) Provisional application No. 62/836,977, filed on Apr. 22, 2019.
(60) Provisional application No. 62/943,679, filed on Dec. 4, 2019.

(51) Int. Cl. A61K 35/17; C07K 14/725; C07K 14/73; C07K 14/705; C07K 14/735; C07K 16/30; C07K 16/28; C07K 16/40; A61P 35/00; C12N 15/85

(52) U.S. Cl. CPC A61K 35/17; C07K 14/7051; C07K 14/70514; C07K 14/70589; C07K 14/70596; C07K 14/70517; C07K 14/70535; C07K 14/70521; C07K 14/70575; C07K 14/70532; C07K 16/3061; C07K 16/3023; C07K 16/2866; C07K 16/2863; C07K 16/3092; C07K 16/40; A61P 35/00; C12N 15/85; C07K 2319/33; C07K 2319/03; C12N 2840/203; C07K 2319/02; A61K 2039/5158

(57) ABSTRACT

Provided herein are recombinant nucleic acids encoding T cell receptor (TCR) fusion proteins (TFPs), modified human immune cells expressing the encoded molecules, and methods of use thereof for the treatment of diseases, including cancer.

### CROSS-REFERENCE

This application claims the benefit of U.S. Provisional Application No. 62/836,977, filed Apr. 22, 2019, and U.S. Provisional Application No. 62/943,679, filed Dec. 4, 2019, each of which is incorporated herein by reference in its entirety.

### BACKGROUND

Cancer is a leading cause of death in the United States and elsewhere. Depending on the type of cancer, it is typically treated with surgery, chemotherapy, and/or radiation. These treatments often fail, and it is clear that new therapies are necessary, to be used alone or in combination with current standards of care.

Most patients with hematological malignancies or with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy.

The success of engineered T cell therapies at clearing solid tumors hinges on the ability of said T cells to function in the immune suppressive tumor microenvironment (TME). Numerous strategies to overcome the TME have been deployed or are in development. A plethora of approaches directly modify and engineer T cells to express multiple co-stimulatory domains (for example constitutive expression of cd40), to secrete pro-inflammatory cytokines such as IL-12 and/or antibodies that block inhibitory signals (such as anti-PD1) (see, e.g., Yeku & Brentjens, 2016).

For example, the generation of tumor-specific T cells by genetic modification to express chimeric antigen receptors (CARs) has gained traction generating powerful antitumor effects (Jena et al., 2010, Blood. 116:1035-1044; Bonini et al., 2011, Biol Blood Marrow Transplant 17(1 Suppl):515-20; Restifo et al., 2012, Nat Rev Immunol 12:269-281; Kohn et al., 2011, Mol Ther 19:432-438; Savoldo et al., 2011, J Clin Invest 121:1822-1825; Ertl et al., 2011, Cancer Res 71:3175-3181).

The clinical results with CD19-specific CAR T cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma. More recent approach is to improve genetically engineered T cells to more broadly act against various human malignancies. Novel fusion proteins of TCR subunits, including CD3 epsilon, CD3gamma and CD3 delta, and of TCR alpha and TCR beta chains with binding domains specific for cell surface antigens have shown the potential over limitations of existing approaches. See, e.g., copending International Application Nos. PCT/US2016/033146, filed May 18, 2016; PCT/US2017/045159, filed Aug. 2, 2017; and PCT/US2018/037387, filed Jun. 13, 2018, each of which is hereby incorporated by reference.

Unfortunately, many of these approaches have been faced with technical limitations of delivering pharmaceutical compositions within T-cells in order to achieve effective protein expression.

Technical limitations and challenges are posed by the large size of genes encoding proteins to engineer T-cells, such as fusion proteins. Encoding these large genes into viable vectors necessary to generate T cells is complex. Furthermore, even when a vector is successfully generated, T cell transduction efficiency and stable protein expression have not always been observed. This may be associated with the long and complicated process associated with transcription, translation and final protein secretion. Speculatively, this may further be compounded by the fact that many of these proteins are not endogenously expressed by T cells.

For example, there are multiple steps which may occur after delivery of recombinant DNA into the cells but before the encoded protein is made which can affect protein expression. Once inside the cell, DNA may be transported into the nucleus where it is transcribed into mRNA. The mRNA transcribed from DNA may then enter the cytoplasm where it is translated into protein. Not only do the multiple processing steps from administered DNA to protein create lag times before the generation of the functional protein, each step represents an opportunity for error and damage to the cell. Further, it is known to be difficult to obtain DNA expression in cells as frequently DNA enters a cell but is not expressed or not expressed at reasonable rates or concentrations. This can be a particular problem when DNA is introduced into primary cells or modified cell lines.

### SUMMARY

Recognized herein is a need for the delivery of biological modalities to address pitfalls surrounding the modulation of intracellular translation and processing of nucleic acids encoding polypeptides and therefore optimizing protein expression from the delivered modalities.

There is a clear need to improve genetically engineered T cells to more broadly act against various human malignancies, e.g., a cancer.

The compositions and methods in the following disclosure have been designed to addresses this need by delivering compositions comprising nucleic acids such as circular RNA (circRNA).

Thus, in one aspect, disclosed herein is an isolated recombinant nucleic acid molecule comprising: (A) one or more ribonucleic acid (RNA) sequences encoding a T cell receptor (TCR) fusion protein (TFP) comprising (a) a TCR subunit comprising (i) at least a portion of a TCR extracellular domain, (ii) a transmembrane domain, and (iii) a TCR intracellular domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (b) an antigen binding domain; wherein the TCR subunit and the antigen binding domain are operatively linked; and wherein the TFP incorporates into a TCR when expressed in a T cell; and (B) one or more internal ribosome entry sites (IRES); wherein (A) and (B) are operably linked to form a circular recombinant nucleic acid molecule. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, the isolated recombinant nucleic acid molecule further comprises (C) a nucleic acid spacer sequence proximal to the 5′ end of (A) and the 3′ end of (B), wherein (C) is formed by the circularization of a linear nucleic acid. In one embodiment, the spacer sequence is about 30-100 nucleotides in length. In another embodiment, the circularization of the linear nucleic acid produces a circular RNA molecule. In another embodiment, the circular recombinant nucleic acid molecule is exogenous. In another embodiment, the IRES comprises the IRES sequence from Coxsackievirus B3 (CVB3) or from encephalomyocarditis virus (EMCV). In another embodiment, the circular recombinant nucleic acid molecule is suitable for transfection or transduction into an allogeneic or autologous human immune cell.

In another aspect is provided an isolated recombinant nucleic acid molecule comprising: (A) one or more ribonucleic acid (RNA) sequences encoding a chimeric antigen receptor (CAR) or a T cell receptor (TCR); and (B) one or more internal ribosome entry sites (IRES); wherein (A) and (B) are operably linked to form a circular recombinant nucleic acid molecule. In one embodiment, the isolated recombinant nucleic acid molecule further comprises (C) a nucleic acid spacer sequence proximal to the 5′ end of (A) and the 3′ end of (B), wherein (C) is formed by the circularization of a linear nucleic acid. In one embodiment, the spacer sequence is about 30-100 nucleotides in length. In another embodiment, the isolated recombinant nucleic acid molecule is exogenous. In one embodiment, the IRES further comprises the IRES obtained from Coxsackievirus B3 (CVB3) or from encephalomyocarditis virus (EMCV).

In another aspect is provided an isolated recombinant nucleic acid molecule comprising (A) one or more deoxyribonucleic acid (DNA) sequences encoding a T cell receptor (TCR) fusion protein (TFP) comprising (a) a TCR subunit comprising (i) at least a portion of a TCR extracellular domain, (ii) a transmembrane domain, and (iii) a TCR intracellular domain comprising a stimulatory domain from an intracellular signaling domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (b) an antigen binding domain; wherein the TCR subunit and the antigen binding domain are operatively linked; and wherein the TFP incorporates into a TCR when expressed in a T cell; (B) one or more DNA sequences comprising one or more internal ribosome entry sites (IRES); and (C) one or more DNA sequences comprising a first circularization domain comprising at least one of a 5′ homology sequence and a 3′ permutated intron-exon (PIE) sequence; and (D) one or more DNA sequences comprising a second circularization domain comprising at least one of a 3′ homology sequence and a 5′ PIE sequence, wherein (A) and (B) are operably linked. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, (A)-(D) are operably linked in the orientation (C)-(B)-(A)-(D). In another embodiment, the one or more DNA sequences further comprises at least one spacer sequence. In one embodiment the spacer sequence is at least about 30-100 nucleotides in length. In one embodiment, the nucleic acid molecule is exogenous. In another embodiment, the nucleic acid molecule is a plasmid. In another embodiment, the nucleic acid molecule further comprises an antigen binding domain specific to a tumor associated antigen (TAA). In another embodiment, the IRES comprises the IRES sequence from Coxsackievirus B3 (CVB3) or from encephalomyocarditis virus (EMCV). In one embodiment, the isolated recombinant nucleic acid molecule further comprises at least one additional 5′ homology sequence and one additional 3′ homology sequence.

In another aspect is provided an isolated recombinant nucleic acid molecule comprising (A) one or more deoxyribonucleic acid (DNA) sequences encoding a CAR or TCR; and (B) one or more DNA sequences comprising one or more internal ribosome entry sites (IRES); and (C) one or more DNA sequences comprising a first circularization domain comprising at least one of a 5′ homology sequence and a 3′ permutated intron-exon (PIE) sequence; and (D) one or more DNA sequences comprising a second circularization domain comprising at least one of a 3′ homology sequence and a 5′ PIE sequence, wherein (A) and (B) are operably linked. In one embodiment, (A)-(D) are operably linked in the orientation (C)-(B)-(A)-(D). In another embodiment, the one or more DNA sequences further comprises at least one spacer sequence. In one embodiment, the spacer sequence is at least about 30-100 nucleotides in length. In another embodiment, the nucleic acid molecule is exogenous. In one embodiment, the nucleic acid molecule is a plasmid. In one embodiment, the nucleic acid molecule further comprises an encoded antigen binding domain. In one embodiment, the IRES comprises the IRES sequence from Coxsackievirus B3 (CVB3) or from encephalomyocarditis virus (EMCV). In another embodiment, the isolated recombinant nucleic acid molecule further comprises at least one additional 5′ homology sequence and one additional 3′ homology sequence. In one embodiment, the sequence encoding the antigen binding domain is connected to the sequence encoding the TCR extracellular domain by an encoded linker sequence. In one embodiment, the encoded linker sequence comprises (G₄S)ₙ, wherein n=1 to 4. In another embodiment, the encoded antigen binding domain specifically binds to a tumor associated antigen. In one embodiment, the tumor associated antigen is CD19 or a variant thereof, CD20, CD22, BCMA, MSLN, IL13Ra2, EGFRvIII, MUC16, MUC1, ROR1, PD1, EphA2, or a combination thereof. In one embodiment, the encoded transmembrane domain comprises a transmembrane domain of a protein selected from the group consisting of a TCR alpha chain, a TCR beta chain, a TCR delta chain, a TCR gamma chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto.

In one embodiment, the isolated recombinant nucleic acid molecule further comprises a sequence encoding a costimulatory domain, wherein the encoded costimulatory domain is a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In one embodiment, the at least one but not more than 20 modifications thereto comprise a modification of an amino acid that mediates cell signaling or a modification of an amino acid that is phosphorylated in response to a ligand binding to the encoded TFP or CAR or TCR. In one embodiment, the encoded TFP or CAR or TCR further comprises an immunoreceptor tyrosine-based activation motif (ITAM) or portion thereof, wherein the ITAM or portion thereof is from a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In one embodiment, the ITAM or portion thereof replaces an ITAM of the TCR intracellular domain; wherein the replaced ITAM of the TCR intracellular domain is derived from only CD3 epsilon or CD3 gamma and is different than the ITAM or portion thereof that replaces it. In one embodiment, the encoded TFP molecule is capable of functionally interacting with an endogenous TCR complex, at least one endogenous TCR polypeptide, or a combination thereof. In one embodiment, the antigen binding domain is a scFv or a VHH domain. In one embodiment, the isolated recombinant nucleic acid molecule is comprised in a cell. In one embodiment, the cell is a CD8+ or CD4+ or CD8+CD4+ human immune cell. In one embodiment, the antibody or fragment thereof binds to a cell surface antigen. In one embodiment, the antibody or fragment thereof binds to a cell surface antigen on the surface of a tumor cell. In one embodiment, the isolated recombinant nucleic acid further comprises a sequence encoding a TCR constant domain that incorporates into a functional TCR complex when expressed in a T cell. In another embodiment, the TCR constant domain incorporates into a same functional TCR complex as the functional TCR complex that incorporates the TFP when expressed in a T cell. In one embodiment, the sequence encoding the TFP and the sequence encoding the TCR constant domain are contained within a same nucleic acid molecule. In one embodiment, the sequence encoding the TFP and the sequence encoding the TCR constant domain are contained within different nucleic acid molecules. In one embodiment, the TCR subunit and the antibody domain, the antigen binding domain are operatively linked by an encoded linker sequence.

In one embodiment, the transmembrane domain is a T cell receptor complex transmembrane domain from CD3 epsilon, CD3 gamma, CD3 delta, TCR alpha or TCR beta or TCR gamma or TCR delta. In one embodiment, the intracellular domain is derived from only CD3 epsilon, only CD3 gamma, only CD3 delta, only TCR alpha, only TCR beta, only TCR gamma, or only TCR delta. In one embodiment, the isolated recombinant nucleic acid further comprises a sequence encoding a costimulatory domain. In one embodiment, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In one embodiment, the isolated recombinant nucleic acid molecule further comprises a sequence encoding an antigen binding domain. In one embodiment, the isolated recombinant nucleic acid molecule further comprises a sequence encoding a protein transduction domain or a cell penetrating peptide.

In another aspect is provided a method of producing a modified human immune cell ex vivo, comprising transducing or transfecting the immune cell with one or more of the isolated recombinant nucleic acid molecules disclosed herein. In one embodiment, the immune cell is a T cell. In another embodiment, the immune cell is a human T cell selected from a group comprising a CD4+ cell, a CD8 cell, a naive T cell, a memory stem T cell, a central memory T cell, a double negative T cell, an effector memory T cell, an effector T cell, a Th1 cell, a Tc1 cell, a Th2 cell, a Tc2 cell, a Th17 cell, a Th22 cell, a gamma/delta T cell, a natural killer (NK) cell, a natural killer T (NKT) cell, B cells, a hematopoietic stem cell and a pluripotent stem cell.

In another aspect is provided a method of producing a circular RNA encoding a T cell receptor (TCR) fusion protein (TFP) comprising the steps of: (i) providing one or more vectors comprising: (A) one or more DNA sequences encoding a T cell receptor (TCR) fusion protein (TFP) comprising (a) a TCR subunit comprising (1) at least a portion of a TCR extracellular domain, (2) a transmembrane domain, and (3) a TCR intracellular domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (b) an antigen binding domain; wherein the TCR subunit and the antigen binding domain are operatively linked; and wherein the TFP incorporates into a TCR when expressed in a T cell; (B) one or more DNA sequences comprising one or more internal ribosome entry sites (IRES); and (C) one or more DNA sequences comprising a first circularization domain comprising at least one of a 5′ homology sequence and a 3′ permutated intron-exon (PIE) sequence; and (D) one or more DNA sequences comprising a second circularization domain comprising at least one of a 3′ homology sequence and a 5′ PIE sequence, wherein (A) and (B) are operably linked; (ii) transcribing the one or more vectors to produce one or more linear RNA; and (iii) allowing the linear RNA to self-splice by using a chemical method, an enzymatic method, or a ribozymatic method, thereby producing the circular RNA. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, the vector is a DNA vector. In another embodiment, the circular RNA is produced in vitro or ex vivo. In another embodiment, the circular RNA further comprises at least one spacer sequence. In one embodiment, the spacer sequence is about 30-100 nucleotides in length. In another embodiment, the vector is a plasmid. In one embodiment, the circular RNA is produced by in vitro transcription. In one embodiment, the vector is integrated into the genome of a host cell. In one embodiment, the IRES comprises the IRES sequence from Coxsackievirus B3 (CVB3) or from encephalomyocarditis virus (EMCV). In one embodiment, the vector further comprises at least one additional 5′ homology sequence and one additional 3′ homology sequence. In some embodiments, the vector incorporates into the genome of a target cell. In some embodiments, the vector is administered to a subject as the payload of a delivery vehicle (e.g., a nanoparticle, liposome, endosome, etc.).

In another aspect is provided a method of producing a circular RNA encoding a CAR or a TCR comprising the steps of: (i) providing one or more vectors comprising: (A) one or more DNA sequences encoding a CAR or a (B) one or more DNA sequences comprising one or more internal ribosome entry sites (IRES); and (C) one or more DNA sequences comprising a first circularization domain comprising at least one of a 5′ homology sequence and a 3′ permutated intron-exon (PIE) sequence; and (D) one or more DNA sequences comprising a second circularization domain comprising at least one of a 3′ homology sequence and a 5′ PIE sequence, wherein (A) and (B) are operably linked; (ii) transcribing the one or more vectors to produce one or more linear RNA; and (iii) allowing the linear RNA to self-splice by using a chemical method, an enzymatic method, or a ribozymatic method, thereby producing the circular RNA. In one embodiment, the circular RNA further comprises at least one spacer sequence. In one embodiment, the spacer sequence is about 30-100 nucleotides in length. In one embodiment, the vector is a plasmid. In another embodiment, the circular RNA is produced by in vitro transcription. In one embodiment, the encoded antigen binding domain is specific to a tumor associated antigen. In another embodiment, the IRES comprises the IRES sequence from Coxsackievirus B3 (CVB3) or from encephalomyocarditis virus (EMCV). In one embodiment, the vector further comprises at least one additional 5′ homology sequence and one additional 3′ homology sequence.

In another aspect is provided a method of producing a modified immune cell containing a circular RNA encoding a T cell receptor (TCR) fusion protein (TFP) in a subject, comprising the steps of: (1) providing one or more circular RNA vectors comprising: (A) one or more sequences encoding a T cell receptor (TCR) fusion protein (TFP) comprising (a) a TCR subunit comprising (i) at least a portion of a TCR extracellular domain; a transmembrane domain; (ii) a TCR intracellular domain comprising a stimulatory domain from an intracellular signaling domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (iii) an antigen binding domain; wherein the TCR subunit and the antigen binding domain are operatively linked; and wherein the TFP incorporates into a TCR when expressed in a T cell; (B) one or more DNA sequences comprising one or more internal ribosome entry sites (IRES); and (C) one or more DNA sequences comprising a first circularization domain comprising at least one of a 5′ homology sequence and a 3′ permutated intron-exon (PIE) sequence; and (D) one or more DNA sequences comprising a second circularization domain comprising at least one of a 3′ homology sequence and a 5′ PIE sequence, wherein (A) and (B) are operably linked; and (2) administering the one or more circular RNA vectors to the subject in an amount effective to modify a population of target immune cells. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, the population of target immune cells comprises a human T cell selected from a group comprising a CD4+ cell, a CD8 cell, a naive T cell, a memory stem T cell, a central memory T cell, a double negative T cell, an effector memory T cell, an effector T cell, a Th1 cell, a Tc1 cell, a Th2 cell, a Tc2 cell, a Th17 cell, a Th22 cell, a gamma/delta T cell, a natural killer (NK) cell, a natural killer T (NKT) cell, B cells, a hematopoietic stem cell and a pluripotent stem cell. In one embodiment, the one or more circular RNA vectors further comprise at least one cell targeting ligand that comprises a binding domain for a T-cell receptor motif. In one embodiment, the one or more circular RNA vectors further comprise a delivery vehicle selected from a group consisting essentially of a macromolecule complex, a nanocapsule, a nanoparticle, an exosome, an exosome-lipid conjugate, a microsphere, a bead, an oil-in-water emulsion, a lipid-nanoparticle conjugate, a micelle, mixed micelles, and a liposome. In one embodiment, the delivery vehicle further comprises at least one cell targeting ligand that comprises a binding domain for a T-cell receptor motif. In another embodiment, the cell targeting ligand is chosen from the group comprising a T-cell α chain, a T-cell β chain, a T-cell γ chain, a T-cell δ chain, CCR7, CD1a, CD1b, CD1c, CD1d, CD3, CD4, CD5, CD7, CD8, CD11b, CD11c, CD16, CD19, CD20, CD21, CD22, CD25, CD28, CD34, CD35, CD39, CD40, CD45RA, CD45RO, CD46, CD52, CD56, CD62L, CD68, CD80, CD86, CD95, CD101, CD117, CD127, CD133, CD137 (4-1BB), CD148, CD163, F4/80, IL-4Ra, Sca-1, CTLA-4, GITR, GARP, LAP, granzyme B, LFA-1, transferrin receptor, and combinations thereof.

In another aspect is provided a method of treating cancer in a subject in need thereof, the method comprising administering to the subject an isolated recombinant nucleic acid molecule encoding a T cell receptor fusion protein (TFP) according to claim 1 or claim 15 in a formulation for delivery of the isolated recombinant nucleic acid molecule to the subject, and wherein the isolated recombinant nucleic acid molecule enters the target cell in vivo. In one embodiment, the isolated recombinant nucleic acid molecule is a circular RNA molecule. In another embodiment the recombinant nucleic acid comprises, in 5′ to 3′ order: i) a 3′ portion of an exogenous intron comprising a 3′ splice site, ii) a nucleic acid sequence encoding an RNA exon, and iii) a 5′ portion of an exogenous intron comprising a 5′ splice site, wherein splicing of an RNA produced by transcription of the recombinant nucleic acid results in production of the circular RNA in the subject. In one embodiment, wherein the circular RNA is encoded by a DNA vector. In one embodiment, the circular RNA is conjugated to a targeting moiety. In another embodiment, the circular RNA comprises a protein transduction domain or a cell penetrating peptide. In another embodiment, the formulation comprises a nanoparticle. In one embodiment, the nanoparticle is an exosome, a liposome, or an exosome-liposome hybrid. In another embodiment, the nanoparticle comprises at least one targeting moiety. In another embodiment, the targeting moiety is a binding ligand or a murine antibody or a human or humanized antibody or fragment thereof. In another embodiment, the targeting moiety binds specifically to CD3, CD4, or CD8. In one embodiment, the target cell is a human immune cell. In another embodiment, the target cell is a human T cell selected from a group comprising a CD4+ cell, a CD8 cell, a naive T cell, a memory stem T cell, a central memory T cell, a double negative T cell, an effector memory T cell, an effector T cell, a Th1 cell, a Tc1 cell, a Th2 cell, a Tc2 cell, a Th17 cell, a Th22 cell, a gamma/delta T cell, a natural killer (NK) cell, a natural killer T (NKT) cell, a hematopoietic stem cell and a pluripotent stem cell.

In another aspect is provided a pharmaceutical formulation comprising: (a) a human immune cell containing a circular RNA in an amount sufficient to treat a cancer in a subject, wherein the circular RNA encodes a T cell receptor (TCR) fusion protein (TFP) comprising (A) a TCR subunit comprising (i) at least a portion of a TCR extracellular domain; a transmembrane domain; (ii) a TCR intracellular domain comprising a stimulatory domain from an intracellular signaling domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (iii) an antigen binding domain; wherein the TCR subunit and the antigen binding domain are operatively linked; and wherein the TFP incorporates into a TCR when expressed in a T cell; and (b) a pharmaceutically acceptable carrier. In one embodiment, the TCR intracellular domain comprises a stimulatory domain derived from CD3 epsilon or CD3 gamma or CD3 delta. In another embodiment, the antigen binding domain comprise an antibody or antibody fragment.

In another aspect is provided a method of treating cancer in a subject in need thereof, the method comprising administering to the subject an effective amount of a pharmaceutical formulation comprising a human immune cell containing a circular RNA, wherein the circular RNA encodes a T cell receptor (TCR) fusion protein (TFP) comprising (A) a TCR subunit comprising (i) at least a portion of a TCR extracellular domain; a transmembrane domain; (ii) a TCR intracellular domain comprising a stimulatory domain from an intracellular signaling domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (iii) an antigen binding domain; wherein the TCR subunit and the antigen binding domain are operatively linked; and wherein the TFP incorporates into a TCR when expressed in a T cell; and a pharmaceutically acceptable carrier. In one embodiment, the method comprises a single administration of the formulation. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, the method comprises more than one administration of the formulation. In one embodiment, the cell is an allogeneic T cell. In another embodiment, the cell is an autologous T cell.

In another aspect is provided a pharmaceutical formulation comprising: a pharmaceutical formulation comprising a circular RNA, wherein the circular RNA encodes a T cell receptor (TCR) fusion protein (TFP) comprising (A) a TCR subunit comprising (i) at least a portion of a TCR extracellular domain; a transmembrane domain; (ii) a TCR intracellular domain comprising a stimulatory domain from an intracellular signaling domain, wherein the extracellular, transmembrane, and/or intracellular signaling domains of the TCR subunit are derived from CD3 epsilon or CD3 gamma or CD3 delta or TCR alpha or TCR beta or TCR delta or TCR gamma; and (iii) an antigen binding domain; wherein the TCR subunit and the antigen binding domain are operatively linked; and wherein the TFP incorporates into a TCR when expressed in a T cell; and a pharmaceutically acceptable carrier. In one embodiment, the circular RNA is conjugated to a targeting moiety. In another embodiment, the circular RNA comprises one or more of: a protein transduction domain, a cell penetrating peptide, or an endosomolytic peptide. In one embodiment, the TCR intracellular domain comprises a stimulatory domain derived from CD3 epsilon or CD3 gamma or CD3 delta. In another embodiment, the antigen binding domain comprise an antibody or antibody fragment. In one embodiment, the formulation comprises a nanoparticle. In another embodiment, the nanoparticle is an exosome, a liposome, or an exosome—liposome hybrid. In one embodiment, the nanoparticle comprises at least one targeting moiety. In another embodiment, the targeting moiety is a binding ligand or a murine antibody or a human or humanized antibody or fragment thereof. In one embodiment, the targeting moiety binds specifically to CD3, CD4, or CD8.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic illustration of the use of circRNA encoding a TFP to transduce T cells in vivo, resulting in expression of the TFP. As is shown, the circRNA is generated from a precursor that comprises an IRES sequence upstream of the sequence encoding the TFP. The IRES linked to the TFP is then flanked on either end by an internal homology sequence followed by a permutated intron-exon sequence, followed by an external homology sequence, moving distally. The construct is then able to self-splice, resulting in the circRNA. circRNA can persist and remain functional longer than mRNA. circRNAs can encode several kilobase (kb) of coding sequence (CDS). This schematic illustration is adapted from Wesselhoeft et. al., Nat. Commun., 9:26-29., 2018.

FIG. 2 is a schematic illustration of the linear form and three-dimensional structure of the precursor RNA encoded by SEQ ID NO: 146 that is able to form the GFP circRNA described herein.

FIG. 3 is an image of an agarose gel showing the products of an in vitro transcription reaction for generating the RNA precursors and circRNAs for GFP circRNAs having both the CVB3 and EMCV IRESs.

FIG. 4 is a graphical representation of flow cytometry data showing the proportion of Jurkat cells transduced with GFP circRNA expressing GFP and GFP splicing mutant (SEQ ID NO: 147). In this example, the circRNAs were delivered to Jurkat cells by electroporation.

FIG. 5 is a schematic illustration of the linear form and three-dimensional structure of the precursor RNA encoded by SEQ ID NO: 148 that is able to form the antiCD19-TFP circRNA described herein.

FIG. 6 is an image of an agarose gel showing the products of an in vitro transcription reaction for generating the RNA precursor and circRNA for antiCD19-TFP circRNA generated in Example 11. This example shows CVB3 anti-CD19 TFP circRNA circularization.

FIG. 7 is a graphical representation of flow cytometry data showing the proportion of Jurkat cells transduced with antiCD19-TFP circRNA expressing antiCD19-TFP.

FIG. 8 is a schematic illustration of the linear form and three-dimensional structure of the precursor RNA encoded by SEQ ID NO: 149 that is able to form the antiMSLN-TFP circRNA described herein.

FIG. 9 is an image of an agarose gel showing antiMSLN-TFP circRNA generated in Example 12. This example shows CVB3 anti-MSLN TFP circRNA circularization.

FIG. 10 is a graphical representation of flow cytometry data showing the proportion of Jurkat cells transduced with antiMSLN-TFP circRNA expressing antiMSLN-TFP. The circRNAs were delivered to Jurkat cells by electroporation.

FIG. 11 is a graphical representation of flow cytometry data showing the proportion of activated T cells transduced with antiMSLN-TFP circRNA expressing antiMSLN-TFP. The circRNAs were delivered to activated T cells by electroporation.

FIG. 12 is a graphical representation of a cytotoxicity assay comparing the % effector cell killing seen with cells transduced with antiMSLN-TFP circRNA compared to lentiviral antiMSLN-TFP or an untransduced control showing the proportion of activated T cells transduced with antiMSLN-TFP circRNA expressing antiMSLN-TFP.

FIG. 13 is a graphical representation of flow cytometry data detecting CD3 epsilon and GFP or VHH on the surface of cells electroporated with a GFP circRNA, the TAA(X).TFP circRNAs shown, or non-electroporated controls.

FIG. 14 is a graphical representation of a cytotoxicity assay comparing the % effector cell lysis seen with T cells electroporated with GFP circRNA or TAA(X).TFP circRNAs, compared to T cells transduced with lentiviral TAA(X).TFPs or an untransduced control. For each transduced/electroporated construct and for untransduced controls, shown from left to right is the % effector cell lysis at an effector:target cell ratio of 9:1, 3:1, and 1:1.

FIG. 15 is an image of an agarose gel showing the products of an in vitro transcription reaction for generating the RNA precursors and circRNAs for antiMSLN-TFP circRNAs with 0%, 10% and 100% m6A.

FIG. 16 is a series of graphs showing antiMSLN-TFP expression and MFI in cells electroporated with circRNA having 0%, 10% or 100% m6A or controls.

### DETAILED DESCRIPTION OF THE INVENTION

Provided herein are recombinant nucleic acids for use in treating a subject, e.g., a subject having a cancer. In certain aspects, the recombinant nucleic acids comprise a circular RNA sequence. In certain aspects, the recombinant nucleic acids comprise DNA encoding a circular RNA sequence. In some embodiments the recombinant nucleic acid sequence encodes a chimeric antigen receptor (CAR), a T cell receptor (TCR), or a T cell receptor (TCR) fusion protein (TFP), wherein the TFP comprises (a) a TCR subunit comprising (i) at least a portion of a TCR extracellular domain, (ii) a transmembrane domain, and (iii) a TCR intracellular domain of CD3 epsilon, CD3 gamma, CD3 delta, TCR alpha. TCR beta, TCR gamma, or TCR delta; and (b) an antigen binding domain; wherein the TCR subunit and the antigen binding domain are operatively linked; wherein the TFP functionally incorporates into a TCR when expressed in a T cell. In some embodiments, the intracellular signaling domain comprises a stimulatory domain, e.g., from CD3 epsilon, CD3 gamma, or CD3 delta.

### Certain Terminology

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the present disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

“Major histocompatibility complex (MHC) molecules are typically bound by TCRs as part of peptide:MHC complex. The MHC molecule may be an MHC class I or II molecule. The complex may be on the surface of an antigen presenting cell, such as a dendritic cell or a B cell, or any other cell, including cancer cells, or it may be immobilized by, for example, coating on to a bead or plate.

An “intracellular signaling domain,” as the term is used herein, refers to an intracellular portion of a molecule. The intracellular signaling domain generates a signal that promotes an immune effector function of the TFP containing cell, e.g., a modified T-T cell. Examples of immune effector function, e.g., in a modified T-T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that can be used for an efficient immune response. Costimulatory molecules include but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” refers to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs. A camelid “V_{H}H” domain is a heavy chain comprising a single variable antibody domain.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample or might be macromolecule besides a polypeptide. Such a biological sample can include, but is not limited to a tissue sample, a tumor sample, a cell or a fluid with other biological components.

As used herein, the term “CD19” refers to the Cluster of Differentiation 19 protein, which is an antigenic determinant detectable on B cell leukemia precursor cells, other malignant B cells and most cells of the normal B cell lineage.

As used herein, the term “BCMA” refers to the B-cell maturation antigen also known as tumor necrosis factor receptor superfamily member 17 (TNFRSF17) and Cluster of Differentiation 269 protein (CD269) is a protein that in humans is encoded by the TNFRSF17 gene. TNFRSF17 is a cell surface receptor of the TNF receptor superfamily which recognizes B-cell activating factor (BAFF) (see, e.g., Laabi et al., EMBO 11 (11): 3897-904 (1992). This receptor is expressed in mature B lymphocytes and may be important for B-cell development and autoimmune response.

As used herein, the term “CD16” (also known as FcγRIII) refers to a cluster of differentiation molecule found on the surface of natural killer cells, neutrophil polymorphonuclear leukocytes, monocytes and macrophages. CD16 has been identified as Fc receptors FcγRIIIa (CD16a) and FcγRIIIb (CD16b), which participate in signal transduction. CD16 is a molecule of the immunoglobulin superfamily (IgSF) involved in antibody-dependent cellular cytotoxicity (ADCC).

“NKG2D,” as used herein, refers to a transmembrane protein belonging to the CD94/NKG2 family of C-type lectin-like receptors. In humans, NKG2D is expressed by NK cells, γδ T cells and CD8+αβ T cells. NKG2D recognizes induced-self proteins from MIC and RAET1/ULBP families which appear on the surface of stressed, malignant transformed, and infected cells.

Mesothelin (MSLN) refers to a tumor differentiation antigen that is normally present on the mesothelial cells lining the pleura, peritoneum and pericardium. Mesothelin is over expressed in several human tumors, including mesothelioma and ovarian and pancreatic adenocarcinoma.

Tyrosine-protein kinase transmembrane receptor ROR1, also known as neurotrophic tyrosine kinase, receptor-related 1 (NTRKR1) is a member of the receptor tyrosine kinase-like orphan receptor (ROR) family. It plays a role in metastasis of cancer.

The term “MUC16”, also known as “mucin 16, cell-surface associated” or “ovarian cancer-related tumor marker CA125” is a membrane-tethered mucin that contains an extracellular domain at its amino terminus, a large tandem repeat domain, and a transmembrane domain with a short cytoplasmic domain. Products of this gene have been used as a marker for different cancers, with higher expression levels associated with poorer outcomes.

The term “CD22,” also known as sialic acid binding Ig-like lectin 2, SIGLEC-2, T cell surface antigen leu-14, and B cell receptor CD22, is a protein that mediates B cell/B cell interactions, and is thought to be involved in the localization of B cells in lymphoid tissues, and is associated with diseases including refractory hematologic cancer and hairy cell leukemia. A fully human anti-CD22 monoclonal antibody (“M971”) suitable for use with the methods disclosed herein is described, e.g., in Xiao et al., MAbs. 2009 May-June; 1(3): 297-303.

The “CD79α” and “CD79β” genes encode proteins that make up the B lymphocyte antigen receptor, a multimeric complex that includes the antigen-specific component, surface immunoglobulin (Ig). Surface Ig non-covalently associates with two other proteins, Ig-alpha and Ig-beta (encoded by CD79α and its paralog CD79β, respectively) which are necessary for expression and function of the B-cell antigen receptor. Functional disruption of this complex can lead to, e.g., human B-cell chronic lymphocytic leukemias.

B cell activating factor, or “BAFF” is a cytokine that belongs to the tumor necrosis factor (TNF) ligand family. This cytokine is a ligand for receptors TNFRSF13B/TACI, TNFRSF17/BCMA, and TNFRSF13C/BAFF-R. This cytokine is expressed in B cell lineage cells, and acts as a potent B cell activator. It has been also shown to play an important role in the proliferation and differentiation of B cells.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

The term “autologous” refers to any material derived from the same individual to whom it is later to be re-introduced into the individual.

The term “allogeneic” or, alternatively, “allogenic,” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “functional disruption” refers to a physical or biochemical change to a specific (e.g., target) nucleic acid (e.g., gene, RNA transcript, of protein encoded thereby) that prevents its normal expression and/or behavior in the cell. In one embodiment, a functional disruption refers to a modification of the gene via a gene editing method. In one embodiment, a functional disruption prevents expression of a target gene (e.g., an endogenous gene).

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the present disclosure by standard techniques known in the art, such as site-directed mutagenesis and PCR-mediated mutagenesis. Conservative amino acid substitutions are ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the present disclosure can be replaced with other amino acid residues from the same side chain family and the altered TFP can be tested using the functional assays described herein.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide can contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, that can be used to initiate the specific transcription of a polynucleotide sequence.

The term “promoter/regulatory sequence” refers to a nucleic acid sequence which is used for expression of a gene product operably linked to the promoter/regulatory sequence. In some instances, this sequence may be the core promoter sequence and in other instances, this sequence may also include an enhancer sequence and other regulatory elements which are used for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the present disclosure are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are used for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., CD19) present in a sample, but which does not necessarily and substantially recognize or bind other molecules in the sample.

As used herein, the term “meganuclease” refers to an endonuclease that binds double-stranded DNA at a recognition sequence that is greater than 12 base pairs. Preferably, the recognition sequence for a meganuclease of the present disclosure is 22 base pairs. A meganuclease can be an endonuclease that is derived from I-Crel and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art (e.g., WO 2007/047859). A meganuclease as used herein binds to double-stranded DNA as a heterodimer or as a “single-chain meganuclease” in which a pair of DNA-binding domains are joined into a single polypeptide using a peptide linker. The term “homing endonuclease” is synonymous with the term “meganuclease.” Meganucleases of the present disclosure are substantially non-toxic when expressed in cells, particularly in human T cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising 16-22 TAL domain repeats fused to any portion of the Fokl nuclease domain.

As used herein, the term “Compact TALEN” refers to an endonuclease comprising a DNA-binding domain with 16-22 TAL domain repeats fused in any orientation to any catalytically active portion of nuclease domain of the I-Tevl homing endonuclease.

As used herein, the term “CRISPR” refers to a caspase-based endonuclease comprising a caspase, such as Cas9, and a guide RNA that directs DNA cleavage of the caspase by hybridizing to a recognition site in the genomic DNA.

As used herein, the term “megaTAL” refers to a single-chain nuclease comprising a transcription activator-like effector (TALE) DNA binding domain with an engineered, sequence-specific homing endonuclease.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### DESCRIPTION

Disclosed herein, in some embodiments, are recombinant nucleic acids comprising a sequence encoding a TFP, a CAR, a TCR or combination thereof. In more preferred embodiments, provided herein are circRNAs comprising a sequence encoding a TFP, a CAR, a TCR or combination thereof. According to one aspect, provided herein are transfer vector comprising a sequence encoding a TFP, a CAR, a TCR or combination thereof.

Provided herein are compositions of matter and methods of use for the treatment of a disease such as cancer, using modified human immune cells comprising a T cell receptor (TCR) fusion protein. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

### T Cell Receptor (TCR) Fusion Proteins (TFP)

The present disclosure encompasses recombinant nucleic acid constructs encoding TFPs, wherein the TFP comprises a binding domain, e.g., an antigen binding domain, e.g., comprising an antibody fragment or ligand binding domain, that binds specifically to a tumor associated antigen (TAA), e.g., a human TAA, wherein the sequence of the binding domain is contiguous with and in the same reading frame as a nucleic acid sequence encoding a TCR subunit or portion thereof.

In one aspect, the TFP of the present disclosure comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the present disclosure include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

In one aspect, the portion of the TFP comprising the antigen binding domain comprises an antigen binding domain that targets a tumor associated antigen, e.g., a human tumor associated antigen. In some embodiments the TAA is CD19, CD20, CD22, BCMA, MSLN, IL13Ra2, EGFRvIII, MUC16, ROR1, HER2, BAFF, BAFF receptor, PD-L1, CD79b, or PSMA. In one embodiment, the antigen binding domain comprises an antibody or fragment thereof. In an aspect, the portion of the TFP comprising the antigen binding domain comprises a ligand binding domain such as NKG2D or CD16. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise, a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-TAA binding domain comprises an scFv having one or more (e.g., all three) light chain complementary determining region 1 (LC CDR1), light chain complementary determining region 2 (LC CDR2), and light chain complementary determining region 3 (LC CDR3) of a humanized or human anti-TAA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-TAA binding domain described herein, e.g., a humanized or human anti-TAA binding domain comprising one or more, e.g., all three, LC CDRs and one or more, e.g., all three, HC CDRs. In one embodiment, the humanized or human anti-TAA binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized or human anti-TAA binding domain described herein, e.g., the humanized or human anti-TAA binding domain has two variable heavy chain regions, each comprising a HC CDR1, a HC CDR2 and a HC CDR3 described herein. In one embodiment, the humanized or human anti-TAA binding domain comprises a humanized or human light chain variable region described herein and/or a humanized or human heavy chain variable region described herein. In one embodiment, the humanized or human anti-TAA binding domain comprises a humanized heavy chain variable region described herein, e.g., at least two humanized or human heavy chain variable regions described herein. In one embodiment, the anti-TAA binding domain is a scFv comprising a light chain and a heavy chain of an amino acid sequence provided herein. In an embodiment, the anti-TAA binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-TAA binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-TAA binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 10, e.g., 2 to 4. In some instances, the linker sequence comprises a short linker (SL) sequence. In some instances, the short linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No.

US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the present disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human CD20, CD22, BCMA, MSLN, IL13Ra2, EphA2, NY-ESO-1, PSMA, BAFF, EGFRvIII, MUC16, MUC1, ROR1, or CD19. In some embodiments, a humanized antibody or antibody fragment may have improved affinity and/or specificity of binding to human CD19, CD20, CD22, BCMA, MSLN, IL13Ra2, EphA2, NY-ESO-1, PSMA, BAFF, EGFRvIII, MUC16, MUC1, or ROR1.

In one aspect, the anti-TAA binding domain is characterized by particular functional features or properties of an antibody or antibody fragment. For example, in one aspect, the portion of a TFP composition of the present disclosure that comprises an antigen binding domain specifically binds a tumor associated antigen from the group comprising human CD19, human BCMA, human MSLN, human CD20, human CD22, human ROR1, human BAFF, human MUC16, human EphA2, human NY-ESO-1, human PSMA, human IL13Ra2, and human EGFRvIII. In one aspect, the antigen binding domain has the same or a similar binding specificity to human CD19 as described in Nicholson et al. Mol. Immun 34 (16-17): 1157-1165 (1997). In one aspect, the present disclosure relates to an antigen binding domain comprising an antibody or antibody fragment, wherein the antibody binding domain specifically binds to a TAA protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-TAA binding domain is a fragment, e.g., a single chain variable fragment (scFv). In one aspect, the anti-TAA binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof of the present disclosure binds a TAA protein with wild-type or enhanced affinity. In one aspect, the anti-TAA binding domain is a single domain (sdAb) antibody or fragment thereof. In another aspect, the anti-TAA binding domain is a VHH.

Also provided herein are methods for obtaining an antibody antigen binding domain specific for a target antigen (e.g., a tumor associated antigen (TAA) such as CD19, BCMA, MSLN, or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding can bring the two variable regions together to form a functional epitope binding site. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Patent Application Publication Nos. 20050100543 and 20050175606, U.S. Pat. No. 7,695,936, and PCT publication Nos. WO2006/020258 and WO2007/024715, each of which is incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

### Stability and Mutations

The stability of an anti-TAA binding domain, e.g., scFv molecules (e.g., soluble scFv) can be evaluated in reference to the biophysical properties (e.g., thermal stability) of a conventional control scFv molecule or a full-length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-TAA binding domain is subsequently conferred to the entire CD19-TFP construct, leading to improved therapeutic properties of the anti-TAA TFP construct. The thermal stability of the anti-TAA binding domain, e.g., scFv can be improved by at least about 2° C. or 3° C. as compared to a conventional antibody. In one embodiment, the anti-TAA binding domain, e.g., scFv has a 1° C. improved thermal stability as compared to a conventional antibody. In another embodiment, the anti-TAA binding domain, e.g., scFv or sdAb, has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described in more detail below.

Mutations in scFv (arising through humanization or direct mutagenesis of the soluble scFv) alter the stability of the scFv and improve the overall stability of the scFv and the anti-TAA TFP construct. Stability of the humanized scFv is compared against the murine scFv using measurements such as T_{M}, temperature denaturation and temperature aggregation. In one embodiment, the anti-TAA binding domain, e.g., a scFv, comprises at least one mutation arising from the humanization process such that the mutated scFv confers improved stability to the anti-TAA TFP construct. In another embodiment, the anti-TAA binding domain, e.g., scFv comprises at least 1, 2, 3, 4, 5, 6, 7, 8, 9, 10 mutations arising from the humanization process such that the mutated scFv confers improved stability to the TAA-TFP construct.

In one aspect, the antigen binding domain of the TFP comprises an amino acid sequence that is homologous to an antigen binding domain amino acid sequence described herein, and the antigen binding domain retains the desired functional properties of the anti-TAA antibody fragments described herein. In one specific aspect, the TFP composition of the present disclosure comprises an antibody fragment. In a further aspect, that antibody fragment comprises a scFv. In a further embodiment, the antibody comprises a V_{H} domain.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the present disclosure comprises an antibody fragment. In a further aspect, that antibody fragment comprises a scFv.

It will be understood by one of ordinary skill in the art that the antibody or antibody fragment of the present disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present disclosure contemplates modifications of the starting antibody or fragment (e.g., scFv) amino acid sequence that generate functionally equivalent molecules. For example, the V_{H} or V_{L} of an anti-TAA binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-CD19 binding domain, e.g., scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this present disclosure may include at least the extracellular region(s) of e.g., the alpha, beta, gamma, or delta chain of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. A transmembrane domain of particular use in this present disclosure may include at least the transmembrane region(s) of e.g., the alpha, beta, gamma, or delta chain of the T cell receptor, CD28, CD3 epsilon, CD3 delta, CD3 gamma, CD3 zeta, CD45, CD4, CD5, CD8, CD9, CD16, CD22, CD33, CD37, CD64, CD80, CD86, CD134, CD137, CD154.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. In some cases, the linker may be at least about 5, 6, 7, 8, 9, 10, 11, 12, 13, 14, 15, 16, 17, 18, 19, 20, or more in length. A glycine-serine doublet provides a particularly suitable linker. For example, in one aspect, the linker comprises the amino acid sequence of GGGGSGGGGS (SEQ ID NO: 3). In some embodiments, the linker is encoded by a nucleotide sequence of GGTGGCGGAGGTTCTGGAGGTGGAGGTTCC (SEQ ID NO: 4).

### Cytoplasmic Domain

The cytoplasmic domain of the TFP can include an intracellular domain. In some embodiments, the intracellular domain is from CD3 gamma, CD3 delta, CD3 epsilon, TCR alpha, TCR beta, TCR gamma, or TCR delta. In some embodiments, the intracellular domain comprises a signaling domain, if the TFP contains CD3 gamma, delta or epsilon polypeptides; TCR alpha, TCR beta, TCR gamma, and TCR delta subunits generally have short (e.g., 1-19 amino acids in length) intracellular domains and are generally lacking in a signaling domain. An intracellular signaling domain is generally responsible for activation of at least one of the normal effector functions of the immune cell in which the TFP has been introduced. While the intracellular domains of TCR alpha, TCR beta, TCR gamma, and TCR delta do not have signaling domains, they are able to recruit proteins having a primary intracellular signaling domain described herein, e.g., CD3 zeta, which functions as an intracellular signaling domain. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the present disclosure include the cytoplasmic sequences of the T cell receptor (TCR) and co-receptors are able to act in concert to initiate signal transduction following antigen receptor engagement, as well as any derivative or variant of these sequences and any recombinant sequence that has the same functional capability.

It is known that signals generated through the TCR alone may be insufficient for full activation of naive T cells and that a secondary and/or costimulatory signal may be used. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the present disclosure include those of CD3 zeta, FcR gamma, FcR beta, CD3 gamma, CD3 delta, CD3 epsilon, CD5, CD22, CD79a, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise a CD3 signaling domain, e.g., CD3 epsilon, CD3 delta, CD3 gamma, or CD3 zeta, by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that can lead to an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the vector or circular RNA encoding the TFP or CAR or TCR is expressed in a cell in vitro. In another aspect, the TFP or CAR or TCR is delivered to a cell in vivo. In another aspect, the TFP or CAR or TCR is delivered to a cell ex-vivo.

In one aspect, a TFP-expressing cell described herein can further comprise a second TFP, e.g., a second TFP that includes a different antigen binding domain, e.g., to the same target (the same TAA) or a different target (e.g., CD123). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a V_{HH}.

In another aspect, the TFP-expressing cell described herein can further express another agent, e.g., an agent which enhances the activity of a modified human immune cell. For example, in one embodiment, the agent can be an agent which inhibits an inhibitory molecule. Inhibitory molecules, e.g., PD1, can, in some embodiments, decrease the ability of a modified human immune cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2, have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094) Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-TAA TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having an anti-TAA binding domain described herein, and a second cell expressing a TFP having a different anti-TAA binding domain, e.g., an anti-TAA binding domain described herein that differs from the anti-TAA binding domain in the TFP expressed by the first cell. As another example, the population of TFP-expressing cells can include a first cell expressing a TFP that includes an anti-TAA binding domain, e.g., as described herein, and a second cell expressing a TFP that includes an antigen binding domain to a target other than CD19 or BCMA (e.g., another tumor-associated antigen).

In another aspect, the present disclosure provides a population of cells wherein at least one cell in the population expresses a TFP having an anti-TAA domain described herein, and a second cell expressing another agent, e.g., an agent which enhances the activity of a modified human immune cell. For example, in one embodiment, the agent can be an agent which inhibits an inhibitory molecule. Inhibitory molecules, e.g., can, in some embodiments, decrease the ability of a modified human immune cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Circular RNA (circRNA)

Disclosed herein are methods for producing in vitro or in vivo transcribed RNA encoding TFPs, CARs, TCRs or combination thereof. In preferred embodiments, the RNA is circRNA. In some embodiments, circRNA is exogenous. In other embodiments, circRNA is endogenous. In other embodiments, circRNAs with an internal ribosomal entry site (IRES) can be translated in vitro or ex vivo.

Circular RNAs (circRNAs) are a class of single-stranded RNAs with a contiguous structure that have enhanced stability and a lack of end motifs necessary for interaction with various cellular proteins. CircRNAs are 3-5′ covalently closed RNA rings, and circRNAs do not display Cap or poly(A) tails. CircRNAs lack the free ends necessary for exonuclease-mediated degradation, rendering them resistant to several mechanisms of RNA turnover and granting them extended lifespans as compared to their linear mRNA counterparts. For this reason, circularization may allow for the stabilization of mRNAs that generally suffer from short half-lives and may therefore improve the overall efficacy of mRNA in a variety of applications. Moreover, when transfected into a cell, e.g., a T cell, circRNA may have reduced immunogenicity relative to other forms of RNA, e.g., shRNA or double stranded RNA. CircRNAs are produced by the process of splicing, and circularization occurs using conventional splice sites mostly at annotated exon boundaries (Starke et al., 2015; Szabo et al., 2015). For circularization, splice sites are used in reverse: downstream splice donors are “backspliced” to upstream splice acceptors (see Jeck and Sharpless, 2014; Barrett and Salzman, 2016; Szabo and Salzman, 2016; Holdt et al., 2018 for review).

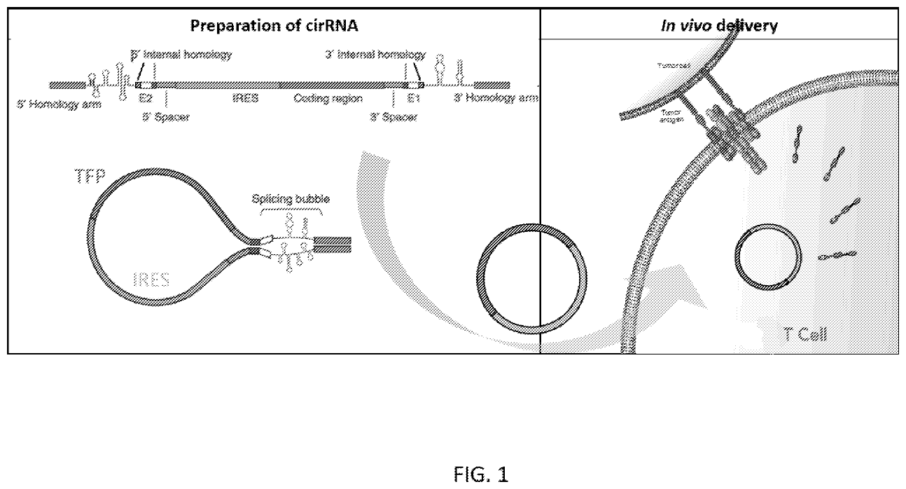

To generate circRNAs that can subsequently be transferred into cells, in vitro production of circRNAs with autocatalytic-splicing introns can be programmed (FIG. 1). A method for generating circRNA can involve in vitro transcription (IVT) of a precursor linear RNA template with specially designed primers. Three general strategies have been reported so far for RNA circularization: chemical methods using cyanogen bromide or a similar condensing agent, enzymatic methods using RNA or DNA ligases, and ribozymatic methods using self-splicing introns. In preferred embodiments, precursor RNA is synthesized by run-off transcription and then heated in the presence of magnesium ions and GTP to promote circularization. RNA so produced can efficiently transfect different kinds of cells. In one aspect, the template includes sequences for the TFP, CAR, and TCR, or combination thereof.

The group I intron of phage T4 thymidylate synthase (td) gene is well characterized to circularize while the exons linearly splice together (Chandry and Bel-fort, 1987; Ford and Ares, 1994; Perriman and Ares, 1998). When the td intron order is permuted flanking any exon sequence, the exon is circularized via two autocatalytic transesterification reactions (Ford and Ares, 1994; Puttaraju and Been, 1995). In preferred embodiments, the group I intron of phage T4 thymidylate synthase (td) gene is used to generate exogenous circRNA.

In some exemplary embodiments, a ribozymatic method utilizing a permuted group I catalytic intron is used. This method may be more applicable to long RNA circularization and may need only the addition of GTP and Mg2+ as cofactors. This permuted intron-exon (PIE) splicing strategy consists of fused partial exons flanked by half-intron sequences. In vitro, these constructs undergo the double transesterification reactions characteristic of group I catalytic introns, but because the exons are fused, they are excised as covalently 5′ and 3′linked circles.

In one aspect, disclosed herein is a sequence containing a full-length encephalomyocarditis virus (such as EMCV) IRES, a gene encoding a TFP, a CAR, a TCR or combination thereof, two short regions corresponding to exon fragments (E1 and E2), and of the PIE construct between the 3′ and 5′ introns of the permuted group I catalytic intron in the thymidylate synthase (Td) gene of the T4 phage or the permuted group I catalytic intron in the pre-tRNA gene of Anabaena. In more preferred embodiments, the mentioned sequence further comprises complementary ‘homology arms’ placed at the 5′ and 3′ends of the precursor RNA with the aim of bringing the 5′ and 3′ splice sites into proximity of one another. To ensure that the major splicing product was circular, the splicing reaction can be treated with RNase R.

In one aspect the anti-TAA TFP is encoded by a circRNA. In one aspect the circRNA encoding the anti-TAA TFP is introduced into a T cell for production of a TFP-T cell. In one embodiment, the in vitro transcribed RNA TFP can be introduced to a cell as a form of transient transfection.

In some aspects, linear precursor RNA is produced by in vitro transcription using a polymerase chain reaction (PCR)-generated template. DNA of interest from any source can be directly converted by PCR into a template for in vitro RNA synthesis using appropriate primers and buffer and RNA polymerase and nucleotides modified or not. The source of the DNA can be, for example, genomic DNA, plasmid DNA, phage DNA, cDNA, digested DNA, synthetic DNA sequence or any other appropriate source of DNA. The desired template for in vitro transcription is a TFP of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In some exemplary embodiments, PCR is used to generate a template for in vitro transcription of linear precursor RNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5′ to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths to achieve optimal RNA stability or/and translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA whereas protein binding motif can increase the stability of mRNA and circRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts but may not be needed for all RNAs to enable efficient translation. Many mRNAs known in the art may comprise Kozak sequences. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of linear precursor RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the RNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps can also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun, 330:958-966 (2005)).

The RNAs (e.g. circRNA) are produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector®-II (Amaxa Biosystems, Cologne, Germany)), ECM 830 (BTX) (Harvard Instruments, Boston, Mass.), Neon Transfection System (ThermoFisher), Cell squeezing (SQZ Biotechnologies) or the Gene Pulser® II (BioRad, Denver, Colo.), Multiporator® (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Choice of PIE Based on Permuted Intron-Exon (PIE) Splicing Strategy

CircRNAs are generally formed from longer-than-average exons and are normally flanked by longer-than-average introns in their associated pre-mRNAs (Jeck et al., 2013; Salzman et al., 2012); such introns are enriched for complementary ALU elements thought to play a role in the biogenesis of many circRNAs in humans (Jeck et al., 2013). Therefore, the permuted intron-exon (PIE) splicing strategy consists of fused partial exons flanked by half-intron sequences [Wesselhoeft et. al., Nat. Commun., 9:26-29., 2018]. CircRNAs can be predicted based on the sequence composition of their flanking introns.

The group I intron of phage T4 thymidylate synthase (td) gene is well characterized to circularize while the exons linearly splice together (Chandry and Bel-fort, 1987; Ford and Ares, 1994; Perriman and Ares, 1998). When the td intron order is permuted (50 half placed at the 30 position and vice versa) flanking any exon sequence, the exon is circularized via two autocatalytic transesterification reactions (Ford and Ares, 1994; Puttaraju and Been, 1995). In preferred embodiments, self-splicing introns [Wesselhoeft et. al., Nat. Commun., 9:26-29., 2018] are used in the design of disclosed circRNAs. In more preferred embodiments, the group I intron is used in the design of disclosed circRNAs to promote self-splicing and circularization.

### IRES

Cap-independent translation is an alternative means of translation initiation in eukaryotes that depends on the presence of particular elements that induce internal initiation, such as an internal ribosome entry site (IRES). IRES sequences were first reported in viral RNAs and bind to eukaryotic ribosomes when internal to the RNA Chen and Sarnow, 1995; Perriman and Ares, 1998). In principle, the key feature of IRES-driven translation is its 5′-end independence, rather than cap-independence.

Unlike linear mRNA, circRNA relies heavily on folded RNA structures, including the permuted group I intron and IRES, for splicing and translation. For example, secondary structures proximal to the IRES, including within the coding region that directly follows the IRES, have the potential to disrupt IRES folding and translation initiation, affecting the circularization efficiency. Therefore, different kind of IRES sequences should be chosen and tested depending on the choice of PIE.

In some embodiments, IRES sequences are chosen from the group comprising viral sequences such as AMPV, CSFV, CVB3, EMCV, EV71, HAV, HRV2, HTLV, and PV (poliovirus). In preferred embodiments, IRES sequences are chosen from Coxsackievirus B3 (CVB3). In other preferred embodiments, IRES sequences are chosen from encephalomyocarditis virus (EMCV).

### Homology Arms to Improve Circularization Efficiency

‘Homology arms’ are complementary sequences placed at the 5′ and 3′ ends of the precursor linear RNA with the aim of bringing the 5′ and 3′ splice sites into proximity of one another. Without homology arms, no base pairing is predicted to occur between the ends of the precursor RNA. The addition of the homology arms has been reported to result in increased splicing efficiency as well as circularization efficiency [Wesselhoeft et. al., Nat. Commun., 9:26-29., 2018]. RNAFold WebServer, a site provided by the University of Vienna, predicts secondary structures of single stranded RNA or DNA sequences. Predictions of precursor RNA secondary structure inform the design homology arms. In some embodiments, RNAfold is used to test sequence variants for homology arm sequences. In some embodiments, the sequence length is chosen in the range of 20 to 150 nucleotides. In some embodiments, more than one homology arm sequences are present at the 5′ and 3′ ends of the precursor linear RNA.

In one aspect, disclosed herein is a precursor linear RNA sequence containing a full-length encephalomyocarditis virus (such as EMCV) IRES, a gene encoding a TFP, a CAR, a TCR or combination thereof, two short regions corresponding to exon fragments (E1 and E2), and of the PIE construct between the 3′ and 5′ introns of the permuted group I catalytic intron in the thymidylate synthase (Td) gene of the T4 phage or the permuted group I catalytic intron in the pre-tRNA gene of Anabaena. In more preferred embodiments, the mentioned sequence further comprises complementary homology arms' placed at the 5′ and 3′ ends of the precursor RNA with the aim of bringing the 5′ and 3′ splice sites into proximity of one another. In another aspect, the resulting circRNA self-spliced from the mentioned linear RNA sequence comprises the region between two exon fragments (E1 and E2) comprising IRES, a gene encoding a TFP, a CAR, a TCR or combination thereof, spacer sequences (optional), and homology arms (optional).

### Spacer Sequences

In order to further improve the efficiency of circRNA generation from the self-splicing precursor RNA, further optimization may be performed [Wesselhoeft et. al., Nat. Commun., 9:26-29., 2018]. Sequences within the IRES may interfere with the folding of the splicing ribozyme, either proximally at the 3′ splice site or distally at the 5′ splice site through long-distance contacts since the 3′ PIE splice site is proximal to the IRES, and because both sequences are highly structured. The addition of spacer sequences has been predicted to permit splicing with increased splicing efficiency [Wesselhoeft et. al., Nat. Commun., 9:26-29., 2018].

In some embodiments, RNAfold is used to test sequence variants for spacer sequences. In some embodiments, a sequence length is chosen to be in the range of 20 to 150 nucleotides.

### Recombinant Nucleic Acid Encoding a TFP

Disclosed herein, in some embodiments, are recombinant nucleic acids comprising a sequence encoding a T cell receptor (TCR) fusion protein (TFP) comprising (i) a TCR subunit comprising (1) at least a portion of a TCR extracellular domain, (2) a transmembrane domain, and (3) an intracellular domain from an intracellular signaling domain of CD3 epsilon, CD3 gamma, CD3 delta, TCR alpha, TCR beta, TCR delta, or TCR gamma, and (ii) an antigen binding domain; wherein the TCR subunit and the antigen binding domain are operatively linked, and wherein the TFP functionally incorporates into a TCR complex when expressed in a T cell. In some embodiments, the intracellular domain comprises a stimulatory domain, e.g., from CD3 epsilon, CD3 gamma, or CD3 delta. In some embodiments, the recombinant nucleic acid further comprises a sequence encoding a TCR constant domain. In some embodiments, the TCR constant domain is a TCR alpha constant domain, a TCR beta constant domain or a TCR alpha constant domain and a TCR beta constant domain. In some embodiments, the TCR constant domain is a TCR gamma constant domain, a TCR delta constant domain or a TCR gamma constant domain and a TCR delta constant domain.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the antibody domain, the antigen domain or the binding ligand or fragment thereof are operatively linked by a linker sequence. In some instances, the linker sequence comprises (G4S)n, wherein n=1 to 4.

In some instances, the transmembrane domain is a TCR transmembrane domain from CD3 epsilon, CD3 gamma, CD3 delta, TCR alpha, TCR beta, TCR gamma, or TCR delta. In some instances, the intracellular domain is derived from only CD3 epsilon, only CD3 gamma, only CD3 delta, only TCR alpha, only TCR beta, only TCR gamma, or only TCR delta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR gamma chain, a TCR delta chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain of CD3 epsilon, CD3 gamma, CD3 delta, TCR alpha, TCR beta, TCR gamma, or TCR delta. In some embodiments, the intracellular domain comprises a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof; (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof; or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the TFP, the TCR gamma constant domain, the TCR delta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR delta, CD3 epsilon, CD3 gamma, CD3 delta, or a combination thereof; (b) the TCR constant domain is a TCR delta constant domain and the TFP functionally integrates into a TCR complex comprising an endogenous subunit of TCR gamma, CD3 epsilon, CD3 gamma, CD3 delta, or a combination thereof; or (c) the TCR constant domain is a TCR gamma constant domain and a TCR delta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some embodiments, the antigen binding domain comprises an antibody or antibody fragment. In some embodiments, the antibody or antibody fragment, is murine, camelid, alpaca, human or humanized. In some instances, the antibody fragment is a scFv, a single domain antibody domain, a VHH, a V_{H} domain or a V_{L} domain. In some instances, the antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-PD-1 binding domain, anti-BAFF or BAFF receptor binding domain, and anti-ROR-1 binding domain.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a anhydrohexitol nucleic acid (HNA), a morpholino, a methylphosphonate nucleotide, a thiolphosphonate nucleotide, and a 2′-fluoro N3-P5′-phosphoramidite.

In some embodiments, the nucleic acid is RNA. In some embodiments, the RNA does not comprise m6A. In some embodiments, the RNA comprises less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% m6A.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

Disclosed herein, in some embodiments, are recombinant nucleic acids comprising a sequence encoding a T cell receptor (TCR) fusion protein (TFP) comprising (i) a TCR subunit comprising (1) at least a portion of a TCR extracellular domain, (2) a transmembrane domain, and (3) an intracellular domain, and (ii) a binding ligand or a fragment thereof that is capable of binding to an antibody or fragment thereof; wherein the TCR subunit and the binding ligand or fragment thereof are operatively linked, and wherein the TFP functionally incorporates into a TCR complex when expressed in a T cell. In some embodiments, the recombinant nucleic acid further comprises a sequence encoding a TCR constant domain. In some embodiments, the TCR constant domain is a TCR alpha constant domain, a TCR beta constant domain or a TCR alpha constant domain and a TCR beta constant domain. In some embodiments, the TCR constant domain is a TCR gamma constant domain, a TCR delta constant domain or a TCR gamma constant domain and a TCR delta constant domain. In some embodiments, the intracellular domain comprises an intracellular domain of TCR alpha or TCR beta. In some embodiments, the intracellular domain comprises a stimulatory domain from an intracellular signaling domain of CD3 epsilon, CD3 gamma, or CD3 delta. In some instances, the binding ligand is capable of binding an Fc domain of the antibody. In some instances, the binding ligand is capable of selectively binding an IgG1 antibody. In some instances, the binding ligand is capable of specifically binding an IgG1 antibody. In some instances, the antibody or fragment thereof binds to a cell surface antigen. In some instances, the antibody or fragment thereof binds to a cell surface antigen on the surface of a tumor cell. In some instances, the binding ligand comprises a monomer, a dimer, a trimer, a tetramer, a pentamer, a hexamer, a heptamer, an octomer, a nonamer, or a decamer. In some instances, the binding ligand does not comprise an antibody or fragment thereof. In some instances, the binding ligand comprises a CD16 polypeptide or fragment thereof. In some instances, the binding ligand comprises a CD16-binding polypeptide. In some instances, the binding ligand is human or humanized. In some instances, the recombinant nucleic acid further comprises a nucleic acid sequence encoding an antibody or fragment thereof capable of being bound by the binding ligand. In some instances, the antibody or fragment thereof is capable of being secreted from a cell.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the binding ligand or fragment thereof are operatively linked by a linker sequence. In some instances, the linker sequence comprises (G₄S)n, wherein n=1 to 4.

In some instances, the transmembrane domain is a TCR transmembrane domain from CD3 epsilon, CD3 gamma, CD3 delta, TCR alpha, TCR beta, TCR gamma, or TCR delta. In some instances, the intracellular domain is derived from only CD3 epsilon, only CD3 gamma, only CD3 delta, only TCR alpha, only TCR beta, only TCR gamma, or only TCR delta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR gamma chain, a TCR delta chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain. In some embodiments, the intracellular domain comprises an intracellular domain of TCR alpha, TCR beta, TCR gamma, or TCR delta, or an amino acid sequence having at least one modification thereto. In some embodiments the TCR intracellular domain comprises a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof; (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof; or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the TFP, the TCR gamma constant domain, the TCR delta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR delta, CD3 epsilon, CD3 gamma, CD3 delta, or a combination thereof; (b) the TCR constant domain is a TCR delta constant domain and the TFP functionally integrates into a TCR complex comprising an endogenous subunit of TCR gamma, CD3 epsilon, CD3 gamma, CD3 delta, or a combination thereof; or (c) the TCR constant domain is a TCR galla constant domain and a TCR delta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain. Alternatively, the recombinant nucleic acid comprises a sequence encoding a TCR gamma or TCR delta domain, e.g., a transmembrane domain.

Disclosed herein, in some embodiments, are recombinant nucleic acids comprising a sequence encoding a T cell receptor (TCR) fusion protein (TFP) comprising (i) a TCR subunit comprising (1) at least a portion of a TCR extracellular domain, (2) a transmembrane domain, and (3) an intracellular domain, and (ii) an antigen domain comprising a ligand or a fragment thereof that binds to a receptor or polypeptide expressed on a surface of a cell; wherein the TCR subunit and the antigen domain are operatively linked, and wherein the TFP functionally incorporates into a TCR complex when expressed in a T cell. In some embodiments, the recombinant nucleic acid further comprises a sequence encoding a TCR constant domain. In some embodiments, the TCR constant domain is a TCR alpha constant domain, a TCR beta constant domain or a TCR alpha constant domain and a TCR beta constant domain. In some embodiments, the TCR constant domain is a TCR gamma constant domain, a TCR delta constant domain or a TCR gamma constant domain and a TCR delta constant domain. In some embodiments, the intracellular domain comprises an intracellular domain from TCR alpha, TCR beta, TCR gamma, or TCR delta. In some embodiments, the intracellular domain comprises a stimulatory domain from an intracellular signaling domain of CD3 delta, CD3 epsilon, or CD3 gamma. In some instances, the antigen domain comprises a ligand. In some instances, the ligand binds to the receptor of a cell. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MHC class I-related glycoprotein. In some instances, the MHC class I-related glycoprotein is selected from the group consisting of MICA, MICB, RAET1E, RAET1G, ULBP1, ULBP2, ULBP3, ULBP4 and combinations thereof. In some instances, the antigen domain comprises a monomer, a dimer, a trimer, a tetramer, a pentamer, a hexamer, a heptamer, an octomer, a nonamer, or a decamer. In some instances, the antigen domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen domain does not comprise an antibody or fragment thereof. In some instances, the antigen domain does not comprise a variable region. In some instances, the antigen domain does not comprise a CDR. In some instances, the ligand or fragment thereof is a Natural Killer Group 2D (NKG2D) ligand or a fragment thereof.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the antigen domain are operatively linked by a linker sequence. In some instances, the linker sequence comprises (G4S)n, wherein n=1 to 4.

In some instances, the transmembrane domain is a TCR transmembrane domain from CD3 epsilon, CD3 gamma, CD3 delta, TCR alpha, TCR beta, TCR gamma, or TCR delta. In some instances, the intracellular domain is derived from only CD3 epsilon, only CD3 gamma, only CD3 delta, only TCR alpha, only TCR beta, only TCR gamma, or only TCR delta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR gamma chain, a TCR delta chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain. In some embodiments, the intracellular domain comprises an intracellular domain of TCR alpha, TCR beta, TCR gamma, or TCR delta, or an amino acid sequence having at least one modification thereto. In some embodiments the TCR intracellular domain comprises a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof; (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof; or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the TFP, the TCR gamma constant domain, the TCR delta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR delta, CD3 epsilon, CD3 gamma, CD3 delta, or a combination thereof; (b) the TCR constant domain is a TCR delta constant domain and the TFP functionally integrates into a TCR complex comprising an endogenous subunit of TCR gamma, CD3 epsilon, CD3 gamma, CD3 delta, or a combination thereof; or (c) the TCR constant domain is a TCR galla constant domain and a TCR delta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some embodiments, the sequence encoding the TFP and the sequence comprising/encoding the circRNA binding site are on the same nucleic acid molecule. In some embodiments, the extracellular and transmembrane domains of the TCR subunit are derived from TCR alpha, TCR beta, TCR gamma, TCR delta, CD3 gamma, CD3 delta, or CD3 epsilon. In some embodiments, the sequence encoding the antigen binding domain is connected to the sequence encoding the TCR extracellular domain by a linker sequence. In some embodiments, the encoded linker sequence comprises (G₄S)ₙ, wherein G is glycine, S is serine, and n=1 to 4. In some embodiments, the antigen binding domain is an anti-tumor associated antigen (TAA) binding domain. In some embodiments, the anti-TAA binding domain binds to an antigen derived from alpha-actinin-4, ARTC1, BCR-ABL fusion protein (b3a2), B-RAF, CASP-5, CASP-8, beta-catenin, Cdc27, CDK4, CDK12, CDKN2A, CLPP, COA-1, CSNK1A1, dek-can fusion protein, EFTUD2, Elongation factor 2, ETV6-AML1 fusion protein, FLT3-ITD, FNDC3B, FN1, GAS7, GPNMB, HAUS3, HSDL1, LDLR-fucosyltransferase AS fusion protein, HLA-A2d, HLA-A11d, hsp70-2, MART2, MATN, MEL MUM-1f, MUM-2, MUM-3, neo-PAP, Myosin class I, NFYC, OGT, OS-9, p53, pml-RARalpha fusion protein, PPP1R3B, PRDX5, PTPRK, K-ras, N-ras, RBAF600, SIRT2, SNRPD1, SYT-SSX1 or -SSX2 fusion protein, TGF-betaRII, triosephosphate isomerase, BAGE-1, D393-CD20n, Cyclin-A1, GAGE-1, GAGE-2, GAGE-8, GAGE-3, GAGE-4, GAGE-5, GAGE-6, GAGE-7, GnTVf, HERV-K-MEL, KK-LC-1, KM-HN-1, LAGE-1, LY6K, MAGE-AL MAGE-A2, MAGE-A3, MAGE-A4, MAGE-A6, MAGE-A9, MAGE-A10, MAGE-A12 m, MAGE-C1, MAGE-C2, mucink, NA88-A, NY-ESO-1/LAGE-2, SAGE, Sp17, SSX-2, SSX-4, TAG-1, TAG-2, TRAG-3, TRP2-INT2g, XAGE-1b/GAGED2a, CEA, gp100/Pmel17, mammaglobin-A, Melan-A/MART-1, NY-BR-1, OAL PAP, PSA, RAB38/NY-MEL-1, TRP-1/gp75, TRP-2, tyrosinase, adipophilin, AIM-2, ALDH1A1, BCLX (L), BING-4, CALCA, CD45, CD274, CPSF, cyclin D1, DKK1, ENAH (hMena), EpCAM, EphA2, EphA3, EZH2, FGF5, glypican-3, G250/MN/CAIX, HER-2/neu, HLA-DOB, Hepsin, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, Midkine, MMP-2, MMP-7, MUC1, MUC16 (CA125), MUC5AC, p53, PAX5, PBF, PRAME, PSMA, RAGE-1, RGS5, RhoC, RNF43, RU2AS, secernin 1, SOX10, STEAP1, survivin, Telomerase, TPBG, VEGF, ROR-1, EGFRvIII, DLL3, C4.4a, BCMA, MSLN, CD19, CD20, CD22, SAP-1, NKG2D, MAGE, ETA, MUC-16 (CA-125), CEA, AFP, EMP-2, or WT1. In some embodiments, the encoded antigen binding domain comprises an anti-CD19 binding domain, an anti-BCMA binding domain, an anti-mesothelin binding domain, or any combination thereof. In some embodiments, the encoded transmembrane domain comprises a transmembrane domain of a protein selected from the group consisting of a TCR alpha chain, a TCR beta chain, a TCR gamma chain, a TCR delta chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto.

In some instances, the nucleic acid is selected from the group consisting of a DNA and an RNA. In some instances, the nucleic acid is an mRNA. In some instances, the nucleic acid is a linear precursor RNA. In some instances, the nucleic acid is a circular RNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

Further disclosed herein, in some embodiments, are vectors comprising the recombinant nucleic acid disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector. According to yet another aspect, provided herein is a vector comprising a nucleic acid molecule encoding a TFP or CAR or TCR molecule of any of the recombinant nucleic acid provided herein. In some embodiments, the vector is selected from the group consisting of a DNA, a RNA, a plasmid, a lentivirus vector, adenoviral vector, or a retrovirus vector. In some embodiments, the vector further comprises a promoter. In some embodiments, the vector is an in vitro transcribed vector. In some embodiments, a nucleic acid sequence in the vector further comprises a sequence encoding a poly(A) tail.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a nucleic acid of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP or TCR or CAR of the present disclosure is an adenoviral vector (A5/35). In another embodiment, the expression of nucleic acids encoding TFPs can be accomplished using of transposons such as sleeping beauty, crisper, CAS9, and zinc finger nucleases. See below June et al. 2009 Nature Reviews Immunology 9.10: 704-716, is incorporated herein by reference.

According to yet another aspect, provided herein is a cell comprising the recombinant nucleic acid of any one of claims provided herein or the vector of any one of vectors provided herein. In some embodiments, the cell is a human immune cell. In some embodiments, the immune cell is a T cell precursor, e.g., a lymphoblast. In some embodiments, the immune cell is a CD8⁺ or CD4⁺ T cell, a CD8+CD4+ T cell, an NK cell, or an NKT cell. According to yet another aspect, provided herein is a method of making a cell comprising transducing a human immune cell with the recombinant nucleic acid provided herein or the vector provided herein. Also provided herein is a method of providing an anti-tumor immunity in a mammal having a disease comprising administering to the mammal an effective amount of a cell comprising a vector provided herein. Also provided herein is a method of providing an anti-tumor immunity in a mammal having a disease comprising administering to the mammal an effective amount of a cell comprising a nucleic acid molecule encoding a TFP and a circRNA provided herein. Also provided herein is a method of providing an anti-tumor immunity in a mammal having a disease comprising administering to the mammal an effective amount of a cell comprising a nucleic acid molecule having a circRNA binding site provided herein.

Also provided herein is a method of providing an anti-tumor immunity in a mammal having a disease comprising administering to the mammal an effective amount of a delivery vehicle such as a liposome or nanoparticle. In some embodiments, the delivery vehicle comprises a payload of an isolated recombinant nucleic acid encoding a TFP, TCR, or CAR. In some embodiments, the isolated recombinant nucleic acid is a circular RNA. In some embodiments, the circular RNA comprises a targeting moiety.

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the present disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP or CAR or TCR polypeptide or portions thereof, the expression vector to be introduced into a cell or a delivery vehicle can also contain either a selectable marker gene or a reporter gene or both to facilitate identification and selection of expressing cells from the population of cells sought to be transfected or infected through viral vectors. In other aspects, the selectable marker may be carried on a separate piece of nucleic acid and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the nucleic acid has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfer Vectors and Methods for Delivery of Nucleic Acids In Vivo/Ex Vivo

The term “transfer vector” refers to a composition of matter which comprises an isolated nucleic acid and which can be used to deliver the isolated nucleic acid to the interior of a cell. In preferred embodiments, mentioned isolated nucleic acid is circRNA. The term “transfer vector” includes non-viral, viral, plasmid, and non-plasmid vectors.

Methods of introducing and expressing genes into a cell are known in the art. The vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A preferred method for the introduction of a polynucleotide into a host cell is calcium phosphate transfection. In some embodiments, mentioned polynucleotide is nucleic acid. In preferred embodiments, the mentioned polynucleotide is circRNA.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, nanoparticles, lipid-nanoparticle conjugates, microspheres, beads, peptide-base polyplexes and lipid-based systems including oil-in-water emulsions, micelles, mixed micelles, lipid nanoparticles and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In some embodiments, a suitable liposome or lipid-nanoparticles conjugates comprises one or more cationic lipids, e.g. cKK-E12. In some embodiments, mentioned polynucleotide is nucleic acid. In preferred embodiments, the mentioned polynucleotide is circRNA. In some embodiments, circRNA is encapsulated within the said colloidal dispersion systems. In other embodiments, circRNA is attached in the vicinity of said colloidal dispersion systems.

In preferred embodiments, the transfer vector is liposome. In other preferred embodiments, the transfer vector is chosen from the group of lipid nanoparticles or lipid-nanoparticle conjugates. In some embodiments, circRNA encoding a protein is encapsulated within a liposome, wherein the liposome comprises a cationic lipid. In preferred embodiments, circRNAs encoding TFPs, CARs, TCRs are encapsulated within a liposome, wherein the liposome comprises a cationic lipid.

In the case where a non-viral delivery system is utilized, an exemplary delivery vehicle is a liposome. The use of lipid formulations is contemplated for the introduction of the nucleic acids into a host cell (in vitro, ex vivo or in vivo). In preferred embodiments, the mentioned nucleic acids are circRNAs. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In some embodiments, a suitable liposome or lipid-nanoparticles conjugates comprises one or more non-cationic lipids, one or more cholesterol-based lipids and/or one or more PEG-modified lipids. In some embodiments, the one or more non-cationic lipids are selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In some embodiments, a suitable liposome or lipid-nanoparticles conjugates comprises lipids selected from the group of cKK-E12, DOPE, Cholesterol, DMG-PEG-2K. In other embodiments, circRNA encoding a protein is encapsulated within the said liposome or lipid-nanoparticles conjugates. In some embodiments, a suitable liposome or lipid-nanoparticles conjugates comprises lipids selected from the group of cKK-E12, 1,2-dioleoyl-sn-glycero-3-phosphoethanolamine (DOPE), cholesterol, and 1,2-dimyristoyl-sn-glycero-3-phosphoethanolamine-N-[methoxy-(polyethyleneglycol)-2000] (ammonium salt) (C14-PEG 2000). In preferred embodiments, circRNA is associated with the said suitable liposome or lipid-nanoparticles conjugates. In more preferred embodiments, circRNA is incorporated into the said suitable liposome or lipid-nanoparticles conjugates. In other preferred embodiments, circRNAs encoding TFPs, CARs, TCRs are incorporated into the said suitable liposome or lipid-nanoparticle conjugates.

In some instances, a non-viral transfer vector is chosen from the group of lipid-based delivery systems comprising Invivofectamine® (IF) 2.0 reagent, Lipofectamine™ MessengerMAX™ (ThermoFisher Scientific), AteloGene® (KOKEN), Lipofectemine®, Lipofectamine® 2000, or Lipofectamine 3000.

Regardless of the method used to introduce exogenous nucleic acids into a host cell or otherwise expose a cell to the inhibitor of the present disclosure, in order to confirm the presence of the circRNA in the host cell, a variety of assays may be performed. Such assays include, for example, “molecular biological” assays well known to those of skill in the art, such as Southern and northern blotting, RT-PCR and PCR; “biochemical” assays, such as detecting the presence or absence of a particular peptide, e.g., by immunological means (ELISAs and western blots) or by assays described herein to identify agents falling within the scope of the present disclosure.

The present disclosure further provides a vector comprising a TFP, a CAR, a TCR encoding nucleic acid molecule. In preferred embodiments, the mentioned nucleic acid molecule is circRNA. In one aspect, a vector can be directly transduced into a cell, e.g., a T cell. In one aspect, the vector is capable of expressing the TFP construct in mammalian T cells. In one aspect, the mammalian T cell is a human T cell.

### Selected Cell Targeting Ligands.

The selected cell targeting ligands of the disclosed transfer vectors selectively bind immune cells of interest within a heterogeneous cell population. In preferred embodiments, the mentioned targeting ligands are associated with the transfer vectors. In other preferred embodiments, the targeting ligands are disposed throughout the surface of the transfer vectors. In more preferred embodiments, the targeting ligands are associated with the transfer vectors comprising circRNAs encoding TFPs, CARs, or TCRs. In other more preferred embodiments, the targeting ligands are disposed throughout the surface of the transfer vectors comprising circRNAs encoding TFPs, CARs, or TCRs.

In particular embodiments, the immune cells of interest are lymphocytes. Lymphocytes include T-cells, B cells, natural killer (NK) cells, monocytes/macrophages and HSCs. In more preferred embodiments, the lymphocytes are T-cells.

“Selective delivery” means that nucleic acids are delivered and expressed by one or more selected lymphocyte populations. In particular embodiments, selective delivery is exclusive to a selected lymphocyte population. In particular embodiments, at least 65%, 70%, 75%, 80%, 85%, 90%, 95% or 99% of administered nucleic acids are delivered and/or expressed by a selected lymphocyte population. In particular embodiments, selective delivery ensures that non-lymphocyte cells do not express delivered nucleic acids. For example, when the targeting agent is a T-cell receptor (TCR) gene, selectivity is ensured because only T cells have the zeta chains for TCR expression. Selective delivery can also be based on lack of nucleic acid uptake into unselected cells or based on the presence of a specific promoter within the nucleic acid sequence. For example, transiently-expressed nucleic acids can include a T-cell-specific CD3-delta promoter. Additional promoters that can achieve selective delivery include: the murine stem cell virus promoter or the distal Lck promoter for T cells or HSCs; the CD45 promoter, WASP promoter or IFN-beta promoter for HSCs; the B29 promoter for B cells; or the CD14 promoter or the CD11b promoter for monocytes/macrophages.

In some embodiments, selected cell targeting ligands can include binding domains for motifs found on lymphocyte cells. Selected cell targeting ligands can also include any selective binding mechanism allowing selective uptake into lymphocytes. In particular embodiments, selected cell targeting ligands include binding domains for T-cell receptor motifs; T-cell α chains; T-cell β chains; T-cell γ chains; T-cell δ chains; CCR7; CD1a; CD1b; CD1c; CD1d; CD3; CD4; CD5; CD7; CD8; CD11b; CD11c; CD16; CD19; CD20; CD21; CD22; CD25; CD28; CD34; CD35; CD39; CD40; CD45RA; CD45RO; CD46, CD52; CD56; CD62L; CD68; CD80; CD86; CD95; CD101; CD117; CD127; CD133; CD137 (4-1BB); CD148; CD163; F4/80; IL-4Ra; Sca-1; CTLA-4; GITR; GARP; LAP; granzyme B; LFA-1; transferrin receptor; and combinations thereof.

In particular embodiments, binding domains can include cell marker ligands, receptor ligands, antibodies, peptides, peptide aptamers, nucleic acids, nucleic acid aptamers, spiegelmers or combinations thereof. Within the context of selected cell targeting ligands, binding domains include any substance that binds to another substance to form a complex capable of mediating endocytosis.

“Antibodies” are one example of binding domains and include whole antibodies or binding fragments of an antibody, e.g., Fv, VHH, Fab, Fab′, F(ab′)2, Fc, and single chain Fv fragments (scFvs) or any biologically effective fragments of an immunoglobulin that bind specifically to a motif expressed by a lymphocyte. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Antibodies from human origin or humanized antibodies have lowered or no immunogenicity in humans and have a lower number of non-immunogenic epitopes compared to non-human antibodies. Antibodies and their fragments will generally be selected to have a reduced level or no antigenicity in human subjects.

Antibodies that specifically bind a motif expressed by a lymphocyte can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to a lymphocyte motif. For example, binding domains may be identified by screening a Fab phage library for Fab fragments that specifically bind to a target of interest (see Hoet et al., Nat. Biotechnol. 23:344, 2005). Phage display libraries of human antibodies are also available. Additionally, traditional strategies for hybridoma development using a target of interest as an immunogen in convenient systems (e.g., mice, HuMAb Mouse®, TCmouse™, KM-Mouse®, llamas, chicken, rats, hamsters, rabbits, etc.) can be used to develop binding domains. In particular embodiments, antibodies specifically bind to motifs expressed by a selected lymphocyte and do not cross react with nonspecific components or unrelated targets. Once identified, the amino acid sequence or nucleic acid sequence coding for the antibody can be isolated and/or determined.

In particular embodiments, binding domains of selected cell targeting ligands include T-cell receptor motif antibodies; T-cell α chain antibodies; T-cell β chain antibodies; T-cell γ chain antibodies; T-cell δ chain antibodies; CCR7 antibodies; CD1a antibodies; CD1b antibodies; CD1c antibodies; CD1d antibodies; CD3 antibodies; CD4 antibodies; CD5 antibodies; CD7 antibodies; CD8 antibodies; CD11 b antibodies; CD11c antibodies; CD16 antibodies; CD19 antibodies; CD20 antibodies; CD21 antibodies; CD22 antibodies; CD25 antibodies; CD28 antibodies; CD34 antibodies; CD35 antibodies; CD39 antibodies; CD40 antibodies; CD45RA antibodies; CD45RO antibodies; CD46 antibodies; CD52 antibodies; CD56 antibodies; CD62L antibodies; CD68 antibodies; CD80 antibodies; CD86 antibodies CD95 antibodies; CD101 antibodies; CD117 antibodies; CD127 antibodies; CD133 antibodies; CD137 (4-1BB) antibodies; CD148 antibodies; CD163 antibodies; F4/80 antibodies; IL-4Ra antibodies; Sca-1 antibodies; CTLA-4 antibodies; GITR antibodies; GARP antibodies; LAP antibodies; granzyme B antibodies; LFA-1 antibodies; or transferrin receptor antibodies. These binding domains also can consist of scFv fragments of the foregoing antibodies.

Peptide aptamers include a peptide loop (which is specific for a target protein) attached at both ends to a protein scaffold. This double structural constraint greatly increases the binding affinity of the peptide aptamer to levels comparable to an antibody. The variable loop length is typically 8 to 20 amino acids (e.g., 8 to 12 amino acids), and the scaffold may be any protein which is stable, soluble, small, and non-toxic (e.g., thioredoxin-A, stefin A triple mutant, green fluorescent protein, eglin C, and cellular transcription factor Spl). Peptide aptamer selection can be made using different systems, such as the yeast two-hybrid system (e.g., Gal4 yeast-two-hybrid system) or the LexA interaction trap system.

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. In particular embodiments, aptamers are small (15 kDa; or between 15-80 nucleotides or between 20-50 nucleotides). Aptamers are generally isolated from libraries consisting of 1014-1015 random oligonucleotide sequences by a procedure termed SELEX (systematic evolution of ligands by exponential enrichment; see, for example, Tuerk et al., Science, 249:505-510, 1990; Green et al., Methods Enzymology. 75-86, 1991; and Gold et al., Annu. Rev. Biochem., 64: 763-797, 1995). Further methods of generating aptamers are described in, for example, U.S. Pat. Nos. 6,344,318; 6,331,398; 6,110,900; 5,817,785; 5,756,291; 5,696,249; 5,670,637; 5,637,461; 5,595,877; 5,527,894; 5,496,938; 5,475,096; and 5,270,16. Spiegelmers are similar to nucleic acid aptamers except that at least one β-ribose unit is replaced by β-D-deoxyribose or a modified sugar unit selected from, for example, β-D-ribose, α-D-ribose, β-L-ribose.

Other agents that can facilitate internalization by and/or transfection of lymphocytes, such as poly(ethyleneimine)/DNA (PEI/DNA) endosomolytic peptides (ELPs) complexes can also be used.

### Modified Human Immune Cells

Disclosed herein, in some embodiments, are modified human immune cells comprising the recombinant nucleic acid disclosed herein, or the vectors disclosed herein; wherein the modified human immune cell comprises a functional disruption of an endogenous TCR. Also disclosed herein, in some embodiments, are modified human immune cells comprising the sequence encoding the TFP of the nucleic acid disclosed herein or a TFP encoded by the sequence of the nucleic acid disclosed herein, wherein the modified human immune cell comprises a functional disruption of an endogenous TCR. Further disclosed herein, in some embodiments, are modified allogenic T cells comprising the sequence encoding the TFP disclosed herein or a TFP encoded by the sequence of the nucleic acid disclosed herein.

In some instances, the immune cell further comprises a heterologous sequence encoding a TCR constant domain, wherein the TCR constant domain is a TCR alpha constant domain, a TCR beta constant domain or a TCR alpha constant domain and a TCR beta constant domain. In some instances, the endogenous TCR that is functionally disrupted is an endogenous TCR alpha chain, an endogenous TCR beta chain, or an endogenous TCR alpha chain and an endogenous TCR beta chain. In some instances, the endogenous TCR that is functionally disrupted has reduced binding to MHC-peptide complex compared to that of an unmodified control immune cell. In some instances, the functional disruption is a disruption of a gene encoding the endogenous TCR. In some instances, the disruption of a gene encoding the endogenous TCR is a removal of a sequence of the gene encoding the endogenous TCR from the genome of an immune cell. In some instances, the immune cell is a human T cell. In some instances, the T cell is a CD8+, a CD4+ T cell, a CD8+CD4+ T cell, an NKT cell, or an NK cell. In some instances, the T cell is an allogenic T cell. In some instances, the modified human immune cells further comprise a nucleic acid encoding an inhibitory molecule that comprises a first polypeptide comprising at least a portion of an inhibitory molecule, associated with a second polypeptide comprising a positive signal from an intracellular signaling domain. In some instances, the inhibitory molecule comprises the first polypeptide comprising at least a portion of PD1 and the second polypeptide comprising a costimulatory domain and primary signaling domain.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T cell lines available in the art, may be used. In certain aspects of the present disclosure, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the present disclosure, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe® 2991 cell processor, the Baxter OncologyCytoMate, or the Haemonetics® Cell Saver® 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

In one aspect, T cells are isolated from peripheral blood lymphocytes by lysing the red blood cells and depleting the monocytes, for example, by centrifugation through a PERCOLL® gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this present disclosure. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present disclosure.

Also contemplated in the context of the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents such as cyclosporin, azathioprine, methotrexate, and mycophenolate, antibodies, or other immunoablative agents such as alemtuzumab, anti-CD3 antibodies, cytoxan, fludarabine, cyclosporin, tacrolimus, rapamycin, mycophenolic acid, steroids, romidepsin, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631.

Generally, the T cells of the present disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-CD19 anti-BCMA, anti-CD22, anti-PD1, anti-MUC16, anti-IL13R2a2, anti-EphA2, anti-EGFRvIII, anti-ROR1, anti-PD-1, or anti-BAFF TFP, CAR, or TCR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-TAA TFP, CAR, or TCR are described in further detail below

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by western blotting using an antibody to a TCR chain. The same T cell subsets are used for SDS-PAGE analysis under non-reducing conditions to permit evaluation of covalent dimer formation.

In vitro expansion of TFP⁺ T cells or CAR+ T cells or TCR+ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0, and transduced with TFP on day 1 using a bicistronic lentiviral vector expressing TFP along with eGFP using a 2A ribosomal skipping sequence. Cultures are re-stimulated with either CD19+K562 cells (K562-CD19), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained CAR+, TCR+, or TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human CD19-specific TFP+ T cells to treat a primary human pre-B ALL in immunodeficient mice can be used (see, e.g., Milone et al., Molecular Therapy 17(8): 1453-1464 (2009)). Very briefly, after establishment of ALL, mice are randomized as to treatment groups. Different numbers of engineered T cells are coinjected at a 1:1 ratio into NOD/SCID/γ−/− mice bearing B-ALL. The number of copies of each vector in spleen DNA from mice is evaluated at various times following T cell injection Animals are assessed for leukemia at weekly intervals. Peripheral blood CD19+B-ALL blast cell counts are measured in mice that are injected with alphaCD19-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with leukemic cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for leukemia at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing leukemia in mice injected on day 21 with TFP T cells, an equivalent number of mock-transduced T cells, or no T cells. Mice from each group are randomly bled for determination of peripheral blood CD19+ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). In one non-limiting example, assessment of TFP-mediated proliferation is performed, e.g. in microtiter plates by mixing washed T cells with K562 cells expressing CD19 (K19) or CD32 and CD137 (KT32-BBL) for a final T cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant CD19 protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur™ flow cytometer (BD Biosciences), and data are analyzed according to the manufacturer's instructions.

Cytotoxicity can be assessed by a standard ⁵¹Cr-release assay (see, e.g., Milone et al., Molecular Therapy 17(8): 1453-1464 (2009)). Target cells (K562 lines and primary pro-B-ALL cells) are loaded with ⁵¹Cr (as NaCrO4, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of Triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). NOD/SCID/γc−/− (NSG) mice are injected IV with Nalm-6 cells (ATCC® CRL-3273™) followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in Nalm-6 xenograft model can be measured as the following: NSG mice are injected with Nalm-6 transduced to stably express firefly luciferase, followed by a single tail-vein injection of T cells electroporated with CD19 TFP 7 days later. Animals are imaged at various time points post injection. For example, photon-density heat maps of firefly luciferase positive leukemia in representative mice at day 5 (2 days before treatment) and day 8 (24 hours post TFP+ PBLs) can be generated.

Other assays, including those described in the Example section herein as well as those that are known in the art can also be used to evaluate the anti-CD19, anti-BCMA, anti-IL13Ra2, MUC16, EphA2 EGFRvIII, anti-CD22, anti-ROR1, anti-PD1, or anti-BAFF TFP constructs disclosed herein.

### Pharmaceutical Compositions

Disclosed herein, in some embodiments, are pharmaceutical compositions comprising: (a) the modified immune cells of the disclosure; and (b) a pharmaceutically acceptable carrier. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the T cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a modified T-T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded modified human immune cells of the present disclosure. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the present disclosure, and one or more subsequent administrations of the TFP T cells of the present disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the present disclosure are administered to the subject (e.g., human) per week, e.g., 2, 3, or 4 administrations of the TFP T cells of the present disclosure are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the present disclosure are administered for at least two, three, four, five, six, seven, eight or more weeks.

In one aspect, anti-TAA TFP T cells or CAR T cells or TCR T cells are generated using lentiviral viral vectors, such as lentivirus. TFP-T cells generated that way will have stable TFP expression. In another aspect, T anti-TAA TFP T cells or CAR T cells or TCR T cells

In one aspect, TFP T cells transiently express TFP vectors for 4, 5, 6, 7, 8, 9, 10, 11, 12, 13, 14, 15 days after transduction. Transient expression of TFPs can be affected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen-day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Methods of Treatment

Disclosed herein, in some embodiments, are methods of treating cancer in a subject in need thereof, the method comprising administering to the subject a therapeutically effective amount of the pharmaceutical compositions and formulations disclosed herein. Further disclosed herein, in some embodiments, are methods of treating cancer in a subject in need thereof, the method comprising administering to the subject a pharmaceutical composition comprising (a) a modified human immune cell produced according to the methods disclosed herein; and (b) a pharmaceutically acceptable carrier. Further disclosed herein, in some embodiments, are methods of treating cancer in a subject in need thereof, the method comprising administering to the subject a pharmaceutical composition comprising (a) a delivery device (e.g., a liposome) containing a payload comprising one of the circular RNA molecules or vectors disclosed herein; and (b) a pharmaceutically acceptable carrier.

In some instances, the modified human immune cell is an allogeneic T cell. In some embodiments, the modified human immune cell is an autologous T cell. In some embodiments, the modified human immune cell is a lymphoblast. In some instances, less cytokines are released in the subject compared a subject administered an effective amount of an unmodified control T cell. In some instances, less cytokines are released in the subject compared a subject administered an effective amount of a modified human immune cell comprising the recombinant nucleic acid disclosed herein, or the vector disclosed herein.

In some instances, the method comprises administering the pharmaceutical formulation in combination with an agent that increases the efficacy of the pharmaceutical formulation. In some instances, the method comprises administering the pharmaceutical formulation in combination with an agent that ameliorates one or more side effects associated with the pharmaceutical composition.

In some instances, the cancer is a solid cancer, a lymphoma or a leukemia. In some embodiments, the cancer is selected from the group consisting of acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

Without wishing to be bound by any particular theory, the anti-tumor immunity response elicited by the modified human immune cells may be an active or a passive immune response, or alternatively may be due to a direct vs indirect immune response.

In one aspect, the modified human immune cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP or TCR or CAR and a TCR alpha, beta, gamma, and/or delta constant domain to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector disclosed herein. The modified human immune cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art; therefore, the present disclosure is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised.

The modified human immune cells of the present disclosure may be administered either alone, or as a pharmaceutical composition in combination with diluents and/or with other components such as IL-2 or other cytokines or cell populations.

### Combination Therapies

A modified human immune cell or targeted circular RNA or targeted or untargeted delivery vehicle (e.g., a liposome) described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a modified human immune cell. Also provided are T cells that express multiple TFPs, which bind to the same or different target antigens, or same or different epitopes on the same target antigen. Also provided are populations of T cells in which a first subset of T cells expresses a first TFP and a TCR alpha and/or beta constant domain and a second subset of T cells express a second TFP and a TCR alpha and/or beta constant domain. Also provided are populations of T cells in which a first subset of T cells expresses a first TFP and a TCR gamma and/or delta constant domain and a second subset of T cells express a second TFP and a TCR gamma and/or delta constant domain.

A modified human immune cell described herein and the at least one additional therapeutic agent can be administered simultaneously, in the same or in separate compositions, or sequentially. For sequential administration, the modified human immune cell described herein can be administered first, and the additional agent can be administered second, or the order of administration can be reversed.

In further aspects, a modified human immune cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of a modified human immune cell. Side effects associated with the administration of a modified human immune cell include but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods disclosed herein can comprise administering a modified human immune cell described herein to a subject and further administering an agent to manage elevated levels of a soluble factor resulting from treatment with a modified human immune cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab.

In one embodiment, the subject can be administered an agent which enhances the activity of a modified human immune cell. For example, in one embodiment, the agent can be an agent which inhibits an inhibitory molecule. Inhibitory molecules, e.g., Programmed Death 1 (PD1), can, in some embodiments, decrease the ability of a modified human immune cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a modified human immune cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a modified human immune cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of a modified human immune cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-TAA TFP.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention and are not to be construed as limiting in any way the remainder of the disclosure.

### Example 1: The Design of Circular RNA (circRNA) Encoding a Protein

Target protein chosen to be expressed for this experiment is GFP. Translation of functional GFPs from circular RNA is achieved by using a ribozyme in a permuted intron-exon (PIE) splicing strategy. To create a circRNA encoding GFPs, an internal ribosome entry site (IRES), following by a GFP coding sequence, are placed between two short fragments of the E2 and E1 exons downstream and upstream of the group I catalytic intron in the thymidylate synthase (Td) gene from phage T4. Alternatively, E2 and E1 exons downstream and upstream of the group I catalytic intron in Anabaena pre-tRNA gene can be used as splicing efficiency of group I catalytic intron is more efficient in Anabaena pre-tRNA gene than in phage T4 Td gene. [Puttaraju, M. & Been, M. Nucleic Acids Res. 20, 5357-5364 (1992)]. Finally, the 3′ half of the group I catalytic intron is cloned upstream of E2 whereas the 5′ half of the group I catalytic intron is placed downstream of E1. A spacer between the 3′ PIE splice site and the IRES are designed. Complementary ‘homology arms’ of 33-35 nucleotides in length are placed at the 5′ and 3′ ends of the precursor RNA with the aim of bringing the 5′ and 3′splice sites into proximity of one another are used during circulation process to increase splitting efficiency.

### Example 2: The Design of CircRNA Encoding TFPs

Translation of functional TFPs from circular RNA (circRNA) can be achieved by using ribozyme in a permuted intron-exon (PIE) splicing strategy. To create a circRNA encoding TFPs, an internal ribosome entry site (IRES), following by a TFP coding sequence (CDS), are placed between two short fragments of the E2 and E1 exons downstream and upstream of the group I catalytic intron in the thymidylate synthase (Td) gene from phage T4. Alternatively, E2 and E1 exons downstream and upstream of the group I catalytic intron in Anabaena pre-tRNA gene can be used as splicing efficiency of group I catalytic intron is more efficient in Anabaena pre-tRNA gene than in phage T4 Td gene. [Puttaraju, M. & Been, M. Nucleic Acids Res. 20, 5357-5364 (1992)]. Finally, the 5′ half of the group I catalytic intron is cloned upstream of E2 whereas the 3′ half of the group I catalytic intron is placed downstream of E1. A spacer between the 3′ PIE splice site and the IRES are designed. Complementary external homology arms' of 33-35 nucleotides in length are placed at the 5′ and 3′ends of the precursor RNA with the aim of bringing the 5′ and 3′splice sites into proximity of one another are used during circulation process to increase splitting efficiency [Wesselhoeft et. al., Nat. Commun., 9:26-29., 2018]. In some embodiments, the protein binding motif is added outside of the TFP coding sequence to increase the half-life of the circRNA. In one embodiment, the protein binding motif is a polyA linker with about 20 nucleotides.

### Example 3: The Design of CircRNA Encoding CARs, TCRs

Translation of functional CARs, TCRs from circular RNA (circRNA) can be achieved by using ribozyme in a permuted intron-exon (PIE) splicing strategy. To create a circRNA encoding CARs, TCRs, an internal ribosome entry site (IRES), following by a CARs, TCRs coding sequence (CDS), are placed between two short fragments of the E2 and E1 exons downstream and upstream of the group I catalytic intron in the thymidylate synthase (Td) gene from phage T4. Alternatively, E2 and E1 exons downstream and upstream of the group I catalytic intron in Anabaena pre-tRNA gene can be used as splicing efficiency of group I catalytic intron is more efficient in Anabaena pre-tRNA gene than in phage T4 Td gene. [Puttaraju, M. & Been, M. Nucleic Acids Res. 20, 5357-5364 (1992)]. Finally, the 5′ half of the group I catalytic intron is cloned upstream of E2 whereas the 3′ half of the group I catalytic intron is placed downstream of E1. A spacer between the 3′ PIE splice site and the IRES are designed. Complementary ‘homology arms’ of 33-35 nucleotides in length are placed at the 5′ and 3′ ends of the precursor RNA with the aim of bringing the 5′ and 3′ splice sites into proximity of one another are used during circulation process to increase splitting efficiency. Accessorily, protein binding motif can be added outside of the TFP coding sequence to increase the half-life of the circRNA such as a polyA linker with A>20 nucleotides [Wesselhoeft et. al., Nat. Commun., 2018].

TABLE 1
Sequences to be used in the design of circular
RNA encoding a payload

| | |
|---|---|
| CVB3 | TTAAAACAGCCTGTGGGTTGATCCCACCCACAGGCCCATTG |
| IRES | GGCGCTAGCACTCTGGTATCACGGTACCTTTGTGCGCCTGT |
| (SEQ | TTTATACCCCCTCCCCCAACTGTAACTTAGAAGTAACACAC |
| ID | ACCGATCAACAGTCAGCGTGGCACACCAGCCACGTTTTGAT |
| NO: | CAAGCACTTCTGTTACCCCGGACTGAGTATCAATAGACTGC |
| 140) | TCACGCGGTTGAAGGAGAAAGCGTTCGTTATCCGGCCAACT |
| | ACTTCGAAAAACCTAGTAACACCGTGGAAGTTGCAGAGTGT |
| | TTCGCTCAGCACTACCCCAGTGTAGATCAGGTCGATGAGTC |
| | ACCGCATTCCCCACGGGCGACCGTGGCGGTGGCTGCGTTGG |
| | CGGCCTGCCCATGGGGAAACCCATGGGACGCTCTAATACAG |
| | ACATGGTGCGAAGAGTCTATTGAGCTAGTTGGTAGTCCTCC |
| | GGCCCCTGAATGCGGCTAATCCTAACTGCGGAGCACACACC |
| | CTCAAGCCAGAGGGCAGTGTGTCGTAACGGGCAACTCTGCA |
| | GCGGAACCGACTACTTTGGGTGTCCGTGTTTCATTTTATTC |
| | CTATACTGGCTGCTTATGGTGACAATTGAGAGATCGTTACC |
| | ATATAGCTATTGGATTGGCCATCCGGTGACTAATAGAGCTA |
| | TTATATATCCCTTTGTTGGGTTTATACCACTTAGCTTGAAA |
| | GAGGTTAAAACATTACAATTCATTGTTAAGTTGAATACAGC |
| | AAA |
| EMCV | TTGCCAGTCTGCTCGATATCGCAGGCTGGGTCCGTGACTAC |
| IRES | CCACTCCCCCTTTCAACGTGAAGGCTACGATAGTGCCAGGG |
| (SEQ | CGGGTACTGCCGTAAGTGCCACCCCAAACAACAACAACAAA |
| ID | ACAAACTCCCCCTCCCCCCCCTTACTATACTGGCCGAAGCC |
| NO: | ACTTGGAATAAGGCCGGTGTGCGTTTGTCTACATGCTATTT |
| 141) | TCTACCGCATTACCGTCTTATGGTAATGTGAGGGTCCAGAA |
| | CCTGACCCTGTCTTCTTGACGAACACTCCTAGGGGTCTTTC |
| | CCCTCTCGACAAAGGAGTGTAAGGTCTGTTGAATGTCGTGA |
| | AGGAAGCAGTTCCTCTGGAAGCTTCTTAAAGACAAACAACG |
| | TCTGTAGCGACCCTTTGCAGGCAGCGGAACCCCCCACCTGG |
| | TGACAGGTGCCTCTGCGGCCAAAAGCCACGTGTATAAGATA |
| | CACCTGCAAAGGCGGCACAACCCCAGTGCCACGTTGTGAGT |
| | TGGATAGTTGTGGAAAGAGTCAAATGGCTCTCCTCAAGCGT |
| | ATTCAACAAGGGGCTGAAGGATGCCCAGAAGGTACCCCATT |
| | GTATGGGATCTGATCTGGGGCCTCGGTGCACGTGCTTTACA |
| | CGTGTTGAGTCGAGGTGAAAAAACGTCTAGGCCCCCCGAAC |
| | CACGGGGACGTGGTTTTCCTTTGAAAACCACGATTACAAT |
| Spacer | GGTAGTGGTGCTACTAACTTCAGCCTGCTGAAGCA |
| (SEQ | |
| ID | |
| NO: | |
| 142) | |
| Ana3.0 | GGGAGACCCTCGACCGTCGATTGTCCACTGGTCAACAATAG |
| (Full) | ATGACTTACAACTAATCGGAAGGTGCAGAGACTCGACGGGA |
| (SEQ | GCTACCCTAACGTCAAGACGAGGGTAAAGAGAGAGTCCAAT |
| ID | TCTCAAAGCCAATAGGCAGTAGCGAAAGCTGCAAGAGAATG |
| NO: | AAAATCCGTTGACCTTAAACGGTCGTGTGGGTTCAAGTCCC |
| 145) | TCCACCCCCACGCCGGAAACGCAATAGCCGAAAAACAAAAA |
| | ACAAAAAAACCCCCCTCTCCCTCCCCCCCTAACGTTACTGG |
| | CCGAAGCCGCTTGGAATAAGGCCGGTGTGCGTTTGTCTATA |
| | TGTTATTTTCCACCATATTGCCGTCTTTTGGCAATGTGAGG |
| | GCCCGGAAACCTGGCCCTGTCTTCTTGACGAGCATTCCTAG |
| | GGGTCTTTCCCCTCTCGCCAAAGGAATGCAAGGTCTGTTGA |
| | ATGTCGTGAAGGAAGCAGTTCCTCTGGAAGCTTCTTGAAGA |
| | CAAACAACGTCTGTAGCGACCCTTTGCAGGCAGCGGAACCC |
| | CCCACCTGGCGACAGGTGCCTCTGCGGCCAAAAGCCACGTG |
| | TATAAGATACACCTGCAAAGGCGGCACAACCCCAGTGCCAC |
| | GTTGTGAGTTGGATAGTTGTGGAAAGAGTCAAATGGCTCTC |
| | CTCAAGCGTATTCAACAAGGGGCTGAAGGATGCCCAGAAGG |
| | TACCCCATTGTATGGGATCTGATCTGGGGCCTCGGTGCACA |
| | TGCTTTACATGTGTTTAGTCGAGGTTAAAAAACGTCTAGGC |
| | CCCCCGAACCACGGGGACGTGGTTTTCCTTTGAAAAACACG |
| | ATGATAATATGGCCACAACCATGGGAGTCAAAGTTCTGTTT |
| | GCCCTGATCTGCATCGCTGTGGCCGAGGCCAAGCCCACCGA |
| | GAACAACGAAGACTTCAACATCGTGGCCGTGGCCAGCAACT |
| | TCGCGACCACGGATCTCGATGCTGACCGCGGGAAGTTGCCC |
| | GGCAAGAAGCTGCCGCTGGAGGTGCTCAAAGAGATGGAAGC |
| | CAATGCCCGGAAAGCTGGCTGCACCAGGGGCTGTCTGATCT |
| | GCCTGTCCCACATCAAGTGCACGCCCAAGATGAAGAAGTTC |
| | ATCCCAGGACGCTGCCACACCTACGAAGGCGACAAAGAGTC |
| | CGCACAGGGCGGCATAGGCGAGGCGATCGTCGACATTCCTG |
| | AGATTCCTGGGTTCAAGGACTTGGAGCCCATGGAGCAGTTC |
| | ATCGCACAGGTCGATCTGTGTGTGGACTGCACAACTGGCTG |
| | CCTCAAAGGGCTTGCCAACGTGCAGTGTTCTGACCTGCTCA |
| | AGAAGTGGCTGCCGCAACGCTGTGCGACCTTTGCCAGCAAG |
| | ATCCAGGGCCAGGTGGACAAGATCAAGGGGGCCGGTGGTGA |
| | CTAAAAAAAACAAAAAACAAAACGGCTATTATGCGTTACCG |
| | GCGAGACGCTACGGACTTAAATAATTGAGCCTTAAAGAAGA |
| | AATTCTTTAAGTGGATGCTCTCAAACTCAGGGAAACCTAAA |
| | TCTAGTTATAGACAAGGCAATCCTGAGCCAAGCCGAAGTAG |
| | TAATTAGTAAGACCAGTGGACAATCGACGGATAACAGCATA |
| | TCTAG |

TABLE 2
Sequences for generating circular RNA encoding a payload

| | |
|---|---|
| Ana-CVB3- | GGGAGACCCTCGACCGTCGATTGTCCACTGGTCAACAATAGATGACT |
| FMC63_CD3ϵ | TACAACTAATCGGAAGGTGCAGAGACTCGACGGGAGCTACCCTAACG |
| (SEQ ID NO: 143) | TCAAGACGAGGGTAAAGAGAGAGTCCAATTCTCAAAGCCAATAGGC |
| | AGTAGCGAAAGCTGCAAGAGAATGAAAATCCGTTGACCTTAAACGGT |
| | CGTGTGGGTTCAAGTCCCTCCACCCCCACGCCGGAAACGCAATAGCC |
| | GAAAAACAAAAAACAAAAAAATTAAAACAGCCTGTGGGTTGATCCC |
| | ACCCACAGGCCCATTGGGCGCTAGCACTCTGGTATCACGGTACCTTTG |
| | TGCGCCTGTTTTATACCCCCTCCCCCAACTGTAACTTAGAAGTAACAC |
| | ACACCGATCAACAGTCAGCGTGGCACACCAGCCACGTTTTGATCAAG |
| | CACTTCTGTTACCCCGGACTGAGTATCAATAGACTGCTCACGCGGTTG |
| | AAGGAGAAAGCGTTCGTTATCCGGCCAACTACTTCGAAAAACCTAGT |
| | AACACCGTGGAAGTTGCAGAGTGTTTCGCTCAGCACTACCCCAGTGT |
| | AGATCAGGTCGATGAGTCACCGCATTCCCCACGGGCGACCGTGGCGG |
| | TGGCTGCGTTGGCGGCCTGCCCATGGGGAAACCCATGGGACGCTCTA |
| | ATACAGACATGGTGCGAAGAGTCTATTGAGCTAGTTGGTAGTCCTCC |
| | GGCCCCTGAATGCGGCTAATCCTAACTGCGGAGCACACACCCTCAAG |
| | CCAGAGGGCAGTGTGTCGTAACGGGCAACTCTGCAGCGGAACCGACT |
| | ACTTTGGGTGTCCGTGTTTCATTTTATTCCTATACTGGCTGCTTATGGT |
| | GACAATTGAGAGATCGTTACCATATAGCTATTGGATTGGCCATCCGGT |
| | GACTAATAGAGCTATTATATATCCCTTTGTTGGGTTTATACCACTTAG |
| | CTTGAAAGAGGTTAAAACATTACAATTCATTGTTAAGTTGAATACAGC |
| | AAAATGCTTCTCCTGGTGACAAGCCTTCTGCTCTGTGAGTTACCACAC |
| | CCAGCATTCCTCCTGATCCCAGACATCCAGATGACACAGACTACATCC |
| | TCCCTGTCTGCCTCTCTGGGAGACAGAGTCACCATCAGTTGCAGGGCA |
| | AGTCAGGACATTAGTAAATATTTAAATTGGTATCAGCAGAAACCAGA |
| | TGGAACTGTTAAACTCCTGATCTACCATACATCAAGATTACACTCAGG |
| | AGTCCCATCAAGGTTCAGTGGCAGTGGGTCTGGAACAGATTATTCTCT |
| | CACCATTAGCAACCTGGAGCAAGAAGATATTGCCACTTACTTTTGCCA |
| | ACAGGGTAATACGCTTCCGTACACGTTCGGAGGGGGGACTAAGTTGG |
| | AAATAACAGGCTCCACCTCTGGATCCGGCAAGCCCGGATCTGGCGAG |
| | GGATCCACCAAGGGCGAGGTGAAACTGCAGGAGTCAGGACCTGGCCT |
| | GGTGGCGCCCTCACAGAGCCTGTCCGTCACATGCACTGTCTCAGGGG |
| | TCTCATTACCCGACTATGGTGTAAGCTGGATTCGCCAGCCTCCACGAA |
| | AGGGTCTGGAGTGGCTGGGAGTAATATGGGGTAGTGAAACCACATAC |
| | TATAATTCAGCTCTCAAATCCAGACTGACCATCATCAAGGACAACTCC |
| | AAGAGCCAAGTTTTCTTAAAAATGAACAGTCTGCAAACTGATGACAC |
| | AGCCATTTACTACTGTGCCAAACATTATTACTACGGTGGTAGCTATGC |
| | TATGGACTACTGGGGTCAAGGAACCTCAGTCACCGTCTCCGCGGCCG |
| | CGGGCGGTGGAGGCAGTGGTGGTGGCGGCTCTGGCGGTGGTGGTAGC |
| | CTCGAGGATGGTAATGAAGAAATGGGTGGTATTACACAGACACCATA |
| | TAAAGTCTCCATCTCTGGAACCACAGTAATATTGACATGCCCTCAGTA |
| | TCCTGGATCTGAAATACTATGGCAACACAATGATAAAAACATAGGCG |
| | GTGATGAGGATGATAAAAACATAGGCAGTGATGAGGATCACCTGTCA |
| | CTGAAGGAATTTTCAGAATTGGAGCAAAGTGGTTATTATGTCTGCTAC |
| | CCCAGAGGAAGCAAACCAGAAGATGCGAACTTTTATCTCTACCTGAG |
| | GGCAAGAGTGTGTGAGAACTGCATGGAGATGGATGTGATGTCGGTGG |
| | CCACAATTGTCATAGTGGACATCTGCATCACTGGGGGCTTGCTGCTGC |
| | TGGTTTACTACTGGAGCAAGAATAGAAAGGCCAAGGCCAAGCCTGTG |
| | ACACGAGGAGCGGGTGCTGGCGGCAGGCAAAGGGGACAAAACAAGG |
| | AGAGGCCACCACCTGTTCCCAACCCAGACTATGAGCCCATCCGGAAA |
| | GGCCAGCGGGACCTGTATTCTGGCCTGAATCAGAGACGCATCTGATA |
| | AAAAAAACAAAAAACAAAACGGCTATTATGCGTTACCGGCGAGACG |
| | CTACGGACTTAAATAATTGAGCCTTAAAGAAGAAATTCTTTAAGTGG |
| | ATGCTCTCAAACTCAGGGAAACCTAAATCTAGTTATAGACAAGGCAA |
| | TCCTGAGCCAAGCCGAAGTAGTAATTAGTAAGACCAGTGGACAATCG |
| | ACGGATAACAGCATATCTAG |
| Ana-EMCV- | GGGAGACCCTCGACCGTCGATTGTCCACTGGTCAACAATAGATGACT |
| FMC63_CD3ϵ | TACAACTAATCGGAAGGTGCAGAGACTCGACGGGAGCTACCCTAACG |
| (SEQ ID NO: 144) | TCAAGACGAGGGTAAAGAGAGAGTCCAATTCTCAAAGCCAATAGGC |
| | AGTAGCGAAAGCTGCAAGAGAATGAAAATCCGTTGACCTTAAACGGT |
| | CGTGTGGGTTCAAGTCCCTCCACCCCCACGCCGGAAACGCAATAGCC |
| | GAAAAACAAAAAACAAAAAAACCCCCCTCTCCCTCCCCCCCTAACGT |
| | TACTGGCCGAAGCCGCTTGGAATAAGGCCGGTGTGCGTTTGTCTATAT |
| | GTTATTTTCCACCATATTGCCGTCTTTTGGCAATGTGAGGGCCCGGAA |
| | ACCTGGCCCTGTCTTCTTGACGAGCATTCCTAGGGGTCTTTCCCCTCTC |
| | GCCAAAGGAATGCAAGGTCTGTTGAATGTCGTGAAGGAAGCAGTTCC |
| | TCTGGAAGCTTCTTGAAGACAAACAACGTCTGTAGCGACCCTTTGCA |
| | GGCAGCGGAACCCCCCACCTGGCGACAGGTGCCTCTGCGGCCAAAAG |
| | CCACGTGTATAAGATACACCTGCAAAGGCGGCACAACCCCAGTGCCA |
| | CGTTGTGAGTTGGATAGTTGTGGAAAGAGTCAAATGGCTCTCCTCAA |
| | GCGTATTCAACAAGGGGCTGAAGGATGCCCAGAAGGTACCCCATTGT |
| | ATGGGATCTGATCTGGGGCCTCGGTGCACATGCTTTACATGTGTTTAG |
| | TCGAGGTTAAAAAACGTCTAGGCCCCCCGAACCACGGGGACGTGGTT |
| | TTCCTTTGAAAAACACGATGATAATATGGCCACAACCATGCTTCTCCT |
| | GGTGACAAGCCTTCTGCTCTGTGAGTTACCACACCCAGCATTCCTCCT |
| | GATCCCAGACATCCAGATGACACAGACTACATCCTCCCTGTCTGCCTC |
| | TCTGGGAGACAGAGTCACCATCAGTTGCAGGGCAAGTCAGGACATTA |
| | GTAAATATTTAAATTGGTATCAGCAGAAACCAGATGGAACTGTTAAA |
| | CTCCTGATCTACCATACATCAAGATTACACTCAGGAGTCCCATCAAGG |
| | TTCAGTGGCAGTGGGTCTGGAACAGATTATTCTCTCACCATTAGCAAC |
| | CTGGAGCAAGAAGATATTGCCACTTACTTTTGCCAACAGGGTAATAC |
| | GCTTCCGTACACGTTCGGAGGGGGGACTAAGTTGGAAATAACAGGCT |
| | CCACCTCTGGATCCGGCAAGCCCGGATCTGGCGAGGGATCCACCAAG |
| | GGCGAGGTGAAACTGCAGGAGTCAGGACCTGGCCTGGTGGCGCCCTC |
| | ACAGAGCCTGTCCGTCACATGCACTGTCTCAGGGGTCTCATTACCCGA |
| | CTATGGTGTAAGCTGGATTCGCCAGCCTCCACGAAAGGGTCTGGAGT |
| | GGCTGGGAGTAATATGGGGTAGTGAAACCACATACTATAATTCAGCT |
| | CTCAAATCCAGACTGACCATCATCAAGGACAACTCCAAGAGCCAAGT |
| | TTTCTTAAAAATGAACAGTCTGCAAACTGATGACACAGCCATTTACTA |
| | CTGTGCCAAACATTATTACTACGGTGGTAGCTATGCTATGGACTACTG |
| | GGGTCAAGGAACCTCAGTCACCGTCTCCGCGGCCGCGGGCGGTGGAG |
| | GCAGTGGTGGTGGCGGCTCTGGCGGTGGTGGTAGCCTCGAGGATGGT |
| | AATGAAGAAATGGGTGGTATTACACAGACACCATATAAAGTCTCCAT |
| | CTCTGGAACCACAGTAATATTGACATGCCCTCAGTATCCTGGATCTGA |
| | AATACTATGGCAACACAATGATAAAAACATAGGCGGTGATGAGGATG |
| | ATAAAAACATAGGCAGTGATGAGGATCACCTGTCACTGAAGGAATTT |
| | TCAGAATTGGAGCAAAGTGGTTATTATGTCTGCTACCCCAGAGGAAG |
| | CAAACCAGAAGATGCGAACTTTTATCTCTACCTGAGGGCAAGAGTGT |
| | GTGAGAACTGCATGGAGATGGATGTGATGTCGGTGGCCACAATTGTC |
| | ATAGTGGACATCTGCATCACTGGGGGCTTGCTGCTGCTGGTTTACTAC |
| | TGGAGCAAGAATAGAAAGGCCAAGGCCAAGCCTGTGACACGAGGAG |
| | CGGGTGCTGGCGGCAGGCAAAGGGGACAAAACAAGGAGAGGCCACC |
| | ACCTGTTCCCAACCCAGACTATGAGCCCATCCGGAAAGGCCAGCGGG |
| | ACCTGTATTCTGGCCTGAATCAGAGACGCATCTGATAAAAAAAACAA |
| | AAAACAAAACGGCTATTATGCGTTACCGGCGAGACGCTACGGACTTA |
| | AATAATTGAGCCTTAAAGAAGAAATTCTTTAAGTGGATGCTCTCAAA |
| | CTCAGGGAAACCTAAATCTAGTTATAGACAAGGCAATCCTGAGCCAA |
| | GCCGAAGTAGTAATTAGTAAGACCAGTGGACAATCGACGGATAACAG |
| | CATATCTAG |
| Ana_CVB3_IRES- | GGGAGACCCTCGACCGTCGATTGTCCACTGGTCAACAATAGATGACT |
| eGFP (SEQ ID | TACAACTAATCGGAAGGTGCAGAGACTCGACGGGAGCTACCCTAACG |
| NO: 146) | TCAAGACGAGGGTAAAGAGAGAGTCCAATTCTCAAAGCCAATAGGC |
| | AGTAGCGAAAGCTGCAAGAGAATGAAAATCCGTTGACCTTAAACGGT |
| | CGTGTGGGTTCAAGTCCCTCCACCCCCACGCCGGAAACGCAATAGCC |
| | GAAAAACAAAAAACAAAAAAATTAAAACAGCCTGTGGGTTGATCCC |
| | ACCCACAGGCCCATTGGGCGCTAGCACTCTGGTATCACGGTACCTTTG |
| | TGCGCCTGTTTTATACCCCCTCCCCCAACTGTAACTTAGAAGTAACAC |
| | ACACCGATCAACAGTCAGCGTGGCACACCAGCCACGTTTTGATCAAG |
| | CACTTCTGTTACCCCGGACTGAGTATCAATAGACTGCTCACGCGGTTG |
| | AAGGAGAAAGCGTTCGTTATCCGGCCAACTACTTCGAAAAACCTAGT |
| | AACACCGTGGAAGTTGCAGAGTGTTTCGCTCAGCACTACCCCAGTGT |
| | AGATCAGGTCGATGAGTCACCGCATTCCCCACGGGCGACCGTGGCGG |
| | TGGCTGCGTTGGCGGCCTGCCCATGGGGAAACCCATGGGACGCTCTA |
| | ATACAGACATGGTGCGAAGAGTCTATTGAGCTAGTTGGTAGTCCTCC |
| | GGCCCCTGAATGCGGCTAATCCTAACTGCGGAGCACACACCCTCAAG |
| | CCAGAGGGCAGTGTGTCGTAACGGGCAACTCTGCAGCGGAACCGACT |
| | ACTTTGGGTGTCCGTGTTTCATTTTATTCCTATACTGGCTGCTTATGGT |
| | GACAATTGAGAGATCGTTACCATATAGCTATTGGATTGGCCATCCGGT |
| | GACTAATAGAGCTATTATATATCCCTTTGTTGGGTTTATACCACTTAG |
| | CTTGAAAGAGGTTAAAACATTACAATTCATTGTTAAGTTGAATACAGC |
| | AAAATGGTGAGCAAGGGCGAGGAGCTGTTCACCGGGGTGGTGCCCAT |
| | CCTGGTCGAGCTGGACGGCGACGTAAACGGCCACAAGTTCAGCGTGT |
| | CCGGCGAGGGCGAGGGCGATGCCACCTACGGCAAGCTGACCCTGAA |
| | GTTCATCTGCACCACCGGCAAGCTGCCCGTGCCCTGGCCCACCCTCGT |
| | GACCACCCTGACCTACGGCGTGCAGTGCTTCAGCCGCTACCCCGACC |
| | ACATGAAGCAGCACGACTTCTTCAAGTCCGCCATGCCCGAAGGCTAC |
| | GTCCAGGAGCGCACCATCTTCTTCAAGGACGACGGCAACTACAAGAC |
| | CCGCGCCGAGGTGAAGTTCGAGGGCGACACCCTGGTGAACCGCATCG |
| | AGCTGAAGGGCATCGACTTCAAGGAGGACGGCAACATCCTGGGGCAC |
| | AAGCTGGAGTACAACTACAACAGCCACAACGTCTATATCATGGCCGA |
| | CAAGCAGAAGAACGGCATCAAGGTGAACTTCAAGATCCGCCACAAC |
| | ATCGAGGACGGCAGCGTGCAGCTCGCCGACCACTACCAGCAGAACAC |
| | CCCCATCGGCGACGGCCCCGTGCTGCTGCCCGACAACCACTACCTGA |
| | GCACCCAGTCCGCCCTGAGCAAAGACCCCAACGAGAAGCGCGATCAC |
| | ATGGTCCTGCTGGAGTTCGTGACCGCCGCCGGGATCACTCTCGGCATG |
| | GACGAGCTGTACAAGTAAAAAAAACAAAAAACAAAACGGCTATTAT |
| | GCGTTACCGGCGAGACGCTACGGACTTAAATAATTGAGCCTTAAAGA |
| | AGAAATTCTTTAAGTGGATGCTCTCAAACTCAGGGAAACCTAAATCT |
| | AGTTATAGACAAGGCAATCCTGAGCCAAGCCGAAGTAGTAATTAGTA |
| | AGACCAGTGGACAATCGACGGATAACAGCATATCTAG |
| Ana_CVB3_IRES- | GGGAGACCCTCGACCGTCGATTGTCCACTGGTCAACAATAGATGACT |
| eGFP-sp1_mut | TACAACTAATCGGAAGGTGCAGAGACTCGACGGGAGCTACCCTAACG |
| (SEQ ID NO: 147) | TCAAGACGAGGGTAAAGAGAGAGTCCAATTCTCAAAGCCAATAGGC |
| | AGTAGGTCGTGTGGGTTCAAGTCCCTCCACCCCCACGCCGGAAACGC |
| | AATAGCCGAAAAACAAAAAACAAAAAAATTAAAACAGCCTGTGGGT |
| | TGATCCCACCCACAGGCCCATTGGGCGCTAGCACTCTGGTATCACGGT |
| | ACCTTTGTGCGCCTGTTTTATACCCCCTCCCCCAACTGTAACTTAGAA |
| | GTAACACACACCGATCAACAGTCAGCGTGGCACACCAGCCACGTTTT |
| | GATCAAGCACTTCTGTTACCCCGGACTGAGTATCAATAGACTGCTCAC |
| | GCGGTTGAAGGAGAAAGCGTTCGTTATCCGGCCAACTACTTCGAAAA |
| | ACCTAGTAACACCGTGGAAGTTGCAGAGTGTTTCGCTCAGCACTACC |
| | CCAGTGTAGATCAGGTCGATGAGTCACCGCATTCCCCACGGGCGACC |
| | GTGGCGGTGGCTGCGTTGGCGGCCTGCCCATGGGGAAACCCATGGGA |
| | CGCTCTAATACAGACATGGTGCGAAGAGTCTATTGAGCTAGTTGGTA |
| | GTCCTCCGGCCCCTGAATGCGGCTAATCCTAACTGCGGAGCACACAC |
| | CCTCAAGCCAGAGGGCAGTGTGTCGTAACGGGCAACTCTGCAGCGGA |
| | ACCGACTACTTTGGGTGTCCGTGTTTCATTTTATTCCTATACTGGCTGC |
| | TTATGGTGACAATTGAGAGATCGTTACCATATAGCTATTGGATTGGCC |
| | ATCCGGTGACTAATAGAGCTATTATATATCCCTTTGTTGGGTTTATAC |
| | CACTTAGCTTGAAAGAGGTTAAAACATTACAATTCATTGTTAAGTTGA |
| | ATACAGCAAAATGGTGAGCAAGGGCGAGGAGCTGTTCACCGGGGTG |
| | GTGCCCATCCTGGTCGAGCTGGACGGCGACGTAAACGGCCACAAGTT |
| | CAGCGTGTCCGGCGAGGGCGAGGGCGATGCCACCTACGGCAAGCTGA |
| | CCCTGAAGTTCATCTGCACCACCGGCAAGCTGCCCGTGCCCTGGCCCA |
| | CCCTCGTGACCACCCTGACCTACGGCGTGCAGTGCTTCAGCCGCTACC |
| | CCGACCACATGAAGCAGCACGACTTCTTCAAGTCCGCCATGCCCGAA |
| | GGCTACGTCCAGGAGCGCACCATCTTCTTCAAGGACGACGGCAACTA |
| | CAAGACCCGCGCCGAGGTGAAGTTCGAGGGCGACACCCTGGTGAACC |
| | GCATCGAGCTGAAGGGCATCGACTTCAAGGAGGACGGCAACATCCTG |
| | GGGCACAAGCTGGAGTACAACTACAACAGCCACAACGTCTATATCAT |
| | GGCCGACAAGCAGAAGAACGGCATCAAGGTGAACTTCAAGATCCGC |
| | CACAACATCGAGGACGGCAGCGTGCAGCTCGCCGACCACTACCAGCA |
| | GAACACCCCCATCGGCGACGGCCCCGTGCTGCTGCCCGACAACCACT |
| | ACCTGAGCACCCAGTCCGCCCTGAGCAAAGACCCCAACGAGAAGCGC |
| | GATCACATGGTCCTGCTGGAGTTCGTGACCGCCGCCGGGATCACTCTC |
| | GGCATGGACGAGCTGTACAAGTAAAAAAAACAAAAAACAAAACGGC |
| | TATTATGCGTTACCGGCGAGACG |
| Ana_CVB3_IRES- | GGGAGACCCTCGACCGTCGATTGTCCACTGGTCAACAATAGATGACT |
| antiCD19 TFP | TACAACTAATCGGAAGGTGCAGAGACTCGACGGGAGCTACCCTAACG |
| (SEQ ID NO: 148) | TCAAGACGAGGGTAAAGAGAGAGTCCAATTCTCAAAGCCAATAGGC |
| | AGTAGCGAAAGCTGCAAGAGAATGAAAATCCGTTGACCTTAAACGGT |
| | CGTGTGGGTTCAAGTCCCTCCACCCCCACGCCGGAAACGCAATAGCC |
| | GAAAAACAAAAAACAAAAAAATTAAAACAGCCTGTGGGTTGATCCC |
| | ACCCACAGGCCCATTGGGCGCTAGCACTCTGGTATCACGGTACCTTTG |
| | TGCGCCTGTTTTATACCCCCTCCCCCAACTGTAACTTAGAAGTAACAC |
| | ACACCGATCAACAGTCAGCGTGGCACACCAGCCACGTTTTGATCAAG |
| | CACTTCTGTTACCCCGGACTGAGTATCAATAGACTGCTCACGCGGTTG |
| | AAGGAGAAAGCGTTCGTTATCCGGCCAACTACTTCGAAAAACCTAGT |
| | AACACCGTGGAAGTTGCAGAGTGTTTCGCTCAGCACTACCCCAGTGT |
| | AGATCAGGTCGATGAGTCACCGCATTCCCCACGGGCGACCGTGGCGG |
| | TGGCTGCGTTGGCGGCCTGCCCATGGGGAAACCCATGGGACGCTCTA |
| | ATACAGACATGGTGCGAAGAGTCTATTGAGCTAGTTGGTAGTCCTCC |
| | GGCCCCTGAATGCGGCTAATCCTAACTGCGGAGCACACACCCTCAAG |
| | CCAGAGGGCAGTGTGTCGTAACGGGCAACTCTGCAGCGGAACCGACT |
| | ACTTTGGGTGTCCGTGTTTCATTTTATTCCTATACTGGCTGCTTATGGT |
| | GACAATTGAGAGATCGTTACCATATAGCTATTGGATTGGCCATCCGGT |
| | GACTAATAGAGCTATTATATATCCCTTTGTTGGGTTTATACCACTTAG |
| | CTTGAAAGAGGTTAAAACATTACAATTCATTGTTAAGTTGAATACAGC |
| | AAAATGCTTCTCCTGGTGACAAGCCTTCTGCTCTGTGAGTTACCACAC |
| | CCAGCATTCCTCCTGATCCCAGACATCCAGATGACACAGACTACATCC |
| | TCCCTGTCTGCCTCTCTGGGAGACAGAGTCACCATCAGTTGCAGGGCA |
| | AGTCAGGACATTAGTAAATATTTAAATTGGTATCAGCAGAAACCAGA |
| | TGGAACTGTTAAACTCCTGATCTACCATACATCAAGATTACACTCAGG |
| | AGTCCCATCAAGGTTCAGTGGCAGTGGGTCTGGAACAGATTATTCTCT |
| | CACCATTAGCAACCTGGAGCAAGAAGATATTGCCACTTACTTTTGCCA |
| | ACAGGGTAATACGCTTCCGTACACGTTCGGAGGGGGGACTAAGTTGG |
| | AAATAACAGGCTCCACCTCTGGATCCGGCAAGCCCGGATCTGGCGAG |
| | GGATCCACCAAGGGCGAGGTGAAACTGCAGGAGTCAGGACCTGGCCT |
| | GGTGGCGCCCTCACAGAGCCTGTCCGTCACATGCACTGTCTCAGGGG |
| | TCTCATTACCCGACTATGGTGTAAGCTGGATTCGCCAGCCTCCACGAA |
| | AGGGTCTGGAGTGGCTGGGAGTAATATGGGGTAGTGAAACCACATAC |
| | TATAATTCAGCTCTCAAATCCAGACTGACCATCATCAAGGACAACTCC |
| | AAGAGCCAAGTTTTCTTAAAAATGAACAGTCTGCAAACTGATGACAC |
| | AGCCATTTACTACTGTGCCAAACATTATTACTACGGTGGTAGCTATGC |
| | TATGGACTACTGGGGTCAAGGAACCTCAGTCACCGTCTCCGCGGCCG |
| | CGGGCGGTGGAGGCAGTGGTGGTGGCGGCTCTGGCGGTGGTGGTAGC |
| | CTCGAGGATGGTAATGAAGAAATGGGTGGTATTACACAGACACCATA |
| | TAAAGTCTCCATCTCTGGAACCACAGTAATATTGACATGCCCTCAGTA |
| | TCCTGGATCTGAAATACTATGGCAACACAATGATAAAAACATAGGCG |
| | GTGATGAGGATGATAAAAACATAGGCAGTGATGAGGATCACCTGTCA |
| | CTGAAGGAATTTTCAGAATTGGAGCAAAGTGGTTATTATGTCTGCTAC |
| | CCCAGAGGAAGCAAACCAGAAGATGCGAACTTTTATCTCTACCTGAG |
| | GGCAAGAGTGTGTGAGAACTGCATGGAGATGGATGTGATGTCGGTGG |
| | CCACAATTGTCATAGTGGACATCTGCATCACTGGGGGCTTGCTGCTGC |
| | TGGTTTACTACTGGAGCAAGAATAGAAAGGCCAAGGCCAAGCCTGTG |
| | ACACGAGGAGCGGGTGCTGGCGGCAGGCAAAGGGGACAAAACAAGG |
| | AGAGGCCACCACCTGTTCCCAACCCAGACTATGAGCCCATCCGGAAA |
| | GGCCAGCGGGACCTGTATTCTGGCCTGAATCAGAGACGCATCTGATA |
| | AAAAAAACAAAAAACAAAACGGCTATTATGCGTTACCGGCGAGACG |
| | CTACGGACTTAAATAATTGAGCCTTAAAGAAGAAATTCTTTAAGTGG |
| | ATGCTCTCAAACTCAGGGAAACCTAAATCTAGTTATAGACAAGGCAA |
| | TCCTGAGCCAAGCCGAAGTAGTAATTAGTAAGACCAGTGGACAATCG |
| | ACGGATAACAGCATATCTAG |
| Ana_CVB3_IRES- | GGGAGACCCAAGCTGGCTAGCGGGAGACCCTCGACCGTCGATTGTCC |
| antiMSLN TFP | ACTGGTCAACAATAGATGACTTACAACTAATCGGAAGGTGCAGAGAC |
| (SEQ ID NO: 149) | TCGACGGGAGCTACCCTAACGTCAAGACGAGGGTAAAGAGAGAGTC |
| | CAATTCTCAAAGCCAATAGGCAGTAGCGAAAGCTGCAAGAGAATGA |
| | AAATCCGTTGACCTTAAACGGTCGTGTGGGTTCAAGTCCCTCCACCCC |
| | CACGCCGGAAACGCAATAGCCGAAAAACAAAAAACAAAAAAATTAA |
| | AACAGCCTGTGGGTTGATCCCACCCACAGGCCCATTGGGCGCTAGCA |
| | CTCTGGTATCACGGTACCTTTGTGCGCCTGTTTTATACCCCCTCCCCCA |
| | ACTGTAACTTAGAAGTAACACACACCGATCAACAGTCAGCGTGGCAC |
| | ACCAGCCACGTTTTGATCAAGCACTTCTGTTACCCCGGACTGAGTATC |
| | AATAGACTGCTCACGCGGTTGAAGGAGAAAGCGTTCGTTATCCGGCC |
| | AACTACTTCGAAAAACCTAGTAACACCGTGGAAGTTGCAGAGTGTTT |
| | CGCTCAGCACTACCCCAGTGTAGATCAGGTCGATGAGTCACCGCATT |
| | CCCCACGGGCGACCGTGGCGGTGGCTGCGTTGGCGGCCTGCCCATGG |
| | GGAAACCCATGGGACGCTCTAATACAGACATGGTGCGAAGAGTCTAT |
| | TGAGCTAGTTGGTAGTCCTCCGGCCCCTGAATGCGGCTAATCCTAACT |
| | GCGGAGCACACACCCTCAAGCCAGAGGGCAGTGTGTCGTAACGGGCA |
| | ACTCTGCAGCGGAACCGACTACTTTGGGTGTCCGTGTTTCATTTTATT |
| | CCTATACTGGCTGCTTATGGTGACAATTGAGAGATCGTTACCATATAG |
| | CTATTGGATTGGCCATCCGGTGACTAATAGAGCTATTATATATCCCTT |
| | TGTTGGGTTTATACCACTTAGCTTGAAAGAGGTTAAAACATTACAATT |
| | CATTGTTAAGTTGAATACAGCAAAATGCTTCTGCTTGTGACGTCCCTC |
| | CTGCTTTGTGAACTCCCGCATCCTGCCTTCCTGCTCATCCCGGAAGTT |
| | CAGCTGGTAGAGTCCGGTGGCGGGCTGGTACAGCCAGGGGGATCTCT |
| | CCGTCTCTCATGTGCTGCCAGTGGGGGAGATTGGTCCGCTAACTTTAT |
| | GTACTGGTATCGTCAAGCACCTGGGAAACAACGCGAGCTGGTTGCAC |
| | GGATCTCTGGACGAGGGGTAGTGGATTACGTGGAAAGTGTGAAAGGA |
| | CGATTCACAATTTCCAGGGACAATTCTAAAAACACCCTTTATTTGCAA |
| | ATGAATAGTCTGAGAGCAGAAGACACAGCCGTATATTATTGTGCAGT |
| | CGCATCCTATTGGGGGCAGGGGACTCTTGTCACAGTCTCGAGTGCTGC |
| | AGCAGGCGGCGGGGGCTCAGGAGGGGGTGGGTCTGGAGGAGGTGGG |
| | AGTCTGGAGGATGGTAATGAAGAAATGGGTGGTATTACACAGACACC |
| | ATATAAAGTCTCCATCTCTGGAACCACAGTAATATTGACATGCCCTCA |
| | GTATCCTGGATCTGAAATACTATGGCAACACAATGATAAAAACATAG |
| | GCGGTGATGAGGATGATAAAAACATAGGCAGTGATGAGGATCACCTG |
| | TCACTGAAGGAATTTTCAGAATTGGAGCAAAGTGGTTATTATGTCTGC |
| | TACCCCAGAGGAAGCAAACCAGAAGATGCGAACTTTTATCTCTACCT |
| | GAGGGCAAGAGTGTGTGAGAACTGCATGGAGATGGATGTGATGTCGG |
| | TGGCCACAATTGTCATAGTGGACATCTGCATCACTGGCGGATTGCTGC |
| | TGCTGGTTTACTACTGGAGCAAGAATAGAAAGGCCAAGGCCAAGCCT |
| | GTGACACGAGGAGCGGGTGCTGGCGGCAGGCAAAGGGGACAAAACA |
| | AGGAGAGGCCACCACCTGTTCCCAACCCAGACTATGAGCCCATCCGG |
| | AAAGGCCAGCGGGACCTGTATTCTGGCCTGAATCAGAGACGCATCTA |
| | AAAAAAACAAAAAACAAAACGGCTATTATGCGTTACCGGCGAGACG |
| | CTACGGACTTAAATAATTGAGCCTTAAAGAAGAAATTCTTTAAGTGG |
| | ATGCTCTCAAACTCAGGGAAACCTAAATCTAGTTATAGACAAGGCAA |
| | TCCTGAGCCAAGCCGAAGTAGTAATTAGTAAGACCAGTGGACAATCG |
| | ACGGATAACAGCATATCTAGGGATC |

### Example 4: The Production of Self-Splicing Linear Precursor RNA

The TFPs encoding locus, Anabaena catalytic intron, and (Coxsackievirus B3 (CVB3) or encephalomyocarditis virus (EMCV)) IRES sequences are chemically synthesized by using Integrated DNA Technologies. The sequences are subsequently cloned into a linearized plasmid vector containing a T7 RNA polymerase promoter by Gibson Assembly® using a NEBuilder® HiFi DNA Assembly kit (New England Biolabs). Spacer regions, homology arms, and other variations are introduced using a Q5® Site-Directed Mutagenesis Kit (New England Biolabs). Linear precursor RNAs are synthesized by in vitro transcription from a linearized plasmid DNA template or PCR product using a T7 High Yield RNA Synthesis Kit (New England Biolabs).

### Example 5: The Production and Purification of CircRNA Encoding TFPs

Linear precursor RNA is treated with DNase I (New England Biolabs) for 20 min after in vitro transcription. The RNA samples are then column purified using a MEGAclear Transcription Clean up kit (Ambion). Linear precursor RNAs are then heated in the presence of magnesium ions and GTP to promote circularization, essentially as described previously for the circularization of shorter RNAs [Ford, E. & Ares, M. Proc. Natl Acad. Sci. 91, 3117-3121 (1994)]: RNA is heated to 70 C for 5 min and then immediately placed on ice for 3 min, after which GTP is added to a final concentration of 2 mM along with a buffer including magnesium (50 mM Tris-HCl, 10 mM MgCl2, 1 mM DTT, pH 7.5; New England Biolabs). RNA is then heated to 55 C for 40 min and then column purified.

Circularity check of the RNA using RNase R: To enrich for circRNA, 20 μg of RNA are diluted in water (88 μL final volume) and then heated at 70 C for 2 min and cooled on ice for 2 min. 20 U RNase R and 10 μL of 10 Å˜ RNase R buffer (Epicenter) are added, and the reaction is incubated at 37 C for 40 min; an additional 10 U RNase R are added halfway through the reaction. RNase R-digested RNA is column purified using Monarch RNA Cleanup Kit (New England Biolabs)

RNA is separated on precast 1.5% TBE agarose gel or precast 2% E-gel EX agarose gels (Invitrogen); ssRNA Ladders (NEB, ThermoFisher Scientific) is used as a standard. Bands are visualized using blue light transillumination. For gel extractions, bands corresponding to the circRNA are excised from the gel and then extracted using a Zymoclean™ Gel RNA Extraction Kit (Zymogen).

Purity of circRNA preparations is another factor essential for maximizing protein production from circRNA and for avoiding innate cellular immune responses [Kariko, K., Muramatsu, H., Ludwig, J. & Weissman, D. Nucleic Acids Res. 39, e142-e142 (2011)]. Therefore, as an alternative column purification method high-performance liquid chromatography (HPLC), fast protein liquid chromatography (FPLC), or size exclusion chromatography is applied. For HPLC 30 μg of RNA is heated at 65° C. for 3 min and then placed on ice for 3 min. RNA is run through a 4.6×300 mm size-exclusion column with a particle size of 5 μm and a pore size of 200 Å (Sepax Technologies; part number: 215980P-4630) on an Agilent 1100 Series HPLC (Agilent). RNA is run in Rnase-free TE buffer (10 mM Tris, 1 mM EDTA, pH:6) at a flow rate of 0.3 mL/minute. RNA is detected by UV absorbance at 260 nm, but is collected without UV detection. Resulting RNA fractions are precipitated with 5 M ammonium acetate, resuspended in water, and then in some cases treated with Rnase R as described above.

RNAs are purified from crude transcription reactions using an AKTA prime FPLC system equipped with a 50 mL superloop and three 5 mL HiTrap DEAE-sepharose FF columns (GE Healthcare) connected in series. The DEAE columns are equilibrated with three column volumes of buffer A (50 mM sodium phosphate [pH 6.5], 150 mM sodium chloride, and 0.2 mM EDTA) at room temperature. Buffer B contains the same components with 2 M sodium chloride. Both buffers can be prepared in large quantities, sterile filtered, and stored at 4° C. (buffer A) or room temperature (buffer B) to avoid precipitation of sodium chloride. The stopped transcription reaction (10-40 mL) is loaded into the 50 mL superloop and weak anion-exchange chromatography is performed using the following gradient, while collecting 10 mL fractions in sterile 15 mL plastic tubes: 0-70 mL (0% B at 1 mL/min) to load the sample onto the DEAE columns, 70-100 mL (0%-10% B at 2 mL/min) to wash remaining rNTPs off the column, 100-380 mL (10%-30% B at 2 mL/min) to separate small abortive transcripts, the desired RNA product, and the plasmid DNA template, 380-410 mL (30%-100% B at 4 mL/min), 410-455 mL (100% B at 4 mL/min), and 455-485 mL (100%-0% B at 4 mL/min) to ish and equilibrate the column for the next purification. For small-scale transcriptions below 1 mL, the reaction mixture is diluted to 2 mL with buffer A to ensure complete loading into the superloop and chromatography performed using a single 1-mL HiTrap DEAE-sepharose FF column and the same gradient profile with buffer volumes reduced to 1/15 collecting 2 mL fractions. Fractions containing desired RNA are precipitated with 5 M ammonium acetate, resuspended in water, and then in some embodiments treated with Rnase R as described above.

For SEC, the AKTA pure system is used connected to a FR-9 fraction collection under control of UNICORN 7.0 software suite. Circular RNA was injected through 0.5 ml sample loop to Superdex 200 increase column (24 ml). The column is equilibrated with PBS (NaCl 0.138M; KCl—0.0027M); pH=7.2 prepared in DEPC treated water. Chromatography is performed at 0.2 mL/min, collecting 0.5 or 0.25 ml fractions. All experiments were performed at 4° C.

### Example 6: Transfections of Jurkat Cells with CircRNA Encoding TFPs by Electroporation

The Jurkat cells are maintained at 0.2×10⁶ cells per mL in RPMI 1640 medium supplemented with 10% Fetal Bovine Serum (FBS) and 300 mg/L L-Glutamine until electroporation. 1-2 μg of circRNAs are mixed with 5×10⁵ T cells and electroporated according to the manufacturer's protocol for the Neon® Transfection System (ThermoFisher). Electroporation is set at 1600V, 10 ms, 3 pulses. After pulse the cells are immediately transferred to warm medium and incubated at 37° C. for three to seven days.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection.

### Example 7: Preparation of T-Cells Transduced with CircRNA Encoding TFPs

### PBMC Isolation

Peripheral blood mononuclear cells (PBMCs) are prepared from either whole blood or buffy coat. Whole blood is collected in 10 mL Heparin vacutainers and either processed immediately or stored overnight at 4° C. Approximately 10 mL of whole anti-coagulated blood is mixed with sterile phosphate buffered saline (PBS) buffer for a total volume of 20 mL in a 50 mL conical centrifuge tube (PBS, pH 7.4, without Ca²⁺/Mg²⁺). 20 mL of this blood/PBS mixture is then gently overlaid onto the surface of 15 mL of Ficoll-Paque® PLUS (GE Healthcare, 17-1440-03) prior to centrifugation at 400 g for 30-40 min at room temperature with no brake application.

Buffy coat is purchased from Research Blood Components (Boston, Mass.). LeucoSep® tubes (Greiner bio-one) are prepared by adding 15 mL Ficoll-Paque® (GE Health Care) and centrifuged at 1000 g for 1 minute. Buffy coat is diluted 1:3 in PBS (pH 7.4, without Ca²⁺ or Mg²⁺). The diluted buffy coat is transferred to LeucoSep tube and centrifuged at 1000 g for 15 minutes with no brake application. The layer of cells containing PBMCs, seen at the diluted plasma/Ficoll interface, is removed carefully to minimize contamination by Ficoll. Residual Ficoll, platelets, and plasma proteins are then removed by washing the PBMCs three times with 40 mL of PBS by centrifugation at 200 g for 10 minutes at room temperature. The cells are then counted with a hemocytometer. The washed PBMC are washed once with CART medium (AIM V-AlbuMAX® (BSA) (Life Technologies), with 5% AB serum and 1.25 μg/mL amphotericin B (Gemini Bioproducts, Woodland, Calif.), 100 U/mL penicillin, and 100 μg/mL streptomycin). Alternatively, the washed PBMC's are transferred to insulated vials and frozen at −80° C. for 24 hours before storing in liquid nitrogen for later use.

### T-Cell Activation

PBMCs prepared from either whole blood or buffy coat are stimulated with anti-human CD28 and CD3 antibody-conjugated magnetic beads for 24 hours prior to transfection. Freshly isolated PBMC are washed once in CAR T medium (AIM V-AlbuMAX (BSA) (Life Technologies), with 5% AB serum and 1.25 μg/mL amphotericin B (Gemini Bioproducts), 100 U/mL penicillin, and 100 μg/mL streptomycin) without huIL-2, before being re-suspended at a final concentration of 1×10⁶ cells/mL in CAR T medium with 300 IU/mL human IL-2 (from a 1000× stock; Invitrogen). If the PBMCs had previously been frozen they are thawed and re-suspended at 1×10⁷ cells/mL in 9 mL of pre-warmed (37° C.) cDMEM media (Life Technologies), in the presence of 10% FBS, 100 U/mL penicillin, and 100 μg/mL streptomycin, at a concentration of 1×10⁶ cells/mL prior to washing once in CAR T medium, re-suspension at 1×10⁶ cells/mL in CART medium, and addition of IL-2 as described above.

Prior to activation, anti-human CD28 and CD3 antibody-conjugated magnetic beads (available from, e.g., Invitrogen, Life Technologies) are washed three times with 1 mL of sterile 1×PBS (pH 7.4), using a magnetic rack to isolate beads from the solution, before re-suspension in CAR T medium, with 300 IU/mL human IL-2, to a final concentration of 4×10⁷ beads/mL. PBMC and beads are then mixed at a 1:1 bead-to-cell ratio, by transferring 25 μL (1×10⁶ beads) of beads to 1 mL of PBMC. The desired number of aliquots are then dispensed to single wells of a 12-well low-attachment or non-treated cell culture plate, and incubated at 37° C., with 5% CO₂, for 24 hours before transfection.

### T-Cell Transfection and Expansion

Following activation of PBMC, cells are incubated for 48 hours at 37° C., 5% CO₂. Then, 40-100 ng of Rnase R-treated splicing reactions or purified TFPs encoding circRNAs are reverse transfected into 10,000 cells/100 μL per well of a 96-well plate using Lipofectamine® MessengerMax™ (Invitrogen) or Lipofectamine® 2000 (Invitrogen, 11668-019) according to the manufacturer's instructions. The used kit is a lipid nanoparticle-based technology. For experiments wherein protein expression is assessed at multiple time points, media is fully removed and replaced at each time point. Sample sizes are chosen based on pilot experiments to determine assay variance and to minimize reagent consumption while allowing for meaningful differences between conditions to be distinguished.

Cells are then grown in the continued presence of 300 IU/mL of human IL-2 for a period of 6-14 days (total incubation time is dependent on the final number of CAR-T-cells). Cell concentrations are analyzed every 2-3 days, with media being added at that time to maintain the cell suspension at 1×10⁶ cells/mL.

In some instances, activated PBMCs are transfected by electroporation. In one embodiment, human PBMCs are stimulated with Dynabeads® (ThermoFisher) at 1-to-1 ratio for 3 days in the presence of 300 IU/ml recombinant human IL-2 (R&D Systems) (other stimulatory reagents such as TransAct® T Cell Reagent from Milyeni Pharmaceuticals may be used). The beads are removed before electroporation. The cells are washed and re-suspended in OPTI-MEM medium (ThermoFisher) at the concentration of 2.5×10⁷ cells/mL. 200 μL of the cell suspension (5×10⁶ cells) are transferred to the 2 mm gap Electroporation Cuvettes Plus™ (Harvard Apparatus BTX) and prechilled on ice. 10 μg of TPFs encoding circRNA is added to the cell suspension. The circRNA/cell mixture is then electroporated at 200 V for 20 milliseconds using ECM830 Electro Square Wave Porator (Harvard Apparatus BTX). Immediately after the electroporation, the cells are transferred to fresh cell culture medium (AIM V AlbuMAX® (BSA) serum free medium+5% human AB serum+300 IU/ml IL-2) and incubated at 37° C.

In some instances, T-cells are transfected by electroporation using a similar protocol as in Example 6. 1-2 μg of circRNAs are mixed with 5×10⁵ T cells and electroporated according to the manufacturer's protocol for the Neon. Transfection System (ThermoFisher). Electroporation is set at 1600V, 10 ms, 3 pulses. After pulse the cells are immediately transferred to warm medium and incubated at 37° C.

### Example 8: Protein Expression Analysis of Transfected Jurkat and T Cells

### Western Blot Analysis of the Translation Reaction

Western blot analysis of TFP expression in T cells and Jurkat cells is used to detect the presence of monomers and dimers (see, e.g., Milone et al., Molecular Therapy 17(8): 1453-1464 (2009)). Very briefly, T-cells or Jurkat cells expressing the TFPs are expanded in vitro for a period of 6-14 days followed by lysis and SDS-PAGE under reducing conditions. TFPs are detected by using an antibody to a TCR chain such as either anti-TCRα, anti-TCRβ, anti-CD3ε, anti-CD3γ, anti-CD3δ, or anti-CD3ζ. The same T-cell subsets are used for sodium dodecyl sulfate polyacrylamide gel electrophoresis (SDS-PAGE) analysis under non-reducing conditions to permit evaluation of covalent dimer formation.

### Flow Cytometry

Following transfection of T-cells and Jurkat Cells with circRNA, expression of TFPs, e.g., a TFP comprising a binding protein specific to mesothelin, CD19, BCMA, PD1, ROR1, CD22, IL13Ra2, etc., or dual specificity TFPs, is confirmed by flow cytometry. T cells are stained using anti-CD3 APC (Clone, UCHT1. BD Biosciences Catalog #340440, Lot #6005787), anti-CD4-Pacific blue (CloneRPAT4, Biolegend, Catalog #300521, Lot #B231611), anti-CD8-APCCY7 (Clone SK1, BD Biosciences, Catalog #557834, Lot #, 6082865), mesothelin antigen (Acro bioscience, Catalog #904x-7289F1-E7, Lot # 904x-3AOS1-4N), CD69-AF 700 (Clone FN50, Catalog #560739, Lot #7051802), Zenon R-Phycoerythrin Human IgG Labeling Kit (Thermofischer Scientific, Catalog number #Z25408, Lot #1863290) and isotype controls, APC Mouse IgG1, k Isotype Control (Clone X40, BD Biosciences, Catalog #340442), Pacific Blue isotype control (Clone MOPC-21, BD Biosciences, Catalog #558120), APCCY7 IgG1 isotype control (BD Biosciences, Catalog #557873), AF700 IgG1 isotype control (Clone 27-35, BD Biosciences, Catalog #560543), human NKG2D/CD314-APC (R&D systems, LOT #LC0061321) and its respective isotype control (BD biosciences). Flow cytometry is performed using BD-LSRII Fortessa® X20 (BD Biosciences) and data are acquired using FACS diva software and are analyzed with FlowJo® (Treestar, Inc. Ashland, Oreg.).

### Example 9: Cytotoxicity of MSTH-TFP

The luciferase-based cytotoxicity assay (“Luc-Cyto” assay) assesses the cytotoxicity of antiMSTH-TFP-expressing T cells by indirectly measuring the luciferase enzymatic activity in the residual live target cells after co-culture.

### Generation of Firefly Luciferase (Luc) Expressing Tumor Cells

The target cells used in the Luc-Cyto assay are Nalm6-Luc (CD19 positive) and K562-Luc (CD19 negative were generated by stably transducing Nalm6 (DSMZ Cat. #ACC 128) and K562 ((ATCC® Cat. #CCL-243™)) cells to express firefly luciferase. The DNA encoding firefly luciferase was synthesized by GeneArt® (ThermoFisher) and inserted into the multiple cloning site of single-promoter lentiviral vector pCDH527A-1 (System Biosciences). The lentivirus was packaged according to manufacturer's instruction. Tumor cells were then transduced with the lentivirus for 24 hours and then selected with puromycin (5 μg/mL). The successful generation of Nalm6-Luc and K562-Luc cells was confirmed by measuring the luciferase enzymatic activity in the cells with Bright-Glo™ Luciferase Assay System (Promega).

### Luc-Cyto Assay Assessing the Cytotoxicity of T Cells

The Luc-Cyto assay is set up by mixing T cells with tumor cells at different effector (T cell) to target (tumor cell) (E-to-T) ratios. The target cells (Nalm6-Luc or K562-Luc) are plated at 10,000 cells per well in 96-well plates with RPMI-1640 medium supplemented with 10% heat-inactivated (HI) FBS. TFP T cells are added to the tumor cells at 10000, 3333 or 1111 cells per well to reach E-to-T ratios of 1-to-1, or 1-to-3 or 1-to-9. The mixtures of cells are incubated for 24 hours at 37 C with 5% CO2. Luciferase enzymatic activity is measured using the Bright-Glo™ Luciferase Assay System (Promega), which measures activity from the residual live target cells in the T cell and tumor cell co-culture.

### Example 10: Generation and Transduction of circRNA Expressing GFP

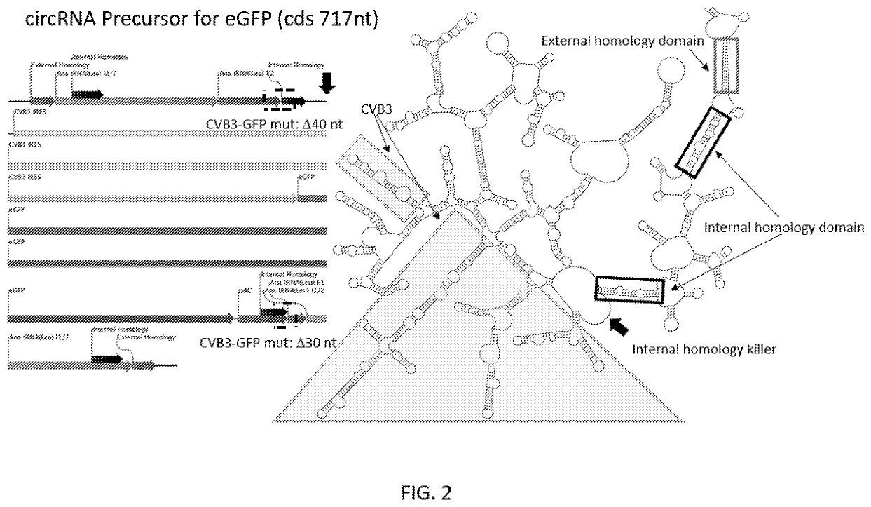

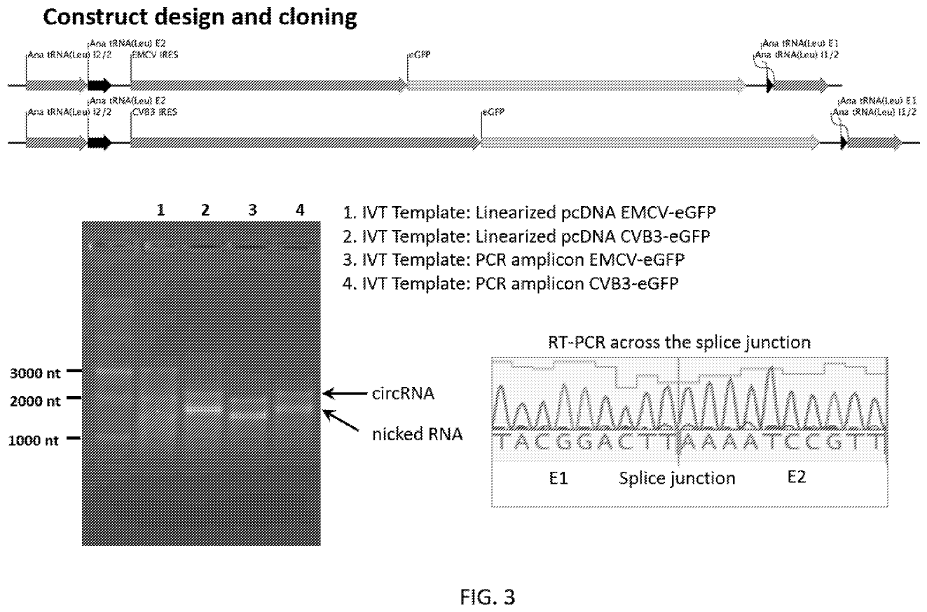

The sequence of a precursor RNA for generating a circRNA expressing GFP having the sequence of SEQ ID NO: 146 was designed according to the methods described in Example 1. As is shown in FIG. 2 and FIG. 3, the CBV3 or EMCV IRES followed by GFP was placed between two short fragments of the E2 and E1 exons downstream and upstream of the group I catalytic intron in Anabaena pre-tRNA gene. The 3′ half of the group I catalytic intron was placed upstream of E2 whereas the 5′ half of the group I catalytic intron was placed downstream of E1. A spacer was also introduced between the 3′ PIE splice site and the IRES. Complementary external homology arms of 33-35 nucleotides in length were also placed at the 5′ and 3′ ends. The sequence of the precursor RNA having the CBV3 IRES is SEQ ID NO: 146. FIG. 2 also shows the three-dimensional structure that is formed by this precursor RNA.

The sequence of the precursor RNA was first cloned into a DNA plasmid according to the methods described in Example 4. The plasmid was then linearized and the RNA precursor was then in vitro transcribed from the DNA template.

CircRNA was then generated from the linear precursor RNA according to the methods described in Example 5. The product was then run on an agarose gel. As is shown in FIG. 3, both the precursor RNA with the EMCV and CBV3 IRES form a circRNA circularized product, visible as a more slowly migrating band.

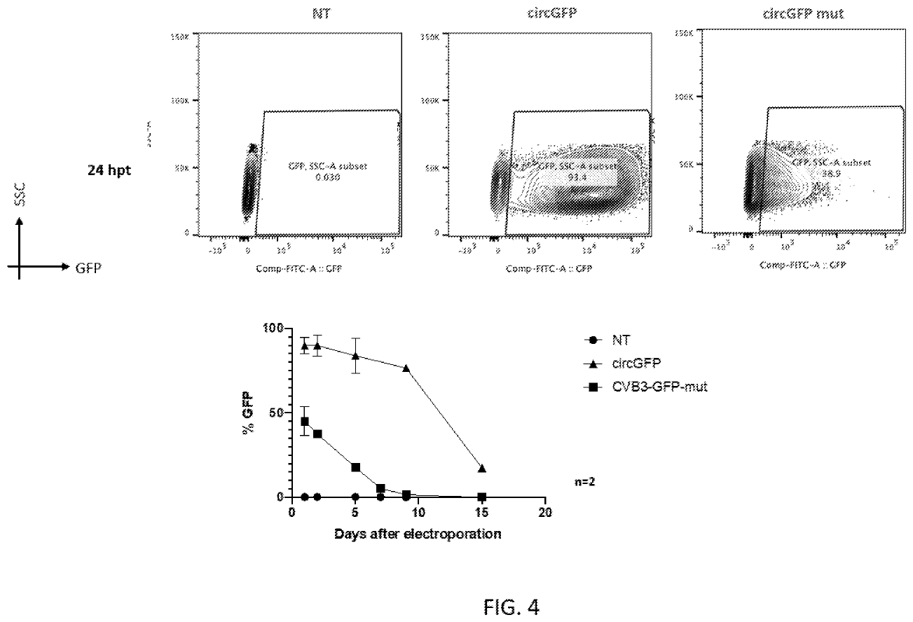

The circRNA was then transfected into Jurkat cells to evaluate the proportion of cells expressing the GFP protein. Jurkat cells were transected by electroporation according to the methods described in Example 6. Cells were untransduced, transduced with GFP circRNA, or transduced with a splice site mutated GFP precursor missing a 40-nucleotide region spanning splice site of the 3′ half of the group I catalytic intron and E2 and 30 nucleotide region spanning splice site of E1 and 5′half of the intron. This mutated GFP precursor was generated from a precursor RNA having the sequence of SEQ ID NO: 147. Protein expression in the transfected cells was measured by flow cytometry at 24 hours post transfection according to the methods described in Example 8. As is shown in FIG. 4, no untransduced (NT) cells expressed GFP, while cells transduced with GFP circRNA expressed GFP for at least 15 days. Cells transduced with mutated GFP circRNA expressed GFP for less than 10 days.

### Example 11: Generation and Transduction of CircRNA Expressing AntiCD19-TFP

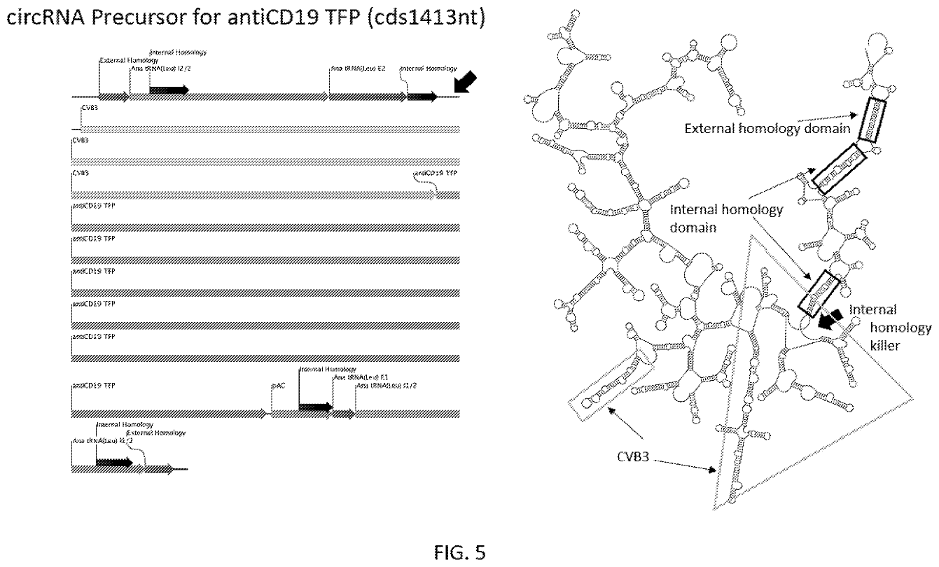

The sequence of a precursor RNA for generating a circRNA expressing antiCD19-TFP having the sequence of SEQ ID NO: 148 was designed according to the methods described in Example 2. As is shown in FIG. 5, the CBV3 IRES followed by antiCD19-TFP was placed between two short fragments of the E2 and E1 exons downstream and upstream of the group I catalytic intron in Anabaena pre-tRNA gene. The 3′ half of the group I catalytic intron was placed upstream of E2 whereas the 5′ half of the group I catalytic intron was placed downstream of E1. A spacer was also introduced between the 3′ PIE splice site and the IRES. Complementary external homology arms of 33-35 nucleotides in length were also placed at the 5′ and 3′ ends. FIG. 5 also shows the three-dimensional structure that is formed by the precursor RNA.

The sequence of the precursor RNA was first cloned into a DNA plasmid according to the methods described in Example 4. The plasmid was then linearized and the RNA precursor was then in vitro transcribed from the DNA template.

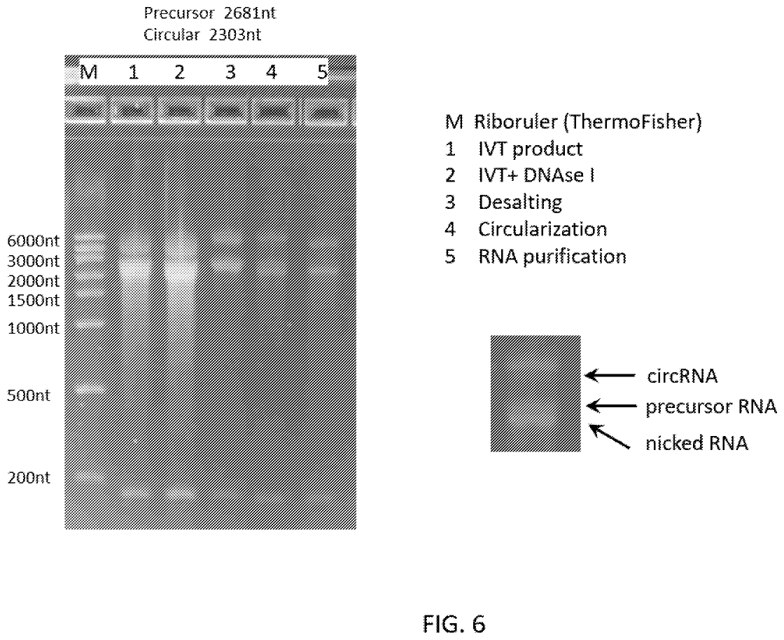

CircRNA was then generated from the linear precursor RNA according to the methods described in Example 5. RNA was visualized on an agarose gel following the IVT reaction and circularization. As is shown in FIG. 6, the precursor RNA forms a circRNA circularized product, visible as a more slowly migrating band.

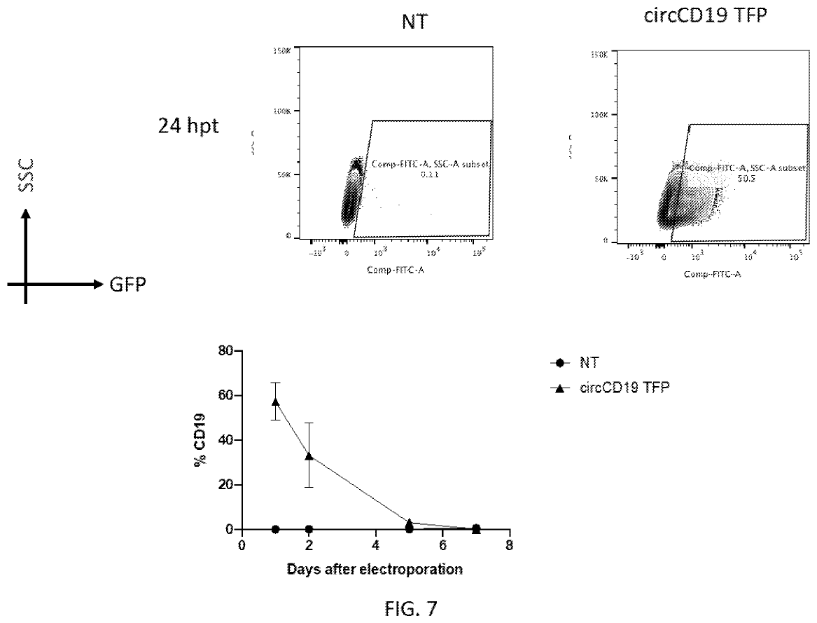

The circRNA was then transfected into Jurkat cells to evaluate the proportion of cells expressing the antiCD19-TFP protein. Jurkat cells were transected by electroporation according to the methods described in Example 6. Cells were untransduced or transduced with antiCD19-TFP circRNA. Protein expression in the transfected cells was measured by flow cytometry at 24 hours post transfection according to the methods described in Example 8. As is shown in FIG. 7, no untransduced cells expressed antiCD19-TFP, while cells transduced with antiCD19-TFP circRNA expressed antiCD19-TFP for at least 5 days.

### Example 12: Generation and Transduction of CircRNA Expressing antiMSLN-TFP

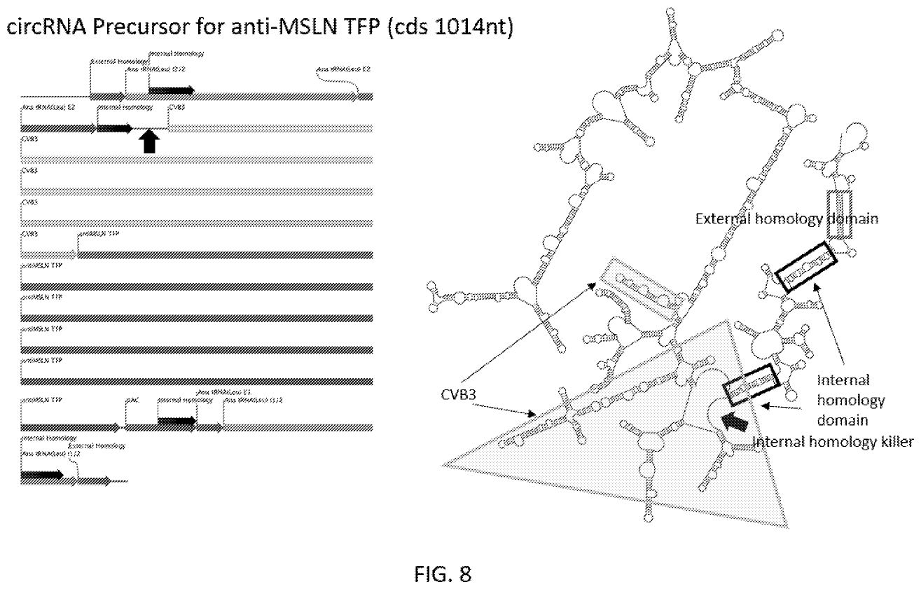

The sequence of a precursor RNA for generating a circRNA expressing antiMSLN-TFP having the sequence of SEQ ID NO: 149 was designed according to the methods described in Example 2. As is shown in FIG. 8, the CBV3 IRES followed by antiMSLN-TFP was placed between two short fragments of the E2 and E1 exons downstream and upstream of the group I catalytic intron in Anabaena pre-tRNA gene. The 3′ half of the group I catalytic intron was placed upstream of E2 whereas the 5′ half of the group I catalytic intron was placed downstream of E1. A spacer was also introduced between the 3′ PIE splice site and the IRES. Complementary external homology arms of 33-35 nucleotides in length were also placed at the 5′ and 3′ ends. FIG. 8 also shows the three-dimensional structure that is formed by the precursor RNA.

The sequence of the precursor RNA was first cloned into a DNA plasmid according to the methods described in Example 4. The plasmid was then linearized and the RNA precursor was then in vitro transcribed from the DNA template.

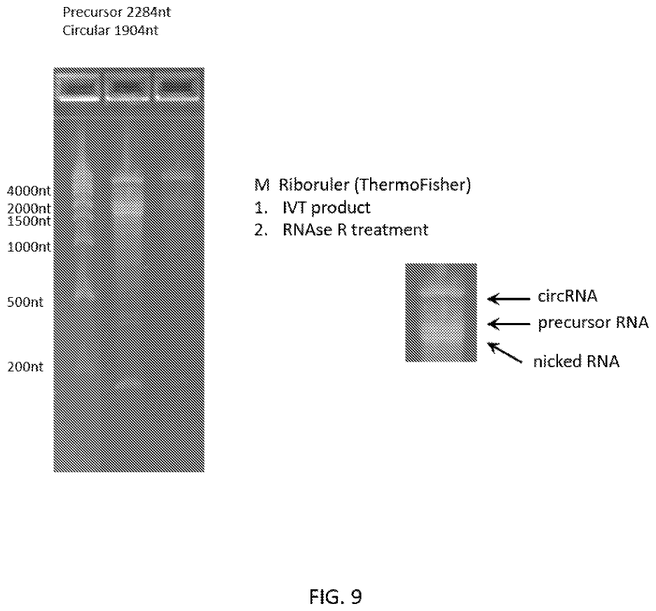

CircRNA was then generated from the linear precursor RNA according to the methods described in Example 5. RNA was visualized on an agarose gel following the IVT reaction and circularization. As is shown in FIG. 9, the precursor RNA forms a circRNA circularized product, visible as a more slowly migrating band. Moreover, this circularized product is more highly resistant to treatment with RNAse R than the linear precursor.

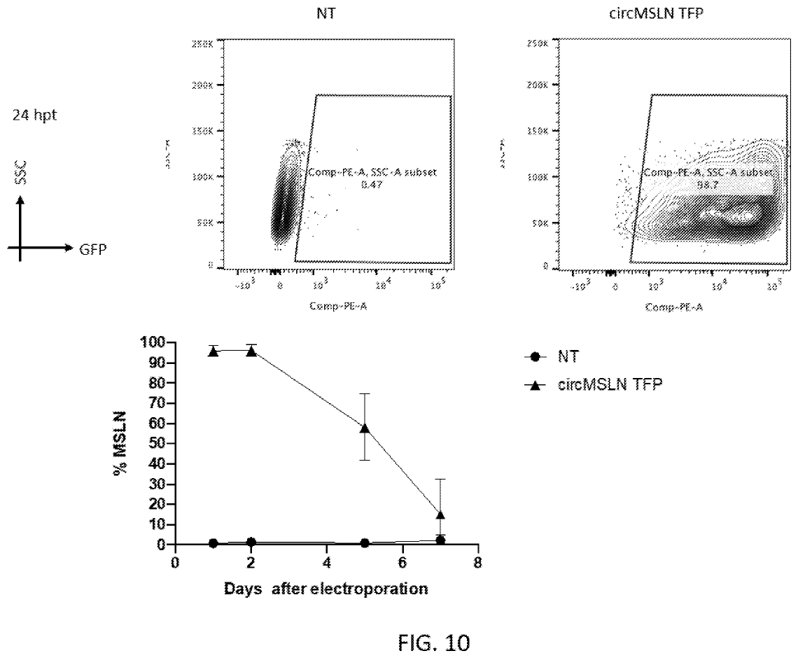

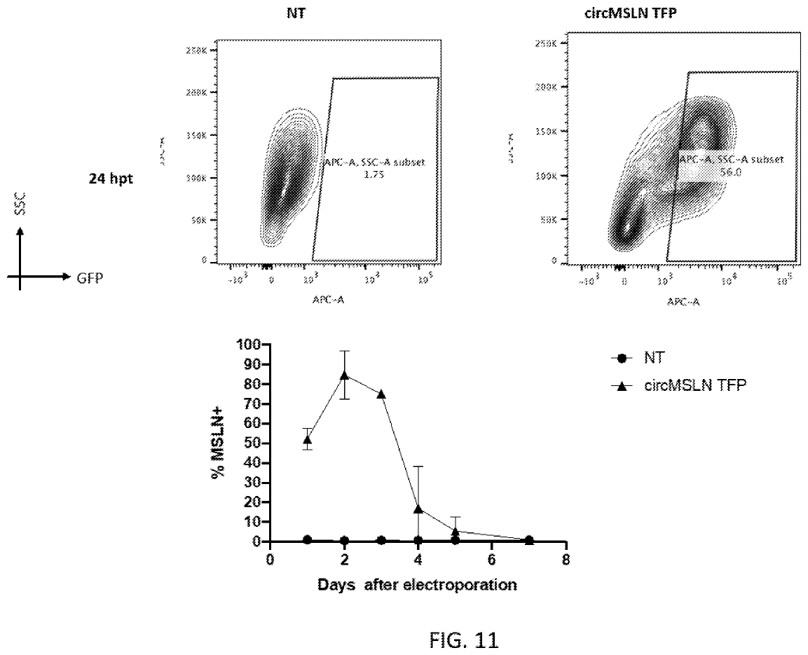

The circRNA was then transfected into Jurkat cells and activated T cells to evaluate the proportion of cells expressing the antiMSLN-TFP protein. Jurkat cells were transected by electroporation according to the methods described in Example 6 and T cells were transected by electroporation according to the methods described in Example 7. Cells were untransduced or transduced with antiMSLN-TFP circRNA. Protein expression in the transfected cells was measured by flow cytometry at 24 hours post transfection according to the methods described in Example 8. As is shown in FIG. 10, in Jurkat cells, no untransduced cells expressed antiMSLN-TFP, while cells transduced with antiMSLN-TFP circRNA expressed antiMSLN-TFP for at least 7 days. As is shown in FIG. 11, in activated T cells, no untransduced cells expressed antiMSLN-TFP, while cells transduced with antiMSLN-TFP circRNA expressed antiMSLN-TFP for at least 5 days.

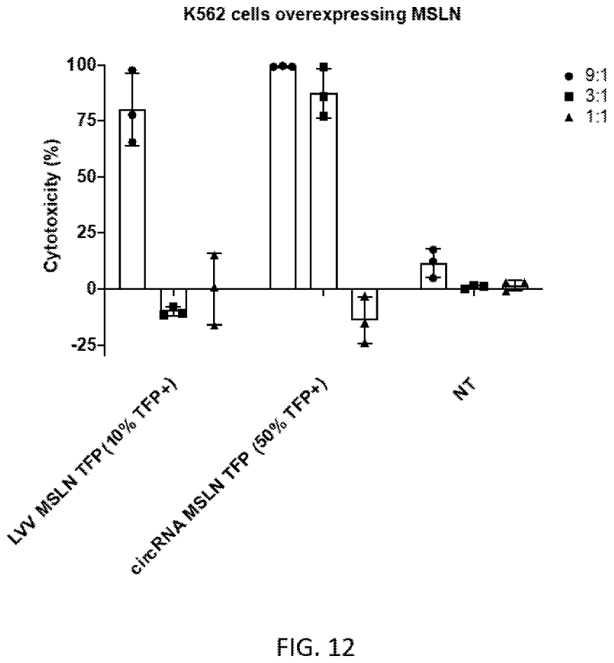

Cytotoxicity of the antiMSLN-TFP expressing T cells was measured according to the methods described in Example 9. The target cell used was K562-Luc cells overexpressing MSLN and the T cells were transduced with antiMSLN-TFP circRNA or an antiMSLN-TFP lentiviral vector. As is shown in FIG. 12, cells transduced with antiMSLN-TFP circRNA demonstrate increased cytotoxicity relative to cells transduced with an antiMSLN-TFP lentiviral vector, particularly at a 3:1 T to E ratio.

### Example 13: Generation and Transduction of CircRNA Expressing TAA(X)-TFP

The sequence of a precursor RNA for generating a circRNA expressing a TFP targeting a third antigen found on the surface of tumor cells, herein identified as TAA(X), for Tumor Associated Antigen X, was designed according to the methods described in Example 2. TFPs having four different binding domains targeting TAA(X) were used (TAA(X)-TFP1-4). The CBV3 IRES followed by the TAA(X)-TFP sequence was placed between two short fragments of the E2 and E1 exons downstream and upstream of the group I catalytic intron in Anabaena pre-tRNA gene. The 3′ half of the group I catalytic intron was placed upstream of E2 whereas the 5′ half of the group I catalytic intron was placed downstream of E1. A spacer was also introduced between the 3′ PIE splice site and the IRES. Complementary external homology arms of 33-35 nucleotides in length were also placed at the 5′ and 3′ ends.

The sequence of the precursor RNA was first cloned into a DNA plasmid according to the methods described in Example 4. The plasmid was then linearized and the RNA precursor was then in vitro transcribed from the DNA template.

CircRNA was then generated from the linear precursor RNA according to the methods described in Example 5.

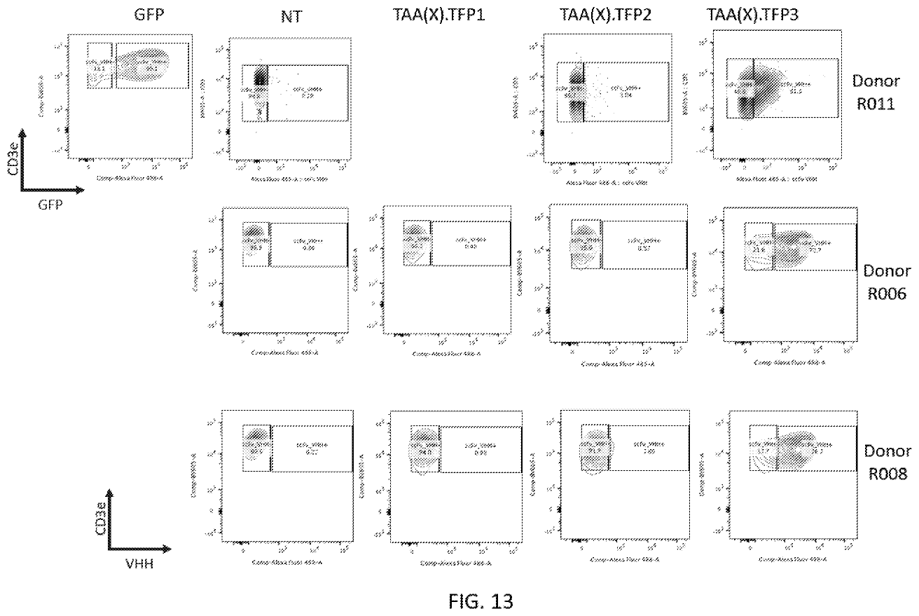

The circRNA was then transfected into T cells from three donors to evaluate the proportion of cells expressing the TAA(X)-TFP protein. T cells were transected by electroporation according to the methods described in Example 7. Cells were untransduced or electroporated with TAA(X)-TFP circRNA. Protein expression in the electroporated cells was measured by flow cytometry at 24 hours post transfection according to the methods described in Example 8. As is shown in FIG. 13, untransduced cells did not express TAA(X)-TFP, while TAA(X)-TFP expression was detected in transduced cells at 24 hours post-transfection.

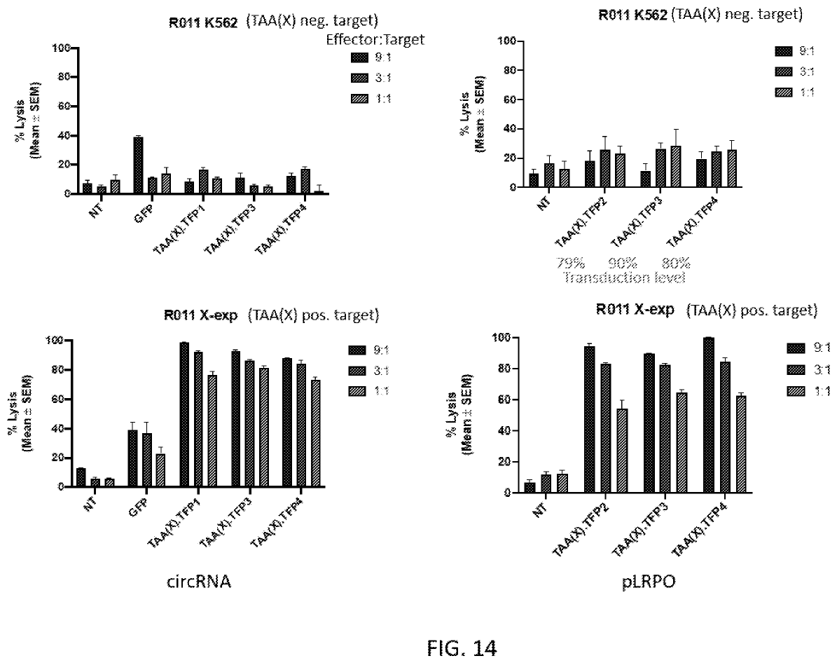

Cytotoxicity of the TAA(X)-TFP expressing T cells was measured according to the methods described in Example 9. The target cell used was control K562-Luc cells or Luc cells the antigen targeted by the TFP, and the T cells were electroporated with TAA(X)-TFP circRNA or transduced with a TAA(X)-TFP lentiviral vector. As is shown in FIG. 14, cells transduced with TAA(X)-TFP circRNA demonstrate similar cytotoxicity relative to cells transduced with an antiMSLN-TFP lentiviral vector.

### Example 14: Effect of m6A Inclusion in CircRNA

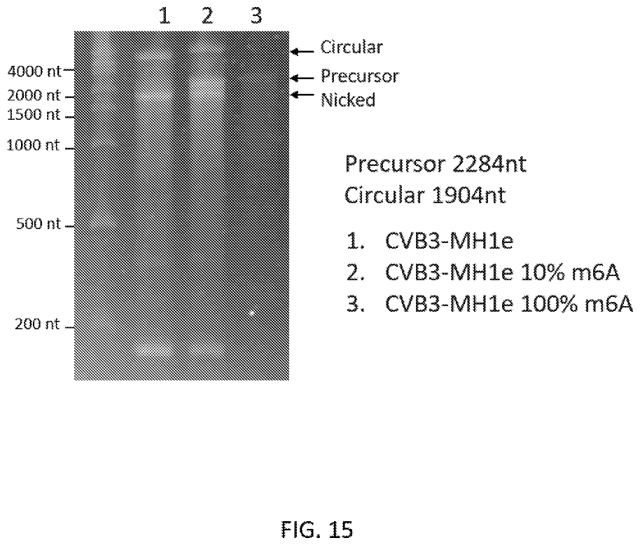

It has previously been reported that N6-methyladenosine (m6A) in circRNA reduces immunogenicity (Chen et al., Molecular Cell 2019). To investigate the role of m6A on TFP T cells generated with circRNA, precursor linear RNA for CVB3-MH1e was generated as previously described by IVT, using 0%, 10% or 100% m6A, and the IVT product was circularized. As is shown in FIG. 15, the IVT and circularization products were visualized on an agarose gel. After the circularization step, constructs containing 10% or 100% m6A had a visible linear product remaining, whereas the construct lacking m6A did not, suggesting m6A inhibits circularization.

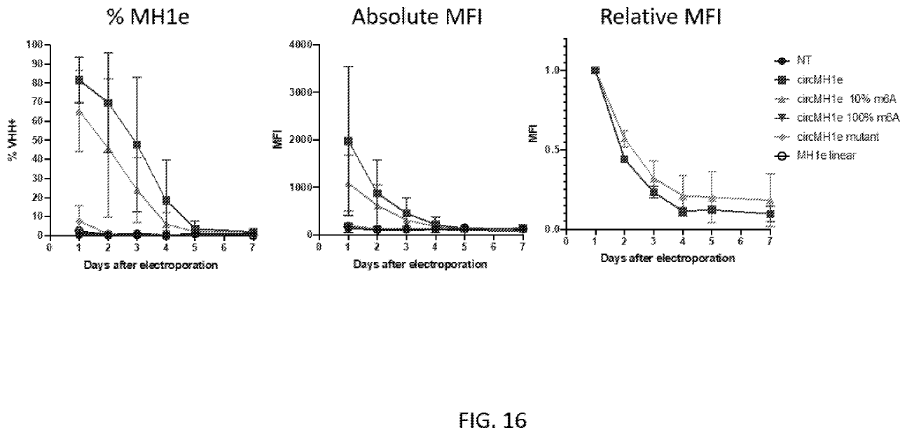

The circRNA constructs were then electroporated into T cells as is described in Example 7. Cell surface expression was measured by flow cytometry over course of 7 days. As is shown in FIG. 16, constructs containing m6A had reduced proportion of TFP positive cells and MFI (mean fluorescence intensity) relative to the construct lacking m6A.

### Example 15: Immunogenicity of CircRNA in T Cells

Cellular immune response was measured in T cells (Table 3) and in THP-1 cells (Table 4) following electroporation by measuring expression of IFN-β1, RANTES, RIG-1, and MDA5 via RT-PCR. Expression levels were normalized to the expression of the housekeeping gene RPL13A. Cells were untransduced, mock electroporated, or electroporated with (1) GFP circRNA, (2) a splice site mutated GFP precursor, (3) linear GFP RNA, (4) Trilink GFP RNA, (5) MH1e (MSLN) circRNA, (6) MH1e (MSLN) circRNA having 10% m6A, (7) a splice site mutated MH1e precursor, (8) linear MH1e RNA, (9), 3p-hairpin RNA at 0.1 ug/ml or 1 ug/ml, or (10) Poly I:C at 1 ug/ml. shRNA is to the RIG-I ligand and Poly I:C is the MDA5 ligand. While ds RNA (poly I:C) and hairpin RNA induces a strong immune response in both cell types, linear GFP RNA, GFP circRNA, and circRNA having 10% m6A induces a significant immune response in THP-1 but not in T-cells.

TABLE 3
IFN-β1, RANTES, RIG-I and MDA-5 transcript induction 24
hours after electroporation of T-cells with indicated RNAs

| | IFNB1 | RANTES | RIG-I | MDA5 |
|---|---|---|---|---|
| NT | 1.00 | 1.00 | 1.00 | 1.00 |
| mock | 0.94 | 1.01 | 2.17 | 1.64 |
| circGFP | 2.63 | 0.79 | 0.95 | 1.00 |
| circGFP mut | 0.91 | 0.77 | 0.96 | 1.08 |
| GFP-linear | 0.86 | 0.85 | 0.93 | 0.89 |
| GFP Trilink | 0.84 | 0.88 | 1.01 | 1.01 |
| circMH1e | 3.28 | 0.95 | 1.57 | 1.17 |
| circMH1e | 1.58 | 1.16 | 1.19 | 1.39 |
| 10% m6A | | | | |
| circMH1e | 1.29 | 1.14 | 1.13 | 1.17 |
| mutant | | | | |
| 3p-phRNA | 2.76 | 1.14 | 2.11 | 2.38 |
| (0.1 ug/ml) | | | | |
| 3p-phRNA | 5.02 | 1.55 | 3.89 | 4.50 |
| (1 ug/ml) | | | | |
| Poly I:C | 1105.57 | 7.07 | 30.49 | 10.43 |
| (1 ug/ul) | | | | |

TABLE 4
IFN-β1, RANTES, RIG-I and MDA-5 transcript induction 24
hours after electroporation of THP-1 cells with indicated RNAs

| | IFNB1 | RANTES | RIG-I | MDA5 |
|---|---|---|---|---|
| NT | 1 | 1 | 1 | 1 |
| Mock | 0.91 | 1.97 | 1.12 | 1.27 |
| circGFP | 8.14 | 6.11 | 19.42 | 16.76 |
| circGFP | 3.81 | 5.86 | 10.90 | 6.65 |
| mut | | | | |
| GFP-linear | 5.94 | 3.69 | 5.42 | 5.93 |
| GFP Trilink | 1.89 | 3.20 | 2.19 | 2.65 |
| circMH1e | 2.75 | 2.70 | 10.70 | 6.86 |
| circMH1e | 5.43 | 4.77 | 18.06 | 12.55 |
| 10% m6A | | | | |
| circMH1e | 1.22 | 1.05 | 2.41 | 2.27 |
| mut | | | | |
| MH1e | 4.42 | 4.51 | 12.96 | 12.99 |
| linear | | | | |
| 3p-phRNA | 33.22 | 44.02 | 81.44 | 71.25 |
| 0.1 ug/ml | | | | |
| 3p-phRNA | 364.56 | 240.49 | 471.91 | 196.38 |
| 1 ug/ml | | | | |
| Poly I:C | 4795.02 | 384.33 | 403.98 | 159.83 |
| 1 ug/ml | | | | |

### Example 16: Delivery of T-Cells Electroporated with CircRNA to Solid Tumor Xenograft Mouse Model

The efficacy of T-cells transfected with TFPs encoding circRNA is tested in immune compromised mouse models bearing subcutaneous solid tumors derived from human BCMA-expressing ALL, CLL or NHL human cell lines. Tumor shrinkage in response to T-cell treatment can be either assessed by caliper measurement of tumor size, or by following the intensity of a GFP fluorescence signal emitted by GFP-expressing tumor cells.

Primary human solid tumor cells can be grown in immune compromised mice without having to culture them in vitro. Exemplary solid cancer cells include solid tumor cell lines, such as provided in The Cancer Genome Atlas (TCGA) and/or the Broad Cancer Cell Line Encyclopedia (CCLE, see Barretina et al., Nature 483:603 (2012)). Exemplary solid cancer cells include primary tumor cells isolated from mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, or stomach cancer. In some embodiments, the cancer to be treated is selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas.

The immunocompromised mice are used to test the efficacy of T-cells electroporated with TFPs encoding circRNA in the human tumor xenograft models (see, e.g., Morton et al., Nat. Procol. 2:247 (2007)). Following an implant or injection of 1×10⁶-1×10⁷ primary cells (collagenase-treated bulk tumor suspensions in EC matrix material) or tumor fragments (primary tumor fragments in EC matrix material) subcutaneously, tumors are allowed to grow to 200-500 mm³ prior to initiation of treatment.

A NOD/SCID (NSG) mouse model is used to conduct an in vivo potency study. Female NOD/SCID/IL-2Rγ−/− (NSG-JAX) mice, at least 6 weeks of age prior to the start of the study, are obtained from The Jackson Laboratory (stock number 005557) and acclimated for 3 days before experimental use. Human BCMA-expressing cell lines for inoculation are maintained in log-phase culture prior to harvesting and counting with trypan blue to determine a viable cell count. On the day of tumor challenge, the cells are centrifuged at 300 g for 5 minutes and re-suspended in pre-warmed sterile PBS at 0.5-1×10⁶ cells/100 μL. 3×10⁶ RPMI-8226-Luc cells are injected subcutaneously (s.c.) into NSG mice. 19 days post tumor inoculation, T-cells transfected with TFPs encoding circRNA are administered at 15×10⁶ cells per mouse i.v. There are 7 animals per group. Bioluminescent imaging is performed on days 3, 7, 14, 21, 28 and 35 of study. Tumor volumes are measured by caliper measurements two days per week. Detailed clinical observations on the animals are recorded daily until euthanasia. Body weight measurements are made on all animals weekly until death or euthanasia. All animals are euthanized 35 days after adoptive transfer of test and control articles. Any animals appearing moribund during the study are euthanized at the discretion of the study director in consultation with a veterinarian.

### Example 17: Delivery of CircRNA Encoding TFPs to Solid Tumor Xenograft Mouse Model

The ideal circRNA in vivo delivery systems are expected to keep their payloads against abundant endonucleases present in the tumor microenvironment, avoid immune detection, prevent nonspecific interactions with proteins or nontarget cells, allow targeted delivery to tissues of interest and promote cell entry efficacy. Delivery strategies involve systemic injection into the vasculature, subcutaneous injection or depots, or local application.

Lipid nanoparticles (LNPs) are prepared by mixing ethanol and aqueous phase at a 1:3 volumetric ratio in a microfluidic device, using syringe pumps as previously described. In brief, ethanol phase is prepared by solubilizing a mixture of ionizable lipidoid cKK-E12, 1,2-dioleoyl-sn-glycero-3-phosphoethanolamine (DOPE), cholesterol, and 1,2-dimyristoyl-sn-glycero-3-phosphoethanolamine-N-[methoxy-(polyethyleneglycol)-2000] (ammonium salt) (C14-PEG 2000) at a molar ratio of 35:16:46.5:2.5. The aqueous phase is prepared in 10 mM citrate buffer (pH 3) with circRNA. LNPs are dialyzed against PBS in a Slide-A-Lyzer G2 Dialysis Cassettes, 20,000 MWCO (Thermo Fisher) for 2 h at RT. The concentration of circRNA encapsulated into LNPs nanoparticles is analyzed using Quant-iT™ RiboGreen® assay (Thermo Fisher) according to the manufacturer's protocol. The efficiency of circRNA encapsulation into LNPs is calculated by comparing measurements in the absence and presence of 1% (v/v) Triton X-100. Nano-particle size, polydispersity (PDI), and z-potential are analyzed by dynamic light scattering (DLS) using Zetasizer Nano ZS (Malvern Instruments, Worcestershire, UK).

1.5 to 150 picomoles of LNP-circRNA in a total volume of 50 mL are intravenously injected within 10 min to ANOD/SCID (NSG) mouse model described in Example 9 [Wu et al. PloS one. 10(3). (2015)]. Bioluminescent imaging is performed on days 3, 7, 14, 21, 28 and 35 after injection. Tumor volumes are measured by caliper measurements two days per week after injection.

### Example 18: LPN Formulation for Delivery of CircRNA Encoding TFPs to Solid Tumor Xenograft Mouse Model

LPNs are formed via standard ethanol injection methods (Ponsa, M.; Foradada, M.; Estelrich, J. “Liposomes obtained by the ethanol injection method” Int. J. Pharm. 1993, 95, 51-56). For the various lipid components, a 50 mg/ml ethanolic stock solutions is prepared and stored at −20° C.

In preparation of the lipid nanoparticle formulation listed in Table 5, each indicated lipid component is added to an ethanol solution to achieve a predetermined final concentration and molar ratio and scaled to a 3 ml final volume of ethanol. Separately, an aqueous buffered solution (10 mM citrate/150 mM NaCl, pH 4.5) of isolated circRNA is prepared from a 1 mg/ml stock. The lipid solution is injected rapidly into the aqueous circRNA solution and shaken to yield a final suspension in 20% ethanol. The resulting nanoparticle suspension is filtered and dialysed against 1×PBS (pH 7.4), concentrated and stored between 2-8° C. Encapsulation of circRNA is calculated by performing the Ribogreen assay with and without the presence of 0.1% Triton-X 100. Particle sizes (dynamic light scattering (DLS)) and zeta potentials are determined using a Malvern Zetasizer instrument in 1×PBS and 1 mM KCl solutions, respectively.

1.5 to 150 picomoles of LNP-circRNA in a total volume of 50 mL are intravenously injected within 10 min to ANOD/SCID (NSG) mouse model described in Example 17 [Wu et al. PloS one. 10(3). (2015)]. Bioluminescent imaging is performed on days 3, 7, 14, 21, 28 and 35 after injection. Tumor volumes are measured by caliper measurements two days per week after injection.

TABLE 5
Lipid nanoparticle formulation in example 18.

| Components of | Molar ratio | Final circRNA |
|---|---|---|
| Formulation | of lipids | concentration |
| cKK-E12 | 40:30:25:5 | 1.8 mg/ml |
| DOPE | 40:30:25:5 | 1.8 mg/ml |
| Cholesterol | 40:30:25:5 | 1.8 mg/ml |
| DMG-PEG-2K | 40:30:25:5 | 1.8 mg/ml |

APPENDIX A
SEQUENCE SUMMARY

| SEQ | | |
|---|---|---|
| ID | | |
| NO. | Name | Sequence |
| 1 | human CD3-ϵ | MQSGTHWRVLGLCLLSVGVWGQDGNEEMGGITQTPYKVSISGTT |
| | | VILTCPQYPGSEILWQHNDKNIGGDEDDKNIGSDEDHLSLKEFSEL |
| | | EQSGYYVCYPRGSKPEDANFYLYLRARVCENCMEMDVMSVATIV |
| | | IVDICITGGLLLLVYYWSKNRKAKAKPVTRGAGAGGRQRGQNKE |
| | | RPPPVPNPDYEPIRKGQRDLYSGLNQRRI |
| 2 | human CD3-γ | MEQGKGLAVLILAIILLQGTLAQSIKGNHLVKVYDYQEDGSVLLT |
| | | CDAEAKNITWFKDGKMIGFLTEDKKKWNLGSNAKDPRGMYQCK |
| | | GSQNKSKPLQVYYRMCQNCIELNAATISGFLFAEIVSIFVLAVGVY |
| | | FIAGQDGVRQSRASDKQTLLPNDQLYQPLKDREDDQYSHLQGNQ |
| | | LRRN |
| 3 | human CD3-δ | MEHSTFLSGLVLATLLSQVSPFKIPIEELEDRVFVNCNTSITWVEGT |
| | | VGTLLSDITRLDLGKRILDPRGIYRCNGTDIYKDKESTVQVHYRMC |
| | | QSCVELDPATVAGIIVTDVIATLLLALGVFCFAGHETGRLSGAADT |
| | | QALLRNDQVYQPLRDRDDAQYSHLGGNWARNKS |
| 4 | human CD3-ζ | MKWKALFTAAILQAQLPITEAQSFGLLDPKLCYLLDGILFIYGVILT |
| | | ALFLRVKFSRSADAPAYQQGQNQLYNELNLGRREEYDVLDKRRG |
| | | RDPEMGGKPQRRKNPQEGLYNELQKDKMAEAYSEIGMKGERRR |
| | | GKGHDGLYQGLSTATKDTYDALHMQALPPR |
| 5 | human TCR α- | MAGTWLLLLLALGCPALPTGVGGTPFPSLAPPIMLLVDGKQQMV |
| | chain | VVCLVLDVAPPGLDSPIWFSAGNGSALDAFTYGPSPATDGTWTNL |
| | | AHLSLPSEELASWEPLVCHTGPGAEGHSRSTQPMHLSGEASTART |
| | | CPQEPLRGTPGGALWLGVLRLLLFKLLLFDLLLTCSCLCDPAGPLP |
| | | SPATTTRLRALGSHRLHPATETGGREATSSPRPQPRDRRWGDTPPG |
| | | RKPGSPVWGEGSYLSSYPTCPAQAWCSRSALRAPSSSLGAFFAGD |
| | | LPPPLQAGA |
| 6 | human TCR α- | PNIQNPDPAVYQLRDSKSSDKSVCLFTDFDSQTNVSQSKDSDVYIT |
| | chain C region | DKTVLDMRSMDFKSNSAVAWSNKSDFACANAFNNSIIPEDTFFPS |
| | | PESSCDVKLVEKSFETDTNLNFQNLSVIGFRILLLKVAGFNLLMTL |
| | | RLWSS |
| 7 | human TCR α- | MAMLLGASVLILWLQPDWVNSQQKNDDQQVKQNSPSLSVQEGRI |
| | chain V region | SILNCDYTNSMFDYFLWYKKYPAEGPTFLISISSIKDKNEDGRFTVF |
| | CTL-L17 | LNKSAKHLSLHIVPSQPGDSAVYFCAAKGAGTASKLTFGTGTRLQ |
| | | VTL |
| 8 | human TCR β- | EDLNKVFPPEVAVFEPSEAEISHTQKATLVCLATGFFPDHVELSW |
| | chain C region | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 9 | human TCR β- | MGTSLLCWMALCLLGADHADTGVSQNPRHNITKRGQNVTFRCDP |
| | chain V region | ISEHNRLYWYRQTLGQGPEFLTYFQNEAQLEKSRLLSDRFSAERPK |
| | CTL-L17 | GSFSTLEIQRTEQGDSAMYLCASSLAGLNQPQHFGDGTRLSIL |
| 10 | human TCR β- | MDSWTFCCVSLCILVAKHTDAGVIQSPRHEVTEMGQEVTLRCKPI |
| | chain V region | SGHNSLFWYRQTMMRGLELLIYFNNNVPIDDSGMPEDRFSAKMP |
| | YT35 | NASFSTLKIQPSEPRDSAVYFCASSFSTCSANYGYTFGSGTRLTVV |
| 11 | MSLN amino acid | MALPTARPLLGSCGTPALGSLLFLLFSLGWVQPSRTLAGETGQEA |
| | sequence: human | APLDGVLANPPNISSLSPRQLLGFPCAEVSGLSTERVRELAVALAQ |
| | mesothelin | KNVKLSTEQLRCLAHRLSEPPEDLDALPLDLLLFLNPDAFSGPQAC |
| | sequence | TRFFSRITKANVDLLPRGAPERQRLLPAALACWGVRGSLLSEADV |
| | (UniProt | RALGGLACDLPGRFVAESAEVLLPRLVSCPGPLDQDQQEAARAAL |
| | Accession No. | QGGGPPYGPPSTWSVSTMDALRGLLPVLGQPIIRSIPQGIVAAWRQ |
| | Q13421) | RSSRDPSWRQPERTILRPRFRREVEKTACPSGKKAREIDESLIFYKK |
| | | WELEACVDAALLATQMDRVNAIPFTYEQLDVLKHKLDELYPQGY |
| | | PESVIQHLGYLFLKMSPEDIRKWNVTSLETLKALLEVNKGHEMSP |
| | | QVATLIDRFVKGRGQLDKDTLDTLTAFYPGYLCSLSPEELSSVPPS |
| | | SIWAVRPQDLDTCDPRQLDVLYPKARLAFQNMNGSEYFVKIQSFL |
| | | GGAPTEDLKALSQQNVSMDLATFMKLRTDAVLPLTVAEVQKLLG |
| | | PHVEGLKAEERHRPVRDWILRQRQDDLDTLGLGLQGGIPNGYLVL |
| | | DLSMQEALSGTPCLLGPGPVLTVLALLLASTLA |
| 12 | p510_anti- | acgcgtgtagtcttatgcaatactcttgtagtcttgcaacatggtaacgatgagt |
| | MSLN_SS1_CD3ϵ | tagcaacatgccttacaaggagagaaaaagcaccgtgcatgccgattggtggaag |
| | DNA | taaggtggtacgatcgtgccttattaggaaggcaacagacgggtctgacatggat |
| | | tggacgaaccactgaattgccgcattgcagagatattgtatttaagtgcctagct |
| | | cgatacaataaacgggtctctctggttagaccagatctgagcctgggagctctct |
| | | ggctaactagggaacccactgcttaagcctcaataaagcttgccttgagtgcttc |
| | | aagtagtgtgtgcccgtctgttgtgtgactctggtaactagagatccctcagacc |
| | | cttttagtcagtgtggaaaatctctagcagtggcgcccgaacagggacctgaaag |
| | | cgaaagggaaaccagagctctctcgacgcaggactcggcttgctgaagcgcgcac |
| | | ggcaagaggcgaggggcggcgactggtgagtacgccaaaaattttgactagcgga |
| | | ggctagaaggagagagatgggtgcgagagcgtcagtattaagcgggggagaatta |
| | | gatcgcgatgggaaaaaattcggttaaggccagggggaaagaaaaaatataaatt |
| | | aaaacatatagtatgggcaagcagggagctagaacgattcgcagttaatcctggc |
| | | ctgttagaaacatcagaaggctgtagacaaatactgggacagctacaaccatccc |
| | | ttcagacaggatcagaagaacttagatcattatataatacagtagcaaccctcta |
| | | ttgtgtgcatcaaaggatagagataaaagacaccaaggaagctttagacaagata |
| | | gaggaagagcaaaacaaaagtaagaccaccgcacagcaagcggccactgatcttc |
| | | agacctggaggaggagatatgagggacaattggagaagtgaattatataaatata |
| | | aagtagtaaaaattgaaccattaggagtagcacccaccaaggcaaagagaagagt |
| | | ggtgcagagagaaaaaagagcagtgggaataggagctttgttccttgggttcttg |
| | | ggagcagcaggaagcactatgggcgcagcctcaatgacgctgacggtacaggcca |
| | | gacaattattgtctggtatagtgcagcagcagaacaatttgctgagggctattga |
| | | ggcgcaacagcatctgttgcaactcacagtctggggcatcaagcagctccaggca |
| | | agaatcctggctgtggaaagatacctaaaggatcaacagctcctggggatttggg |
| | | gttgctctggaaaactcatttgcaccactgctgtgccttggaatgctagttggag |
| | | taataaatctctggaacagattggaatcacacgacctggatggagtgggacagag |
| | | aaattaacaattacacaagcttaatacactccttaattgaagaatcgcaaaacca |
| | | gcaagaaaagaatgaacaagaattattggaattagataaatgggcaagtttgtgg |
| | | aattggtttaacataacaaattggctgtggtatataaaattattcataatgatag |
| | | taggaggcttggtaggtttaagaatagtttttgctgtactttctatagtgaatag |
| | | agttaggcagggatattcaccattatcgtttcagacccacctcccaaccccgagg |
| | | ggacccgacaggcccgaaggaatagaagaagaaggtggagagagagacagagaca |
| | | gatccattcgattagtgaacggatctcgacggtatcggttaacttttaaaagaaa |
| | | aggggggattggggggtacagtgcaggggaaagaatagtagacataatagcaaca |
| | | gacatacaaactaaagaattacaaaaacaaattacaaaattcaaaattttatcga |
| | | tactagtattatgcccagtacatgaccttatgggactttcctacttggcagtaca |
| | | tctacgtattagtcatcgctattaccatggtgatgcggttttggcagtacatcaa |
| | | tgggcgtggatagcggtttgactcacggggatttccaagtctccaccccattgac |
| | | gtcaatgggagtttgttttggcaccaaaatcaacgggactttccaaaatgtcgta |
| | | acaactccgccccattgacgcaaatgggcggtaggcgtgtacggtgggaggttta |
| | | tataagcagagctcgtttagtgaaccgtcagatcgcctggagacgccatccacgc |
| | | tgttttgacctccatagaagattctagagccgccaccatgcttctcctggtgaca |
| | | agccttctgctctgtgagttaccacacccagcattcctcctgatcccagacattc |
| | | agcaggtccagctccagcagtctggccctgaactcgaaaaacctggcgctagcgt |
| | | gaaaatttcctgtaaagcctccggctactcttttactggctacacaatgaattgg |
| | | gtgaaacagtctcacggcaaatccctcgaatggatcggactcatcacaccctaca |
| | | atggcgcctcttcctacaaccagaaattccggggcaaggcaacactcactgtgga |
| | | caaatcatcctctaccgcctacatggatctgctctccctcacatctgaggactcc |
| | | gctgtctacttttgtgcccgaggaggatacgacggacgaggattcgattactggg |
| | | gacagggaacaactgtgaccgtgtctagtggcggcggagggagtggaggcggagg |
| | | atcttctggcgggggatccgatattgaactcacacagtctcccgctatcatgtct |
| | | gcttctcccggcgagaaagtgactatgacttgctctgcttcctcttctgtgtcct |
| | | acatgcactggtaccagcagaaatctggcacatcccctaaacggtggatctacga |
| | | tactagcaaactggcatccggcgtgcctgggcgattctctggctctggctctggc |
| | | aactcttactctctcacaatctcatctgtcgaggctgaggacgatgccacatact |
| | | actgtcagcagtggtctaaacacccactcacattcggcgctggcactaaactgga |
| | | aataaaagcggccgcaggtggcggcggttctggtggcggcggttctggtggcggc |
| | | ggttctctcgaggatggtaatgaagaaatgggtggtattacacagacaccatata |
| | | aagtctccatctctggaaccacagtaatattgacatgccctcagtatcctggatc |
| | | tgaaatactatggcaacacaatgataaaaacataggcggtgatgaggatgataaa |
| | | aacataggcagtgatgaggatcacctgtcactgaaggaattttcagaattggagc |
| | | aaagtggttattatgtctgctaccccagaggaagcaaaccagaagatgcgaactt |
| | | ttatctctacctgagggcaagagtgtgtgagaactgcatggagatggatgtgatg |
| | | tcggtggccacaattgtcatagtggacatctgcatcactgggggcttgctgctgc |
| | | tggtttactactggagcaagaatagaaaggccaaggccaagcctgtgacacgagg |
| | | agcgggtgctggcggcaggcaaaggggacaaaacaaggagaggccaccacctgtt |
| | | cccaacccagactatgagcccatccggaaaggccagcgggacctgtattctggcc |
| | | tgaatcagagacgcatctgataagaattcgatccgcggccgcgaaggatctgcga |
| | | tcgctccggtgcccgtcagtgggcagagcgcacatcgcccacagtccccgagaag |
| | | ttggggggaggggtcggcaattgaacgggtgcctagagaaggtggcgcggggtaa |
| | | actgggaaagtgatgtcgtgtactggctccgcctttttcccgagggtgggggaga |
| | | accgtatataagtgcagtagtcgccgtgaacgttctttttcgcaacgggtttgcc |
| | | gccagaacacagctgaagcttcgaggggctcgcatctctccttcacgcgcccgcc |
| | | gccctacctgaggccgccatccacgccggttgagtcgcgttctgccgcctcccgc |
| | | ctgtggtgcctcctgaactgcgtccgccgtctaggtaagtttaaagctcaggtcg |
| | | agaccgggcctttgtccggcgctcccttggagcctacctagactcagccggctct |
| | | ccacgctttgcctgaccctgcttgctcaactctacgtctttgtttcgttttctgt |
| | | tctgcgccgttacagatccaagctgtgaccggcgcctacgctagatgaccgagta |
| | | caagcccacggtgcgcctcgccacccgcgacgacgtccccagggccgtacgcacc |
| | | ctcgccgccgcgttcgccgactaccccgccacgcgccacaccgtcgatccggacc |
| | | gccacatcgagcgggtcaccgagctgcaagaactcttcctcacgcgcgtcgggct |
| | | cgacatcggcaaggtgtgggtcgcggacgacggcgccgcggtggcggtctggacc |
| | | acgccggagagcgtcgaagcgggggcggtgttcgccgagatcggcccgcgcatgg |
| | | ccgagttgagcggttcccggctggccgcgcagcaacagatggaaggcctcctggc |
| | | gccgcaccggcccaaggagcccgcgtggttcctggccaccgtcggcgtctcgccc |
| | | gaccaccagggcaagggtctgggcagcgccgtcgtgctccccggagtggaggcgg |
| | | ccgagcgcgccggggtgcccgccttcctggagacctccgcgccccgcaacctccc |
| | | cttctacgagcggctcggcttcaccgtcaccgccgacgtcgaggtgcccgaagga |
| | | ccgcgcacctggtgcatgacccgcaagcccggtgcctgagtcgacaatcaacctc |
| | | tggattacaaaatttgtgaaagattgactggtattcttaactatgttgctccttt |
| | | tacgctatgtggatacgctgctttaatgcctttgtatcatgctattgcttcccgt |
| | | atggctttcattttctcctccttgtataaatcctggttgctgtctctttatgagg |
| | | agttgtggcccgttgtcaggcaacgtggcgtggtgtgcactgtgtttgctgacgc |
| | | aacccccactggttggggcattgccaccacctgtcagctcctttccgggactttc |
| | | gctttccccctccctattgccacggcggaactcatcgccgcctgccttgcccgct |
| | | gctggacaggggctcggctgttgggcactgacaattccgtggtgttgtcggggaa |
| | | atcatcgtcctttccttggctgctcgcctgtgttgccacctggattctgcgcggg |
| | | acgtccttctgctacgtcccttcggccctcaatccagcggaccttccttcccgcg |
| | | gcctgctgccggctctgcggcctcttccgcgtcttcgccttcgccctcagacgag |
| | | tcggatctccctttgggccgcctccccgcctggtacctttaagaccaatgactta |
| | | caaggcagctgtagatcttagccactttttaaaagaaaaggggggactggaaggg |
| | | ctaattcactcccaacgaaaataagatctgctttttgcttgtactgggtctctct |
| | | ggttagaccagatctgagcctgggagctctctggctaactagggaacccactgct |
| | | taagcctcaataaagcttgccttgagtgcttcaagtagtgtgtgcccgtctgttg |
| | | tgtgactctggtaactagagatccctcagacccttttagtcagtgtggaaaatct |
| | | ctagcagtagtagttcatgtcatcttattattcagtatttataacttgcaaagaa |
| | | atgaatatcagagagtgagaggaacttgtttattgcagcttataatggttacaca |
| | | aataaagcaatagcatcacaaatttcacaaataaagcatttttttctgcattcta |
| | | gttgtggtttgtccaaactcatcaatgtatcttatcatgtctggctctagctatc |
| | | ccgcccctaactccgcccagttccgcccattctccgccccatggctgactaattt |
| | | tttttatttatgcagaggccgaggccgcctcggcctctgagctattccagaagta |
| | | gtgaggaggcttttttggaggcctagacttttgcagagacggcccaaattcgtaa |
| | | tcatggtcatagctgtttcctgtgtgaaattgttatccgctcacaattccacaca |
| | | acatacgagccggaagcataaagtgtaaagcctggggtgcctaatgagtgagcta |
| | | actcacattaattgcgttgcgctcactgcccgctttccagtcgggaaacctgtcg |
| | | tgccagctgcattaatgaatcggccaacgcgcggggagaggcggtttgcgtattg |
| | | ggcgctcttccgcttcctcgctcactgactcgctgcgctcggtcgttcggctgcg |
| | | gcgagcggtatcagctcactcaaaggcggtaatacggttatccacagaatcaggg |
| | | gataacgcaggaaagaacatgtgagcaaaaggccagcaaaaggccaggaaccgta |
| | | aaaaggccgcgttgctggcgtttttccataggctccgcccccctgacgagcatca |
| | | caaaaatcgacgctcaagtcagaggtggcgaaacccgacaggactataaagatac |
| | | caggcgtttccccctggaagctccctcgtgcgctctcctgttccgaccctgccgc |
| | | ttaccggatacctgtccgcctttctcccttcgggaagcgtggcgctttctcatag |
| | | ctcacgctgtaggtatctcagttcggtgtaggtcgttcgctccaagctgggctgt |
| | | gtgcacgaaccccccgttcagcccgaccgctgcgccttatccggtaactatcgtc |
| | | ttgagtccaacccggtaagacacgacttatcgccactggcagcagccactggtaa |
| | | caggattagcagagcgaggtatgtaggcggtgctacagagttcttgaagtggtgg |
| | | cctaactacggctacactagaaggacagtatttggtatctgcgctctgctgaagc |
| | | cagttaccttcggaaaaagagttggtagctcttgatccggcaaacaaaccaccgc |
| | | tggtagcggtggtttttttgtttgcaagcagcagattacgcgcagaaaaaaagga |
| | | tctcaagaagatcctttgatcttttctacggggtctgacgctcagtggaacgaaa |
| | | actcacgttaagggattttggtcatgagattatcaaaaaggatcttcacctagat |
| | | ccttttaaattaaaaatgaagttttaaatcaatctaaagtatatatgagtaaact |
| | | tggtctgacagttaccaatgcttaatcagtgaggcacctatctcagcgatctgtc |
| | | tatttcgttcatccatagttgcctgactccccgtcgtgtagataactacgatacg |
| | | ggagggcttaccatctggccccagtgctgcaatgataccgcgagacccacgctca |
| | | ccggctccagatttatcagcaataaaccagccagccggaagggccgagcgcagaa |
| | | gtggtcctgcaactttatccgcctccatccagtctattaattgttgccgggaagc |
| | | tagagtaagtagttcgccagttaatagtttgcgcaacgttgttgccattgctaca |
| | | ggcatcgtggtgtcacgctcgtcgtttggtatggcttcattcagctccggttccc |
| | | aacgatcaaggcgagttacatgatcccccatgttgtgcaaaaaagcggttagctc |
| | | cttcggtcctccgatcgttgtcagaagtaagttggccgcagtgttatcactcatg |
| | | gttatggcagcactgcataattctcttactgtcatgccatccgtaagatgctttt |
| | | ctgtgactggtgagtactcaaccaagtcattctgagaatagtgtatgcggcgacc |
| | | gagttgctcttgcccggcgtcaatacgggataataccgcgccacatagcagaact |
| | | ttaaaagtgctcatcattggaaaacgttcttcggggcgaaaactctcaaggatct |
| | | taccgctgttgagatccagttcgatgtaacccactcgtgcacccaactgatcttc |
| | | agcatcttttactttcaccagcgtttctgggtgagcaaaaacaggaaggcaaaat |
| | | gccgcaaaaaagggaataagggcgacacggaaatgttgaatactcatactcttcc |
| | | tttttcaatattattgaagcatttatcagggttattgtctcatgagcggatacat |
| | | atttgaatgtatttagaaaaataaacaaataggggttccgcgcacatttccccga |
| | | aaagtgccacctgacgtctaagaaaccattattatcatgacattaacctataaaa |
| | | ataggcgtatcacgaggccctttcgtctcgcgcgtttcggtgatgacggtgaaaa |
| | | cctctgacacatgcagctcccggagacggtcacagcttgtctgtaagcggatgcc |
| | | gggagcagacaagcccgtcagggcgcgtcagcgggtgttggcgggtgtcggggct |
| | | ggcttaactatgcggcatcagagcagattgtactgagagtgcaccatatgcggtg |
| | | tgaaataccgcacagatgcgtaaggagaaaataccgcatcaggcgccattcgcca |
| | | ttcaggctgcgcaactgttgggaagggcgatcggtgcgggcctcttcgctattac |
| | | gccagctggcgaaagggggatgtgctgcaaggcgattaagttgggtaacgccagg |
| | | gttttcccagtcacgacgttgtaaaacgacggccagtgccaagctg |
| 13 | p510_anti- | MLLLVTSLLLCELPHPAFLLIPDIQQVQLQQSGPELEKPGASVKISC |
| | MSLN_SS1_CD3ϵ | KASGYSFTGYTMNWVKQSHGKSLEWIGLITPYNGASSYNQKFRG |
| | amino acid | KATLTVDKSSSTAYMDLLSLTSEDSAVYFCARGGYDGRGFDYWG |
| | | QGTTVTVSSGGGGSGGGGSSGGGSDIELTQSPAIMSASPGEKVTM |
| | | TCSASSSVSYMHWYQQKSGTSPKRWIYDTSKLASGVPGRFSGSGS |
| | | GNSYSLTISSVEAEDDATYYCQQWSKHPLTFGAGTKLEIKAAAGG |
| | | GGSGGGGSGGGGSLEDGNEEMGGITQTPYKVSISGTTVILTCPQYP |
| | | GSEILWQHNDKNIGGDEDDKNIGSDEDHLSLKEFSELEQSGYYVC |
| | | YPRGSKPEDANFYLYLRARVCENCMEMDVMSVATIVIVDICITGG |
| | | LLLLVYYWSKNRKAKAKPVTRGAGAGGRQRGQNKERPPPVPNPD |
| | | YEPIRKGQRDLYSGLNQRRI* |
| 14 | Anti-MSLN Light | DVVMTQTPLSLPVSLGDQASISCRSSQSLVHSNGNTYLHWYLQKP |
| | Chain amino acid | GQSPKLLIYKVSNRFSGVPDRFSGSGSGTDFTLKITRVEAEDLGVFF |
| | (MHC1445LC.1) | CSQSTHVPFTFGSGTKLEIK |
| 15 | Anti-MSLN Light | gatgttgtgatgacccaaactccactctccctgcctgtcagtcttggagatcaag |
| | Chain DNA | cctccatctcttgcagatctagtcagagccttgtacacagtaatggaaacaccta |
| | (MHC1445LC.1) | tttacattggtacctgcagaagccaggccagtctccaaagctcctgatctacaaa |
| | | gtttccaaccgattttctggggtcccagacaggttcagtggcagtggatcaggga |
| | | ctgatttcacactcaagatcaccagagtggaggctgaggatctgggagttttttt |
| | | ctgctctcaaagtacacatgttccattcacgttcggctcggggacaaagttggaa |
| | | ataaaa |
| 16 | Anti-MSLN Heavy | QVQLQQSGAELVRPGASVTLSCKASGYTFFDYEMHWVKQTPVHG |
| | Chain amino acid | LEWIGAIDPEIDGTAYNQKFKGKAILTADKSSSTAYMELRSLTSED |
| | (MHC1445HC.1) | SAVYYCTDYYGSSYWYFDVWGTGTTVTVSS |
| 17 | Anti-MSLN | caggttcaactgcagcagtctggggctgagctggtgaggcctggggcttcagtga |
| | Heavy Chain DNA | cgctgtcctgcaaggcttcgggctacacattttttgactatgaaatgcactgggt |
| | (MHC1445HC.1) | gaagcagacacctgtgcatggcctggaatggattggagctattgatcctgaaatt |
| | | gatggtactgcctacaatcagaagttcaagggcaaggccatactgactgcagaca |
| | | aatcctccagcacagcctacatggagctccgcagcctgacatctgaggactctgc |
| | | cgtctattactgtacagattactacggtagtagctactggtacttcgatgtctgg |
| | | ggcacagggaccacggtcaccgtctcctc |
| 18 | Anti-MSLN Light | DVMMTQTPLSLPVSLGDQASISCRSSQSLVHSNGNTYLHWFLQKP |
| | Chain amino acid | GQSPKLLIYKVSNRFSGVPDRFSGSGSGTDFTLKISRVEAEDLGVY |
| | (MHC1446LC.1) | FCSQTTHVPLTFGAGTKLELK |
| 19 | Anti-MSLN Light | gatgttatgatgacccaaactccactctccctgcctgtcagtcttggagatcaag |
| | Chain DNA | cctccatctcttgcagatctagtcagagccttgtacacagtaatggaaacaccta |
| | (MHC1446LC.1) | tttacattggttcctgcagaagccaggccagtctccaaagctcctgatctacaaa |
| | | gtttccaaccgattttctggggtcccagacaggttcagtggcagtggatcaggga |
| | | cagatttcacactcaagatcagcagagtggaggctgaggatctgggagtttattt |
| | | ctgctctcaaactacacatgttccgctcacgttcggtgctgggaccaagctggag |
| | | ctgaaa |
| 20 | Anti-MSLN Heavy | QVQLQQSGAELVRPGASVTLSCKASGYTFTDYEMHWVKQTPVHG |
| | Chain amino acid | LEWIGAIDPEIAGTAYNQKFKGKAILTADKSSSTAYMELRSLTSED |
| | (MHC1446HC.3) | SAVYYCSRYGGNYLYYFDYWGQGTTLTVSS |
| 21 | Anti-MSLN Heavy | caggttcaactgcagcagtctggggctgagctggtgaggcctggggcttcagtga |
| | Chain DNA | cgctgtcctgcaaggcttcgggctacacttttactgactatgaaatgcactgggt |
| | (MHC1446HC.3) | gaagcagacacctgtccatggcctggaatggattggagctattgatcctgaaatt |
| | | gctggtactgcctacaatcagaagttcaagggcaaggccatactgactgcagaca |
| | | aatcctccagcacagcctacatggagctccgcagcctgacatctgaggactctgc |
| | | cgtctattactgttcaagatacggtggtaactacctttactactttgactactgg |
| | | ggccaaggcaccactctcacagtctcctca |
| 22 | Anti-MSLN Light | DVLMTQIPLSLPVSLGDQASISCRSSQNIVYSNGNTYLEWYLQKPG |
| | Chain amino acid | QSPKLLIYKVSNRFSGVPDRFSGSGSGTDFTLKISRVEAEDLGVYY |
| | (MHC1447LC.5) | CFQGSHVPFTFGSGTKLEIK |
| 23 | Anti-MSLN Light | gatgttttgatgacccaaattccactctccctgcctgtcagtcttggagatcaag |
| | Chain DNA | cctccatctcttgcagatctagtcagaacattgtgtatagtaatggaaacaccta |
| | (MHC1447LC.5) | tttagagtggtacctgcagaaaccaggccagtctccaaagctcctgatctacaaa |
| | | gtttccaaccgattttctggggtcccagacaggttcagtggcagtggatcaggga |
| | | cagatttcacactcaagatcagcagagtggaggctgaggatctgggagtttatta |
| | | ctgctttcaaggttcacatgttccattcacgttcggctcggggacaaagttggaa |
| | | ataaaa |
| 24 | Anti-MSLN Heavy | QVQLQQSGAELVRPGASVTLSCKASGYTFTDYEMHWVKQTPVHG |
| | Chain amino acid | LEWIGAIDPEIGGSAYNQKFKGRAILTADKSSSTAYMELRSLTSED |
| | (MHC1447HC.5) | SAVYYCTGYDGYFWFAYWGQGTLVTVSS |
| 25 | Anti-MSLN Heavy | caggttcaactgcagcagtccggggctgagctggtgaggcctggggcttcagtga |
| | Chain DNA | cgctgtcctgcaaggcttcgggctacacatttactgactatgaaatgcactgggt |
| | (MHC1447HC.5) | gaagcagacacctgtgcatggcctggaatggattggagctattgatcctgaaatt |
| | | ggtggttctgcctacaatcagaagttcaagggcagggccatattgactgcagaca |
| | | aatcctccagcacagcctacatggagctccgcagcctgacatctgaggactctgc |
| | | cgtctattattgtacgggctatgatggttacttttggtttgcttactggggccaa |
| | | gggactctggtcactgtctcttca |
| 26 | Anti-MSLN Light | ENVLTQSPAIMSASPGEKVTMTCSASSSVSYMHWYQQKSSTSPKL |
| | Chain amino acid | WIYDTSKLASGVPGRFSGSGSGNSYSLTISSMEAEDVATYYCFQGS |
| | (MHC1448LC.4) | GYPLTFGSGTKLEIK |
| 27 | Anti-MSLN Light | gaaaatgttctcacccagtctccagcaatcatgtccgcatctccaggggaaaagg |
| | Chain DNA | tcaccatgacctgcagtgctagctcaagtgtaagttacatgcactggtaccagca |
| | (MHC1448LC.4) | gaagtcaagcacctcccccaaactctggatttatgacacatccaaactggcttct |
| | | ggagtcccaggtcgcttcagtggcagtgggtctggaaactcttactctctcacga |
| | | tcagcagcatggaggctgaagatgttgccacttattactgttttcaggggagtgg |
| | | gtacccactcacgttcggctcggggacaaagttggaaaataaaa |
| 28 | Anti-MSLN Heavy | QVQLQQSGAELVRPGASVTLSCKASGYTFTDYEMHWVKQTPVHG |
| | Chain amino acid | LEWIGGIDPETGGTAYNQKFKGKAILTADKSSSTAYMELRSLTSED |
| | (MHC1448HC.3) | SAVYYCTSYYGSRVFWGTGTTVTVSS |
| 29 | Anti-MSLN Heavy | caggttcaactgcagcagtctggggctgagctggtgaggcctggggcttcagtga |
| | Chain DNA | cgctgtcctgcaaggcttcgggctacacatttactgactatgaaatgcactgggt |
| | (MHC1448HC.3) | gaaacagacacctgtgcatggcctggaatggattggaggtattgatcctgaaact |
| | | ggtggtactgcctacaatcagaagttcaagggtaaggccatactgactgcagaca |
| | | aatcctccagcacagcctacatggagctccgcagcctgacatctgaggactctgc |
| | | cgtctattactgtacaagttactatggtagtagagtcttctggggcacagggacc |
| | | acggtcaccgtctcctca |
| 30 | Anti-MSLN Light | QIVLSQSPAILSAFPGEKVTMTCRASSSVSYMHWYQQKPGSSPKP |
| | Chain amino acid | WIYATSNLASGVPARFSGSGSGTSYSLTISSVEAEDAATYYCQQWS |
| | (MHC1449LC.3) | SNPPTLTFGAGTKLELK |
| 31 | Anti-MSLN Light | caaattgttctctcccagtctccagcaatcctgtctgcatttccaggggagaagg |
| | Chain DNA | tcactatgacttgcagggccagctcaagtgtaagttacatgcactggtaccagca |
| | (MHC1449LC.3) | gaagccaggatcctcccccaaaccctggatttatgccacatccaacctggcttct |
| | | ggagtccctgctcgcttcagtggcagtgggtctgggacctcttactctctcacaa |
| | | tcagcagtgtggaggctgaagatgctgccacttattactgccagcagtggagtag |
| | | taacccacccacgctcacgttcggtgctgggaccaagctggagctgaaa |
| 32 | Anti-MSLN Heavy | QVQLQQSGAELARPGASVKLSCKASGYTFTSYGISWVKQRTGQG |
| | Chain amino acid | LEWIGEIYPRSGNTYYNESFKGKVTLTADKSSGTAYMELRSLTSED |
| | (MHC1449HC.3) | SAVYFCARWGSYGSPPFYYGMDYWGQGTSVTVSS |
| 33 | Anti-MSLN Heavy | caggttcagctgcagcagtctggagctgagctggcgaggcctggggcttcagtga |
| | Chain DNA | agctgtcctgcaaggcttctggctacaccttcacaagctatggtataagctgggt |
| | (MHC1449HC.3) | gaagcagaggactggacagggccttgagtggattggagagatttatcctagaagt |
| | | ggtaatacttactacaatgagagcttcaagggcaaggtcacactgaccgcagaca |
| | | aatcttccggcacagcgtacatggagctccgcagcctgacatctgaggactctgc |
| | | ggtctatttctgtgcaagatggggctcctacggtagtccccccttttactatggt |
| | | atggactactggggtcaaggaacctcagtcaccgtctcctca |
| 34 | Anti-MSLN Light | DVLMTQTPLSLPVSLGNQASISCRSSQSIVHSSGSTYLEWYLQKPG |
| | Chain amino acid | QSPKLLIYKVSNRFSGVPDRFSGSGSGTDFTLKISRVEAEDLGVYY |
| | (MHC1450LC.3) | CFQGSHVPYTFGGGTKLEIK |
| 35 | Anti-MSLN Light | gatgttttgatgacccaaactccactctccctgcctgtcagtcttggaaatcaag |
| | Chain DNA | cctccatctcttgcagatctagtcagagcattgtacatagtagtggaagcaccta |
| | (MHC1450LC.3) | tttagaatggtacctgcagaaaccaggccagtctccaaagctcctgatctacaaa |
| | | gtttccaaccgattttctggggtcccagacaggttcagtggcagtggatcaggga |
| | | cagatttcacactcaagatcagcagagtggaggctgaggatctgggagtttatta |
| | | ctgctttcaaggctcacatgttccatacacgttcggaggggggaccaagctggaa |
| | | ataaaa |
| 36 | Anti-MSLN Heavy | QVQLQQSGAELARPGTSVKVSCKASGYTFTSYGISWVKQRIGQGL |
| | Chain amino acid | EWIGEIHPRSGNSYYNEKIRGKATLTADKSSSTAYMELRSLISEDSA |
| | (MHC1450HC.5) | VYFCARLITTVVANYYAMDYWGQGTSVTVSS |
| 37 | Anti-MSLN Heavy | caggttcagctgcagcagtctggagctgagctggcgaggcctgggacttcagtga |
| | Chain DNA | aggtgtcctgcaaggcttctggctataccttcacaagttatggtataagctgggt |
| | (MHC1450HC.5) | gaagcagagaattggacagggccttgagtggattggagagattcatcctagaagt |
| | | ggtaatagttactataatgagaagatcaggggcaaggccacactgactgcagaca |
| | | aatcctccagcacagcgtacatggagctccgcagcctgatatctgaggactctgc |
| | | ggtctatttctgtgcaaggctgattactacggtagttgctaattactatgctatg |
| | | gactactggggtcaaggaacctcagtcaccgtctcctca |
| 38 | Anti-MSLN Light | DIVMSQSPSSLAVSAGEKVTMSCKSSQSLLNSRTRKNYLAWYQQK |
| | Chain amino acid | PGQSPKLLIYWASTRESGVPDRFTGSGSGTDFTLTISSVQAEDLAV |
| | (MHC1451LC.1) | YYCKQSYNLVTFGAGTKLELK |
| 39 | Anti-MSLN Light | gacattgtgatgtcacagtctccatcctccctggctgtgtcagcaggagagaagg |
| | Chain DNA | tcactatgagctgcaaatccagtcagagtctgctcaacagtagaacccgaaagaa |
| | (MHC1451LC.1) | ctacttggcttggtaccagcagaaaccagggcagtctcctaaactgctgatctac |
| | | tgggcatccactagggaatctggggtccctgatcgcttcacaggcagtggatctg |
| | | ggacagatttcactctcaccatcagcagtgtgcaggctgaagacctggcagttta |
| | | ttactgcaaacaatcttataatctggtcacgttcggtgctgggaccaagctggag |
| | | ctgaaa |
| 40 | Anti-MSLN Heavy | QVQLQQSGAELVRPGASVTLSCKASGYTFFDYEMHWVKQTPVHG |
| | Chain amino acid | LEWIGAIDPEIDGTAYNQKFKGKAILTADKSSSTAYMELRSLTSED |
| | (MHC1451HC.2) | SAVYYCTDYYGSSYWYFDVWGTGTTVTVSS |
| 41 | Anti-MSLN Heavy | caggttcaactgcagcagtctggggctgagctggtgaggcctggggcttcagtga |
| | Chain DNA | cgctgtcctgcaaggcttcgggctacacattttttgactatgaaatgcactgggt |
| | (MHC1451HC.2) | gaagcagacacctgtgcatggcctggaatggattggagctattgatcctgaaatt |
| | | gatggtactgcctacaatcagaagttcaagggcaaggccatactgactgcagaca |
| | | aatcctccagcacagcctacatggagctccgcagcctgacatctgaggactctgc |
| | | cgtctattactgtacagattactacggtagtagctactggtacttcgatgtctgg |
| | | ggcacagggaccacggtcaccgtctcctc |
| 42 | Anti-MSLN Light | QIVLTQSPAIMSASPGEKVTISCSASSSVSYMYWYQQKPGSSPKPW |
| | Chain amino acid | IYRTSNLASGVPARFSGSGSGTSYSLTISSMEAEDAATYYCQQYHS |
| | (MHC1452LC.1) | YPLTFGAGTKLELK |
| 43 | Anti-MSLN Light | caaattgttctcacccagtctccagcaatcatgtctgcatctccaggggagaagg |
| | Chain DNA | tcaccatatcctgcagtgccagctcaagtgtaagttacatgtactggtaccagca |
| | (MHC1452LC.1) | gaagccaggatcctcccccaaaccctggatttatcgcacatccaacctggcttct |
| | | ggagtccctgctcgcttcagtggcagtgggtctgggacctcttactctctcacaa |
| | | tcagcagcatggaggctgaagatgctgccacttattactgccagcagtatcatag |
| | | ttacccactcacgttcggtgctgggaccaagctggagctgaaa |
| 44 | Anti-MSLN Light | QIVLTQSPAIMSASPGERVTMTCSASSSVSSSYLYWYQQKSGSSPK |
| | Chain amino acid | LWIYSISNLASGVPARFSGSGSGTSYSLTINSMEAEDAATYYCQQW |
| | (MHC1452LC.6) | SSNPQLTFGAGTKLELK |
| 45 | Anti-MSLN Light | caaattgttctcacccagtctccagcaatcatgtctgcatctcctggggaacggg |
| | Chain DNA | tcaccatgacctgcagtgccagctcaagtgtaagttccagctacttgtactggta |
| | (MHC1452LC.6) | ccagcagaagtcaggatcctccccaaaactctggatttatagcatatccaacctg |
| | | gcttctggagtcccagctcgcttcagtggcagtgggtctgggacctcttactctc |
| | | tcacaatcaacagcatggaggctgaagatgctgccacttattactgccagcagtg |
| | | gagtagtaacccacagctcacgttcggtgctgggaccaagctggagctgaaa |
| 46 | Anti-MSLN Heavy | QVQLKQSGAELVKPGASVKISCKASGYTFTDYYINWVKQRPGQG |
| | Chain amino acid | LEWIGKIGPGSGSTYYNEKFKGKATLTADKSSSTAYMQLSSLTSED |
| | (MHC1452HC.2) | SAVYFCARTGYYVGYYAMDYWGQGTSVTVSS |
| 47 | Anti-MSLN Heavy | caggtccagctgaagcagtctggagctgagctggtgaagcctggggcttcagtga |
| | Chain DNA | agatatcctgcaaggcttctggctacaccttcactgactactatataaactgggt |
| | (MHC1452HC.2) | gaagcagaggcctggacagggccttgagtggattggaaagattggtcctggaagt |
| | | ggtagtacttactacaatgagaagttcaagggcaaggccacactgactgcagaca |
| | | aatcctccagcacagcctacatgcagctcagcagcctgacatctgaggactctgc |
| | | agtctatttctgtgcaagaactggttactacgttggttactatgctatggactac |
| | | tggggtcaaggaacctcagtcaccgtctcctca |
| 48 | Anti-MSLN Heavy | QVQLQQSGAELARPGASVKLSCKASGYTFTIYGISWVKQRTGQGL |
| | Chain amino acid | EWIGEIYPRSDNTYYNEKFKGKATLTADKSSSTAYMELRSLTSEDS |
| | (MHC1452HC.4) | AVYFCARWYSFYAMDYWGQGTSVTVSS |
| 49 | Anti-MSLN Heavy | caggttcagctgcagcagtctggagctgagctggcgaggcctggggcttcagtga |
| | Chain DNA | agctgtcctgcaaggcttctggctacaccttcacaatctatggtataagctgggt |
| | (MHC1452HC.4) | gaaacagagaactggacagggccttgagtggattggagagatttatcctagaagt |
| | | gataatacttactacaatgagaagttcaagggcaaggccacactgactgcagaca |
| | | aatcctccagcacagcgtacatggagctccgcagcctgacatctgaggactctgc |
| | | ggtctatttctgtgcaagatggtactcgttctatgctatggactactggggtcaa |
| | | ggaacctcagtcaccgtctcctca |
| | Single domain | EVQLVESGGGLVQPGGSLRLSCAASGGDWSANFMYWYRQAPGK |
| | anti-MSLN | QRELVARISGRGVVDYVESVKGRFTISRDNSKNTLYLQMNSLRAE |
| | binder 1 (SD1) | DTAVYYCAVASYWGQGTLVTVSS |
| 50 | Single domain | EVQLVESGGGLVQPGGSLRLSCAASGSTSSINTMYWYRQAPGKER |
| | anti-MSLN | ELVAFISSGGSTNVRDSVKGRFTISRDNSKNTLYLQMNSLRAEDTA |
| | binder 4 (SD4) | VYYCNTYIPYGGTLHDFWGQGTLVTVSS |
| 51 | Single domain | QVQLVESGGGVVQAGGSLRLSCAASGSTFSIRAMRWYRQAPGTE |
| | anti-MSLN | RDLVAVIYGSSTYYADAVKGRFTISRDNSKNTLYLQMNSLRAEDT |
| | binder 6 (SD6) | AVYYCNADTIGTARDYWGQGTLVTVSS |
| 52 | p514_BCMA | ACGCGTGTAGTCTTATGCAATACTCTTGTAGTCTTGCAACATGG |
| | (DNA) | TAACGATGAGTTAGCAACATGCCTTACAAGGAGAGAAAAAGCA |
| | | CCGTGCATGCCGATTGGTGGAAGTAAGGTGGTACGATCGTGCC |
| | | TTATTAGGAAGGCAACAGACGGGTCTGACATGGATTGGACGAA |
| | | CCACTGAATTGCCGCATTGCAGAGATATTGTATTTAAGTGCCTA |
| | | GCTCGATACATAAACGGGTCTCTCTGGTTAGACCAGATCTGAG |
| | | CCTGGGAGCTCTCTGGCTAACTAGGGAACCCACTGCTTAAGCCT |
| | | CAATAAAGCTTGCCTTGAGTGCTTCAAGTAGTGTGTGCCCGTCT |
| | | GTTGTGTGACTCTGGTAACTAGAGATCCCTCAGACCCTTTTAGT |
| | | CAGTGTGGAAAATCTCTAGCAGTGGCGCCCGAACAGGGACCTG |
| | | AAAGCGAAAGGGAAACCAGAGCTCTCTCGACGCAGGACTCGG |
| | | CTTGCTGAAGCGCGCACGGCAAGAGGCGAGGGGCGGCGACTG |
| | | GTGAGTACGCCAAAAATTTTGACTAGCGGAGGCTAGAAGGAGA |
| | | GAGATGGGTGCGAGAGCGTCAGTATTAAGCGGGGGAGAATTA |
| | | GATCGCGATGGGAAAAAATTCGGTTAAGGCCAGGGGGAAAGA |
| | | AAAAATATAAATTAAAACATATAGTATGGGCAAGCAGGGAGCT |
| | | AGAACGATTCGCAGTTAATCCTGGCCTGTTAGAAACATCAGAA |
| | | GGCTGTAGACAAATACTGGGACAGCTACAACCATCCCTTCAGA |
| | | CAGGATCAGAAGAACTTAGATCATTATATAATACAGTAGCAAC |
| | | CCTCTATTGTGTGCATCAAAGGATAGAGATAAAAGACACCAAG |
| | | GAAGCTTTAGACAAGATAGAGGAAGAGCAAAACAAAAGTAAG |
| | | ACCACCGCACAGCAAGCGGCCACTGATCTTCAGACCTGGAGGA |
| | | GGAGATATGAGGGACAATTGGAGAAGTGAATTATATAAATATA |
| | | AAGTAGTAAAAATTGAACCATTAGGAGTAGCACCCACCAAGGC |
| | | AAAGAGAAGAGTGGTGCAGAGAGAAAAAAGAGCAGTGGGAAT |
| | | AGGAGCTTTGTTCCTTGGGTTCTTGGGAGCAGCAGGAAGCACT |
| | | ATGGGCGCAGCGTCAATGACGCTGACGGTACAGGCCAGACAAT |
| | | TATTGTCTGGTATAGTGCAGCAGCAGAACAATTTGCTGAG |
| | | GGCTATTGAGGCGCAACAGCATCTGTTGCAACTCACAGTCTGG |
| | | GGCATCAAGCAGCTCCAGGCAAGAATCCTGGCTGTGGAAAGAT |
| | | ACCTAAAGGATCAACAGCTCCTGGGGATTTGGGGTTGCTCTGG |
| | | AAAACTCATTTGCACCACTGCTGTGCCTTGGAATGCTAGTTGGA |
| | | GTAATAAATCTCTGGAACAGATTTGGAATCACACGACCTGGAT |
| | | GGAGTGGGACAGAGAAATTAACAATTACACAAGCTTAATACAC |
| | | TCCTTAATTGAAGAATCGCAAAACCAGCAAGAAAAGAATGAAC |
| | | AAGAATTATTGGAATTAGATAAATGGGCAAGTTTGTGGAATTG |
| | | GTTTAACATAACAAATTGGCTGTGGTATATAAAATTATTCATAA |
| | | TGATAGTAGGAGGCTTGGTAGGTTTAAGAATAGTTTTTGCTGTA |
| | | CTTTCTATAGTGAATAGAGTTAGGCAGGGATATTCACCATTATC |
| | | GTTTCAGACCCACCTCCCAACCCCGAGGGGACCCGACAGGCCC |
| | | GAAGGAATAGAAGAAGAAGGTGGAGAGAGAGACAGAGACAG |
| | | ATCCATTCGATTAGTGAACGGATCTCGACGGTATCGGTTAACTT |
| | | TTAAAAGAAAAGGGGGGATTGGGGGGTACAGTGCAGGGGAAA |
| | | GAATAGTAGACATAATAGCAACAGACATACAAACTAAAGAATT |
| | | ACAAAAACAAATTACAAAATTCAAAATTTTATCGATACTAGTA |
| | | TTATGCCCAGTACATGACCTTATGGGACTTTCCTACTTGGCAGT |
| | | ACATCTACGTATTAGTCATCGCTATTACCATGGTGATGCGGTTT |
| | | TGGCAGTACATCAATGGGCGTGGATAGCGGTTTGACTCACGGG |
| | | GATTTCCAAGTCTCCACCCCATTGACGTCAATGGGAGTTTGTTT |
| | | TGGCACCAAAATCAACGGGACTTTCCAAAATGTCGTAACAACT |
| | | CCGCCCCATTGACGCAAATGGGCGGTAGGCGTGTACGGTGGGA |
| | | GGTTTATATAAGCAGAGCTCGTTTAGTGAACCGTCAGATCGCCT |
| | | GGAGACGCCATCCACGCTGTTTTGACCTCCATAGAAGATTCTAG |
| | | AGCTAGCGCCGCCACCATGCTCCAGATGGCTGGCCAGTGCAGC |
| | | CAGAACGAGTACTTCGACAGCCTGCTGCACGCCTGCATCCCTTG |
| | | CCAGCTGCGGTGCAGCAGCAACACCCCACCCCTGACCTGCCAG |
| | | CGGTACTGCAACGCCAGCGTGACCAACAGCGTGAAGGGCACCA |
| | | ACGCCATCCTGTGGACCTGCCTGGGCCTGAGCCTGATCATCAGC |
| | | CTGGCCGTGTTCGTGCTGATGTTCCTGCTGCGGAAGATCAACAG |
| | | CGAGCCCCTGAAGGACGAGTTCAAGAACACCGGCAGCGGCCTG |
| | | CTGGGCATGGCCAACATCGACCTGGAAAAGAGCCGGACCGGCG |
| | | ACGAGATCATCCTGCCCAGAGGCCTGGAGTACACCGTGGAAGA |
| | | GTGTACCTGCGAGGACTGCATCAAGAGCAAGCCCAAGGTGGAC |
| | | AGCGACCACTGCTTCCCTCTGCCCGCCATGGAAGAGGGCGCCA |
| | | CCATCCTGGTGACAACAAAGACCAACGACTACTGCAAGAGCCT |
| | | GCCTGCCGCCCTGAGCGCCACCGAGATCGAGAAGTCCATCAGC |
| | | GCCAGATGAGGATCCGCGGCCGCAAGGATCTGCGATCGCTCCG |
| | | GTGCCCGTCAGTGGGCAGAGCGCACATCGCCCACAGTCCCCGA |
| | | GAAGTTGGGGGGAGGGGTCGGCAATTGAACGGGTGCCTAGAG |
| | | AAGGTGGCGCGGGGTAAACTGGGAAAGTGATGTCGTGTACTGG |
| | | CTCCGCCTTTTTCCCGAGGGTGGGGGAGAACCGTATATAAGTG |
| | | CAGTAGTCGCCGTGAACGTTCTTTTTCGCAACGGGTTTGCCGCC |
| | | AGAACACAGCTGAAGCTTCGAGGGGCTCGCATCTCTCCTTCAC |
| | | GCGCCCGCCGCCCTACCTGAGGCCGCCATCCACGCCGGTTGAG |
| | | TCGCGTTCTGCCGCCTCCCGCCTGTGGTGCCTCCTGAACTGCGT |
| | | CCGCCGTCTAGGTAAGTTTAAAGCTCAGGTCGAGACCGGGCCT |
| | | TTGTCCGGCGCTCCCTTGGAGCCTACCTAGACTCAGCCGGCTCT |
| | | CCACGCTTTGCCTGACCCTGCTTGCTCAACTCTACGTCTTTGTTT |
| | | CGTTTTCTGTTCTGCGCCGTTACAGATCCAAGCTGTGACCGGCG |
| | | CCTACGTCGAGATGATTGAACAAGATGGATTGCACGCAGGTTC |
| | | TCCGGCCGCTTGGGTGGAGAGGCTATTCG |
| | | GCTATGACTGGGCACAACAGACAATCGGCTGCTCTGATGCCGC |
| | | CGTGTTCCGGCTGTCAGCGCAGGGGCGCCCGGTTCTTTTTGTCA |
| | | AGACCGACCTGTCCGGTGCCCTGAATGAACTGCAGGACGAGGC |
| | | AGCGCGGCTATCGTGGCTGGCCGCGACGGGCGTTCCTTGCGCA |
| | | GCTGTGCTCGACGTTGTCACTGAAGCGGGAAGGGACTGGCTGC |
| | | TATTGGGCGAAGTGCCGGGGCAGGATCTCCTGTCATCTCACCTT |
| | | GCTCCTGCCGAGAAAGTATCCATCATGGCTGATGCAATGCGGC |
| | | GGCTGCATACGCTTGATCCGGCTACCTGCCCATTCGACCACCAA |
| | | GCGAAACATCGCATCGAGCGAGCACGTACTCGGATGGAAGCCG |
| | | GTCTTGTCGATCAGGATGATCTGGACGAAGAGCATCAGGGGCT |
| | | CGCGCCAGCCGAACTGTTCGCCAGGCTCAAGGCGCGCATGCCC |
| | | GACGGCGAGGATCTCGTCGTGACCCATGGCGATGCCTGCTTGC |
| | | CGAATATCATGGTGGAAAATGGCCGCTTTTCTGGATTCATCGAC |
| | | TGTGGCCGGCTGGGTGTGGCGGACCGCTATCAGGACATAGCGT |
| | | TGGCTACCCGTGATATTGCTGAAGAGCTTGGCGGCGAATGGGC |
| | | TGACCGCTTCCTCGTGCTTTACGGTATCGCCGCTCCCGATTCGC |
| | | AGCGCATCGCCTTCTATCGCCTTCTTGACGAGTTCTTCTGACTC |
| | | GACAATCAACCTCTGGATTACAAAATTTGTGAAAGATTGACTG |
| | | GTATTCTTAACTATGTTGCTCCTTTTACGCTATGTGGATACGCTG |
| | | CTTTAATGCCTTTGTATCATGCTATTGCTTCCCGTATGGCTTTCA |
| | | TTTTCTCCTCCTTGTATAAATCCTGGTTGCTGTCTCTTTATGAGG |
| | | AGTTGTGGCCCGTTGTCAGGCAACGTGGCGTGGTGTGCACTGT |
| | | GTTTGCTGACGCAACCCCCACTGGTTGGGGCATTGCCACCACCT |
| | | GTCAGCTCCTTTCCGGGACTTTCGCTTTCCCCCTCCCTATTGCCA |
| | | CGGCGGAACTCATCGCCGCCTGCCTTGCCCGCTGCTGGACAGG |
| | | GGCTCGGCTGTTGGGCACTGACAATTCCGTGGTGTTGTCGGGG |
| | | AAATCATCGTCCTTTCCTTGGCTGCTCGCCTGTGTTGCCACCTG |
| | | GATTCTGCGCGGGACGTCCTTCTGCTACGTCCCTTCGGCCCTCA |
| | | ATCCAGCGGACCTTCCTTCCCGCGGCCTGCTGCCGGCTCTGCGG |
| | | CCTCTTCCGCGTCTTCGCCTTCGCCCTCAGACGAGTCGGATCTC |
| | | CCTTTGGGCCGCCTCCCCGCCTGGTACCTTTAAGACCAATGACT |
| | | TACAAGGCAGCTGTAGATCTTAGCCACTTTTTAAAAGAAAAGG |
| | | GGGGACTGGAAGGGCTAATTCACTCCCAACGAAGATAAGATCT |
| | | GCTTTTTGCTTGTACTGGGTCTCTCTGGTTAGACCAGATCTGAG |
| | | CCTGGGAGCTCTCTGGCTAACTAGGGAACCCACTGCTTAAGCCT |
| | | CAATAAAGCTTGCCTTGAGTGCTTCAAGTAGTGTGTGCCCGTCT |
| | | GTTGTGTGACTCTGGTAACTAGAGATCCCTCAGACCCTTTTAGT |
| | | CAGTGTGGAAAATCTCTAGCAGTAGTAGTTCATGTCATCTTATT |
| | | ATTCAGTATTTATAACTTGCAAAGAAATGAATATCAGAGAGTG |
| | | AGAGGAACTTGTTTATTGCAGCTTATAATGGTTACAAATAAAG |
| | | CAATAGCATCACAAATTTCACAAATAAAGCATTTTTTTCACTGC |
| | | ATTCTAGTTGTGGTTTGTCCAAACTCATCAATGTATCTTATCAT |
| | | GTCTGGCTCTAGCTATCCCGCCCCTAACTCCGCCCATCCCGCCC |
| | | CTAACTCCGCCCAGTTCCGCCCATTCTCCGCCCCATGGCTGACT |
| | | AATTTTTTTTATTTATGCAGAGGCCGAGGCCGCCTCGGCCTCTG |
| | | AGCTATTCCAGAAGTAGTGAGGAGGCTTTTTTGGAGGCCTAGA |
| | | CTTTTGCAGAGACGGCCCAAATTCGTAATCATGGTCATAGCTGT |
| | | TTCCTGTGTGAAATTGTTATCCGCTCACAATTCCACACAACATA |
| | | CGAGCCGGAAGCATAAAGTGTAAAGCCTGGGGTGCCTAATGAG |
| | | TGAGCTAACTCACATTAATTGCGTTGCGCTCACTGCCCGCTTTC |
| | | CAGTCGGGAAACCTGTCGTGCCAGCTGCATTAATGAATCGGCC |
| | | AACGCGCGGGGAGAGGCGGTTTGCGTATTGGGCGCTCTTCCGC |
| | | TTCCTCGCTCACTGACTCGCTGCGCTCGGTCGTTCGGCTGCGGC |
| | | GAGCGGTATCAGCTCACTCAAAGGCGGTAATACGGTTATCCAC |
| | | AGAATCAGGGGATAACGCAGGAAAGAACATGTGAGCAAAAGG |
| | | CCAGCAAAAGGCCAGGAACCGTAAAAAGGCCGCGTTGCTGGC |
| | | GTTTTTCCATAGGCTCCGCCCCCCTGACGAGCATCACAAAAATC |
| | | GACGCTCAAGTCAGAGGTGGCGAAACCCGACAGGACTATAAA |
| | | GATACCAGGCGTTTCCCCCTGGAAGCTCCCTCGTGCGCTCTCCT |
| | | GTTCCGACCCTGCCGCTTACCGGATACCTGTCCGCCTTTCTCCC |
| | | TTCGGGAAGCGTGGCGCTTTCTCATAGCTCACGCTGTAGGTATC |
| | | TCAGTTCGGTGTAGGTCGTTCGCTCCAAGCTGGGCTGTGTGCAC |
| | | GAACCCCCCGTTCAGCCCGACCGCTGCGCCTTATCCGGTAACTA |
| | | TCGTCTTGAGTCCAACCCGGTAAGACACGACTTATCGCCACTGG |
| | | CAGCAGCCACTGGTAACAGGATTAGCAGAGCGAGGTATGTAGG |
| | | CGGTGCTACAGAGTTCTTGAAGTGGTGGCCTAACTACGGCTAC |
| | | ACTAGAAGGACAGTATTTGGTATCTGCGCTCTGCTGAAGCCAG |
| | | TTACCTTCGGAAAAAGAGTTGGTAGCTCTTGATCCGGCAAACA |
| | | AACCACCGCTGGTAGCGGTGGTTTTTTTGTTTGCAAGCAGCAGA |
| | | TTACGCGCAGAAAAAAAGGATCTCAAGAAGATCCTTTGATCTT |
| | | TTCTACGGGGTCTGACGCTCAGTGGAACGAAAACTCACGTTAA |
| | | GGGATTTTGGTCATGAGATTATCAAAAAGGATCTTCACCTAGAT |
| | | CCTTTTAAATTAAAAATGAAGTTTTAAATCAATCTAAAGTATAT |
| | | ATGAGTAAACTTGGTCTGACAGTTACCAATGCTTAATCAGTGA |
| | | GGCACCTATCTCAGCGATCTGTCTATTTCGTTCATCCATAGTTG |
| | | CCTGACTCCCCGTCGTGTAGATAACTACGATACGGGAGGGCTT |
| | | ACCATCTGGCCCCAGTGCTGCAATGATACCGCGAGACCCACGC |
| | | TCACCGGCTCCAGATTTATCAGCAATAAACCAGCCAGCCGGAA |
| | | GGGCCGAGCGCAGAAGTGGTCCTGCAACTTTATCCGCCTCCAT |
| | | CCAGTCTATTAATTGTTGCCGGGAAGCTAGAGTAAGTAGTTCGC |
| | | CAGTTAATAGTTTGCGCAACGTTGTTGCCATTGCTACAGGCATC |
| | | GTGGTGTCACGCTCGTCGTTTGGTATGGCTTCATTCAGCTCCGG |
| | | TTCCCAACGATCAAGGCGAGTTACATGATCCCCCATGTTGTGCA |
| | | AAAAAGCGGTTAGCTCCTTCGGTCCTCCGATCGTTGTCAGAAGT |
| | | AAGTTGGCCGCAGTGTTATCACTCATGGTTATGGCAGCACTGCA |
| | | TAATTCTCTTACTGTCATGCCATCCGTAAGATGCTTTTCTGTGAC |
| | | TGGTGAGTACTCAACCAAGTCATTCTGAGAATAGTGTATGCGG |
| | | CGACCGAGTTGCTCTTGCCCGGCGTCAATACGGGATAATACCG |
| | | CGCCACATAGCAGAACTTTAAAAGTGCTCATCATTGGAAAACG |
| | | TTCTTCGGGGCGAAAACTCTCAAGGATCTTACCGCTGTTGAGAT |
| | | CCAGTTCGATGTAACCCACTCGTGCACCCAACTGATCTTCAGCA |
| | | TCTTTTACTTTCACCAGCGTTTCTGGGTGAGCAAAAACAGGAAG |
| | | GCAAAATGCCGCAAAAAAGGGAATAAGGGCGACACGGAAATG |
| | | TTGAATACTCATACTCTTCCTTTTTCAATATTATTGAAGCATTTA |
| | | TCAGGGTTATTGTCTCATGAGCGGATACATATTTGAATGTATTT |
| | | AGAAAAATAAACAAATAGGGGTTCCGCGCACATTTCCCCGAAA |
| | | AGTGCCACCTGACGTCTAAGAAACCATTATTATCATGACATTAA |
| | | CCTATAAAAATAGGCGTATCACGAGGCCCTTTCGTCTCGCGCGT |
| | | TTCGGTGATGACGGTGAAAACCTCTGACACATGCAGCTCCCGG |
| | | AGACGGTCACAGCTTGTCTGTAAGCGGATGCCGGGAGCAGACA |
| | | AGCCCGTCAGGGCGCGTCAGCGGGTGTTGGCGGGTGTCGGGGC |
| | | TGGCTTAACTATGCGGCATCAGAGCAGATTGTACTGAGAGTGC |
| | | ACCATATGCGGTGTGAAATACCGCACAGATGCGTAAGGAGAAA |
| | | ATACCGCATCAGGC |
| | | GCCATTCGCCATTCAGGCTGCGCAACTGTTGGGAAGGGCGATC |
| | | GGTGCGGGCCTCTTCGCTATTACGCCAGCTGGCGAAAGGGGGA |
| | | TGTGCTGCAAGGCGATTAAGTTGGGTAACGCCAGGGTTTTCCC |
| | | AGTCACGACGTTGTAAAACGACGGCCAGTGCCAAGCTG |
| 53 | anti-BCMA scFv 1 | AAAAGCAGCCAGAGCCTGGTGCATAGCAACGGCAACACCTATC |
| | light chain CDR1 | TGCAT |
| | (DNA) | |
| 54 | anti-BCMA scFv 1 | KSSQSLVHSNGNTYLH |
| | light chain CDR1 | |
| | (amino acid) | |
| 55 | anti-BCMA scFv 1 | AAAGTGAGCAACCGCTTTAGC |
| | light chain CDR2 | |
| | (DNA) | |
| 56 | anti-BCMA scFv 1 | KVSNRFS |
| | light chain CDR2 | |
| | (amino acid) | |
| 57 | anti-BCMA scFv 1 | GCGGAAACCAGCCATGTGCCGTGGACC |
| | light chain CDR3 | |
| | (DNA) | |
| 58 | anti-BCMA scFv 1 | AETSHVPWT |
| | light chain CDR3 | |
| | (amino acid) | |
| 59 | anti-BCMA scFv 1 | AAAGCGAGCGGCTATAGCTTTCCGGATTATTATATTAAC |
| | heavy chain CDR1 | |
| | (DNA) | |
| 60 | anti-BCMA scFv 1 | KASGYSFPDYYIN |
| | heavy chain CDR1 | |
| | (amino acid) | |
| 61 | anti-BCMA scFv 1 | TGGATTTATTTTGCGAGCGGCAACAGCGAATATAACCAGAAAT |
| | heavy chain CDR2 | TTACCGGC |
| | (DNA) | |
| 62 | anti-BCMA scFv 1 | WIYFASGNSEYNQKFTG |
| | heavy chain CDR2 | |
| | (amino acid) | |
| 63 | anti-BCMA scFv 1 | CTGTATGATTATGATTGGTATTTTGATGTG |
| | heavy chain CDR3 | |
| | (DNA) | |
| 64 | anti-BCMA scFv 1 | LYDYDWYFDV |
| | heavy chain CDR3 | |
| | (amino acid) | |
| 65 | Anti-BCMA light | GATATTGTGATGACCCAGACCCCGCTGAGCCTGAGCGTGACCC |
| | chain variable | CGGGCGAACCGGCGAGCATTAGCTGCAAAAGCAGCCAGAGCCT |
| | region (DNA) | GGTGCATAGCAACGGCAACACCTATCTGCATTGGTATCTGCAG |
| | | AAACCGGGCCAGAGCCCGCAGCTGCTGATTTATAAAGTGAGCA |
| | | ACCGCTTTAGCGGCGTGCCGGATCGCTTTAGCGGCAGCGGCAG |
| | | CGGCGCGGATTTTACCCTGAAAATTAGCCGCGTGGAAGCGGAA |
| | | GATGTGGGCGTGTATTATTGCGCGGAAACCAGCCATGTGCCGT |
| | | GGACCTTTGGCCAGGGCACCAAACTGGAAATTAAAAGC |
| 66 | Anti-BCMA light | DIVMTQTPLSLSVTPGEPASISCKSSQSLVHSNGNTYLHWYLQKPG |
| | chain variable | QSPQLLIYKVSNRFSGVPDRFSGSGSGADFTLKISRVEAEDVGVYY |
| | region (amino | CAETSHVPWTFGQGTKLEIKS |
| | acid) | |
| 67 | Anti-BCMA heavy | CAGGTGCAGCTGGTGCAGAGCGGCGCGGAAGTGAAAAAACCG |
| | chain variable | GGCGCGAGCGTGAAAGTGAGCTGCAAAGCGAGCGGCTATAGCT |
| | region (DNA) | TTCCGGATTATTATATTAACTGGGTGCGCCAGGCGCCGGGCCA |
| | | GGGCCTGGAATGGATGGGCTGGATTTATTTTGCGAGCGGCAAC |
| | | AGCGAATATAACCAGAAATTTACCGGCCGCGTGACCATGACCC |
| | | GCGATACCAGCAGCAGCACCGCGTATATGGAACTGAGCAGCCT |
| | | GCGCAGCGAAGATACCGCGGTGTATTTTTGCGCGAGCCTGTAT |
| | | GATTATGATTGGTATTTTGATGTGTGGGGCCAGGGCACCATGGT |
| | | GACCGTGAGCAGC |
| 68 | Anti-BCMA heavy | QVQLVQSGAEVKKPGASVKVSCKASGYSFPDYYINWVRQAPGQG |
| | chain variable | LEWMGWIYFASGNSEYNQKFTGRVTMTRDTSSSTAYMELSSLRSE |
| | region (amino | DTAVYFCASLYDYDWYFDVWGQGTMVTVSS |
| | acid) | |
| 69 | Anti-BCMA V_{H}H 1 | ATGGCGGTGGTCCTGGCTGCTCTACTACAAGGTGTCCAGGCTCA |
| | DNA | GGTGCACCCGGTGGAGTCTGGGGGAGGCTTGGTGCAGACTGGG |
| | | GGGTCTCTGAGACTCTCCTGTGCAGCCTCTGCTGGTATCTTCAG |
| | | TATCAATGTCATGGGCTGGTACCGCCAGGCTCCAGGGAAGCAG |
| | | CGCGAATTGGTCGCGAGTATAACTAGTCGTGGTGATACAACGT |
| | | ATGCGAACTCCGTGAAGGGCCGATTCACCATCTCCAGAGACAA |
| | | CGCCAAGAACACGGTATATCTGCAAATGAACGCCCTGAAACCT |
| | | GAGGACACAGCCGTCTATTACTGTAATTTAAAGGGGACAGACT |
| | | ATAGTGGTACATCCACCCAGACCTTCGACAGACAGGGCCAGGG |
| | | GACCCAGGTCACCGTCTCTTCGGAACCCAAGACACCAAAACCA |
| | | CAACCACAACCACAACCACAACCACAACCCAATCCTACAACAG |
| | | AATCCAAGTGTCCCAAATGTCCAGCCCCTGAGCTCCTGGGAGG |
| | | GCCCTCAGTCTTCATCTTCCCCCCGAAACCCAAGGACGTCCTCT |
| | | CCATC |
| 70 | Anti-BCMA V_{H}H 1 | MAVVLAALLQGVQAQVHPVESGGGLVQTGGSLRLSCAASAGIFSI |
| | Amino Acid | NVMGWYRQAPGKQRELVASITSRGDTTYANSVKGRFTISRDNAK |
| | | NTVYLQMNALKPEDTAVYYCNLKGTDYSGTSTQTFDRQGQGTQ |
| | | VTVSSEPKTPKPQPQPQPQPQPNPTTESKCPKCPAPELLGGPSVFIFP |
| | | PKPKDVLSI |
| 71 | Anti-BCMA V_{H}H1 | INVMG |
| | CDR1 | |
| 72 | Anti-BCMA V_{H}H1 | SITSRGDTTYANSVKG |
| | CDR2 | |
| 73 | Anti-BCMA V_{H}H1 | LKGTDYSGTSTQTFDR |
| | CDR3 | |
| 74 | Anti-BCMA V_{H}H 2 | QVQLVESGGGLVQPGESLRLSCAASTNIFSISPMGWYRQAPGKQR |
| | Amino Acid | ELVAAIHGFSTLYADSVKGRFTISRDNAKNTIYLQMNSLKPEDTAV |
| | | YYCNKVPWGDYHPRNVYWGQGTQVTVSSEPKTPKPQPQPQPQPQ |
| | | PQPNPTTESKCPKCPAPELLGGPSVFIFPPKPKDVLSI |
| 75 | Anti-BCMA V_{H}H2 | ISPMG |
| | CDR1 | |
| 76 | Anti-BCMA V_{H}H2 | AIHGFSTLYADSVKG |
| | CDR2 | |
| 77 | Anti-BCMA V_{H}H2 | VPWGDYHPRNVY |
| | CDR3 | |
| 78 | NKG2D type II | MGWIRGRRSRHSWEMSEFHNYNLDLKKSDFSTRWQKQRCPVVK |
| | integral membrane | SKCRENASPFFFCCFIAVAMGIRFIIMVAIWSAVFLNSLFNQEVQIPL |
| | protein, UniProt | TESYCGPCPKNWICYKNNCYQFFDESKNWYESQASCMSQNASLL |
| | Accession No. | KVYSKEDQDLLKLVKSYHWMGLVHIPTNGSWQWEDGSILSPNLL |
| | P26718-1 | TIIEMQKGDCALYASSFKGYIENCSTPNTYICMQRTV |
| 79 | p502_NKG2D_CD | NSLFNQEVQIPLTESYCGPCPKNWICYKNNCYQFFDESKNWYESQ |
| | 3epsilon | ASCMSQNASLLKVYSKEDQDLLKLVKSYHWMGLVHIPTNGSWQ |
| | extracellular | WEDGSILSPNLLTIIEMQKGDCALYASSFKGYIENCSTPNTYICMQR |
| | domain dimer | TVGGGGSGGGGSGGGGSLENSLFNQEVQIPLTESYCGPCPKNWIC |
| | (ECD) | YKNNCYQFFDESKNWYESQASCMSQNASLLKVYSKEDQDLLKLV |
| | | KSYHWMGLVHIPTNGSWQWEDGSILSPNLLTIIEMQKGDCALYAS |
| | | SFKGYIENCSTPNTYICMQRTVAAAGGGGSGGGGSGGGGSLEDGN |
| | | EEMGGITQTPYKVSISGTTVILTCPQYPGSEILWQHNDKNIGGDED |
| | | DKNIGSDEDHLSLKEFSELEQSGYYVCYPRGSKPEDANFYLYLRA |
| | | RVCENCMEMDVMS |
| 80 | p502_NKG2D_CD | NSLFNQEVQIPLTESYCGPCPKNWICYKNNCYQFFDESKNWYESQ |
| | 3epsilon ORF, | ASCMSQNASLLKVYSKEDQDLLKLVKSYHWMGLVHIPTNGSWQ |
| | dimer (amino acid | WEDGSILSPNLLTIIEMQKGDCALYASSFKGYIENCSTPNTYICMQR |
| | sequence) | TVGGGGSGGGGSGGGGSLENSLFNQEVQIPLTESYCGPCPKNWIC |
| | | YKNNCYQFFDESKNWYESQASCMSQNASLLKVYSKEDQDLLKLV |
| | | KSYHWMGLVHIPTNGSWQWEDGSILSPNLLTIIEMQKGDCALYAS |
| | | SFKGYIENCSTPNTYICMQRTVAAAGGGGSGGGGSGGGGSLEDGN |
| | | EEMGGITQTPYKVSISGTTVILTCPQYPGSEILWQHNDKNIGGDED |
| | | DKNIGSDEDHLSLKEFSELEQSGYYVCYPRGSKPEDANFYLYLRA |
| | | RVCENCMEMDVMSVATIVIVDICITGGLLLLVYYWSKNRKAKAK |
| | | PVTRGAGAGGRQRGQNKERPPPVPNPDYEPIRKGQRDLYSGLNQR |
| | | RI* |
| 81 | p502 NKG2D | ACGCGTGTAGTCTTATGCAATACTCTGTAGTCTTGCAACATGGT |
| | monomer DNA | AACGATGAGTTAGCAACATGCCTTACAAGGAGAGAAAAAGCA |
| | Sequence | CCGTGCATGCCGATTGGTGGAAGTAAGGTGGTACGATCGTGCC |
| | | TTATTAGGAAGGCAACAGACGGGTCTGACATGGATTGGACGAA |
| | | CCACTGAATTGCCGCATTGCAGAGATATTGTATTTAAGTGCCTA |
| | | GCTCGATACATAAACGGGTCTCTCTGGTTAGACCAGATCTGAG |
| | | CCTGGGAGCTCTCTGGCTAACTAGGGAACCCACTGCTTAAGCCT |
| | | CAATAAAGCTTGCCTTGAGTGCTTCAAGTAGTGTGTGCCCGTCT |
| | | GTTGTGTGACTCTGGTAACTAGAGATCCCTCAGACCCTTTTAGT |
| | | CAGTGTGGAAAATCTCTAGCAGTGGCGCCCGAACAGGGACTTG |
| | | AAAGCGAAAGGGAAACCAGAGGAGCTCTCTCGACGCAGGACT |
| | | CGGCTTGCTGAAGCGCGCACGGCAAGAGGCGAGGGGCGGCGA |
| | | CTGGTGAGTACGCCAAAAATTTTGACTAGCGGAGGCTAGAAGG |
| | | AGAGAGATGGGTGCGAGAGCGTCAGTATTAAGCGGGGGAGAA |
| | | TTAGATCGCGATGGGAAAAAATTCGGTTAAGGCCAGGGGGAAA |
| | | GAAAAAATATAAATTAAAACATATAGTATGGGCAAGCAGGGA |
| | | GCTAGAACGATTCGCAGTTAATCCTGGCCTGTTAGAAACATCA |
| | | GAAGGCTGTAGACAAATACTGGGACAGCTACAACCATCCCTTC |
| | | AGACAGGATCAGAAGAACTTAGATCATTATATAATACAGTAGC |
| | | AACCCTCTATTGTGTGCATCAAAGGATAGAGATAAAAGACACC |
| | | AAGGAAGCTTTAGACAAGATAGAGGAAGAGCAAAACAAAAGT |
| | | AAGACCACCGCACAGCAAGCGGCCACTGATCTTCAGACCTGGA |
| | | GGAGGAGATATGAGGGACAATTGGAGAAGTGAATTATATAAAT |
| | | ATAAAGTAGTAAAAATTGAACCATTAGGAGTAGCACCCACCAA |
| | | GGCAAAGAGAAGAGTGGTGCAGAGAGAAAAAAGAGCAGTGGG |
| | | AATAGGAGCTTTGTTCCTTGGGTTCTTGGGAGCAGCAGGAAGC |
| | | ACTATGGGCGCAGCGTCAATGACGCTGACGGTACAGGCCAGAC |
| | | AATTATTGTCTGGTATAGTGCAGCAGCAGAACAATTTGCTGAG |
| | | GGCTATTGAGGCGCAACAGCATCTGTTGCAACTCACAGTCTGG |
| | | GGCATCAAGCAGCTCCAGGCAAGAATCCTGGCTGTGGAAAGAT |
| | | ACCTAAAGGATCAACAGCTCCTGGGGATTTGGGGTTGCTCTGG |
| | | AAAACTCATTTGCACCACTGCTGTGCCTTGGAATGCTAGTTGGA |
| | | GTAATAAATCTCTGGAACAGATTTGGAATCACACGACCTGGAT |
| | | GGAGTGGGACAGAGAAATTAACAATTACACAAGCTTAATACAC |
| | | TCCTTAATTGAAGAATCGCAAAACCAGCAAGAAAAGAATGAAC |
| | | AAGAATTATTGGAATTAGATAAATGGGCAAGTTTGTGGAATTG |
| | | GTTTAACATAACAAATTGGCTGTGGTATATAAAATTATTCATAA |
| | | TGATAGTAGGAGGCTTGGTAGGTTTAAGAATAGTTTTTGCTGTA |
| | | CTTTCTATAGTGAATAGAGTTAGGCAGGGATATTCACCATTATC |
| | | GTTTCAGACCCACCTCCCAACCCCGAGGGGACCCGACAGGCCC |
| | | GAAGGAATAGAAGAAGAAGGTGGAGAGAGAGACAGAGACAG |
| | | ATCCATTCGATTAGTGAACGGATCTCGACGGTATCGGTTAACTT |
| | | TTAAAAGAAAAGGGGGGATTGGGGGGTACAGTGCAGGGGAAA |
| | | GAATAGTAGACATAATAGCAACAGACATACAAACTAAAGAATT |
| | | ACAAAAACAAATTACAAAATTCAAAATTTTATCGATACTAGTG |
| | | GATCTGCGATCGCTCCGGTGCCCGTCAGTGGGCAGAGCGCACA |
| | | TCGCCCACAGTCCCCGAGAAGTTGGGGGGAGGGGTCGGCAATT |
| | | GAACGGGTGCCTAGAGAAGGTGGCGCGGGGTAAACTGGGAAA |
| | | GTGATGTCGTGTACTGGCTCCGCCTTTTTCCCGAGGGTGGGGGA |
| | | GAACCGTATATAAGTGCAGTAGTCGCCGTGAACGTTCTTTTTCG |
| | | CAACGGGTTTGCCGCCAGAACACAGCTGAAGCTTCGAGGGGCT |
| | | CGCATCTCTCCTTCACGCGCCCGCCGCCCTACCTGAGGCCGCCA |
| | | TCCACGCCGGTTGAGTCGCGTTCTGCCGCCTCCCGCCTGTGGTG |
| | | CCTCCTGAACTGCGTCCGCCGTCTAGGTAAGTTTAAAGCTCAGG |
| | | TCGAGACCGGGCCTTTGTCCGGCGCTCCCTTGGAGCCTACCTAG |
| | | ACTCAGCCGGCTCTCCACGCTTTGCCTGACCCTGCTTGCTCAAC |
| | | TCTACGTCTTTGTTTCGTTTTCTGTTCTGCGCCGTTACAGATCCA |
| | | AGCTGTGACCGGCGCCTACTCTAGAGCCGCCACCATGCTTCTCC |
| | | TGGTGACAAGCCTTCTGCTCTGTGAGTTACCACACCCAGCATTC |
| | | CTCCTGATCCCAAACTCCCTCTTCAACCAGGAGGTGCAGATCCC |
| | | CCTCACAGAGAGCTACTGCGGGCCCTGTCCAAAGAATTGGATA |
| | | TGTTACAAGAACAATTGCTACCAGTTCTTCGATGAGTCAAAAA |
| | | ATTGGTATGAGAGCCAAGCTTCCTGCATGTCTCAGAATGCCAG |
| | | CCTTCTGAAGGTGTACTCAAAAGAAGACCAGGACTTGCTGAAA |
| | | CTGGTCAAGTCTTACCACTGGATGGGGCTCGTGCACATTCCAAC |
| | | GAACGGTAGCTGGCAGTGGGAAGATGGCTCCATATTGTCTCCT |
| | | AACCTTCTCACCATAATAGAGATGCAGAAGGGTGATTGCGCTC |
| | | TGTACGCTAGTAGCTTCAAGGGCTATATTGAGAATTGTAGTACA |
| | | CCCAACACATACATTTGTATGCAGAGAACCGTGGGAGGTGGTG |
| | | GCAGCGGTGGCGGTGGAAGTGGTGGCGGCGGTTCTCTCGAGGA |
| | | TGGTAATGAAGAAATGGGTGGTATTACACAGACACCATATAAA |
| | | GTCTCCATCTCTGGAACCACAGTAATATTGACATGCCCTCAGTA |
| | | TCCTGGATCTGAAATACTATGGCAACACAATGATAAAAACATA |
| | | GGCGGTGATGAGGATGATAAAAACATAGGCAGTGATGAGGAT |
| | | CACCTGTCACTGAAGGAATTTTCAGAATTGGAGCAAAGTGGTT |
| | | ATTATGTCTGCTACCCCAGAGGAAGCAAACCAGAAGATGCGAA |
| | | CTTTTATCTCTACCTGAGGGCAAGAGTGTGTGAGAACTGCATGG |
| | | AGATGGATGTGATGTCGGTGGCCACAATTGTCATAGTGGACAT |
| | | CTGCATCACTGGGGGCTTGCTGCTGCTGGTTTACTACTGGAGCA |
| | | AGAATAGAAAGGCCAAGGCCAAGCCTGTGACACGAGGAGCGG |
| | | GTGCTGGCGGCAGGCAAAGGGGACAAAACAAGGAGAGGCCAC |
| | | CACCTGTTCCCAACCCAGACTATGAGCCCATCCGGAAAGGCCA |
| | | GCGGGACCTGTATTCTGGCCTGAATCAGAGACGCATCTGATAA |
| | | GAATTCGAATTTAAATCGGATCCGCGGCCGCGTCGACAATCAA |
| | | CCTCTGGATTACAAAATTTGTGAAAGATTGACTGGTATTCTTAA |
| | | CTATGTTGCTCCTTTTACGCTATGTGGATACGCTGCTTTAATGCC |
| | | TTTGTATCATGCTATTGCTTCCCGTATGGCTTTCATTTTCTCCTC |
| | | CTTGTATAAATCCTGGTTGCTGTCTCTTTATGAGGAGTTGTGGC |
| | | CCGTTGTCAGGCAACGTGGCGTGGTGTGCACTGTGTTTGCTGAC |
| | | GCAACCCCCACTGGTTGGGGCATTGCCACCACCTGTCAGCTCCT |
| | | TTCCGGGACTTTCGCTTTCCCCCTCCCTATTGCCACGGCGGAAC |
| | | TCATCGCCGCCTGCCTTGCCCGCTGCTGGACAGGGGCTCGGCTG |
| | | TTGGGCACTGACAATTCCGTGGTGTTGTCGGGGAAATCATCGTC |
| | | CTTTCCTTGGCTGCTCGCCTGTGTTGCCACCTGGATTCTGCGCG |
| | | GGACGTCCTTCTGCTACGTCCCTTCGGCCCTCAATCCAGCGGAC |
| | | CTTCCTTCCCGCGGCCTGCTGCCGGCTCTGCGGCCTCTTCCGCG |
| | | TCTTCGCCTTCGCCCTCAGACGAGTCGGATCTCCCTTTGGGCCG |
| | | CCTCCCCGCCTGGTACCTTTAAGACCAATGACTTACAAGGCAGC |
| | | TGTAGATCTTAGCCACTTTTTAAAAGAAAAGGGGGGACTGGAA |
| | | GGGCTAATTCACTCCCAACGAAGATAAGATCTGCTTTTTGCTTG |
| | | TACTGGGTCTCTCTGGTTAGACCAGATCTGAGCCTGGGAGCTCT |
| | | CTGGCTAACTAGGGAACCCACTGCTTAAGCCTCAATAAAGCTT |
| | | GCCTTGAGTGCTTCAAGTAGTGTGTGCCCGTCTGTTGTGTGACT |
| | | CTGGTAACTAGAGATCCCTCAGACCCTTTTAGTCAGTGTGGAAA |
| | | ATCTCTAGCAGTAGTAGTTCATGTCATCTTATTATTCAGTATTTA |
| | | TAACTTGCAAAGAAATGAATATCAGAGAGTGAGAGGAACTTGT |
| | | TTATTGCAGCTTATAATGGTTACAAATAAAGCAATAGCATCAC |
| | | AAATTTCACAAATAAAGCATTTTTTTCACTGCATTCTAGTTGTG |
| | | GTTTGTCCAAACTCATCAATGTATCTTATCATGTCTGGCTCTAG |
| | | CTATCCCGCCCCTAACTCCGCCCATCCCGCCCCTAACTCCGCCC |
| | | AGTTCCGCCCATTCTCCGCCCCATGGCTGACTAATTTTTTTTATT |
| | | TATGCAGAGGCCGAGGCCGCCTCGGCCTCTGAGCTATTCCAGA |
| | | AGTAGTGAGGAGGCTTTTTTGGAGGCCTAGACTTTTGCAGAGA |
| | | CGGCCCAAATTCGTAATCATGGTCATAGCTGTTTCCTGTGTGAA |
| | | ATTGTTATCCGCTCACAATTCCACACAACATACGAGCCGGAAG |
| | | CATAAAGTGTAAAGCCTGGGGTGCCTAATGAGTGAGCTAACTC |
| | | ACATTAATTGCGTTGCGCTCACTGCCCGCTTTCCAGTCGGGAAA |
| | | CCTGTCGTGCCAGCTGCATTAATGAATCGGCCAACGCGCGGGG |
| | | AGAGGCGGTTTGCGTATTGGGCGCTCTTCCGCTTCCTCGCTCAC |
| | | TGACTCGCTGCGCTCGGTCGTTCGGCTGCGGCGAGCGGTATCA |
| | | GCTCACTCAAAGGCGGTAATACGGTTATCCACAGAATCAGGGG |
| | | ATAACGCAGGAAAGAACATGTGAGCAAAAGGCCAGCAAAAGG |
| | | CCAGGAACCGTAAAAAGGCCGCGTTGCTGGCGTTTTTCCATAG |
| | | GCTCCGCCCCCCTGACGAGCATCACAAAAATCGACGCTCAAGT |
| | | CAGAGGTGGCGAAACCCGACAGGACTATAAAGATACCAGGCG |
| | | TTTCCCCCTGGAAGCTCCCTCGTGCGCTCTCCTGTTCCGACCCT |
| | | GCCGCTTACCGGATACCTGTCCGCCTTTCTCCCTTCGGGAAGCG |
| | | TGGCGCTTTCTCATAGCTCACGCTGTAGGTATCTCAGTTCGGTG |
| | | TAGGTCGTTCGCTCCAAGCTGGGCTGTGTGCACGAACCCCCCGT |
| | | TCAGCCCGACCGCTGCGCCTTATCCGGTAACTATCGTCTTGAGT |
| | | CCAACCCGGTAAGACACGACTTATCGCCACTGGCAGCAGCCAC |
| | | TGGTAACAGGATTAGCAGAGCGAGGTATGTAGGCGGTGCTACA |
| | | GAGTTCTTGAAGTGGTGGCCTAACTACGGCTACACTAGAAGGA |
| | | CAGTATTTGGTATCTGCGCTCTGCTGAAGCCAGTTACCTTCGGA |
| | | AAAAGAGTTGGTAGCTCTTGATCCGGCAAACAAACCACCGCTG |
| | | GTAGCGGTGGTTTTTTTGTTTGCAAGCAGCAGATTACGCGCAGA |
| | | AAAAAAGGATCTCAAGAAGATCCTTTGATCTTTTCTACGGGGTC |
| | | TGACGCTCAGTGGAACGAAAACTCACGTTAAGGGATTTTGGTC |
| | | ATGAGATTATCAAAAAGGATCTTCACCTAGATCCTTTTAAATTA |
| | | AAAATGAAGTTTTAAATCAATCTAAAGTATATATGAGTAAACT |
| | | TGGTCTGACAGTTACCAATGCTTAATCAGTGAGGCACCTATCTC |
| | | AGCGATCTGTCTATTTCGTTCATCCATAGTTGCCTGACTCCCCG |
| | | TCGTGTAGATAACTACGATACGGGAGGGCTTACCATCTGGCCC |
| | | CAGTGCTGCAATGATACCGCGAGACCCACGCTCACCGGCTCCA |
| | | GATTTATCAGCAATAAACCAGCCAGCCGGAAGGGCCGAGCGCA |
| | | GAAGTGGTCCTGCAACTTTATCCGCCTCCATCCAGTCTATTAAT |
| | | TGTTGCCGGGAAGCTAGAGTAAGTAGTTCGCCAGTTAATAGTTT |
| | | GCGCAACGTTGTTGCCATTGCTACAGGCATCGTGGTGTCACGCT |
| | | CGTCGTTTGGTATGGCTTCATTCAGCTCCGGTTCCCAACGATCA |
| | | AGGCGAGTTACATGATCCCCCATGTTGTGCAAAAAAGCGGTTA |
| | | GCTCCTTCGGTCCTCCGATCGTTGTCAGAAGTAAGTTGGCCGCA |
| | | GTGTTATCACTCATGGTTATGGCAGCACTGCATAATTCTCTTAC |
| | | TGTCATGCCATCCGTAAGATGCTTTTCTGTGACTGGTGAGTACT |
| | | CAACCAAGTCATTCTGAGAATAGTGTATGCGGCGACCGAGTTG |
| | | CTCTTGCCCGGCGTCAATACGGGATAATACCGCGCCACATAGC |
| | | AGAACTTTAAAAGTGCTCATCATTGGAAAACGTTCTTCGGGGC |
| | | GAAAACTCTCAAGGATCTTACCGCTGTTGAGATCCAGTTCGATG |
| | | TAACCCACTCGTGCACCCAACTGATCTTCAGCATCTTTTACTTT |
| | | CACCAGCGTTTCTGGGTGAGCAAAAACAGGAAGGCAAAATGCC |
| | | GCAAAAAAGGGAATAAGGGCGACACGGAAATGTTGAATACTC |
| | | ATACTCTTCCTTTTTCAATATTATTGAAGCATTTATCAGGGTTAT |
| | | TGTCTCATGAGCGGATACATATTTGAATGTATTTAGAAAAATAA |
| | | ACAAATAGGGGTTCCGCGCACATTTCCCCGAAAAGTGCCACCT |
| | | GACGTCTAAGAAACCATTATTATCATGACATTAACCTATAAAA |
| | | ATAGGCGTATCACGAGGCCCTTTCGTCTCGCGCGTTTCGGTGAT |
| | | GACGGTGAAAACCTCTGACACATGCAGCTCCCGGAGACGGTCA |
| | | CAGCTTGTCTGTAAGCGGATGCCGGGAGCAGACAAGCCCGTCA |
| | | GGGCGCGTCAGCGGGTGTTGGCGGGTGTCGGGGCTGGCTTAAC |
| | | TATGCGGCATCAGAGCAGATTGTACTGAGAGTGCACCATATGC |
| | | GGTGTGAAATACCGCACAGATGCGTAAGGAGAAAATACCGCAT |
| | | CAGGCGCCATTCGCCATTCAGGCTGCGCAACTGTTGGGAAGGG |
| | | CGATCGGTGCGGGCCTCTTCGCTATTACGCCAGCTGGCGAAAG |
| | | GGGGATGTGCTGCAAGGCGATTAAGTTGGGTAACGCCAGGGTT |
| | | TTCCCAGTCACGACGTTGTAAAACGACGGCCAGTGCCAAGCTG |
| 82 | p502_NKG2D | ACGCGTGTAGTCTTATGCAATACTCTGTAGTCTTGCAACATGGT |
| | dimer_ | AACGATGAGTTAGCAACATGCCTTACAAGGAGAGAAAAAGCA |
| | CD3epsilon (DNA | CCGTGCATGCCGATTGGTGGAAGTAAGGTGGTACGATCGTGCC |
| | Sequence) | TTATTAGGAAGGCAACAGACGGGTCTGACATGGATTGGACGAA |
| | | CCACTGAATTGCCGCATTGCAGAGATATTGTATTTAAGTGCCTA |
| | | GCTCGATACATAAACGGGTCTCTCTGGTTAGACCAGATCTGAG |
| | | CCTGGGAGCTCTCTGGCTAACTAGGGAACCCACTGCTTAAGCCT |
| | | CAATAAAGCTTGCCTTGAGTGCTTCAAGTAGTGTGTGCCCGTCT |
| | | GTTGTGTGACTCTGGTAACTAGAGATCCCTCAGACCCTTTTAGT |
| | | CAGTGTGGAAAATCTCTAGCAGTGGCGCCCGAACAGGGACTTG |
| | | AAAGCGAAAGGGAAACCAGAGGAGCTCTCTCGACGCAGGACT |
| | | CGGCTTGCTGAAGCGCGCACGGCAAGAGGCGAGGGGCGGCGA |
| | | CTGGTGAGTACGCCAAAAATTTTGACTAGCGGAGGCTAGAAGG |
| | | AGAGAGATGGGTGCGAGAGCGTCAGTATTAAGCGGGGGAGAA |
| | | TTAGATCGCGATGGGAAAAAATTCGGTTAAGGCCAGGGGGAAA |
| | | GAAAAAATATAAATTAAAACATATAGTATGGGCAAGCAGGGA |
| | | GCTAGAACGATTCGCAGTTAATCCTGGCCTGTTAGAAACATCA |
| | | GAAGGCTGTAGACAAATACTGGGACAGCTACAACCATCCCTTC |
| | | AGACAGGATCAGAAGAACTTAGATCATTATATAATACAGTAGC |
| | | AACCCTCTATTGTGTGCATCAAAGGATAGAGATAAAAGACACC |
| | | AAGGAAGCTTTAGACAAGATAGAGGAAGAGCAAAACAAAAGT |
| | | AAGACCACCGCACAGCAAGCGGCCACTGATCTTCAGACCTGGA |
| | | GGAGGAGATATGAGGGACAATTGGAGAAGTGAATTATATAAAT |
| | | ATAAAGTAGTAAAAATTGAACCATTAGGAGTAGCACCCACCAA |
| | | GGCAAAGAGAAGAGTGGTGCAGAGAGAAAAAAGAGCAGTGGG |
| | | AATAGGAGCTTTGTTCCTTGGGTTCTTGGGAGCAGCAGGAAGC |
| | | ACTATGGGCGCAGCGTCAATGACGCTGACGGTACAGGCCAGAC |
| | | AATTATTGTCTGGTATAGTGCAGCAGCAGAACAATTTGCTGAG |
| | | GGCTATTGAGGCGCAACAGCATCTGTTGCAACTCACAGTCTGG |
| | | GGCATCAAGCAGCTCCAGGCAAGAATCCTGGCTGTGGAAAGAT |
| | | ACCTAAAGGATCAACAGCTCCTGGGGATTTGGGGTTGCTCTGG |
| | | AAAACTCATTTGCACCACTGCTGTGCCTTGGAATGCTAGTTGGA |
| | | GTAATAAATCTCTGGAACAGATTTGGAATCACACGACCTGGAT |
| | | GGAGTGGGACAGAGAAATTAACAATTACACAAGCTTAATACAC |
| | | TCCTTAATTGAAGAATCGCAAAACCAGCAAGAAAAGAATGAAC |
| | | AAGAATTATTGGAATTAGATAAATGGGCAAGTTTGTGGAATTG |
| | | GTTTAACATAACAAATTGGCTGTGGTATATAAAATTATTCATAA |
| | | TGATAGTAGGAGGCTTGGTAGGTTTAAGAATAGTTTTTGCTGTA |
| | | CTTTCTATAGTGAATAGAGTTAGGCAGGGATATTCACCATTATC |
| | | GTTTCAGACCCACCTCCCAACCCCGAGGGGACCCGACAGGCCC |
| | | GAAGGAATAGAAGAAGAAGGTGGAGAGAGAGACAGAGACAG |
| | | ATCCATTCGATTAGTGAACGGATCTCGACGGTATCGGTTAACTT |
| | | TTAAAAGAAAAGGGGGGATTGGGGGGTACAGTGCAGGGGAAA |
| | | GAATAGTAGACATAATAGCAACAGACATACAAACTAAAGAATT |
| | | ACAAAAACAAATTACAAAATTCAAAATTTTATCGATACTAGTG |
| | | GATCTGCGATCGCTCCGGTGCCCGTCAGTGGGCAGAGCGCACA |
| | | TCGCCCACAGTCCCCGAGAAGTTGGGGGGAGGGGTCGGCAATT |
| | | GAACGGGTGCCTAGAGAAGGTGGCGCGGGGTAAACTGGGAAA |
| | | GTGATGTCGTGTACTGGCTCCGCCTTTTTCCCGAGGGTGGGGGA |
| | | GAACCGTATATAAGTGCAGTAGTCGCCGTGAACGTTCTTTTTCG |
| | | CAACGGGTTTGCCGCCAGAACACAGCTGAAGCTTCGAGGGGCT |
| | | CGCATCTCTCCTTCACGCGCCCGCCGCCCTACCTGAGGCCGCCA |
| | | TCCACGCCGGTTGAGTCGCGTTCTGCCGCCTCCCGCCTGTGGTG |
| | | CCTCCTGAACTGCGTCCGCCGTCTAGGTAAGTTTAAAGCTCAGG |
| | | TCGAGACCGGGCCTTTGTCCGGCGCTCCCTTGGAGCCTACCTAG |
| | | ACTCAGCCGGCTCTCCACGCTTTGCCTGACCCTGCTTGCTCAAC |
| | | TCTACGTCTTTGTTTCGTTTTCTGTTCTGCGCCGTTACAGATCCA |
| | | AGCTGTGACCGGCGCCTACTCTAGAGCCGCCACCATGCTTCTCC |
| | | TGGTGACAAGCCTTCTGCTCTGTGAGTTACCACACCCAGCATTC |
| | | CTCCTGATCCCAAACTCCCTCTTCAACCAGGAGGTGCAGATCCC |
| | | CCTCACAGAGAGCTACTGCGGGCCCTGTCCAAAGAATTGGATA |
| | | TGTTACAAGAACAATTGCTACCAGTTCTTCGATGAGTCAAAAA |
| | | ATTGGTATGAGAGCCAAGCTTCCTGCATGTCTCAGAATGCCAG |
| | | CCTTCTGAAGGTGTACTCAAAAGAAGACCAGGACTTGCTGAAA |
| | | CTGGTCAAGTCTTACCACTGGATGGGGCTCGTGCACATTCCAAC |
| | | GAACGGTAGCTGGCAGTGGGAAGATGGCTCCATATTGTCTCCT |
| | | AACCTTCTCACCATAATAGAGATGCAGAAGGGTGATTGCGCTC |
| | | TGTACGCTAGTAGCTTCAAGGGCTATATTGAGAATTGTAGTACA |
| | | CCCAACACATACATTTGTATGCAGAGAACCGTGGGAGGTGGTG |
| | | GCAGCGGTGGCGGTGGAAGTGGTGGCGGTGGCAGTCTCGAGAA |
| | | CTCATTATTCAACCAAGAAGTTCAAATTCCCTTGACCGAAAGTT |
| | | ACTGTGGCCCATGTCCTAAAAACTGGATATGTTACAAAAATAA |
| | | CTGCTACCAATTTTTTGATGAGAGTAAAAACTGGTATGAGAGC |
| | | CAGGCTTCTTGTATGTCTCAAAATGCCAGCCTTCTGAAAGTATA |
| | | CAGCAAAGAGGACCAGGATTTACTTAAACTGGTGAAGTCATAT |
| | | CATTGGATGGGACTAGTACACATTCCAACAAATGGATCTTGGC |
| | | AGTGGGAAGATGGCTCCATTCTCTCACCCAACCTACTAACAAT |
| | | AATTGAAATGCAGAAGGGAGACTGTGCACTCTATGCCTCTAGC |
| | | TTTAAAGGCTATATAGAAAACTGTTCAACTCCAAATACATACAT |
| | | CTGCATGCAAAGGACTGTGGCGGCCGCAGGTGGCGGCGGTTCT |
| | | GGTGGCGGCGGTTCTGGTGGCGGCGGTTCTCTCGAGGATGGTA |
| | | ATGAAGAAATGGGTGGTATTACACAGACACCATATAAAGTCTC |
| | | CATCTCTGGAACCACAGTAATATTGACATGCCCTCAGTATCCTG |
| | | GATCTGAAATACTATGGCAACACAATGATAAAAACATAGGCGG |
| | | TGATGAGGATGATAAAAACATAGGCAGTGATGAGGATCACCTG |
| | | TCACTGAAGGAATTTTCAGAATTGGAGCAAAGTGGTTATTATGT |
| | | CTGCTACCCCAGAGGAAGCAAACCAGAAGATGCGAACTTTTAT |
| | | CTCTACCTGAGGGCAAGAGTGTGTGAGAACTGCATGGAGATGG |
| | | ATGTGATGTCGGTGGCCACAATTGTCATAGTGGACATCTGCATC |
| | | ACTGGGGGCTTGCTGCTGCTGGTTTACTACTGGAGCAAGAATA |
| | | GAAAGGCCAAGGCCAAGCCTGTGACACGAGGAGCGGGTGCTG |
| | | GCGGCAGGCAAAGGGGACAAAACAAGGAGAGGCCACCACCTG |
| | | TTCCCAACCCAGACTATGAGCCCATCCGGAAAGGCCAGCGGGA |
| | | CCTGTATTCTGGCCTGAATCAGAGACGCATCTGATAAGAATTCG |
| | | AATTTAAATCGGATCCGCGGCCGCGTCGACAATCAACCTCTGG |
| | | ATTACAAAATTTGTGAAAGATTGACTGGTATTCTTAACTATGTT |
| | | GCTCCTTTTACGCTATGTGGATACGCTGCTTTAATGCCTTTGTAT |
| | | CATGCTATTGCTTCCCGTATGGCTTTCATTTTCTCCTCCTTGTAT |
| | | AAATCCTGGTTGCTGTCTCTTTATGAGGAGTTGTGGCCCGTTGT |
| | | CAGGCAACGTGGCGTGGTGTGCACTGTGTTTGCTGACGCAACC |
| | | CCCACTGGTTGGGGCATTGCCACCACCTGTCAGCTCCTTTCCGG |
| | | GACTTTCGCTTTCCCCCTCCCTATTGCCACGGCGGAACTCATCG |
| | | CCGCCTGCCTTGCCCGCTGCTGGACAGGGGCTCGGCTGTTGGGC |
| | | ACTGACAATTCCGTGGTGTTGTCGGGGAAATCATCGTCCTTTCC |
| | | TTGGCTGCTCGCCTGTGTTGCCACCTGGATTCTGCGCGGGACGT |
| | | CCTTCTGCTACGTCCCTTCGGCCCTCAATCCAGCGGACCTTCCT |
| | | TCCCGCGGCCTGCTGCCGGCTCTGCGGCCTCTTCCGCGTCTTCG |
| | | CCTTCGCCCTCAGACGAGTCGGATCTCCCTTTGGGCCGCCTCCC |
| | | CGCCTGGTACCTTTAAGACCAATGACTTACAAGGCAGCTGTAG |
| | | ATCTTAGCCACTTTTTAAAAGAAAAGGGGGGACTGGAAGGGCT |
| | | AATTCACTCCCAACGAAGATAAGATCTGCTTTTTGCTTGTACTG |
| | | GGTCTCTCTGGTTAGACCAGATCTGAGCCTGGGAGCTCTCTGGC |
| | | TAACTAGGGAACCCACTGCTTAAGCCTCAATAAAGCTTGCCTTG |
| | | AGTGCTTCAAGTAGTGTGTGCCCGTCTGTTGTGTGACTCTGGTA |
| | | ACTAGAGATCCCTCAGACCCTTTTAGTCAGTGTGGAAAATCTCT |
| | | AGCAGTAGTAGTTCATGTCATCTTATTATTCAGTATTTATAACT |
| | | TGCAAAGAAATGAATATCAGAGAGTGAGAGGAACTTGTTTATT |
| | | GCAGCTTATAATGGTTACAAATAAAGCAATAGCATCACAAATT |
| | | TCACAAATAAAGCATTTTTTTCACTGCATTCTAGTTGTGGTTTGT |
| | | CCAAACTCATCAATGTATCTTATCATGTCTGGCTCTAGCTATCC |
| | | CGCCCCTAACTCCGCCCATCCCGCCCCTAACTCCGCCCAGTTCC |
| | | GCCCATTCTCCGCCCCATGGCTGACTAATTTTTTTTATTTATGCA |
| | | GAGGCCGAGGCCGCCTCGGCCTCTGAGCTATTCCAGAAGTAGT |
| | | GAGGAGGCTTTTTTGGAGGCCTAGACTTTTGCAGAGACGGCCC |
| | | AAATTCGTAATCATGGTCATAGCTGTTTCCTGTGTGAAATTGTT |
| | | ATCCGCTCACAATTCCACACAACATACGAGCCGGAAGCATAAA |
| | | GTGTAAAGCCTGGGGTGCCTAATGAGTGAGCTAACTCACATTA |
| | | ATTGCGTTGCGCTCACTGCCCGCTTTCCAGTCGGGAAACCTGTC |
| | | GTGCCAGCTGCATTAATGAATCGGCCAACGCGCGGGGAGAGGC |
| | | GGTTTGCGTATTGGGCGCTCTTCCGCTTCCTCGCTCACTGACTC |
| | | GCTGCGCTCGGTCGTTCGGCTGCGGCGAGCGGTATCAGCTCACT |
| | | CAAAGGCGGTAATACGGTTATCCACAGAATCAGGGGATAACGC |
| | | AGGAAAGAACATGTGAGCAAAAGGCCAGCAAAAGGCCAGGAA |
| | | CCGTAAAAAGGCCGCGTTGCTGGCGTTTTTCCATAGGCTCCGCC |
| | | CCCCTGACGAGCATCACAAAAATCGACGCTCAAGTCAGAGGTG |
| | | GCGAAACCCGACAGGACTATAAAGATACCAGGCGTTTCCCCCT |
| | | GGAAGCTCCCTCGTGCGCTCTCCTGTTCCGACCCTGCCGCTTAC |
| | | CGGATACCTGTCCGCCTTTCTCCCTTCGGGAAGCGTGGCGCTTT |
| | | CTCATAGCTCACGCTGTAGGTATCTCAGTTCGGTGTAGGTCGTT |
| | | CGCTCCAAGCTGGGCTGTGTGCACGAACCCCCCGTTCAGCCCG |
| | | ACCGCTGCGCCTTATCCGGTAACTATCGTCTTGAGTCCAACCCG |
| | | GTAAGACACGACTTATCGCCACTGGCAGCAGCCACTGGTAACA |
| | | GGATTAGCAGAGCGAGGTATGTAGGCGGTGCTACAGAGTTCTT |
| | | GAAGTGGTGGCCTAACTACGGCTACACTAGAAGGACAGTATTT |
| | | GGTATCTGCGCTCTGCTGAAGCCAGTTACCTTCGGAAAAAGAG |
| | | TTGGTAGCTCTTGATCCGGCAAACAAACCACCGCTGGTAGCGG |
| | | TGGTTTTTTTGTTTGCAAGCAGCAGATTACGCGCAGAAAAAAA |
| | | GGATCTCAAGAAGATCCTTTGATCTTTTCTACGGGGTCTGACGC |
| | | TCAGTGGAACGAAAACTCACGTTAAGGGATTTTGGTCATGAGA |
| | | TTATCAAAAAGGATCTTCACCTAGATCCTTTTAAATTAAAAATG |
| | | AAGTTTTAAATCAATCTAAAGTATATATGAGTAAACTTGGTCTG |
| | | ACAGTTACCAATGCTTAATCAGTGAGGCACCTATCTCAGCGATC |
| | | TGTCTATTTCGTTCATCCATAGTTGCCTGACTCCCCGTCGTGTAG |
| | | ATAACTACGATACGGGAGGGCTTACCATCTGGCCCCAGTGCTG |
| | | CAATGATACCGCGAGACCCACGCTCACCGGCTCCAGATTTATC |
| | | AGCAATAAACCAGCCAGCCGGAAGGGCCGAGCGCAGAAGTGG |
| | | TCCTGCAACTTTATCCGCCTCCATCCAGTCTATTAATTGTTGCCG |
| | | GGAAGCTAGAGTAAGTAGTTCGCCAGTTAATAGTTTGCGCAAC |
| | | GTTGTTGCCATTGCTACAGGCATCGTGGTGTCACGCTCGTCGTT |
| | | TGGTATGGCTTCATTCAGCTCCGGTTCCCAACGATCAAGGCGAG |
| | | TTACATGATCCCCCATGTTGTGCAAAAAAGCGGTTAGCTCCTTC |
| | | GGTCCTCCGATCGTTGTCAGAAGTAAGTTGGCCGCAGTGTTATC |
| | | ACTCATGGTTATGGCAGCACTGCATAATTCTCTTACTGTCATGC |
| | | CATCCGTAAGATGCTTTTCTGTGACTGGTGAGTACTCAACCAAG |
| | | TCATTCTGAGAATAGTGTATGCGGCGACCGAGTTGCTCTTGCCC |
| | | GGCGTCAATACGGGATAATACCGCGCCACATAGCAGAACTTTA |
| | | AAAGTGCTCATCATTGGAAAACGTTCTTCGGGGCGAAAACTCT |
| | | CAAGGATCTTACCGCTGTTGAGATCCAGTTCGATGTAACCCACT |
| | | CGTGCACCCAACTGATCTTCAGCATCTTTTACTTTCACCAGCGT |
| | | TTCTGGGTGAGCAAAAACAGGAAGGCAAAATGCCGCAAAAAA |
| | | GGGAATAAGGGCGACACGGAAATGTTGAATACTCATACTCTTC |
| | | CTTTTTCAATATTATTGAAGCATTTATCAGGGTTATTGTCTCATG |
| | | AGCGGATACATATTTGAATGTATTTAGAAAAATAAACAAATAG |
| | | GGGTTCCGCGCACATTTCCCCGAAAAGTGCCACCTGACGTCTA |
| | | AGAAACCATTATTATCATGACATTAACCTATAAAAATAGGCGT |
| | | ATCACGAGGCCCTTTCGTCTCGCGCGTTTCGGTGATGACGGTGA |
| | | AAACCTCTGACACATGCAGCTCCCGGAGACGGTCACAGCTTGT |
| | | CTGTAAGCGGATGCCGGGAGCAGACAAGCCCGTCAGGGCGCGT |
| | | CAGCGGGTGTTGGCGGGTGTCGGGGCTGGCTTAACTATGCGGC |
| | | ATCAGAGCAGATTGTACTGAGAGTGCACCATATGCGGTGTGAA |
| | | ATACCGCACAGATGCGTAAGGAGAAAATACCGCATCAGGCGCC |
| | | ATTCGCCATTCAGGCTGCGCAACTGTTGGGAAGGGCGATCGGT |
| | | GCGGGCCTCTTCGCTATTACGCCAGCTGGCGAAAGGGGGATGT |
| | | GCTGCAAGGCGATTAAGTTGGGTAACGCCAGGGTTTTCCCAGT |
| | | CACGACGTTGTAAAACGACGGCCAGTGCCAAGCTG |
| 83 | human R0R1, | MHRPRRRGTRPPLLALLAALLLAARGAAAQETELSVSAELVPTSS |
| | Isoform 1 | WNISSELNKDSYLTLDEPMNNITTSLGQTAELHCKVSGNPPPTIRW |
| | (canonical) | FKNDAPVVQEPRRLSFRSTIYGSRLRIRNLDTTDTGYFQCVATNGK |
| | UniProt Acc # | EVVSSTGVLFVKFGPPPTASPGYSDEYEEDGFCQPYRGIACARFIG |
| | Q01973-1 | NRTVYMESLHMQGEIENQITAAFTMIGTSSHLSDKCSQFAIPSLCH |
| | | YAFPYCDETSSVPKPRDLCRDECEILENVLCQTEYIFARSNPMILM |
| | | RLKLPNCEDLPQPESPEAANCIRIGIPMADPINKNHKCYNSTGVDY |
| | | RGTVSVTKSGRQCQPWNSQYPHTHTFTALRFPELNGGHSYCRNPG |
| | | NQKEAPWCFTLDENFKSDLCDIPACDSKDSKEKNKMEILYILVPSV |
| | | AIPLAIALLFFFICVCRNNQKSSSAPVQRQPKHVRGQNVEMSMLNA |
| | | YKPKSKAKELPLSAVRFMEELGECAFGKIYKGHLYLPGMDHAQL |
| | | VAIKTLKDYNNPQQWTEFQQEASLMAELHHPNIVCLLGAVTQEQP |
| | | VCMLFEYINQGDLHEFLIMRSPHSDVGCSSDEDGTVKSSLDHGDF |
| | | LHIAIQIAAGMEYLSSHFFVHKDLAARNILIGEQLHVKISDLGLSREI |
| | | YSADYYRVQSKSLLPIRWMPPEAIMYGKFSSDSDIWSFGVVLWEIF |
| | | SFGLQPYYGFSNQEVIEMVRKRQLLPCSEDCPPRMYSLMTECWNE |
| | | IPSRRPRFKDIHVRLRSWEGLSSHTSSTTPSGGNATTQTTSLSASPV |
| | | SNLSNPRYPNYMFPSQGITPQGQIAGFIGPPIPQNQRFIPINGYPIPPG |
| | | YAAFPAAHYQPTGPPRVIQHCPPPKSRSPSSASGSTSTGHVTSLPSS |
| | | GSNQEANIPLLPHMSIPNHPGGMGITVFGNKSQKPYKIDSKQASLL |
| | | GDANIHGHTESMISAEL |
| 84 | human ROR1 | MHRPRRRGTRPPLLALLAALLLAARGAAAQETELSVSAELVPTSS |
| | Isoform 2 | WNISSELNKDSYLTLDEPMNNITTSLGQTAELHCKVSGNPPPTIRW |
| | | FKNDAPWQEPRRLSFRSTIYGSRLRIRNLDTTDTGYFQCVATNGK |
| | | EVVSSTGVLFVKFGPPPTASPGYSDEYEEDGFCQPYRGIACARFIG |
| | | NRTVYMESLHMQGEIENQITAAFTMIGTSSHLSDKCSQFAIPSLCH |
| | | YAFPYCDETSSVPKPRDLCRDECEILENVLCQTEYIFARSNPMILM |
| | | RLKLPNCEDLPQPESPEAANCIRIGIPMADPINKNHKCYNSTGVDY |
| | | RGTVSVTKSGRQCQPWNSQYPHTHTFTALRFPELNGGHSYCRNPG |
| | | NQKEAPWCFTLDENFKSDLCDIPACGK |
| 85 | human ROR1 | MNNITTSLGQTAELHCKVSGNPPPTIRWFKNDAPVVQEPRRLSF |
| | Isoform 3 | RSTIYGSRLRIRNLDTTDTGYFQCVATNGKEVVSSTGVLFVKFGPP |
| | | PTASPGYSDEYEEDGFCQPYRGIACARFIGNRTVYMESLHMQGEIE |
| | | NQITAAFTMIGTSSHLSDKCSQFAIPSLCHYAFPYCDETSSVPKPRD |
| | | LCRDECEILENVLCQTEYIFARSNPMILMRLKLPNCEDLPQPESPEA |
| | | ANCIRIGIPMADPINKNHKCYNSTGVDYRGTVSVTKSGRQCQPWN |
| | | SQYPHTHTFTALRFPELNGGHSYCRNPGNQKEAPWCFTLDENFKS |
| | | DLCDIPACDSKDSKEKNKMEILYILVPSVAIPLAIALLFFFICVCRNN |
| | | QKSSSAPVQRQPKHVRGQNVEMSMLNAYKPKSKAKELPLSAVRF |
| | | MEELGECAFGKIYKGHLYLPGMDHAQLVAIKTLKDYNNPQQWTE |
| | | FQQEASLMAELHHPNIVCLLGAVTQEQPVCMLFEYINQGDLHEFLI |
| | | MRSPHSDVGCSSDEDGTVKSSLDHGDFLHIAIQIAAGMEYLSSHFF |
| | | VHKDLAARNILIGEQLHVKISDLGLSREIYSADYYRVQSKSLLPIR |
| | | WMPPEAIMYGKFSSDSDIWSFGVVLWEIFSFGLQPYYGFSNQEVIE |
| | | MVRKRQLLPCSEDCPPRMYSLMTECWNEIPSRRPRFKDIHVRLRS |
| | | WEGLSSHTSSTTPSGGNATTQTTSLSASPVSNLSNPRYPNYMFPSQ |
| | | GITPQGQIAGFIGPPIPQNQRFIPINGYPIPPGYAAFPAAHYQPTGPPR |
| | | VIQHCPPPKSRSPSSASGSTSTGHVTSLPSSGSNQEANIPLLPHMSIP |
| | | NHPGGMGITVFGNKSQKPYKIDSKQASLLGDANIHGHTESMISAEL |
| 86 | CD16 Isoform A, | MWQLLLPTALLLLVSAGMRTEDLPKAVVFLEPQWYRVLEKDSVT |
| | UniProt | LKCQGAYSPEDNSTQWFHNESLISSQASSYFIDAATVDDSGEYRCQ |
| | Accession | TNLSTLSDPVQLEVHIGWLLLQAPRWVFKEEDPIHLRCHSWKNTA |
| | No. P08637 | LHKVTYLQNGKGRKYFHHNSDFYIPKATLKDSGSYFCRGLFGSKN |
| | | VSSETVNITITQGLAVSTISSFFPPGYQVSFCLVMVLLFAVDTGLYF |
| | | SVKTNIRSSTRDWKDHKFKWRKDPQDK |
| 87 | CD16 V158 | MWQLLLPTALLLLVSAGMRTEDLPKAVVFLEPQWYRVLEKDSVT |
| | variant Of | LKCQGAYSPEDNSTQWFHNESLISSQASSYFIDAATVDDSGEYRCQ |
| | SEQ ID NO: 23 | TNLSTLSDPVQLEVHIGWLLLQAPRWVFKEEDPIHLRCHSWKNTA |
| | | LHKVTYLQNGKGRKYFHHNSDFYIPKATLKDSGSYFCRGLVGSK |
| | | NVSSETVNITITQGLAVSTISSFFPPGYQVSFCLVMVLLFAVDTGLY |
| | | FSVKTNIRSSTRDWKDHKFKWRKDPQDK |
| 88 | Anti-CD19 light | AGGGCAAGTCAGGACATTAGTAAA |
| | chain CDR1 (DNA) | |
| 89 | Anti-CD19 light | RASQDISK |
| | chain CDR1 | |
| | (protein) | |
| 90 | Anti-CD19 light | ATCTACCATACATCAAGATTA |
| | chain CDR2 (DNA) | |
| 91 | Anti-CD19 light | IYHTSRL |
| | chain CDR2 | |
| | (protein) | |
| 92 | Anti-CD19 light | CAACAGGGTAATACGCTTCCGTACACG |
| | chain CDR3 (DNA) | |
| 93 | Anti-CD19 light | QQGNTLPYT |
| | chain CDR3 | |
| | (protein) | |
| 94 | Anti-CD19 heavy | GGGGTCTCATTACCCGACTATGGTGTAAGC |
| | chain CDR1 (DNA) | |
| 95 | Anti-CD19 heavy | GVSLPDYGVS |
| | chain CDR1 | |
| | (protein) | |
| 96 | Anti-CD19 heavy | GTAATATGGGGTAGTGAAACCACATACTATAATTCAGCTCTC |
| | chain CDR2 (DNA) | |
| 97 | Anti-CD19 heavy | VIWGSETTYYNSAL |
| | chain CDR2 | |
| | (protein) | |
| 98 | Anti-CD19 heavy | CATTATTACTACGGTGGTAGCTATGCTATGGACTAC |
| | chain CDR3 (DNA) | |
| 99 | Anti-CD19 heavy | HYYYGGSYAMDY |
| | chain CDR3 | |
| | (protein) | |
| 100 | Anti-CD19 light | GACATCCAGATGACACAGACTACATCCTCCCTGTCTGCCTCTCT |
| | chain variable | GGGAGACAGAGTCACCATCAGTTGCAGGGCAAGTCAGGACATT |
| | region (DNA) | AGTAAATATTTAAATTGGTATCAGCAGAAACCAGATGGAACTG |
| | | TTAAACTCCTGATCTACCATACATCAAGATTACACTCAGGAGTC |
| | | CCATCAAGGTTCAGTGGCAGTGGGTCTGGAACAGATTATTCTCT |
| | | CACCATTAGCAACCTGGAGCAAGAAGATATTGCCACTTACTTTT |
| | | GCCAACAGGGTAATACGCTTCCGTACACGTTCGGAGGGGGGAC |
| | | TAAGTTGGAAATAACA |
| 101 | Anti-CD19 light | DIQMTQTTSSLSASLGDRVTISCRASQDISKYLNWYQQKPDGTVK |
| | chain variable | LLIYHTSRLHSGVPSRFSGSGSGTDYSLTISNLEQEDIATYFCQQGN |
| | region (protein) | TLPYTFGGGTKLEIT |
| 102 | Anti-CD19 heavy | GAGGTGAAACTGCAGGAGTCAGGACCTGGCCTGGTGGCGCCCT |
| | chain variable | CACAGAGCCTGTCCGTCACATGCACTGTCTCAGGGGTCTCATTA |
| | region (DNA) | CCCGACTATGGTGTAAGCTGGATTCGCCAGCCTCCACGAAAGG |
| | | GTCTGGAGTGGCTGGGAGTAATATGGGGTAGTGAAACCACATA |
| | | CTATAATTCAGCTCTCAAATCCAGACTGACCATCATCAAGGAC |
| | | AACTCCAAGAGCCAAGTTTTCTTAAAAATGAACAGTCTGCAAA |
| | | CTGATGACACAGCCATTTACTACTGTGCCAAACATTATTACTAC |
| | | GGTGGTAGCTATGCTATGGACTACTGGGGTCAAGGAACCTCAG |
| | | TCACCGTCTCCTCA |
| 103 | Anti-CD19 heavy | EVKLQESGPGLVAPSQSLSVTCTVSGVSLPDYGVSWIRQPPRKGLE |
| | chain variable | WLGVIWGSETTYYNSALKSRLTIIKDNSKSQVFLKMNSLQTDDTAI |
| | region (protein) | YYCAKHYYYGGSYAMDYWGQGTSVTVSS |
| 104 | Anti-BCMA light | AAAAGCAGCCAGAGCCTGGTGCATAGCAACGGCAACACCTATC |
| | chain CDR1 (DNA) | TGCAT |
| 105 | Anti-BCMA light | KSSQSLVHSNGNTYLH |
| | chain CDR1 | |
| | (protein) | |
| 106 | Anti-BCMA light | AAAGTGAGCAACCGCTTTAGC |
| | chain CDR2 (DNA) | |
| 107 | Anti-BCMA light | KVSNRFS |
| | chain CDR2 | |
| | (protein) | |
| 108 | Anti-BCMA light | GCGGAAACCAGCCATGTGCCGTGGACC |
| | chain CDR3 (DNA) | |
| 109 | Anti-BCMA light | AETSHVPWT |
| | chain CDR3 | |
| | (protein) | |
| 110 | Anti-BCMA heavy | AAAGCGAGCGGCTATAGCTTTCCGGATTATTATATTAAC |
| | chain CDR1 (DNA) | |
| 111 | Anti-BCMA heavy | KASGYSFPDYYIN |
| | chain CDR1 | |
| | (protein) | |
| 112 | Anti-BCMA heavy | TGGATTTATTTTGCGAGCGGCAACAGCGAATATAACCAGAAAT |
| | chain CDR2 (DNA) | TTACCGGC |
| 113 | Anti-BCMA heavy | WIYFASGNSEYNQKFTG |
| | chain CDR2 | |
| | (protein) | |
| 114 | Anti-BCMA heavy | CTGTATGATTATGATTGGTATTTTGATGTG |
| | chain CDR3 (DNA) | |
| 115 | Anti-BCMA heavy | LYDYDWYFDV |
| | chain CDR3 | |
| | (protein) | |
| 116 | Anti-BCMA heavy | CAGGTGCAGCTGGTGCAGAGCGGCGCGGAAGTGAAAAAACCG |
| | chain variable | GGCGCGAGCGTGAAAGTGAGCTGCAAAGCGAGCGGCTATAGCT |
| | region (DNA) | TTCCGGATTATTATATTAACTGGGTGCGCCAGGCGCCGGGCCA |
| | | GGGCCTGGAATGGATGGGCTGGATTTATTTTGCGAGCGGCAAC |
| | | AGCGAATATAACCAGAAATTTACCGGCCGCGTGACCATGACCC |
| | | GCGATACCAGCAGCAGCACCGCGTATATGGAACTGAGCAGCCT |
| | | GCGCAGCGAAGATACCGCGGTGTATTTTTGCGCGAGCCTGTAT |
| | | GATTATGATTGGTATTTTGATGTGTGGGGCCAGGGCACCATGGT |
| | | GACCGTGAGCAGC |
| 117 | Anti-BCMA heavy | QVQLVQSGAEVKKPGASVKVSCKASGYSFPDYYINWVRQAPGQG |
| | chain variable | LEWMGWIYFASGNSEYNQKFTGRVTMTRDTSSSTAYMELSSLRSE |
| | region (protein) | DTAVYFCASLYDYDWYFDVWGQGTMVTVSS |
| 118 | Anti-BCMA light | GATATTGTGATGACCCAGACCCCGCTGAGCCTGAGCGTGACCC |
| | chain variable | CGGGCGAACCGGCGAGCATTAGCTGCAAAAGCAGCCAGAGCCT |
| | region (DNA) | GGTGCATAGCAACGGCAACACCTATCTGCATTGGTATCTGCAG |
| | | AAACCGGGCCAGAGCCCGCAGCTGCTGATTTATAAAGTGAGCA |
| | | ACCGCTTTAGCGGCGTGCCGGATCGCTTTAGCGGCAGCGGCAG |
| | | CGGCGCGGATTTTACCCTGAAAATTAGCCGCGTGGAAGCGGAA |
| | | GATGTGGGCGTGTATTATTGCGCGGAAACCAGCCATGTGCCGT |
| | | GGACCTTTGGCCAGGGCACCAAACTGGAAATTAAAAGC |
| 119 | Anti-BCMA light | DIVMTQTPLSLSVTPGEPASISCKSSQSLVHSNGNTYLHWYLQKPG |
| | chain variable | QSPQLLIYKVSNRFSGVPDRFSGSGSGADFTLKISRVEAEDVGVYY |
| | region (protein) | CAETSHVPWTFGQGTKLEIKS |
| 120 | Anti-CD22 light | QDIHGY |
| | chain CDR1 | |
| 121 | Anti-CD22 light | YTS |
| | chain CDR2 | |
| 122 | Anti-CD22 light | QQGNTLPWT |
| | chain CDR3 | |
| 123 | Anti-CD22 heavy | GFAFSIYD |
| | chain CDR1 | |
| 124 | Anti-CD22 heavy | ISSGGGTT |
| | chain CDR2 | |
| 125 | Anti-CD22 heavy | ARHSGYGTHWGVLFAY |
| | chain CDR3 | |
| 126 | Anti-CD22 light | DIQMTQSPSSLSASVGDRVTITCRASQTIWSYLNWYQQRPGKAPN |
| | chain variable | LLIYAASSLQSGVPSRFSGRGSGTDFTLTISSLQAEDFATYYCQQSY |
| | region | SIPQTFGQGTKLEIKEVQLVESGGGLVKPGGSLKLSCAASGFAFSIY |
| | | DMSWVRQTPEKRLEWVAYISSGGGTTYYPDTVKGRFTISRDNAK |
| | | NTLYLQMSSLKSEDTAMYYCARHSGYGTHWGVLFAYWQGTLVT |
| | | VSA |
| 127 | Anti-CD22 heavy | QVQLQQSGPGLVKPSQTLSLTCAISGDSVSSNSAAWNWIRQSPSRG |
| | chain variable | LEWLGRTYYRSKWYNDYAVSVKSRITINPDTSKNQFSLQLNSVTP |
| | region | EDTAVYYCAREVTGDLEDAFDIWGQGTMVTVSSGGSLAALTAHQ |
| | | ACHLPLETFTRHRQPRGWEQLEQCGYPVQRLVALYLAARLSWNQ |
| | | VDQVIRNALASPGSGGDLGEAIREQPEQARLALTLAAAESERFVR |
| | | QGTGNDEAGAANGPADSGDALLERNYPTGAEFLGDGGDVSFSTR |
| | | GTQNWTVERLLQAHRQLEERGYVFVGYHGTFLEAAQSIVFGGVR |
| | | ARSQDLDAIWRGFYIAGDPALAYGYAQDQEPDAAGRIRNGALLR |
| | | VYVPRSSLPGFYRTSLTLAAPEAAGEVERLIGHPLPLRLDAITGPEE |
| | | EGGRLETILGWPLAERTVVIPSAIPTDPRNVGGDLDPSSIPDKEQAIS |
| | | ALPDYASQPGKPPREDLK |
| 128 | Anti-ROR1 scFv | GAAGTGCAGCTGCTGGAAAGCGGCGGTGGTCTGGTTCAGCCGG |
| | 2-7 VH_linker | GTGGCAGCCTGCGTCTGAGCTGTGCGGCGAGCGGCTTTACCTTT |
| | 4_anti- | AGCAGCTATGCCATGAGCTGGGTGCGTCAGGCACCGGGTAAAG |
| | ROR1 2-7 VL | GCCTGGAATGGGTGAGCGCGATTAGCGGCAGCGGCGGCAGCAC |
| | (DNA) | CTATTATGCGGATAGCGTGAAAGGCCGTTTTACCATTAGCCGTG |
| | | ATAACAGCAAAAACACCCTGTATCTGCAGATGAACAGCCTGCG |
| | | TGCGGAAGATACCGCGGTGTATTATTGCGCGAAAGATAAGGGT |
| | | TGGTTTAACTGGCAATTCGATTATTGGGGCCAGGGCACCCTGGT |
| | | TACCGTTAGCAGCGGTGGAGGCGGTTCTGGTGGAGGCGGTTCG |
| | | GATGGCGGAGGTTCAGAAATTGTGCTGACCCAGAGCCCGGGCA |
| | | CGCTGTCTCTGAGCCCGGGTGAACGTGCGACCCTGAGCTGTCGT |
| | | GCGAGCCAAAGCGTGAGCAGCAGCTATCTGGCCTGGTATCAgC |
| | | AGAAACCGGGCCAGGCACCGCGTCTGCTGATTTATGGCGCGAG |
| | | CAGCCgTGCGACCGGCATTCCGGATCGTTTTAGCGGCAGCGGTA |
| | | GCGGCACCGATTTTACCCTGACCATTAGCCGTCTGGAACCGGA |
| | | AGATTTTGCGGTGTATTATTGCCAGCAGTATGGCAGCAGCCCGT |
| | | GGACCTTTGGCCAGGGCACCAAAGTGGAAATTAAA |
| 129 | anti-ROR1 | GAAGTGCAACTTCTCGAGAGCGGTGGGGGACTCGTCCAGCCGG |
| | scFv 2-7 | GAGGTTCCCTGCGACTCAGCTGTGCAGCCTCAGGCTTTACCTTT |
| | VH (DNA) | TCCAGTTACGCAATGAGTTGGGTCCGGCAGGCGCCTGGTAAAG |
| | | GACTCGAGTGGGTGAGTGCAATCAGCGGAAGTGGCGGGTCTAC |
| | | ATACTATGCGGACTCTGTTAAAGGCAGGTTCACTATTTCAAGGG |
| | | ACAATTCCAAGAACACGCTCTACCTGCAGATGAATAGCCTTAG |
| | | AGCTGAAGACACGGCCGTGTACTATTGTGCCAAAGACAAGGGA |
| | | TGGTTCAACTGGCAGTTCGACTACTGGGGGCAGGGAACTCTCG |
| | | TCACCGTGAGCTCC |
| 130 | anti-ROR1 | GAAATTGTTCTCACACAGTCACCCGGAACCCTTTCATTGTCCCC |
| | scFv 2-7 | CGGCGAGCGCGCCACCCTCAGCTGTCGGGCCAGTCAGAGCGTG |
| | VL (DNA) | TCTAGCTCTTACCTGGCCTGGTACCAGCAGAAACCTGGGCAAG |
| | | CTCCCAGACTCCTGATATATGGGGCCAGCAGCCGGGCCACTGG |
| | | CATTCCGGACAGGTTTAGTGGATCAGGCTCTGGCACTGATTTTA |
| | | CACTGACGATTTCAAGGTTGGAACCCGAAGACTTCGCAGTGTA |
| | | CTATTGTCAGCAGTATGGGTCTAGCCCGTGGACTTTCGGGCAAG |
| | | GCACCAAGGTGGAAATCAAG |
| 131 | anti-ROR1 2-9 | GAAGTGCAGCTGCTGGAAAGCGGCGGTGGTCTGGTTCAGCCGG |
| | VH_Linker4_anti- | GTGGCAGCCTGCGTCTGAGCTGTGCGGCGAGCGGCTTTACCTTT |
| | ROR1 2-9 VL | AGCAGCTATGCCATGAGCTGGGTGCGTCAGGCACCGGGTAAAG |
| | | GCCTGGAATGGGTGAGCGCGATTAGCGGCAGCGGCGGCAGCAC |
| | | CTATTATGCGGATAGCGTGAAAGGCCGTTTTACCATTAGCCGTG |
| | | ATAACAGCAAAAACACCCTGTATCTGCAGATGAACAGCCTGCG |
| | | TGCGGAAGATACCGCGGTGTATTATTGCGCGAAAAAACAATAT |
| | | CACTTCGATTATTGGGGCCAGGGCACCCTGGTTACCGTTAGCAG |
| | | CGGTGGAGGCGGTTCTGGTGGAGGCGGTTCGGGTGGCGGAGGT |
| | | TCAGAAATTGTGCTGACCCAGAGCCCGGGCACGCTGTCTCTGA |
| | | GCCCGGGTGAACGTGCGACCCTGAGCTGTCGTGCGAGCCAGAG |
| | | CGTGAGCAGCAGCTATCTGGCCTGGTATCAGCAGAAACCGGGC |
| | | CAGGCACCGCGTCTGCTGATTTATGGCGCGAGCAGCCGTGCGA |
| | | CCGGCATTCCGGATCGTTTTAGCGGCAGCGGTAGCGGCACCGA |
| | | TTTTACCCTGACCATTAGCCGTCTGGAACCGGAAGATTTTGCGG |
| | | TGTATTATTGCCAGCAGTATGGCAGCAGCCCGTGGACCTTTGGC |
| | | CAGGGCACCAAAGTGGAAATTAAA |
| 132 | anti-ROR1 | GAAGTGCAACTTCTCGAGAGCGGTGGGGGACTCGTCCAGCCGG |
| | scFv 2-9 | GAGGTTCCCTGCGACTCAGCTGTGCAGCCTCAGGCTTTACCTTT |
| | VH (DNA) | TCCAGTTACGCAATGAGTTGGGTCCGGCAGGCGCCTGGTAAAG |
| | | GACTCGAGTGGGTGAGTGCAATCAGCGGAAGTGGCGGGTCTAC |
| | | ATACTATGCGGACTCTGTTAAAGGCAGGTTCACTATTTCAAGGG |
| | | ACAATTCCAAGAACACGCTCTACCTGCAGATGAATAGCCTTAG |
| | | AGCTGAAGACACGGCCGTGTACTATTGTGCCAAAAAGCAGTAC |
| | | CATTTCGACTACTGGGGGCAGGGAACTCTCGTCACCGTGAGCT |
| | | CC |
| 133 | anti-ROR1 | GAAATTGTTCTCACACAGTCACCCGGAACCCTTTCATTGTCCCC |
| | scFv 2-9 | CGGCGAGCGCGCCACCCTCAGCTGTCGGGCCAGTCAGAGCGTG |
| | VL (DNA) | TCTAGCTCTTACCTGGCCTGGTACCAGCAGAAACCTGGGCAAG |
| | | CTCCCAGACTCCTGATATATGGGGCCAGCAGCCGGGCCACTGG |
| | | CATTCCGGACAGGTTTAGTGGATCAGGCTCTGGCACTGATTTTA |
| | | CACTGACGATTTCAAGGTTGGAACCCGAAGACTTCGCAGTGTA |
| | | CTATTGTCAGCAGTATGGGTCTAGCCCGTGGACTTTCGGGCAAG |
| | | GCACCAAGGTGGAAATCAAG |
| 134 | anti-ROR1 | CAGTCTGCCCTGACTCAGCCTGCCTCCGTGTCTGGGTCTCCTGG |
| | scFv 3-6 | ACAGTCGATCACCATCTCCTGCACTGGAACCAGCAGTGACGTT |
| | (DNA) | GGTGGTTATAACTATGTCTCCTGGTACCAACAGCACCCAGGCA |
| | | AAGCCCCCAAAGTCATGATTTATGATGTCAGTAATCGGCCCTCA |
| | | GGGGTTTCTGATCGCTTCTCTGGCTCCAAGTCTGGCAACACGGC |
| | | CTCCCTGACCATCTCTGGGCTCCAGGTTGAGGACGAGGCTGATT |
| | | ATTACTGCAGCTCATATTCAACCAGCATCACCCCAGTTTTCGGC |
| | | GGGGGGACCAAGCTCACCGTCCTAGGAGAGGGTAAATCTTCCG |
| | | GATCTGGTTCCGAAAGCAAGGCTAGCCAGGTCCAGCTGGTGCA |
| | | GTCTGGAGCAGAGGTGAAAAAGCCCGGGGAGTCTCTGAAGATC |
| | | TCCTGTGAGGCTTCTGGATACAGCTTTACCAGCTACTGGATCGG |
| | | CTGGGTGCGCCAGATGCCCGGGAAAGGCCTGGAGTGGATGGGG |
| | | ATCATCTATCCTGGTGACTCTGATACCAGATACAGCCCGTCCTT |
| | | CCAAGGCCAGGTCACCATCTCAGCCGACAAGTCCATCAGCACC |
| | | GCCTACCTGCAGTGGAGCAGCCTGAAGGCCTCGGACACCGCCA |
| | | TGTATTACTGTGCGAGACTGGAACTCGGTTACTACTACTACGGT |
| | | ATGGACGTCTGGGGCCAAGGAACCACGGTCACCGTCTCCTCA |
| 135 | Linker 5 (DNA) | GGAGAGGGTAAATCTTCCGGATCTGGTTCCGAAAGCAAGGCTA |
| | | GC |
| 136 | anti-ROR1 | CAGTCAGCTCTGACCCAACCTGCCTCCGTCTCTGGGAGTCCAGG |
| | scFv 3-6 | CCAGAGTATCACAATTTCTTGTACAGGCACCTCATCTGATGTCG |
| | VL (DNA) | GCGGTTACAATTACGTTAGTTGGTATCAGCAACATCCAGGTAA |
| | | GGCTCCAAAGGTGATGATCTATGACGTCTCAAATAGACCCTCT |
| | | GGCGTCAGCGACAGGTTTAGTGGTAGCAAATCCGGGAACACAG |
| | | CTTCACTTACAATTAGCGGCCTCCAAGTAGAAGACGAAGCTGA |
| | | CTATTACTGCTCTAGTTATTCAACGTCAATTACCCCTGTGTTTGG |
| | | TGGCGGTACAAAACTCACAGTGCTT |
| 137 | anti-ROR1 | CAAGTGCAACTTGTGCAATCAGGAGCTGAAGTCAAAAAGCCGG |
| | scFv 3-6 | GAGAATCCCTGAAAATAAGCTGCGAAGCAAGTGGTTACTCTTT |
| | VH (DNA) | TACTTCTTACTGGATTGGATGGGTTCGGCAGATGCCCGGAAAG |
| | | GGACTCGAGTGGATGGGAATTATTTACCCTGGAGACAGCGACA |
| | | CAAGATACAGCCCTTCATTCCAGGGGCAGGTGACCATTTCTGCT |
| | | GACAAATCAATCAGTACAGCCTATCTGCAATGGAGTTCCCTCA |
| | | AAGCCAGTGACACTGCTATGTATTACTGCGCGCGACTGGAACT |
| | | GGGATACTACTACTACGGAATGGACGTATGGGGACAGGGAACC |
| | | ACCGTTACTGTTAGTAGC |
| 139 | pLRPO_anti ROR1 | CCAATTAACCAATTCTGAttagaaaaactcatcgagcatcaaatgaaactgcaat |
| | 2-7 | ttattcacatcaggattatcaataccatatttttgaaaaagccgtttctgtaatg |
| | CD3epsilon_T2A- | aaggagaaaactcaccgaggcagttccataggatggcaagatcctggtatcggtc |
| | eGFP | tgcgattccgactcgtccaacatcaatacaacctattaatttcccctcgtcaaaa |
| | | ataaggttatcaagtgagaaatcaccatgagtgacgactgaatccggtgagaatg |
| | | gcaaaagtttatgcatttctttccagacttgttcaacaggccagccattacgctc |
| | | gtcatcaaaatcactcgcatcaaccaaaccgttattcattcgtgattgcgcctga |
| | | gcaagacgaaatacgcgatcgctgttaaaaggacaattacaaacaggaatcgaat |
| | | gcaaccggcgcaggaacactgccagcgcatcaacaatattttcacctgaatcagg |
| | | atattcttctaatacctggaatgctgtttttccggggatcgcagtggtgagtaac |
| | | catgcatcatcaggagtacggataaaatgcttgatggtcggaagaggcataaatt |
| | | ccgtcagccagtttagtctgaccatctcatctgtaacatcattggcaacgctacc |
| | | tttgccatgtttcagaaacaactctggcgcatcgggcttcccatacaagcgatag |
| | | attgtcgcacctgattgcccgacattatcgcgagcccatttatacccatataaat |
| | | cagcatccatgttggaatttaatcgcggcctcgacgtttcccgttgaatatggct |
| | | catAACACCCCTTGTATTACTGTTTATGTAAGCAGACAGTTTTATTGTTCATGAT |
| | | GATATATTTTTATCTTGTGCAATGTAACATCAGAGATTTTGAGACACAACG |
| | | TGGCTTTCCCCCCCCCCCCCATGACATTAACCTATAAAAATAGG |
| | | CGTATCACGAGGCCAGCTTGGGAAACCATAAGACCGAGATAGA |
| | | GTTGAGTGTTGTTCCAGTTTGGAACAAGAGTCCACTATTAAAGA |
| | | ACGTGGACTCCAACGTCAAAGGGCGAAAAACCGTCTATCAGGG |
| | | CGATGGCCCACTACGTGAACCATCACCCAAATCAAGTTTTTTGG |
| | | GGTCGAGGTGCCGTAAAGCACTAAATCGGAACCCTAAAGGGAG |
| | | CCCCCGATTTAGAGCTTGACGGGGAAAGCCGGCGAACGTGGCG |
| | | AGAAAGGAAGGGAAGAAAGCGAAAGGAGCGGGCGCTAAGGC |
| | | GCTGGCAAGTGTAGCGGTCACGCTGCGCGTAACCACCACACCC |
| | | GCCGCGCTTAATGCGCCGCTACAGGGCGCGTACTATGGTTGCTT |
| | | TGACGTATGCGGTGTGAAATACCGCACAGATGCGTAAGGAGAA |
| | | AATACCGCATCAGGCGccattcgccattcaggctgcgcaactgttgggaagggcg |
| | | tacggtgcgggcctcttcgctattacgccaGCTGGCGAAAGGGGGATGTGCTGCA |
| | | AGGCGATTAAGTTGGGTAACGCCAGGGTTTTCCCAGTCACGACGTT |
| | | GTAAAACGACGGCCAGTGAATTGATCGAGATCGTGATCCGGAT |
| | | CAAGATCCAGATCGAATTGGAGGCTACAGTCAGTGGAGAGGAC |
| | | TTTCACTGACTGACTGACTGCGTCTCAACCTcctaggggacattgattattga |
| | | ctagttattaatagtaatcaattacggggtcattagttcatagcccatatatgga |
| | | gttccgcgttacataacttacggtaaatggcccgcctggctgaccgcccaacgac |
| | | ccccgcccattgacgtcaataatgacgtatgttcccatagtaacgccaataggga |
| | | ctttccattgacgtcaatgggtggagtatttacggtaaactgcccacttggcagt |
| | | acatcaagtgtatcatatgccaagtacgccccctattgacgtcaatgacggtaaa |
| | | tggcccgcctggcattatgcccagtacatgaccttatgggactttcctacttggc |
| | | agtacatctacgtattagtcatcgctattaccatggtgatgcggttttggcagta |
| | | catcaatgggcgtggatagcggtttgactcacggggatttccaagtctccacccc |
| | | attgacgtcaatgggagtttgttttggcaccaaaatcaacgggactttccaaaat |
| | | gtcgtaacaactccgccccattgacgcaaatgggcggtaggcgtgtacggtggga |
| | | ggtctatataagcagagctcgtttagtgaaccgggtctctctggttagaccagat |
| | | ctgagcctgggagctctctggctaactagggaacccactgcttaagcctcaataa |
| | | agcttgccttgagtgctcaaagtagtgtgtgcccgtctgttgtgtgactctggta |
| | | actagagatccctcagacccttttagtcagtgtggaaaatctctagcagtggcgc |
| | | ccgaacagggacttgaaagcgaaagtaaagccagaggagatctctcgacgcagga |
| | | ctcggcttgctgaagcgcgcacggcaagaggcgaggggcggcgactggtgagtac |
| | | gccaaaaattttgactagcggaggctagaaggagagagtagggtgcgagagcgtc |
| | | ggtattaagcgggggagaattagataaatgggaaaaaattcggttaaggccaggg |
| | | ggaaagaaacaatataaactaaaacatatagttagggcaagcagggagctagaac |
| | | gattcgcagttaatcctggccttttagagacatcagaaggctgtagacaaatact |
| | | gggacagctacaaccatcccttcagacaggatcagaagaacttagatcattatat |
| | | aatacaatagcagtcctctattgtgtgcatcaaaggatagatgtaaaagacacca |
| | | aggaagccttagataagatagaggaagagcaaaacaaaagtaagaaaaaggcaca |
| | | gcaagcgatcttcagacctggaggaggcaggaggcgatatgagggacaattggag |
| | | aagtgaattatataaatataaagtagtaaaaattgaaccattaggagtagcaccc |
| | | accaaggcaaagagaagagtggtgcagagagaaaaaagagcagtgggaataggag |
| | | ctttgttccttgggttcttgggagcagcaggaagcactatgggcgcagcgtcaat |
| | | gacgctgacggtacaggccagacaattattgtctgatatagtgcagcagcagaac |
| | | aatttgctgagggctattgaggcgcaacagcatctgttgcaactcacagtctggg |
| | | gcatcaaacagctccaggcaagaatcctggctgtggaaagatacctaaaggatca |
| | | acagctcctggggatttggggttgctctggaaaactcatttgcaccactgctgtg |
| | | ccttggaatgctagttggagtaataaatctctggaacagatttggaataacatga |
| | | cctggatggagtgggacagagaaattaacaattacacaagcttaatacactcctt |
| | | aattgaagaatcgcaaaaccagcaagaaaagaatgaacaagaattattggaatta |
| | | gataaatgggcaagtttgtggaattggtttaacataacaaattggctgtggtata |
| | | taaaattattcataatgatagtaggaggcttggtaggtttaagaatagtttttgc |
| | | tgtactttctatagtgaatagagttaggcagggatattcaccattatcgtttcag |
| | | acccacctcccaatcccgaggggaccacgcgtacaaatggcagtattcatccaca |
| | | attttaaaagaaaaggggggattggggggtacagtgcaggggaaagaatagtaga |
| | | cataatagcaacagacatacaaactaaagaattacaaaaacaaattacaaaaatt |
| | | caaaattttcgggtttattacagggacagcagaaatccactttggaaagctgagc |
| | | atccggctccggtgcccgtcagtgggcagagcgcacatcgcccacagtccccgag |
| | | aagttggggggaggggtcggcaattgaaccggtgcctagagaaggtggcgcgggg |
| | | taaactgggaaagtgatgtcgtgtactggctccgcctttttcccgagggtggggg |
| | | agaaccgtatataagtgcagtagtcgccgtgaacgttctttttcgcaacgggttt |
| | | gccgccagaacacaggtaagtgccgtgtgtggttcccgcgggcctggcctcttta |
| | | cgggttatggcccttgcgtgccttgaattacttccacgcccctggctgcagtacg |
| | | tgattcttgatcccgagcttcgggttggaagtgggtgggagagttcgaggccttg |
| | | cgcttaaggagccccttcgcctcgtgcttgagttgaggcctggcctgggcgctgg |
| | | ggccgccgcgtgcgaatctggtggcaccttcgcgcctgtctcgctgctttcgata |
| | | agtctctagccatttaaaatttttgatgacctgctgcgacgctttttttctggca |
| | | agatagtcttgtaaatgcgggccaagatctgcacactggtatttcggtttttggg |
| | | gccgcgggcggcgacggggcccgtgcgtcccagcgcacatgttcggcgaggcggg |
| | | gcctgcgagcgcggccaccgagaatcggacgggggtagtctcaagctggccggcc |
| | | tgctctggtgcctggcctcgcgccgccgtgtatcgccccgccctgggcggcaagg |
| | | ctggcccggtcggcaccagttgcgtgagcggaaagatggccgcttcccggccctg |
| | | ctgcagggagctcaaaatggaggacgcggcgctcgggagagcgggcgggtgagtc |
| | | acccacacaaaggaaaagggcctttccgtcctcagccgtcgcttcatgtgactcc |
| | | acggagtaccgggcgccgtccaggcacctcgattagttctcgagcttttggagta |
| | | cgtcgtctttaggttggggggaggggttttatgcgatggagtttccccacactga |
| | | gtgggtggagactgaagttaggccagcttggcacttgatgtaattctccttggaa |
| | | tttgccctttttgagtttggatcttggttcattctcaagcctcagacagtggttc |
| | | aaagtttttttcttccatttcaggtgtcgtgaaaactacccctctagagccg |
| | | ccaccATGCTCCTCCTCGTGACTAGCCTTCTCCTGTGCGAGCTCCC |
| | | ACACCCTGCATTCCTCCTGATCCCAGAAGTGCAACTTCTCGAGA |
| | | GCGGTGGGGGACTCGTCCAGCCGGGAGGTTCCCTGCGACTCAG |
| | | CTGTGCAGCCTCAGGCTTTACCTTTTCCAGTTACGCAATGAGTT |
| | | GGGTCCGGCAGGCGCCTGGTAAAGGACTCGAGTGGGTGAGTGC |
| | | AATCAGCGGAAGTGGCGGGTCTACATACTATGCGGACTCTGTT |
| | | AAAGGCAGGTTCACTATTTCAAGGGACAATTCCAAGAACACGC |
| | | TCTACCTGCAGATGAATAGCCTTAGAGCTGAAGACACGGCCGT |
| | | GTACTATTGTGCCAAAGACAAGGGATGGTTCAACTGGCAGTTC |
| | | GACTACTGGGGGCAGGGAACTCTCGTCACCGTGAGCTCCGGCG |
| | | GAGGTGGAAGCGGGGGAGGGGGCTCCGGTGGTGGGGGATCAG |
| | | AAATTGTTCTCACACAGTCACCCGGAACCCTTTCATTGTCCCCC |
| | | GGCGAGCGCGCCACCCTCAGCTGTCGGGCCAGTCAGAGCGTGT |
| | | CTAGCTCTTACCTGGCCTGGTACCAGCAGAAACCTGGGCAAGC |
| | | TCCCAGACTCCTGATATATGGGGCCAGCAGCCGGGCCACTGGC |
| | | ATTCCGGACAGGTTTAGTGGATCAGGCTCTGGCACTGATTTTAC |
| | | ACTGACGATTTCAAGGTTGGAACCCGAAGACTTCGCAGTGTAC |
| | | TATTGTCAGCAGTATGGGTCTAGCCCGTGGACTTTCGGGCAAG |
| | | GCACCAAGGTGGAAATCAAGGCAGCTGCTGGAGGTGGGGGAA |
| | | GTGGCGGTGGTGGCTCAGGCGGCGGGGGGAGCCTCGAGGACG |
| | | GTAATGAAGAGATGGGGGGCATTACACAAACCCCGTACAAGGT |
| | | CTCTATCAGTGGGACGACTGTGATTCTGACATGCCCACAGTATC |
| | | CAGGTTCAGAAATCCTGTGGCAGCATAATGACAAGAACATCGG |
| | | TGGGGATGAGGATGATAAGAATATCGGAAGCGACGAAGACCA |
| | | CCTGTCTCTCAAAGAGTTTAGCGAGCTGGAGCAGAGTGGGTAT |
| | | TATGTCTGCTATCCTAGAGGTAGCAAGCCAGAGGACGCAAACT |
| | | TTTACCTTTACCTCAGAGCCAGGGTCTGCGAGAACTGCATGGA |
| | | AATGGACGTGATGAGTGTTGCAACTATAGTGATAGTTGACATTT |
| | | GCATCACCGGGGGTCTGCTCCTGCTGGTTTACTATTGGAGCAAG |
| | | AACCGCAAGGCTAAAGCCAAGCCAGTAACACGGGGCGCAGGC |
| | | GCGGGAGGCAGGCAGCGAGGGCAGAATAAGGAGCGCCCCCCA |
| | | CCCGTCCCGAATCCGGATTACGAACCCATTCGGAAAGGCCAGA |
| | | GGGACTTGTACTCAGGGCTCAACCAAAGACGGATCGAGGGGCG |
| | | AGGATCCTTGCTGACATGTGGTGACGTGGAGGAGAATCCTGGT |
| | | CCTTCTCGCgccgccaccATGGTGTCTAAAGGCGAAGAGCTGTTCAC |
| | | CGGTGTGGTGCCGATTCTTGTAGAGCTGGATGGAGATGTTAAT |
| | | GGTCACAAGTTTTCAGTGTCTGGGGAGGGCGAAGGCGACGCGA |
| | | CCTATGGTAAACTCACGCTTAAGTTTATCTGCACCACAGGGAA |
| | | GCTCCCTGTTCCATGGCCAACCCTTGTGACAACACTTACTTACG |
| | | GCGTGCAGTGTTTCAGCAGGTATCCTGACCATATGAAGCAGCA |
| | | CGATTTCTTCAAGTCTGCAATGCCCGAGGGGTACGTACAAGAG |
| | | CGGACAATTTTCTTCAAGGACGACGGAAATTACAAAACTAGGG |
| | | CAGAGGTTAAGTTCGAAGGGGATACACTTGTTAATAGGATCGA |
| | | ACTGAAAGGCATTGATTTCAAGGAGGATGGAAACATACTCGGG |
| | | CACAAACTGGAATATAACTACAATTCACATAATGTGTATATCAT |
| | | GGCTGATAAGCAGAAAAACGGTATCAAAGTGAACTTTAAGATC |
| | | CGGCATAACATTGAAGACGGTAGCGTGCAGCTCGCTGACCACT |
| | | ACCAGCAGAACACTCCAATCGGGGACGGGCCGGTCCTCCTGCC |
| | | CGACAACCACTACCTCAGCACCCAGAGCGCACTTAGCAAAGAC |
| | | CCAAACGAGAAGAGAGACCATATGGTGCTGCTGGAGTTCGTTA |
| | | CCGCAGCCGGAATCACCTTGGGCATGGACGAGCTCTATAAATG |
| | | Agaattcgaacggatatcgagcatcttaccgccatttatacccatatttgttctg |
| | | tttttcttgatttgggtatacatttaaatgttaataaaacaaaatggtggggcaa |
| | | tcatttacatttttagggatatgtaattactagttcaggtgtattgccacaagac |
| | | aaacatgttaagaaactttcccgttatttacgctctgttcctgttaatcaacctc |
| | | tggattacaaaatttgtgaaagattgactgatattcttaactatgttgctccttt |
| | | tacgctgtgtggatatgctgctttatagcctctgtatctagctattgcttcccgt |
| | | acggctttcgttttctcctccttgtataaatcctggttgctgtctcttttagagg |
| | | agttgtggcccgttgtccgtcaacgtggcgtggtgtgctctgtgtttgctgacgc |
| | | aacccccactggctggggcattgccaccacctgtcaactcctttctgggactttc |
| | | gctttccccctcccgatcgccacggcagaactcatcgccgcctgccttgcccgct |
| | | gctggacaggggctaggttgctgggcactgataattccgtggtgttgtcagtact |
| | | ggtacctttaagaccaatgacttacaaggcagctgtagatcttagccacttttta |
| | | aaagaaaaggggggactggaagggctaattcactcccaaagaagacaagatctgc |
| | | tttttgcctgtactgggtctctctggttagaccagatctgagcctgggagctctc |
| | | tggctaactagggaacccactgcttaagcctcaataaagcttgccttgagtgctt |
| | | caatgatcataatcaagccatatcacatctgtagaggtttacttgctttaaaaaa |
| | | cctccacacctccccctgaacctgaaacataaaatgaatgcaattgttgttgtta |
| | | acttgtttattgcagcttataatggttacaaataaagcaatagcatcacaaattt |
| | | cacaaataaagcatttttttcactgcattctagttgtggtttgtccaaactcatc |
| | | aatgtatcttatcatgtctggatctgcgtcgacACGAAGAGACGACTGACT |
| | | GACTGACTGGAAAGAGGAAGGGCTGGAAGAGGAAGGAGCTTG |
| | | ATCCAGATCCCGATCTCGATCCAGATCCGGATCGCAGCTTGGC |
| | | GTAATCATGGTCATAGCTGTTTCCTGTGTGAAATTGTTATCCGC |
| | | TCACAATTCCACACAACATACGAGCCGGAAGCATAAAGTGTAA |
| | | AGCCTGGGGTGCCTAATGAGTGAGCTAACTCACATTAATTGCG |
| | | TTGCGCTCACTGCCCGCTTTCCAGTCGGGAAACCTGTCGTGCCA |
| | | GCTGCATTAATGAATCGGCCAACGCGCGGGGAGAGGCGGTTTG |
| | | CGTATTGGGCGCTCTTCCGCTTCCTCGCTCACTGACTCGCTGCG |
| | | CTCGGTCGTTCGGCTGCGGCGAGCGGTATCAGCTCACTCAAAG |
| | | GCGGTAATACGGTTATCCACAGAATCAGGGGATAACGCAGGAA |
| | | AGAACATGTGAGCAAAAGGCCAGCAAAAGGCCAGGAACCGTA |
| | | AAAAGGCCGCGTTGCTGGCGTTTTTCCATAGGCTCCGCCCCCCT |
| | | GACGAGCATCACAAAAATCGACGCTCAAGTCAGAGGTGGCGA |
| | | AACCCGACAGGACTATAAAGATACCAGGCGTTTCCCCCTGGAA |
| | | GCTCCCTCGTGCGCTCTCCTGTTCCGACCCTGCCGCTTACCGGA |
| | | TACCTGTCCGCCTTTCTCCCTTCGGGAAGCGTGGCGCTTTCTCA |
| | | TAGCTCACGCTGTAGGTATCTCAGTTCGGTGTAGGTCGTTCGCT |
| | | CCAAGCTGGGCTGTGTGCACGAACCCCCCGTTCAGCCCGACCG |
| | | CTGCGCCTTATCCGGTAACTATCGTCTTGAGTCCAACCCGGTAA |
| | | GACACGACTTATCGCCACTGGCAGCAGCCACTGGTAACAGGAT |
| | | TAGCAGAGCGAGGTATGTAGGCGGTGCTACAGAGTTCTTGAAG |
| | | TGGTGGCCTAACTACGGCTACACTAGAAGAACAGTATTTGGTA |
| | | TCTGCGCTCTGCTGAAGCCAGTTACCTTCGGAAAAAGAGTTGGT |
| | | AGCTCTTGATCCGGCAAACAAACCACCGCTGGTAGCGGTGGTT |
| | | TTTTTGTTTGCAAGCAGCAGATTACGCGCAGAAAAAAAGGATC |
| | | TCAAGAAGATCCTTTGATCTTTTCTACGGGGTCTGACGCTCAGT |
| | | GGAACGAAAACTCACGTTAAGGGATTTTGGTCATGAGATTATC |
| | | AAAAAGGATCTTCACCTAGATCCTTTTAAATTAAAAATGAAGTT |
| | | TTAAATCAATCTAAAGTATATATGAGTAAACTTGGTCTGACAGT |
| | | TACCAATGCTTAATCAGTGAGGCACCTATCTCAGCGATCTGTCT |
| | | ATTTCGTTCATCCATAGTTGCCTGACTCCCCGTCGTGTAGATAA |
| | | CTACGATACGGGAGGGCTTACCATCTGGCCCCAGTGCTGCAAT |
| | | GATACCGCAGCTTGGGAAACCATAAGAGCTGAAGCCAGTTACC |
| | | TTCGGAAAAAGAGTTGGTAGCTCTTGATCCGGCAAACAAACCA |
| | | CCGCTGGTAGCGGTGGTTTTTTTGTTTGCAAGCAGCAGATTACG |
| | | CGCAGAAAAAAAGGATCTCAAGAAGATCCTTTGATCTTTTCTA |
| | | CGGGGTCTGACGCTCAGTGGAACGAAAACTCACGTTAAGGGAT |
| | | TTTGGTCATGAGCTTGCGCCGTCCCGTCAAGTCAGCGTAATGCT |
| | | CTGCCAGTGTTACAA |
| | IRES from | |
| | different viral | |
| | transcripts | |
| 140 | CVB3 | TTAAAACAGCCTGTGGGTTGATCCCACCCACAGGCCCATTGGG |
| | | CGCTAGCACTCTGGTATCACGGTACCTTTGTGCGCCTGTTTTAT |
| | | ACCCCCTCCCCCAACTGTAACTTAGAAGTAACACACACCGATC |
| | | AACAGTCAGCGTGGCACACCAGCCACGTTTTGATCAAGCACTT |
| | | CTGTTACCCCGGACTGAGTATCAATAGACTGCTCACGCGGTTGA |
| | | AGGAGAAAGCGTTCGTTATCCGGCCAACTACTTCGAAAAACCT |
| | | AGTAACACCGTGGAAGTTGCAGAGTGTTTCGCTCAGCACTACC |
| | | CCAGTGTAGATCAGGTCGATGAGTCACCGCATTCCCCACGGGC |
| | | GACCGTGGCGGTGGCTGCGTTGGCGGCCTGCCCATGGGGAAAC |
| | | CCATGGGACGCTCTAATACAGACATGGTGCGAAGAGTCTATTG |
| | | AGCTAGTTGGTAGTCCTCCGGCCCCTGAATGCGGCTAATCCTAA |
| | | CTGCGGAGCACACACCCTCAAGCCAGAGGGCAGTGTGTCGTAA |
| | | CGGGCAACTCTGCAGCGGAACCGACTACTTTGGGTGTCCGTGTT |
| | | TCATTTTATTCCTATACTGGCTGCTTATGGTGACAATTGAGAGA |
| | | TCGTTACCATATAGCTATTGGATTGGCCATCCGGTGACTAATAG |
| | | AGCTATTATATATCCCTTTGTTGGGTTTATACCACTTAGCTTGA |
| | | AAGAGGTTAAAACATTACAATTCATTGTTAAGTTGAATACAGC |
| | | AAA |
| 141 | EMCV | TTGCCAGTCTGCTCGATATCGCAGGCTGGGTCCGTGACTACCCA |
| | | CTCCCCCTTTCAACGTGAAGGCTACGATAGTGCCAGGGCGGGT |
| | | ACTGCCGTAAGTGCCACCCCAAACAACAACAACAAAACAAACT |
| | | CCCCCTCCCCCCCCTTACTATACTGGCCGAAGCCACTTGGAATA |
| | | AGGCCGGTGTGCGTTTGTCTACATGCTATTTTCTACCGCATTAC |
| | | CGTCTTATGGTAATGTGAGGGTCCAGAACCTGACCCTGTCTTCT |
| | | TGACGAACACTCCTAGGGGTCTTTCCCCTCTCGACAAAGGAGT |
| | | GTAAGGTCTGTTGAATGTCGTGAAGGAAGCAGTTCCTCTGGAA |
| | | GCTTCTTAAAGACAAACAACGTCTGTAGCGACCCTTTGCAGGC |
| | | AGCGGAACCCCCCACCTGGTGACAGGTGCCTCTGCGGCCAAAA |
| | | GCCACGTGTATAAGATACACCTGCAAAGGCGGCACAACCCCAG |
| | | TGCCACGTTGTGAGTTGGATAGTTGTGGAAAGAGTCAAATGGC |
| | | TCTCCTCAAGCGTATTCAACAAGGGGCTGAAGGATGCCCAGAA |
| | | GGTACCCCATTGTATGGGATCTGATCTGGGGCCTCGGTGCACGT |
| | | GCTTTACACGTGTTGAGTCGAGGTGAAAAAACGTCTAGGCCCC |
| | | CCGAACCACGGGGACGTGGTTTTCCTTTGAAAACCACGATTAC |
| | | AAT |
| 142 | Spacer | GGTAGTGGTGCTACTAACTTCAGCCTGCTGAAGCA |
| 143 | Ana-CVB3- | GGGAGACCCTCGACCGTCGATTGTCCACTGGTCAACAATAGAT |
| | FMC63_CD3ϵ | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGCGAAAGCTGCAAGAGAATGAAAATCC |
| | | GTTGACCTTAAACGGTCGTGTGGGTTCAAGTCCCTCCACCCCCA |
| | | CGCCGGAAACGCAATAGCCGAAAAACAAAAAACAAAAAAATT |
| | | AAAACAGCCTGTGGGTTGATCCCACCCACAGGCCCATTGGGCG |
| | | CTAGCACTCTGGTATCACGGTACCTTTGTGCGCCTGTTTTATAC |
| | | CCCCTCCCCCAACTGTAACTTAGAAGTAACACACACCGATCAA |
| | | CAGTCAGCGTGGCACACCAGCCACGTTTTGATCAAGCACTTCTG |
| | | TTACCCCGGACTGAGTATCAATAGACTGCTCACGCGGTTGAAG |
| | | GAGAAAGCGTTCGTTATCCGGCCAACTACTTCGAAAAACCTAG |
| | | TAACACCGTGGAAGTTGCAGAGTGTTTCGCTCAGCACTACCCC |
| | | AGTGTAGATCAGGTCGATGAGTCACCGCATTCCCCACGGGCGA |
| | | CCGTGGCGGTGGCTGCGTTGGCGGCCTGCCCATGGGGAAACCC |
| | | ATGGGACGCTCTAATACAGACATGGTGCGAAGAGTCTATTGAG |
| | | CTAGTTGGTAGTCCTCCGGCCCCTGAATGCGGCTAATCCTAACT |
| | | GCGGAGCACACACCCTCAAGCCAGAGGGCAGTGTGTCGTAACG |
| | | GGCAACTCTGCAGCGGAACCGACTACTTTGGGTGTCCGTGTTTC |
| | | ATTTTATTCCTATACTGGCTGCTTATGGTGACAATTGAGAGATC |
| | | GTTACCATATAGCTATTGGATTGGCCATCCGGTGACTAATAGAG |
| | | CTATTATATATCCCTTTGTTGGGTTTATACCACTTAGCTTGAAA |
| | | GAGGTTAAAACATTACAATTCATTGTTAAGTTGAATACAGCAA |
| | | AATGCTTCTCCTGGTGACAAGCCTTCTGCTCTGTGAGTTACCAC |
| | | ACCCAGCATTCCTCCTGATCCCAGACATCCAGATGACACAGAC |
| | | TACATCCTCCCTGTCTGCCTCTCTGGGAGACAGAGTCACCATCA |
| | | GTTGCAGGGCAAGTCAGGACATTAGTAAATATTTAAATTGGTA |
| | | TCAGCAGAAACCAGATGGAACTGTTAAACTCCTGATCTACCAT |
| | | ACATCAAGATTACACTCAGGAGTCCCATCAAGGTTCAGTGGCA |
| | | GTGGGTCTGGAACAGATTATTCTCTCACCATTAGCAACCTGGAG |
| | | CAAGAAGATATTGCCACTTACTTTTGCCAACAGGGTAATACGCT |
| | | TCCGTACACGTTCGGAGGGGGGACTAAGTTGGAAATAACAGGC |
| | | TCCACCTCTGGATCCGGCAAGCCCGGATCTGGCGAGGGATCCA |
| | | CCAAGGGCGAGGTGAAACTGCAGGAGTCAGGACCTGGCCTGGT |
| | | GGCGCCCTCACAGAGCCTGTCCGTCACATGCACTGTCTCAGGG |
| | | GTCTCATTACCCGACTATGGTGTAAGCTGGATTCGCCAGCCTCC |
| | | ACGAAAGGGTCTGGAGTGGCTGGGAGTAATATGGGGTAGTGAA |
| | | ACCACATACTATAATTCAGCTCTCAAATCCAGACTGACCATCAT |
| | | CAAGGACAACTCCAAGAGCCAAGTTTTCTTAAAAATGAACAGT |
| | | CTGCAAACTGATGACACAGCCATTTACTACTGTGCCAAACATTA |
| | | TTACTACGGTGGTAGCTATGCTATGGACTACTGGGGTCAAGGA |
| | | ACCTCAGTCACCGTCTCCGCGGCCGCGGGCGGTGGAGGCAGTG |
| | | GTGGTGGCGGCTCTGGCGGTGGTGGTAGCCTCGAGGATGGTAA |
| | | TGAAGAAATGGGTGGTATTACACAGACACCATATAAAGTCTCC |
| | | ATCTCTGGAACCACAGTAATATTGACATGCCCTCAGTATCCTGG |
| | | ATCTGAAATACTATGGCAACACAATGATAAAAACATAGGCGGT |
| | | GATGAGGATGATAAAAACATAGGCAGTGATGAGGATCACCTGT |
| | | CACTGAAGGAATTTTCAGAATTGGAGCAAAGTGGTTATTATGT |
| | | CTGCTACCCCAGAGGAAGCAAACCAGAAGATGCGAACTTTTAT |
| | | CTCTACCTGAGGGCAAGAGTGTGTGAGAACTGCATGGAGATGG |
| | | ATGTGATGTCGGTGGCCACAATTGTCATAGTGGACATCTGCATC |
| | | ACTGGGGGCTTGCTGCTGCTGGTTTACTACTGGAGCAAGAATA |
| | | GAAAGGCCAAGGCCAAGCCTGTGACACGAGGAGCGGGTGCTG |
| | | GCGGCAGGCAAAGGGGACAAAACAAGGAGAGGCCACCACCTG |
| | | TTCCCAACCCAGACTATGAGCCCATCCGGAAAGGCCAGCGGGA |
| | | CCTGTATTCTGGCCTGAATCAGAGACGCATCTGATAAAAAAAA |
| | | CAAAAAACAAAACGGCTATTATGCGTTACCGGCGAGACGCTAC |
| | | GGACTTAAATAATTGAGCCTTAAAGAAGAAATTCTTTAAGTGG |
| | | ATGCTCTCAAACTCAGGGAAACCTAAATCTAGTTATAGACAAG |
| | | GCAATCCTGAGCCAAGCCGAAGTAGTAATTAGTAAGACCAGTG |
| | | GACAATCGACGGATAACAGCATATCTAG |
| 144 | Ana-EMCV- | GGGAGACCCTCGACCGTCGATTGTCCACTGGTCAACAATAGAT |
| | FMC63_CD3ϵ | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGCGAAAGCTGCAAGAGAATGAAAATCC |
| | | GTTGACCTTAAACGGTCGTGTGGGTTCAAGTCCCTCCACCCCCA |
| | | CGCCGGAAACGCAATAGCCGAAAAACAAAAAACAAAAAAACC |
| | | CCCCTCTCCCTCCCCCCCTAACGTTACTGGCCGAAGCCGCTTGG |
| | | AATAAGGCCGGTGTGCGTTTGTCTATATGTTATTTTCCACCATA |
| | | TTGCCGTCTTTTGGCAATGTGAGGGCCCGGAAACCTGGCCCTGT |
| | | CTTCTTGACGAGCATTCCTAGGGGTCTTTCCCCTCTCGCCAAAG |
| | | GAATGCAAGGTCTGTTGAATGTCGTGAAGGAAGCAGTTCCTCT |
| | | GGAAGCTTCTTGAAGACAAACAACGTCTGTAGCGACCCTTTGC |
| | | AGGCAGCGGAACCCCCCACCTGGCGACAGGTGCCTCTGCGGCC |
| | | AAAAGCCACGTGTATAAGATACACCTGCAAAGGCGGCACAACC |
| | | CCAGTGCCACGTTGTGAGTTGGATAGTTGTGGAAAGAGTCAAA |
| | | TGGCTCTCCTCAAGCGTATTCAACAAGGGGCTGAAGGATGCCC |
| | | AGAAGGTACCCCATTGTATGGGATCTGATCTGGGGCCTCGGTG |
| | | CACATGCTTTACATGTGTTTAGTCGAGGTTAAAAAACGTCTAGG |
| | | CCCCCCGAACCACGGGGACGTGGTTTTCCTTTGAAAAACACGA |
| | | TGATAATATGGCCACAACCATGCTTCTCCTGGTGACAAGCCTTC |
| | | TGCTCTGTGAGTTACCACACCCAGCATTCCTCCTGATCCCAGAC |
| | | ATCCAGATGACACAGACTACATCCTCCCTGTCTGCCTCTCTGGG |
| | | AGACAGAGTCACCATCAGTTGCAGGGCAAGTCAGGACATTAGT |
| | | AAATATTTAAATTGGTATCAGCAGAAACCAGATGGAACTGTTA |
| | | AACTCCTGATCTACCATACATCAAGATTACACTCAGGAGTCCCA |
| | | TCAAGGTTCAGTGGCAGTGGGTCTGGAACAGATTATTCTCTCAC |
| | | CATTAGCAACCTGGAGCAAGAAGATATTGCCACTTACTTTTGCC |
| | | AACAGGGTAATACGCTTCCGTACACGTTCGGAGGGGGGACTAA |
| | | GTTGGAAATAACAGGCTCCACCTCTGGATCCGGCAAGCCCGGA |
| | | TCTGGCGAGGGATCCACCAAGGGCGAGGTGAAACTGCAGGAGT |
| | | CAGGACCTGGCCTGGTGGCGCCCTCACAGAGCCTGTCCGTCAC |
| | | ATGCACTGTCTCAGGGGTCTCATTACCCGACTATGGTGTAAGCT |
| | | GGATTCGCCAGCCTCCACGAAAGGGTCTGGAGTGGCTGGGAGT |
| | | AATATGGGGTAGTGAAACCACATACTATAATTCAGCTCTCAAA |
| | | TCCAGACTGACCATCATCAAGGACAACTCCAAGAGCCAAGTTT |
| | | TCTTAAAAATGAACAGTCTGCAAACTGATGACACAGCCATTTA |
| | | CTACTGTGCCAAACATTATTACTACGGTGGTAGCTATGCTATGG |
| | | ACTACTGGGGTCAAGGAACCTCAGTCACCGTCTCCGCGGCCGC |
| | | GGGCGGTGGAGGCAGTGGTGGTGGCGGCTCTGGCGGTGGTGGT |
| | | AGCCTCGAGGATGGTAATGAAGAAATGGGTGGTATTACACAGA |
| | | CACCATATAAAGTCTCCATCTCTGGAACCACAGTAATATTGACA |
| | | TGCCCTCAGTATCCTGGATCTGAAATACTATGGCAACACAATG |
| | | ATAAAAACATAGGCGGTGATGAGGATGATAAAAACATAGGCA |
| | | GTGATGAGGATCACCTGTCACTGAAGGAATTTTCAGAATTGGA |
| | | GCAAAGTGGTTATTATGTCTGCTACCCCAGAGGAAGCAAACCA |
| | | GAAGATGCGAACTTTTATCTCTACCTGAGGGCAAGAGTGTGTG |
| | | AGAACTGCATGGAGATGGATGTGATGTCGGTGGCCACAATTGT |
| | | CATAGTGGACATCTGCATCACTGGGGGCTTGCTGCTGCTGGTTT |
| | | ACTACTGGAGCAAGAATAGAAAGGCCAAGGCCAAGCCTGTGA |
| | | CACGAGGAGCGGGTGCTGGCGGCAGGCAAAGGGGACAAAACA |
| | | AGGAGAGGCCACCACCTGTTCCCAACCCAGACTATGAGCCCAT |
| | | CCGGAAAGGCCAGCGGGACCTGTATTCTGGCCTGAATCAGAGA |
| | | CGCATCTGATAAAAAAAACAAAAAACAAAACGGCTATTATGCG |
| | | TTACCGGCGAGACGCTACGGACTTAAATAATTGAGCCTTAAAG |
| | | AAGAAATTCTTTAAGTGGATGCTCTCAAACTCAGGGAAACCTA |
| | | AATCTAGTTATAGACAAGGCAATCCTGAGCCAAGCCGAAGTAG |
| | | TAATTAGTAAGACCAGTGGACAATCGACGGATAACAGCATATC |
| | | TAG |
| 145 | Ana3.0 (Full) | GGGAGACCCTCGACCGTCGATTGTCCACTGGTCAACAATAGAT |
| | | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGCGAAAGCTGCAAGAGAATGAAAATCC |
| | | GTTGACCTTAAACGGTCGTGTGGGTTCAAGTCCCTCCACCCCCA |
| | | CGCCGGAAACGCAATAGCCGAAAAACAAAAAACAAAAAAACC |
| | | CCCCTCTCCCTCCCCCCCTAACGTTACTGGCCGAAGCCGCTTGG |
| | | AATAAGGCCGGTGTGCGTTTGTCTATATGTTATTTTCCACCATA |
| | | TTGCCGTCTTTTGGCAATGTGAGGGCCCGGAAACCTGGCCCTGT |
| | | CTTCTTGACGAGCATTCCTAGGGGTCTTTCCCCTCTCGCCAAAG |
| | | GAATGCAAGGTCTGTTGAATGTCGTGAAGGAAGCAGTTCCTCT |
| | | GGAAGCTTCTTGAAGACAAACAACGTCTGTAGCGACCCTTTGC |
| | | AGGCAGCGGAACCCCCCACCTGGCGACAGGTGCCTCTGCGGCC |
| | | AAAAGCCACGTGTATAAGATACACCTGCAAAGGCGGCACAACC |
| | | CCAGTGCCACGTTGTGAGTTGGATAGTTGTGGAAAGAGTCAAA |
| | | TGGCTCTCCTCAAGCGTATTCAACAAGGGGCTGAAGGATGCCC |
| | | AGAAGGTACCCCATTGTATGGGATCTGATCTGGGGCCTCGGTG |
| | | CACATGCTTTACATGTGTTTAGTCGAGGTTAAAAAACGTCTAGG |
| | | CCCCCCGAACCACGGGGACGTGGTTTTCCTTTGAAAAACACGA |
| | | TGATAATATGGCCACAACCATGGGAGTCAAAGTTCTGTTTGCCC |
| | | TGATCTGCATCGCTGTGGCCGAGGCCAAGCCCACCGAGAACAA |
| | | CGAAGACTTCAACATCGTGGCCGTGGCCAGCAACTTCGCGACC |
| | | ACGGATCTCGATGCTGACCGCGGGAAGTTGCCCGGCAAGAAGC |
| | | TGCCGCTGGAGGTGCTCAAAGAGATGGAAGCCAATGCCCGGAA |
| | | AGCTGGCTGCACCAGGGGCTGTCTGATCTGCCTGTCCCACATCA |
| | | AGTGCACGCCCAAGATGAAGAAGTTCATCCCAGGACGCTGCCA |
| | | CACCTACGAAGGCGACAAAGAGTCCGCACAGGGCGGCATAGG |
| | | CGAGGCGATCGTCGACATTCCTGAGATTCCTGGGTTCAAGGAC |
| | | TTGGAGCCCATGGAGCAGTTCATCGCACAGGTCGATCTGTGTGT |
| | | GGACTGCACAACTGGCTGCCTCAAAGGGCTTGCCAACGTGCAG |
| | | TGTTCTGACCTGCTCAAGAAGTGGCTGCCGCAACGCTGTGCGA |
| | | CCTTTGCCAGCAAGATCCAGGGCCAGGTGGACAAGATCAAGGG |
| | | GGCCGGTGGTGACTAAAAAAAACAAAAAACAAAACGGCTATT |
| | | ATGCGTTACCGGCGAGACGCTACGGACTTAAATAATTGAGCCT |
| | | TAAAGAAGAAATTCTTTAAGTGGATGCTCTCAAACTCAGGGAA |
| | | ACCTAAATCTAGTTATAGACAAGGCAATCCTGAGCCAAGCCGA |
| | | AGTAGTAATTAGTAAGACCAGTGGACAATCGACGGATAACAGC |
| | | ATATCTAG |
| 146 | Ana_CVB3_IRES- | GGGAGACCCTCGACCGTCGATTGTCCACTGGTCAACAATAGAT |
| | eGFP | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGCGAAAGCTGCAAGAGAATGAAAATCC |
| | | GTTGACCTTAAACGGTCGTGTGGGTTCAAGTCCCTCCACCCCCA |
| | | CGCCGGAAACGCAATAGCCGAAAAACAAAAAACAAAAAAATT |
| | | AAAACAGCCTGTGGGTTGATCCCACCCACAGGCCCATTGGGCG |
| | | CTAGCACTCTGGTATCACGGTACCTTTGTGCGCCTGTTTTATAC |
| | | CCCCTCCCCCAACTGTAACTTAGAAGTAACACACACCGATCAA |
| | | CAGTCAGCGTGGCACACCAGCCACGTTTTGATCAAGCACTTCTG |
| | | TTACCCCGGACTGAGTATCAATAGACTGCTCACGCGGTTGAAG |
| | | GAGAAAGCGTTCGTTATCCGGCCAACTACTTCGAAAAACCTAG |
| | | TAACACCGTGGAAGTTGCAGAGTGTTTCGCTCAGCACTACCCC |
| | | AGTGTAGATCAGGTCGATGAGTCACCGCATTCCCCACGGGCGA |
| | | CCGTGGCGGTGGCTGCGTTGGCGGCCTGCCCATGGGGAAACCC |
| | | ATGGGACGCTCTAATACAGACATGGTGCGAAGAGTCTATTGAG |
| | | CTAGTTGGTAGTCCTCCGGCCCCTGAATGCGGCTAATCCTAACT |
| | | GCGGAGCACACACCCTCAAGCCAGAGGGCAGTGTGTCGTAACG |
| | | GGCAACTCTGCAGCGGAACCGACTACTTTGGGTGTCCGTGTTTC |
| | | ATTTTATTCCTATACTGGCTGCTTATGGTGACAATTGAGAGATC |
| | | GTTACCATATAGCTATTGGATTGGCCATCCGGTGACTAATAGAG |
| | | CTATTATATATCCCTTTGTTGGGTTTATACCACTTAGCTTGAAA |
| | | GAGGTTAAAACATTACAATTCATTGTTAAGTTGAATACAGCAA |
| | | AATGGTGAGCAAGGGCGAGGAGCTGTTCACCGGGGTGGTGCCC |
| | | ATCCTGGTCGAGCTGGACGGCGACGTAAACGGCCACAAGTTCA |
| | | GCGTGTCCGGCGAGGGCGAGGGCGATGCCACCTACGGCAAGCT |
| | | GACCCTGAAGTTCATCTGCACCACCGGCAAGCTGCCCGTGCCCT |
| | | GGCCCACCCTCGTGACCACCCTGACCTACGGCGTGCAGTGCTTC |
| | | AGCCGCTACCCCGACCACATGAAGCAGCACGACTTCTTCAAGT |
| | | CCGCCATGCCCGAAGGCTACGTCCAGGAGCGCACCATCTTCTTC |
| | | AAGGACGACGGCAACTACAAGACCCGCGCCGAGGTGAAGTTC |
| | | GAGGGCGACACCCTGGTGAACCGCATCGAGCTGAAGGGCATCG |
| | | ACTTCAAGGAGGACGGCAACATCCTGGGGCACAAGCTGGAGTA |
| | | CAACTACAACAGCCACAACGTCTATATCATGGCCGACAAGCAG |
| | | AAGAACGGCATCAAGGTGAACTTCAAGATCCGCCACAACATCG |
| | | AGGACGGCAGCGTGCAGCTCGCCGACCACTACCAGCAGAACAC |
| | | CCCCATCGGCGACGGCCCCGTGCTGCTGCCCGACAACCACTAC |
| | | CTGAGCACCCAGTCCGCCCTGAGCAAAGACCCCAACGAGAAGC |
| | | GCGATCACATGGTCCTGCTGGAGTTCGTGACCGCCGCCGGGAT |
| | | CACTCTCGGCATGGACGAGCTGTACAAGTAAAAAAAACAAAAA |
| | | ACAAAACGGCTATTATGCGTTACCGGCGAGACGCTACGGACTT |
| | | AAATAATTGAGCCTTAAAGAAGAAATTCTTTAAGTGGATGCTC |
| | | TCAAACTCAGGGAAACCTAAATCTAGTTATAGACAAGGCAATC |
| | | CTGAGCCAAGCCGAAGTAGTAATTAGTAAGACCAGTGGACAAT |
| | | CGACGGATAACAGCATATCTAG |
| 147 | Ana_CVB3_IRES- | GGGAGACCCTCGACCGTCGATTGTCCACTGGTCAACAATAGAT |
| | eGFP-sp1_mut | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGGTCGTGTGGGTTCAAGTCCCTCCACCC |
| | | CCACGCCGGAAACGCAATAGCCGAAAAACAAAAAACAAAAAA |
| | | ATTAAAACAGCCTGTGGGTTGATCCCACCCACAGGCCCATTGG |
| | | GCGCTAGCACTCTGGTATCACGGTACCTTTGTGCGCCTGTTTTA |
| | | TACCCCCTCCCCCAACTGTAACTTAGAAGTAACACACACCGATC |
| | | AACAGTCAGCGTGGCACACCAGCCACGTTTTGATCAAGCACTT |
| | | CTGTTACCCCGGACTGAGTATCAATAGACTGCTCACGCGGTTGA |
| | | AGGAGAAAGCGTTCGTTATCCGGCCAACTACTTCGAAAAACCT |
| | | AGTAACACCGTGGAAGTTGCAGAGTGTTTCGCTCAGCACTACC |
| | | CCAGTGTAGATCAGGTCGATGAGTCACCGCATTCCCCACGGGC |
| | | GACCGTGGCGGTGGCTGCGTTGGCGGCCTGCCCATGGGGAAAC |
| | | CCATGGGACGCTCTAATACAGACATGGTGCGAAGAGTCTATTG |
| | | AGCTAGTTGGTAGTCCTCCGGCCCCTGAATGCGGCTAATCCTAA |
| | | CTGCGGAGCACACACCCTCAAGCCAGAGGGCAGTGTGTCGTAA |
| | | CGGGCAACTCTGCAGCGGAACCGACTACTTTGGGTGTCCGTGTT |
| | | TCATTTTATTCCTATACTGGCTGCTTATGGTGACAATTGAGAGA |
| | | TCGTTACCATATAGCTATTGGATTGGCCATCCGGTGACTAATAG |
| | | AGCTATTATATATCCCTTTGTTGGGTTTATACCACTTAGCTTGA |
| | | AAGAGGTTAAAACATTACAATTCATTGTTAAGTTGAATACAGC |
| | | AAAATGGTGAGCAAGGGCGAGGAGCTGTTCACCGGGGTGGTGC |
| | | CCATCCTGGTCGAGCTGGACGGCGACGTAAACGGCCACAAGTT |
| | | CAGCGTGTCCGGCGAGGGCGAGGGCGATGCCACCTACGGCAAG |
| | | CTGACCCTGAAGTTCATCTGCACCACCGGCAAGCTGCCCGTGCC |
| | | CTGGCCCACCCTCGTGACCACCCTGACCTACGGCGTGCAGTGCT |
| | | TCAGCCGCTACCCCGACCACATGAAGCAGCACGACTTCTTCAA |
| | | GTCCGCCATGCCCGAAGGCTACGTCCAGGAGCGCACCATCTTC |
| | | TTCAAGGACGACGGCAACTACAAGACCCGCGCCGAGGTGAAGT |
| | | TCGAGGGCGACACCCTGGTGAACCGCATCGAGCTGAAGGGCAT |
| | | CGACTTCAAGGAGGACGGCAACATCCTGGGGCACAAGCTGGAG |
| | | TACAACTACAACAGCCACAACGTCTATATCATGGCCGACAAGC |
| | | AGAAGAACGGCATCAAGGTGAACTTCAAGATCCGCCACAACAT |
| | | CGAGGACGGCAGCGTGCAGCTCGCCGACCACTACCAGCAGAAC |
| | | ACCCCCATCGGCGACGGCCCCGTGCTGCTGCCCGACAACCACT |
| | | ACCTGAGCACCCAGTCCGCCCTGAGCAAAGACCCCAACGAGAA |
| | | GCGCGATCACATGGTCCTGCTGGAGTTCGTGACCGCCGCCGGG |
| | | ATCACTCTCGGCATGGACGAGCTGTACAAGTAAAAAAAACAAA |
| | | AAACAAAACGGCTATTATGCGTTACCGGCGAGACG |
| 148 | Ana_CVB3_IRES- | GGGAGACCCTCGACCGTCGATTGTCCACTGGTCAACAATAGAT |
| | antiCD19 TFP | GACTTACAACTAATCGGAAGGTGCAGAGACTCGACGGGAGCTA |
| | | CCCTAACGTCAAGACGAGGGTAAAGAGAGAGTCCAATTCTCAA |
| | | AGCCAATAGGCAGTAGCGAAAGCTGCAAGAGAATGAAAATCC |
| | | GTTGACCTTAAACGGTCGTGTGGGTTCAAGTCCCTCCACCCCCA |
| | | CGCCGGAAACGCAATAGCCGAAAAACAAAAAACAAAAAAATT |
| | | AAAACAGCCTGTGGGTTGATCCCACCCACAGGCCCATTGGGCG |
| | | CTAGCACTCTGGTATCACGGTACCTTTGTGCGCCTGTTTTATAC |
| | | CCCCTCCCCCAACTGTAACTTAGAAGTAACACACACCGATCAA |
| | | CAGTCAGCGTGGCACACCAGCCACGTTTTGATCAAGCACTTCTG |
| | | TTACCCCGGACTGAGTATCAATAGACTGCTCACGCGGTTGAAG |
| | | GAGAAAGCGTTCGTTATCCGGCCAACTACTTCGAAAAACCTAG |
| | | TAACACCGTGGAAGTTGCAGAGTGTTTCGCTCAGCACTACCCC |
| | | AGTGTAGATCAGGTCGATGAGTCACCGCATTCCCCACGGGCGA |
| | | CCGTGGCGGTGGCTGCGTTGGCGGCCTGCCCATGGGGAAACCC |
| | | ATGGGACGCTCTAATACAGACATGGTGCGAAGAGTCTATTGAG |
| | | CTAGTTGGTAGTCCTCCGGCCCCTGAATGCGGCTAATCCTAACT |
| | | GCGGAGCACACACCCTCAAGCCAGAGGGCAGTGTGTCGTAACG |
| | | GGCAACTCTGCAGCGGAACCGACTACTTTGGGTGTCCGTGTTTC |
| | | ATTTTATTCCTATACTGGCTGCTTATGGTGACAATTGAGAGATC |
| | | GTTACCATATAGCTATTGGATTGGCCATCCGGTGACTAATAGAG |
| | | CTATTATATATCCCTTTGTTGGGTTTATACCACTTAGCTTGAAA |
| | | GAGGTTAAAACATTACAATTCATTGTTAAGTTGAATACAGCAA |
| | | AATGCTTCTCCTGGTGACAAGCCTTCTGCTCTGTGAGTTACCAC |
| | | ACCCAGCATTCCTCCTGATCCCAGACATCCAGATGACACAGAC |
| | | TACATCCTCCCTGTCTGCCTCTCTGGGAGACAGAGTCACCATCA |
| | | GTTGCAGGGCAAGTCAGGACATTAGTAAATATTTAAATTGGTA |
| | | TCAGCAGAAACCAGATGGAACTGTTAAACTCCTGATCTACCAT |
| | | ACATCAAGATTACACTCAGGAGTCCCATCAAGGTTCAGTGGCA |
| | | GTGGGTCTGGAACAGATTATTCTCTCACCATTAGCAACCTGGAG |
| | | CAAGAAGATATTGCCACTTACTTTTGCCAACAGGGTAATACGCT |
| | | TCCGTACACGTTCGGAGGGGGGACTAAGTTGGAAATAACAGGC |
| | | TCCACCTCTGGATCCGGCAAGCCCGGATCTGGCGAGGGATCCA |
| | | CCAAGGGCGAGGTGAAACTGCAGGAGTCAGGACCTGGCCTGGT |
| | | GGCGCCCTCACAGAGCCTGTCCGTCACATGCACTGTCTCAGGG |
| | | GTCTCATTACCCGACTATGGTGTAAGCTGGATTCGCCAGCCTCC |
| | | ACGAAAGGGTCTGGAGTGGCTGGGAGTAATATGGGGTAGTGAA |
| | | ACCACATACTATAATTCAGCTCTCAAATCCAGACTGACCATCAT |
| | | CAAGGACAACTCCAAGAGCCAAGTTTTCTTAAAAATGAACAGT |
| | | CTGCAAACTGATGACACAGCCATTTACTACTGTGCCAAACATTA |
| | | TTACTACGGTGGTAGCTATGCTATGGACTACTGGGGTCAAGGA |
| | | ACCTCAGTCACCGTCTCCGCGGCCGCGGGCGGTGGAGGCAGTG |
| | | GTGGTGGCGGCTCTGGCGGTGGTGGTAGCCTCGAGGATGGTAA |
| | | TGAAGAAATGGGTGGTATTACACAGACACCATATAAAGTCTCC |
| | | ATCTCTGGAACCACAGTAATATTGACATGCCCTCAGTATCCTGG |
| | | ATCTGAAATACTATGGCAACACAATGATAAAAACATAGGCGGT |
| | | GATGAGGATGATAAAAACATAGGCAGTGATGAGGATCACCTGT |
| | | CACTGAAGGAATTTTCAGAATTGGAGCAAAGTGGTTATTATGT |
| | | CTGCTACCCCAGAGGAAGCAAACCAGAAGATGCGAACTTTTAT |
| | | CTCTACCTGAGGGCAAGAGTGTGTGAGAACTGCATGGAGATGG |
| | | ATGTGATGTCGGTGGCCACAATTGTCATAGTGGACATCTGCATC |
| | | ACTGGGGGCTTGCTGCTGCTGGTTTACTACTGGAGCAAGAATA |
| | | GAAAGGCCAAGGCCAAGCCTGTGACACGAGGAGCGGGTGCTG |
| | | GCGGCAGGCAAAGGGGACAAAACAAGGAGAGGCCACCACCTG |
| | | TTCCCAACCCAGACTATGAGCCCATCCGGAAAGGCCAGCGGGA |
| | | CCTGTATTCTGGCCTGAATCAGAGACGCATCTGATAAAAAAAA |
| | | CAAAAAACAAAACGGCTATTATGCGTTACCGGCGAGACGCTAC |
| | | GGACTTAAATAATTGAGCCTTAAAGAAGAAATTCTTTAAGTGG |
| | | ATGCTCTCAAACTCAGGGAAACCTAAATCTAGTTATAGACAAG |
| | | GCAATCCTGAGCCAAGCCGAAGTAGTAATTAGTAAGACCAGTG |
| | | GACAATCGACGGATAACAGCATATCTAG |
| 149 | Ana_CVB3_IRES- | GGGAGACCCAAGCTGGCTAGCGGGAGACCCTCGACCGTCGATT |
| | antiMSLN TFP | GTCCACTGGTCAACAATAGATGACTTACAACTAATCGGAAGGT |
| | | GCAGAGACTCGACGGGAGCTACCCTAACGTCAAGACGAGGGTA |
| | | AAGAGAGAGTCCAATTCTCAAAGCCAATAGGCAGTAGCGAAA |
| | | GCTGCAAGAGAATGAAAATCCGTTGACCTTAAACGGTCGTGTG |
| | | GGTTCAAGTCCCTCCACCCCCACGCCGGAAACGCAATAGCCGA |
| | | AAAACAAAAAACAAAAAAATTAAAACAGCCTGTGGGTTGATCC |
| | | CACCCACAGGCCCATTGGGCGCTAGCACTCTGGTATCACGGTA |
| | | CCTTTGTGCGCCTGTTTTATACCCCCTCCCCCAACTGTAACTTAG |
| | | AAGTAACACACACCGATCAACAGTCAGCGTGGCACACCAGCCA |
| | | CGTTTTGATCAAGCACTTCTGTTACCCCGGACTGAGTATCAATA |
| | | GACTGCTCACGCGGTTGAAGGAGAAAGCGTTCGTTATCCGGCC |
| | | AACTACTTCGAAAAACCTAGTAACACCGTGGAAGTTGCAGAGT |
| | | GTTTCGCTCAGCACTACCCCAGTGTAGATCAGGTCGATGAGTCA |
| | | CCGCATTCCCCACGGGCGACCGTGGCGGTGGCTGCGTTGGCGG |
| | | CCTGCCCATGGGGAAACCCATGGGACGCTCTAATACAGACATG |
| | | GTGCGAAGAGTCTATTGAGCTAGTTGGTAGTCCTCCGGCCCCTG |
| | | AATGCGGCTAATCCTAACTGCGGAGCACACACCCTCAAGCCAG |
| | | AGGGCAGTGTGTCGTAACGGGCAACTCTGCAGCGGAACCGACT |
| | | ACTTTGGGTGTCCGTGTTTCATTTTATTCCTATACTGGCTGCTTA |
| | | TGGTGACAATTGAGAGATCGTTACCATATAGCTATTGGATTGGC |
| | | CATCCGGTGACTAATAGAGCTATTATATATCCCTTTGTTGGGTT |
| | | TATACCACTTAGCTTGAAAGAGGTTAAAACATTACAATTCATTG |
| | | TTAAGTTGAATACAGCAAAATGCTTCTGCTTGTGACGTCCCTCC |
| | | TGCTTTGTGAACTCCCGCATCCTGCCTTCCTGCTCATCCCGGAA |
| | | GTTCAGCTGGTAGAGTCCGGTGGCGGGCTGGTACAGCCAGGGG |
| | | GATCTCTCCGTCTCTCATGTGCTGCCAGTGGGGGAGATTGGTCC |
| | | GCTAACTTTATGTACTGGTATCGTCAAGCACCTGGGAAACAAC |
| | | GCGAGCTGGTTGCACGGATCTCTGGACGAGGGGTAGTGGATTA |
| | | CGTGGAAAGTGTGAAAGGACGATTCACAATTTCCAGGGACAAT |
| | | TCTAAAAACACCCTTTATTTGCAAATGAATAGTCTGAGAGCAG |
| | | AAGACACAGCCGTATATTATTGTGCAGTCGCATCCTATTGGGG |
| | | GCAGGGGACTCTTGTCACAGTCTCGAGTGCTGCAGCAGGCGGC |
| | | GGGGGCTCAGGAGGGGGTGGGTCTGGAGGAGGTGGGAGTCTG |
| | | GAGGATGGTAATGAAGAAATGGGTGGTATTACACAGACACCAT |
| | | ATAAAGTCTCCATCTCTGGAACCACAGTAATATTGACATGCCCT |
| | | CAGTATCCTGGATCTGAAATACTATGGCAACACAATGATAAAA |
| | | ACATAGGCGGTGATGAGGATGATAAAAACATAGGCAGTGATG |
| | | AGGATCACCTGTCACTGAAGGAATTTTCAGAATTGGAGCAAAG |
| | | TGGTTATTATGTCTGCTACCCCAGAGGAAGCAAACCAGAAGAT |
| | | GCGAACTTTTATCTCTACCTGAGGGCAAGAGTGTGTGAGAACT |
| | | GCATGGAGATGGATGTGATGTCGGTGGCCACAATTGTCATAGT |
| | | GGACATCTGCATCACTGGCGGATTGCTGCTGCTGGTTTACTACT |
| | | GGAGCAAGAATAGAAAGGCCAAGGCCAAGCCTGTGACACGAG |
| | | GAGCGGGTGCTGGCGGCAGGCAAAGGGGACAAAACAAGGAGA |
| | | GGCCACCACCTGTTCCCAACCCAGACTATGAGCCCATCCGGAA |
| | | AGGCCAGCGGGACCTGTATTCTGGCCTGAATCAGAGACGCATC |
| | | TAAAAAAAACAAAAAACAAAACGGCTATTATGCGTTACCGGCG |
| | | AGACGCTACGGACTTAAATAATTGAGCCTTAAAGAAGAAATTC |
| | | TTTAAGTGGATGCTCTCAAACTCAGGGAAACCTAAATCTAGTTA |
| | | TAGACAAGGCAATCCTGAGCCAAGCCGAAGTAGTAATTAGTAA |
| | | GACCAGTGGACAATCGACGGATAACAGCATATCTAGGGATC |

What is claimed is:

1.-125. (canceled)

126. An isolated recombinant nucleic acid molecule comprising:
(A) one or more ribonucleic acid (RNA) sequences encoding:
(1) a T cell receptor (TCR) fusion protein (TFP) comprising:
(a) a TCR subunit comprising:
(i) at least a portion of a TCR extracellular domain,
(ii) a transmembrane domain, and
(iii) a TCR intracellular domain,
wherein the extracellular, transmembrane, and/or intracellular domains of the TCR subunit are derived from CD3 epsilon, CD3 gamma, CD3 delta, TCR alpha, TCR beta, TCR delta, or TCR gamma; and
(b) an antigen binding domain;
wherein the TCR subunit and the antigen binding domain are operatively linked;
and wherein the TFP incorporates into a TCR when expressed in a T cell; or
(2) a chimeric antigen receptor (CAR) or a T cell receptor (TCR); and
(B) one or more internal ribosome entry sites (IRES);
wherein (A) and (B) are operably linked to form a circular recombinant nucleic acid molecule.

127. The isolated recombinant nucleic acid molecule of claim 126, wherein:
(I) the TCR intracellular domain comprises a stimulatory domain derived from CD3 epsilon, CD3 gamma, or CD3 delta; and/or
(II) the transmembrane domain comprises a transmembrane domain of a protein selected from the group consisting of a TCR alpha chain, a TCR beta chain, a TCR delta chain, a TCR gamma chain, a CD3 zeta TCR subunit, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications thereto.

128. The isolated recombinant nucleic acid molecule of claim 127, wherein the TCR intracellular domain is derived from only CD3 epsilon, only CD3 gamma, only CD3 delta, only TCR alpha, only TCR beta, only TCR delta or only TCR gamma.

129. The isolated recombinant nucleic acid molecule of claim 126, wherein:
a) the antigen binding domain comprises an antibody or antibody fragment; and/or
b) the IRES comprises the IRES sequence from Coxsackievirus B3 (CVB3) or from encephalomyocarditis virus (EMCV).

130. The isolated recombinant nucleic acid molecule of claim 126, wherein:
(I) the isolated recombinant nucleic acid molecule is exogenous;
(II) the isolated recombinant nucleic acid molecule further comprises (C) a nucleic acid spacer sequence proximal to the 5′ end of (A) and the 3′ end of (B),
wherein (C) is formed by the circularization of a linear nucleic acid;
wherein the spacer sequence is about 30-100 nucleotides in length; and/or
wherein the circularization of the linear nucleic acid produces a circular RNA molecule; and/or
(III) the isolated recombinant nucleic acid molecule is suitable for transfection or transduction into an allogeneic or autologous human immune cell.

131. The isolated recombinant nucleic acid molecule of claim 126,
(I) wherein the antigen binding domain is a scFv or a VI-11-1 domain;
(II) wherein the antigen binding domain binds to a cell surface antigen; and/or
(III) wherein the antigen binding domain specifically binds to a tumor associated antigen.

132. The isolated recombinant nucleic acid molecule of claim 131, wherein the tumor associated antigen is CD19 or a variant thereof, CD20, CD22, BCMA, MSLN, IL13Ra2, EGFRvIII, MUC16, MUC1, ROR1, or a combination thereof.

133. The isolated recombinant nucleic acid molecule of claim 126, wherein:
(I) the isolated recombinant nucleic acid molecule further comprises a sequence encoding a costimulatory domain, wherein the costimulatory domain is a functional signaling domain of a protein selected from the group consisting of OX40, CD2, CD27, CD28, CD5, ICAM-1, LFA-1 (CD11a/CD18), ICOS (CD278), 4-1BB (CD137), and amino acid sequences thereof having at least one but not more than 20 modifications thereto;
(II) the isolated recombinant nucleic acid molecule further comprises a sequence encoding a TCR constant domain, wherein the TCR constant domain incorporates into a functional TCR complex when expressed in a T cell;
(III) the isolated recombinant nucleic acid molecule further comprises a sequence encoding a protein transduction domain or a cell penetrating peptide; and/or
(IV) wherein the TFP molecule is capable of functionally interacting with an endogenous TCR complex, at least one endogenous TCR polypeptide, or a combination thereof.

134. The isolated recombinant nucleic acid molecule of claim 133,
(I) wherein the TCR constant domain incorporates into a same functional TCR complex as the functional TCR complex that incorporates the TFP when expressed in a T cell;
and/or (II) wherein the sequence encoding the TFP and the sequence encoding the TCR constant domain are contained within a same nucleic acid molecule.

135. The isolated recombinant nucleic acid molecule of claim 126, wherein the TFP, CAR or TCR further comprises an immunoreceptor tyrosine-based activation motif (ITAM) or portion thereof, wherein the ITAM or portion thereof is from a protein selected from the group consisting of CD3 zeta TCR subunit, CD3 epsilon TCR subunit, CD3 gamma TCR subunit, CD3 delta TCR subunit, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto.

136. The isolated recombinant nucleic acid molecule of claim 135, wherein the ITAM or portion thereof replaces an ITAM of the TCR intracellular domain, wherein the replaced ITAM of the TCR intracellular domain is derived from only CD3 epsilon or CD3 gamma and is different than the ITAM or portion thereof that replaces it.

137. The isolated recombinant nucleic acid molecule of claim 126, wherein the circular recombinant nucleic acid molecule is less immunogenic than an shRNA, or a double stranded RNA or analog thereof, when transduced or transfected into a T cell.

138. An immune cell comprising the isolated recombinant nucleic acid molecule of claim 126.

139. The immune cell of claim 138, wherein the immune cell is a human T cell selected from the group consisting of a CD4+ cell, a CD8+ cell, a naive T cell, a memory stem T cell, a central memory T cell, a double negative T cell, an effector memory T cell, an effector T cell, a Th1 cell, a Tc1 cell, a Th2 cell, a Tc2 cell, a Th17 cell, a Th22 cell, a gamma/delta T cell, a natural killer (NK) cell, a natural killer T (NKT) cell, B cells, a hematopoietic stem cell, and a pluripotent stem cell; and/or
wherein the immune cell is a CD8+ or CD4+ or CD8+CD4+ human immune cell.

140. A method of treating cancer in a subject in need thereof comprising administering an immune cell comprising the isolated recombinant nucleic acid molecule of claim 126 to the subject in an amount therapeutically effective to treat cancer.

141. The method of claim 140, wherein the immune cell is a human T cell selected from the group consisting of a CD4+ cell, a CD8 cell, a naive T cell, a memory stem T cell, a central memory T cell, a double negative T cell, an effector memory T cell, an effector T cell, a Th1 cell, a Tc1 cell, a Th2 cell, a Tc2 cell, a Th17 cell, a Th22 cell, a gamma/delta T cell, a natural killer (NK) cell, a natural killer T (NKT) cell, a hematopoietic stem cell, and a pluripotent stem cell.

142. The method of claim 140, wherein the immune cell is an allogeneic T cell or an autologous T cell.

143. The method of claim 140, wherein the method comprises a single administration or more than one administration.

144. A method of producing a circular RNA encoding a T cell receptor (TCR) fusion protein (TFP), or a circular RNA encoding a CAR or a TCR comprising the steps of:
(i) providing one or more vectors comprising:
(A) (I) one or more sequences encoding a T cell receptor (TCR) fusion protein (TFP) comprising:
(a) a TCR subunit comprising:
(1) at least a portion of a TCR extracellular domain,
(2) a transmembrane domain, and
(3) a TCR intracellular domain, wherein the extracellular, transmembrane, and/or intracellular domains of the TCR subunit are derived from CD3 epsilon, CD3 gamma, CD3 delta, TCR alpha, TCR beta, TCR gamma, or TCR delta; and
(b) an antigen binding domain;
wherein the TCR subunit and the antigen binding domain are operatively linked; and wherein the TFP incorporates into a TCR when expressed in a T cell; or
(II) one or more sequences encoding a CAR or a TCR;
(B) one or more DNA sequences comprising one or more internal ribosome entry sites (IRES);
(C) one or more DNA sequences comprising a first circularization domain comprising at least one of a 5′ homology sequence and a 3′ permutated intron-exon (PIE) sequence; and
(D) one or more DNA sequences comprising a second circularization domain comprising at least one of a 3′ homology sequence and a 5′ PIE sequence, wherein (A) and (B) are operably linked;
(ii) transcribing the one or more vectors to produce one or more linear RNA; and
(iii) allowing the linear RNA to self-splice by using a chemical method, an enzymatic method, or a ribozymatic method, thereby producing the circular RNA.

145. A method of producing a modified immune cell containing a circular RNA encoding a T cell receptor (TCR) fusion protein (TFP) in a subject comprising:
administering at least one circular RNA vector to the subject in an amount effective to modify a population of target immune cells;
wherein the at least one circular RNA vector comprises:
(A) one or more sequences encoding a T cell receptor (TCR) fusion protein (TFP) comprising:
(a) a TCR subunit comprising:
(i) at least a portion of a TCR extracellular domain; a transmembrane domain; and
(ii) a TCR intracellular domain, wherein the extracellular, transmembrane, and/or intracellular domains of the TCR subunit are derived from CD3 epsilon, CD3 gamma, CD3 delta, TCR alpha, TCR beta, TCR gamma, or TCR delta;
(b) an antigen binding domain;
wherein the TCR subunit and the antigen binding domain are operatively linked; and
wherein the TFP incorporates into a TCR when expressed in a T cell;
(B) one or more DNA sequences comprising one or more internal ribosome entry sites (IRES);
(C) one or more DNA sequences comprising a first circularization domain comprising at least one of a 5′ homology sequence and a 3′ permutated intron-exon (PIE) sequence; and
(D) one or more DNA sequences comprising a second circularization domain comprising at least one of a 3′ homology sequence and a 5′ PIE sequence, wherein (A) and (B) are operably linked.
